(19) United States
(12) Patent Application Publication
(10) Pub. No.: US 2025/0388634 A1
(43) Pub. Date: Dec. 25, 2025

(54) ENGINEERED GENE EFFECTORS, COMPOSITIONS, AND METHODS OF USE THEREOF

(71) Applicant: Epicrispr Biotechnologies, Inc., South San Francisco, CA (US)

(72) Inventors: Giovanni CAROSSO, South San Francisco, CA (US); Lei S. QI, South San Francisco, CA (US); Daniel O. HART, South San Francisco, CA (US); Robin W. YEO, South San Francisco, CA (US); Timothy DALEY, South San Francisco, CA (US); Muhammad Zaki JAWAID, South San Francisco, CA (US)

(21) Appl. No.: 18/842,718

(22) PCT Filed: Mar. 23, 2023
(86) PCT No.: PCT/US2023/064887

§ 371 Date: Aug. 29, 2024

Related U.S. Application Data
(60) Provisional application No. 63/323,248, filed on Mar. 24, 2022.
(60) Provisional application No. 63/381,255, filed on Oct. 27, 2022.
(60) Provisional application No. 63/489,873, filed on Mar. 13, 2023.

(51) Int. Cl. C07K 14/47; C12N 9/22

(52) U.S. Cl. CPC C07K 14/4705; C12N 9/226

(57) ABSTRACT

The present disclosure provides one or more engineered gene effectors and systems, compositions, and methods of use thereof, wherein the one or more engineered gene effectors can be used to effect regulation of a target gene in a cell (e.g., an endogenous target gene in a cell). The one or more engineered gene effectors can be operatively coupled to a heterologous endonuclease, such as a CRISPR/Cas protein.

### CROSS-REFERENCE

This application claims the benefit of U.S. Provisional Application No. 63/489,873, filed Mar. 13, 2023, U.S. Provisional Application No. 63/381,255, filed Oct. 27, 2022, and U.S. Provisional Application No. 63/323,248, filed Mar. 24, 2022, each of which is incorporated herein by reference in its entirety.

### INCORPORATION BY REFERENCE OF SEQUENCE LISTING

The present application is being filed along with a Sequence Listing in electronic format. The Sequence Listing is provided as a file entitled 55176-727_601_SL.xml, created Mar. 10, 2023, which is 132 kilobytes in size. The information in the electronic format of the Sequence Listing is incorporated by reference in its entirety.

### BACKGROUND

Various effectors (e.g., transcriptional regulators) can be utilized to regulate expression or activity of a target gene in the cell. For example, a heterologous gene effector can be introduced (e.g., delivered, expressed, etc.) to the cell, and the heterologous gene effector, either alone or along with an additional agent, can effect such regulation of the target gene. In some examples, the additional agent can comprise a clustered regularly interspaced short palindromic repeats (CRISPR)/CRISPR-associated protein (Cas) for specifically binding to the target gene (e.g., a target deoxyribonucleic acid (DNA) sequence or ribonucleic acid (RNA) sequence (e.g., foreign DNA sequence or RNA sequence) of the target gene), while the heterologous gene effector can regulate expression or activity level of the target gene. Such gene effectors can be utilized, e.g., as gene therapy to treat or ameliorate a condition (e.g., a disease) of a subject.

### SUMMARY

VP64-p65-Rta fusion polypeptide (VPR) is a benchmark gene effector capable of activating a target gene in a cell. However, in some cases, the VPR may not be optimal or sufficient for regulating all genes. Alternatively or in addition to, a size of the VPR (e.g., about 518 amino acid residues) may not be small enough to package them along with at least one additional agent (e.g., one or more guide RNAs, a transgene encoding a therapeutic polynucleotide or protein, various types of Cas enzymes, etc.) in a delivery mode (e.g., viral vectors, such as adeno-associated virus (AAV) vectors). Thus, various aspects of the present disclosure, for example, provide engineered effectors that are not identical to VPR, yet comparably effective as the VPR in activating expression or activity level of one or more target genes, compositions thereof, and methods of use thereof.

Krueppel-associated box (KRAB) is a domain (e.g. having about 75 amino acid residues or less) that can be found in eukaryotic Krueppel-type C2H2 zinc finger proteins (ZFPs). The KRAB is a benchmark gene effector capable of repressing a target gene in a cell. However, in some cases, the KRAB may not be optimal or sufficient for regulating all genes. Thus, various aspects of the present disclosure, for example, provide engineered effectors that are not identical to the KRAB, yet comparably effective as the KRAB in reducing expression or activity level of one or more target genes, compositions thereof, and methods of use thereof.

Disclosed herein is an engineered gene effector comprising a polypeptide, wherein: the polypeptide is heterologous to any of the members selected from the group consisting of VP16, VP64, p65, and Rta; the engineered gene effector has a size of at most about 500 amino acid residues; and the engineered gene effector is capable of activating expression level of a target gene in a cell, wherein the expression level of the target gene that is activated via the engineered gene effector is at least about 80% as compared to that activated by a VP64-p65-Rta fusion polypeptide (VPR) in a control cell.

Disclosed herein is an engineered gene effector comprising a polypeptide coupled to an additional polypeptide, wherein: the polypeptide comprises an amino acid sequence having at least about 70% sequence identity to the polypeptide sequence of SEQ ID NO: 1; the additional polypeptide comprises at least a portion of one or more members selected from the group consisting of VP16, VP64, p65, and Rta; and the engineered gene effector has a size less than or equal to about 250 amino acid residues.

Disclosed herein is an engineered gene effector comprising a polypeptide, wherein the polypeptide comprises an amino acid sequence having at least about 70% sequence identity to the polypeptide sequence of SEQ ID NO: 1, wherein the engineered gene effector is not identical to any one of SEQ ID NO: 2, SEQ ID NO: 3, and SEQ ID NO: 4.

Disclosed herein is an engineered gene effector comprising a plurality of polypeptide domains, wherein each polypeptide domain of the plurality of polypeptide domain comprises a polypeptide comprising an amino acid sequence that exhibits at least about 70% sequence identity to the polypeptide sequence of SEQ ID NO: 1. A system comprising the engineered gene effector of any one of the preceding claims.

Disclosed herein is one or more polynucleotides encoding the system disclosed herein.

Disclosed herein is a cell comprising the system disclosed herein.

Disclosed herein is a method of controlling a target gene in a cell, the method comprising contacting the cell with the system disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates example structures of engineered effectors.

FIG. 2 schematically illustrates an example operation of the engineered effector that is in a complex with a heterologous endonuclease and a guide nucleic acid molecule, to modulate a target gene.

FIG. 3 shows correlation between two genes that are regulated by engineered effector candidates.

FIG. 4 shows a predicted active polypeptide sequence for generating an engineered effector.

FIG. 5 schematically illustrates examples of engineered effectors.

FIG. 6 schematically illustrates additional examples of engineered effectors comprising a flexible linker.

FIG. 7 schematically illustrates additional examples of engineered effectors comprising a plurality of active domains.

FIG. 8 schematically illustrates different examples of engineered effectors comprising a plurality of active domains.

FIG. 9 schematically illustrates additional examples of engineered effectors with varied lengths.

FIG. 10 schematically illustrates different examples of engineered effectors with varied lengths.

FIG. 11 schematically illustrates different examples of engineered effectors.

FIG. 12 shows activation of a heterologous target gene by engineered effectors.

FIG. 13 shows activation of an endogenous gene encoding IFN by engineered effectors.

FIG. 14 shows activation of a heterologous target gene by engineered effectors at a different time point.

FIG. 15A and FIG. 15B show activation of an endogenous target gene encoding CXCR4 by engineered effectors, at varied time points.

FIG. 16 shows activation of an endogenous gene encoding CD2 by engineered effectors.

FIG. 17 shows activation of an endogenous gene encoding CD45 by engineered effectors.

FIG. 18A shows activation of a heterologous target gene by engineered gene effectors, each comprising an active domain at different positions.

FIG. 18B shows activation of an endogenous target gene by engineered gene effectors, each comprising an active domain at different positions.

FIG. 19 shows various expression levels of a heterologous target gene, upon activation by engineered gene effectors.

FIG. 20 shows various expression levels of an endogenous gene encoding IFN, upon activation by engineered gene effectors.

FIG. 21 shows various expression levels of an endogenous gene encoding CXCR4, upon activation by engineered gene effectors.

FIG. 22 shows performance of engineered gene effectors for modulating heterologous and endogenous genes, as compared to control gene effectors.

FIG. 23 shows performance of engineered gene effectors for modulating endogenous genes, as compared to control gene effectors.

FIG. 24 shows expression levels of CXCR4 at 3 days post transfection, upon activation by engineered gene effectors.

FIG. 25 shows expression levels of CXCR4 at 7 days post transfection, upon activation by engineered gene effectors.

FIG. 26 shows activation of endogenous and heterologous target genes by engineered gene effectors.

FIG. 27 shows expression levels of CXCR4 at 28 days post transfection, upon repression by engineered gene effectors.

FIG. 28 shows expression levels of CXCR4 at 40 days post transfection, upon repression by engineered gene effectors.

FIG. 29 shows JQ1 or GNE049 mediated reversal of gene expressions by engineered gene effectors.

FIG. 30 shows persistent and sustainable gene modulation by engineered gene effectors.

FIG. 31 shows activation level of CD45, IFNG (IFN gamma) and CXCR4 by engineered gene effectors at 3 days post transfection (dpt).

FIG. 32 shows persistent activation (e.g., durable activation) of CD45, IFNG, CXCR4, and CD81 by engineered gene effectors at various timepoints (e.g., 9-18 days post transfection.

FIG. 33 shows a correlation between max activation (3 dpt) and activation (9-18 dpt) by engineered gene effectors.

FIG. 34 shows different size of VPR, p300, Rta, p65, VP64, and engineered gene effectors.

FIG. 35 shows activation of CD45 at 9, 12 and 18 days post transfection by engineered gene effectors.

FIG. 36 illustrates the persistent activation (e.g., durable activation) of CD45, CXCR4, FIN-Y, and CD81 by engineered gene effectors.

FIG. 37 illustrates activation robustness and potency of CD45, CXCR4, FIN-y, and CD81 by engineered gene effectors.

FIG. 38 shows CD45 activations by engineered gene effectors at different time points after transfection.

FIG. 39 shows IFNG activations by engineered gene effectors at different time points after transfection.

FIG. 40 shows CXCR4 activations by engineered gene effectors at different time points after transfection.

FIG. 41 shows CD81 activations by engineered gene effectors at different time points after transfection.

FIGS. 42 and 43 show changes in engineered gene effectors (mCherry) and sgRNA (BFP) expression (in MFI) by engineered gene effectors at different time points after transfection.

FIGS. 44A-44C shows activation of CD45 and IFNG by engineered gene effectors at 3 days (FIG. 44A), 6 days (FIG. 44B) and 9 days (FIG. 44C) post transfection.

FIG. 45 illustrates the predicted structure of CC.2, CC.4, and CC.5.

FIG. 46 illustrates the predicted structure of XV1.48 aligned to CC.2.

FIG. 47 illustrates the predicted structure of CC.32.

FIG. 48 illustrates the predicted structure of Cas protein coupled to XV1.32.

FIG. 49 shows a conserved sequence motif from distinct human protein families.

FIG. 50 shows a conserved sequence motif from distinct viral protein families.

FIG. 51 illustrates the process of amino acid screening using a regularized logistic regression model (ElasticNet).

FIG. 52 shows the feature importance for the identified 20 amino acids.

FIG. 53 shows a peptide-level motif enrichment by different properties (e.g., charge).

FIG. 54 illustrates the predicted structure of VPR.

FIG. 55 illustrates the predicted structure of XV1.1

FIG. 56 shows a volcano plot illustrating the screening results of engineered gene effectors.

FIG. 57 shows the predicted structure of engineered gene effectors (XV1.32 and XV1.33)

FIG. 58 shows a barrel view of a coiled coil variant.

FIG. 59 shows activations of multiple target genes by engineered gene effectors.

FIG. 60 shows activation of IFNG by coding length (bp) of engineered gene effectors.

FIG. 61 shows activation of CD45 by an engineered gene effector at various timepoints.

FIG. 62 shows engineered gene effectors' potency and durability for CD45 activation.

FIG. 63 shows activation of IFNG by an engineered gene effector at various timepoints.

FIG. 64 shows engineered gene effectors' potency and durability for IFNG activation.

FIG. 65 shows activation of CXCR4 by an engineered gene effector at various timepoints.

FIG. 66 shows engineered gene effectors' potency and durability for CXCR4 activation.

FIG. 67 shows activation of CD81 by an engineered gene effector at various timepoints.

FIG. 68 shows engineered gene effectors' potency and durability for CD81 activation.

FIG. 69 shows activation of CXCR4 mRNA level at Day 40 by engineered gene effectors.

FIG. 70 shows activation of CD45 mRNA level at Day 27 by engineered gene effectors.

FIG. 71 schematically illustrates epigenetic mechanisms of persistent activation by engineered gene effectors.

FIG. 72 shows CD45 expression by engineered gene effectors at Day 3.

FIG. 73 shows CD45 expression by engineered gene effectors at Day 9.

FIG. 74 shows CD45 expression by engineered gene effectors 96 hours after administering inhibitors.

FIG. 75 shows mitotically durable gene activation by engineered gene effectors.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “heterologous,” when used herein with reference to a polypeptide sequence or a nucleic acid sequence, indicates that the polypeptide sequence or the nucleic acid sequence is (1) disposed (e.g., in an environment, such as a cell, a virus, or a fusion polypeptide molecule or a fusion polynucleotide molecule) where it is not normally found (e.g., not normally found in nature); or (2) comprises two or more subsequences that are not found in the same relationship to each other as normally found in nature. For example, a polypeptide can comprise a first polypeptide sequence and a second polypeptide sequence that are not found together in a single polypeptide in nature, and thus the first polypeptide sequence and the second polypeptide sequence can be heterologous to each other. In another example, a polynucleotide can comprise a first polynucleotide sequence and a second polynucleotide sequence that are not found together in a single polynucleotide in nature, and thus the first polynucleotide sequence and the second polynucleotide sequence can be heterologous to each other.

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G] dCTP, [TAMRA] dCTP, [JOE] ddATP, [R6G] ddATP, [FAM] ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “expression profile” generally refers to quantitative (e.g., abundance) and qualitative expression of one or more genes in a sample (e.g., a cell). The one or more genes can be expressed and ascertained in the form of a nucleic acid molecule (e.g., an mRNA or other RNA transcript). Alternatively or in addition to, the one or more genes can be expressed and ascertained in the form of a polypeptide (e.g., a protein measured via Western blot). An expression profile of a gene may be defined as a shape of an expression level of the gene over a time period (e.g., at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 16 hours, at least or up to about 18 hours, at least or up to about 24 hours, at least or up to about 36 hours, at least or up to about 48 hours, at least up to about 3 days, at least up to about 4 days, at least up to about 5 days, at least up to about 6 days, at least up to about 7 days, at least up to about 8 days, at least up to about 9 days, at least up to about 10 days, at least up to about 11 days, at least up to about 12 days, at least up to about 13 days, at least up to about 14 days, etc.). Alternatively, an expression profile of a gene may be defined as an expression level of the gene at a time point of interest (e.g., the expression level of the gene measured at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 16 hours, at least or up to about 18 hours, at least or up to about 24 hours, at least or up to about 36 hours, at least or up to about 48 hours, at least up to about 3 days, at least up to about 4 days, at least up to about 5 days, at least up to about 6 days, at least up to about 7 days, at least up to about 8 days, at least up to about 9 days, at least up to about 10 days, at least up to about 11 days, at least up to about 12 days, at least up to about 13 days, or at least up to about 14 days after treating a cell to induce such expression level.)

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety. For example, an heterologous endonuclease (e.g., an engineered Cas protein) as disclosed herein is not a naturally occurring nuclease (e.g., not a naturally occurring Cas protein). In another example, an engineered gene effector as disclosed herein is not a naturally occurring gene effector.

The terms “engineered” and “modified” are used interchangeably herein. The terms “engineering” and “modifying” are used interchangeably herein. The terms “engineered cell” or “modified cell” are used interchangeably herein. The terms “engineered characteristic” and “modified characteristic” are used interchangeably herein.

The term “enhanced expression,” “increased expression,” or “upregulated expression” generally refers to production of a moiety of interest (e.g., a polynucleotide or a polypeptide) to a level that is above a normal level of expression of the moiety of interest in a host strain (e.g., a host cell). The normal level of expression can be substantially zero (or null) or higher than zero. The moiety of interest can comprise an endogenous gene or polypeptide construct of the host strain. The moiety of interest can comprise a heterologous gene or polypeptide construct that is introduced to or into the host strain. For example, a heterologous gene encoding a polypeptide of interest can be knocked-in (KI) to a genome of the host strain for enhanced expression of the polypeptide of interest in the host strain.

The term “enhanced activity,” “increased activity,” or “upregulated activity” generally refers to activity of a moiety of interest (e.g., a polynucleotide or a polypeptide) that is modified to a level that is above a normal level of activity of the moiety of interest in a host strain (e.g., a host cell). The normal level of activity can be substantially zero (or null) or higher than zero. The moiety of interest can comprise a polypeptide construct of the host strain. The moiety of interest can comprise a heterologous polypeptide construct that is introduced to or into the host strain. For example, a heterologous gene encoding a polypeptide of interest can be knocked-in (KI) to a genome of the host strain for enhanced activity of the polypeptide of interest in the host strain.

The term “reduced expression,” “decreased expression,” or “downregulated expression” generally refers to a production of a moiety of interest (e.g., a polynucleotide or a polypeptide) to a level that is below a normal level of expression of the moiety of interest in a host strain (e.g., a host cell). The normal level of expression is higher than zero. The moiety of interest can comprise an endogenous gene or polypeptide construct of the host strain. In some cases, the moiety of interest can be knocked-out or knocked-down in the host strain. In some examples, reduced expression of the moiety of interest can include a complete inhibition of such expression in the host strain.

The term “reduced activity,” “decreased activity,” or “downregulated activity” generally refers to activity of a moiety of interest (e.g., a polynucleotide or a polypeptide) that is modified to a level that is below a normal level of activity of the moiety of interest in a host strain (e.g., a host cell). The normal level of activity is higher than zero. The moiety of interest can comprise an endogenous gene or polypeptide construct of the host strain. In some cases, the moiety of interest can be knocked-out or knocked-down in the host strain. In some examples, reduced activity of the moiety of interest can include a complete inhibition of such activity in the host strain.

The term “subject,” “individual,” or “patient,” as used interchangeably herein, generally refers to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The term “treatment” or “treating” generally refers to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” generally refers to the quantity of a composition, for example a composition comprising heterologous polypeptides, heterologous polynucleotides, and/or modified cells (e.g., modified stem cells), that is sufficient to result in a desired activity upon administration to a subject in need thereof. Within the context of the present disclosure, the term “therapeutically effective” generally refers to that quantity of a composition that is sufficient to delay the manifestation, arrest the progression, relieve or alleviate at least one symptom of a disorder treated by the methods of the present disclosure.

### Overview

Various aspects of the present disclosure can provide engineered effectors (or engineered gene effectors, as used interchangeably herein) capable of regulating (e.g., activating or reducing) expression or activity level of a target gene in a cell (e.g., an endogenous target gene, a heterologous target gene, etc.), compositions thereof, and methods of use thereof. Such engineered effectors can work in conjunction with a heterologous endonuclease (e.g., engineered CRISPR/CAs nuclease, or a deactivated variant thereof) to, for example, effect manipulation of the expression or activity level of the target gene in the cell, e.g., to treat or ameliorate a condition (e.g., a disease) of a subject. Gene expression can underpin various physiological and pathological effects in cells and tissues, contributing to many diseases and conditions, and thus compositions and methods utilizing the engineered gene effectors of the present disclosure can modulate expression of specific genes in a desirable way to have therapeutic benefit.

### Engineered Gene Effectors, Compositions, and Methods Thereof

In some aspects, the present disclosure provides an engineered gene effector (e.g., an engineered gene activator, such as a transcriptional activator) that is not identical to VP16 (e.g., comprising the polypeptide sequence of SEQ ID NO: 5), VP64 (e.g., comprising the polypeptide sequence of SEQ ID NO: 6), p65 (e.g., comprising the polypeptide sequence of SEQ ID NO: 7), Rta (e.g., comprising the polypeptide sequence of SEQ ID NO: 8), or VP64-p65-Rta fusion polypeptide (VPR) (e.g., comprising the polypeptide sequence of SEQ ID NO: 9). In some aspects, the present disclosure provides an engineered gene effector (e.g., an engineered gene repressor, such as a transcriptional repressor) that is not identical to Krueppel-associated box (KRAB) (e.g., comprising the polypeptide sequence of SEQ ID NO: 64).

In some embodiments, the engineered gene effector as disclosed herein can have a size of at least or up to about 500 amino acid residues, at least or up to about 480 amino acid residues, at least or up to about 460 amino acid residues, at least or up to about 450 amino acid residues, at least or up to about 440 amino acid residues, at least or up to about 420 amino acid residues, at least or up to about 400 amino acid residues, at least or up to about 380 amino acid residues, at least or up to about 360 amino acid residues, at least or up to about 350 amino acid residues, at least or up to about 340 amino acid residues, at least or up to about 320 amino acid residues, at least or up to about 300 amino acid residues, at least or up to about 290 amino acid residues, at least or up to about 280 amino acid residues, at least or up to about 270 amino acid residues, at least or up to about 260 amino acid residues, at least or up to about 250 amino acid residues, at least or up to about 240 amino acid residues, at least or up to about 230 amino acid residues, at least or up to about 220 amino acid residues, at least or up to about 210 amino acid residues, at least or up to about 200 amino acid residues, at least or up to about 190 amino acid residues, at least or up to about 180 amino acid residues, at least or up to about 170 amino acid residues, at least or up to about 160 amino acid residues, at least or up to about 150 amino acid residues, at least or up to about 140 amino acid residues, at least or up to about 130 amino acid residues, at least or up to about 120 amino acid residues, at least or up to about 110 amino acid residues, at least or up to about 100 amino acid residues, at least or up to about 95 amino acid residues, at least or up to about 90 amino acid residues, at least or up to about 85 amino acid residues, at least or up to about 80 amino acid residues, at least or up to about 70 amino acid residues, at least or up to about 60 amino acid residues, at least or up to about 50 amino acid residues, at least or up to about 40 amino acid residues, at least or up to about 30 amino acid residues, or at least or up to about 20 amino acid residues.

In some embodiments, the size of the engineered gene effector as disclosed herein can be between about 20 amino acid residues and about 200 amino acid residues, between about 40 amino acid residues and about 180 amino acid residues, between about 50 amino acid residues and about 150 amino acid residues, between about 60 amino acid residues and about 140 amino acid residues, between about 60 amino acid residues and about 130 amino acid residues, between about 60 amino acid residues and about 120 amino acid residues, between about 60 amino acid residues and about 110 amino acid residues, between about 60 amino acid residues and about 100 amino acid residues, between about 70 amino acid residues and about 120 amino acid residues, between about 70 amino acid residues and about 110 amino acid residues, between about 70 amino acid residues and about 100 amino acid residues, between about 80 amino acid residues and about 120 amino acid residues, between about 80 amino acid residues and about 110 amino acid residues, or between about 80 amino acid residues and about 100 amino acid residues.

In some embodiments, the size of the engineered gene effector as disclosed herein can be less than about 680 amino acid residues, less than about 650 amino acid residues, less than about 600 amino acid residues, less than about 500 amino acid residues, less than about 400 amino acid residues, less than about 300 amino acid residues, less than about 200 amino acid residues, less than about 150 amino acid residues, less than about 140 amino acid residues, less than about 130 amino acid residues, less than about 120 amino acid residues, less than about 110 amino acid residues, or less than about 100 amino acid residues.

In some embodiments, the size of the engineered gene effector as disclosed herein can be less than or equal to about 100 amino acid residues, less than or equal to about 95 amino acid residues, less than or equal to about 90 amino acid residues, less than or equal to about 85 amino acid residues, less than or equal to about 80 amino acid residues, less than or equal to about 75 amino acid residues, less than or equal to about 74 amino acid residues, less than or equal to about 73 amino acid residues, less than or equal to about 72 amino acid residues, less than or equal to about 71 amino acid residues, less than or equal to about 70 amino acid residues, less than or equal to about 69 amino acid residues, less than or equal to about 68 amino acid residues, less than or equal to about 67 amino acid residues, less than or equal to about 66 amino acid residues, less than or equal to about 65 amino acid residues, less than or equal to about 64 amino acid residues, less than or equal to about 63 amino acid residues, less than or equal to about 62 amino acid residues, less than or equal to about 61 amino acid residues, less than or equal to about 60 amino acid residues, less than or equal to about 59 amino acid residues, less than or equal to about 58 amino acid residues, less than or equal to about 57 amino acid residues, less than or equal to about 56 amino acid residues, less than or equal to about 55 amino acid residues, less than or equal to about 54 amino acid residues, less than or equal to about 53 amino acid residues, less than or equal to about 52 amino acid residues, less than or equal to about 51 amino acid residues, less than or equal to about 50 amino acid residues, less than or equal to about 45 amino acid residues, less than or equal to about 40 amino acid residues, or less than or equal to about 35 amino acid residues.

In some embodiments, the engineered gene effector as disclosed herein can comprise a polypeptide.

In some embodiments, the polypeptide of the engineered gene effector as disclosed herein can comprise an amino acid sequence, and the amino acid sequence of the polypeptide of the engineered gene effector can be at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 1. For example, the amino acid sequence of the engineered gene effector can be between about 80% and about 100% identical to the polypeptide sequence of SEQ ID NO: 1.

In some embodiments, the amino acid sequence of said polypeptide can comprise C4, when aligned to the polypeptide sequence of SEQ ID No: 1. In some embodiments, the amino acid sequence of said polypeptide can comprise L5, when aligned to the polypeptide sequence of SEQ ID No: 1. In some embodiments, the amino acid sequence of said polypeptide can comprise M7, when aligned to the polypeptide sequence of SEQ ID No: 1. In some embodiments, the amino acid sequence of said polypeptide can comprise L19, when aligned to the polypeptide sequence of SEQ ID No: 1. In some embodiments, the amino acid sequence of said polypeptide can comprise at least one, at least two, at least three or at least four members selected from the group consisting of C4, L5, M7, and L19, when aligned to the polypeptide sequence of SEQ ID NO: 1. In some embodiments, the amino acid sequence of said polypeptide can comprise one or more members selected from the group consisting of C4, L5, M7, and L19, when aligned to the polypeptide sequence of SEQ ID NO: 1.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 2. For example, the amino acid sequence of the polypeptide of the engineered gene effector may not and need not be identical to the polypeptide sequence of SEQ ID NO: 2.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 3. For example, the amino acid sequence of the polypeptide of the engineered gene effector may not and need not be identical to the polypeptide sequence of SEQ ID NO: 3.

In some embodiments, the polypeptide of the engineered gene effector as disclosed herein can have a size of at least or up to about 250 amino acid residues, at least or up to about 240 amino acid residues, at least or up to about 230 amino acid residues, at least or up to about 220 amino acid residues, at least or up to about 210 amino acid residues, at least or up to about 200 amino acid residues, at least or up to about 190 amino acid residues, at least or up to about 180 amino acid residues, at least or up to about 170 amino acid residues, at least or up to about 160 amino acid residues, at least or up to about 150 amino acid residues, at least or up to about 140 amino acid residues, at least or up to about 130 amino acid residues, at least or up to about 120 amino acid residues, at least or up to about 110 amino acid residues, at least or up to about 100 amino acid residues, at least or up to about 95 amino acid residues, at least or up to about 90 amino acid residues, at least or up to about 85 amino acid residues, at least or up to about 80 amino acid residues, at least or up to about 70 amino acid residues, at least or up to about 60 amino acid residues, at least or up to about 50 amino acid residues, at least or up to about 45 amino acid residues, at least or up to about 40 amino acid residues, at least or up to about 35 amino acid residues, at least or up to about 30 amino acid residues, at least or up to about 25 amino acid residues, at least or up to about 20 amino acid residues, at least or up to about 15 amino acid residues, or at least or up to about 10 amino acid residues.

In some embodiments, the size of the polypeptide of the engineered gene effector as disclosed herein can be between about 10 amino acid residues and about 100 amino acid residues, between about 10 amino acid residues and about 90 amino acid residues, between about 10 amino acid residues and about 80 amino acid residues, between about 10 amino acid residues and about 70 amino acid residues, between about 10 amino acid residues and about 60 amino acid residues, between about 10 amino acid residues and about 50 amino acid residues, between about 10 amino acid residues and about 40 amino acid residues, between about 15 amino acid residues and about 100 amino acid residues, between about 15 amino acid residues and about 90 amino acid residues, between about 15 amino acid residues and about 80 amino acid residues, between about 15 amino acid residues and about 70 amino acid residues, between about 15 amino acid residues and about 60 amino acid residues, between about 15 amino acid residues and about 50 amino acid residues, between about 15 amino acid residues and about 40 amino acid residues, between about 20 amino acid residues and about 100 amino acid residues, between about 20 amino acid residues and about 90 amino acid residues, between about 20 amino acid residues and about 80 amino acid residues, between about 20 amino acid residues and about 70 amino acid residues, between about 20 amino acid residues and about 60 amino acid residues, between about 20 amino acid residues and about 50 amino acid residues, between about 20 amino acid residues and about 40 amino acid residues, or between about 25 amino acid residues and about 35 amino acid residues.

In some embodiments, the engineered gene effector can comprise only one of the polypeptide as disclosed herein.

In some embodiments, the engineered gene effector can comprise a plurality (e.g., at least 2, at least 3, at least 4, at least 5, at least 6, at least 7, at least 8, at least 9, at least 10, or more) of such polypeptide as disclosed herein. The plurality of such polypeptide may be substantially the same. Alternatively or in addition to, the plurality of such polypeptide may be different from one another. In some embodiments, the plurality of such polypeptide can comprise a first polypeptide (e.g., exhibiting at least about 70% sequence identity to the polypeptide sequence of SEQ ID NO: 1) and a second polypeptide (e.g., exhibiting at least about 70% sequence identity to the polypeptide sequence of SEQ ID NO: 1). The first polypeptide and the second polypeptide can be coupled to each other, e.g., directly or indirectly (e.g., via a linker). For example, the first polypeptide and the second polypeptide can be fused to each other, e.g., directly or indirectly (e.g., via the linker). In another example, the first polypeptide and the second polypeptide can be non-covalently coupled to each other, e.g., via ionic bonds, hydrogen bonds, interactions mediated by oligomerization or dimerization domains, etc.

In some embodiments of any of the engineered gene effectors disclosed herein, a central amino acid sequence of the polypeptide can be closer to C-terminus of the engineered gene effector, as compared to N-terminus of the engineered gene effector, by at least or up to about 1 amino acid residue, at least or up to about 2 amino acid residues, at least or up to about 3 amino acid residues, at least or up to about 4 amino acid residues, at least or up to about 5 amino acid residues, at least or up to about 10 amino acid residues, at least or up to about 15 amino acid residues, at least or up to about 20 amino acid residues, at least or up to about 25 amino acid residues, at least or up to about 30 amino acid residues, at least or up to about 35 amino acid residues, at least or up to about 40 amino acid residues, at least or up to about 45 amino acid residues, at least or up to about 50 amino acid residues, at least or up to about 60 amino acid residues, at least or up to about 70 amino acid residues, at least or up to about 80 amino acid residues, at least or up to about 90 amino acid residues, at least or up to about 100 amino acid residues, at least or up to about 110 amino acid residues, at least or up to about 120 amino acid residues, at least or up to about 130 amino acid residues, at least or up to about 140 amino acid residues, at least or up to about 150 amino acid residues, or at least or up to about 200 amino acid residues.

In some embodiments of any of the engineered gene effectors disclosed herein, a central amino acid sequence of the polypeptide can be closer to N-terminus of the engineered gene effector, as compared to C-terminus of the engineered gene effector, by at least or up to about 1 amino acid residue, at least or up to about 2 amino acid residues, at least or up to about 3 amino acid residues, at least or up to about 4 amino acid residues, at least or up to about 5 amino acid residues, at least or up to about 10 amino acid residues, at least or up to about 15 amino acid residues, at least or up to about 20 amino acid residues, at least or up to about 25 amino acid residues, at least or up to about 30 amino acid residues, at least or up to about 35 amino acid residues, at least or up to about 40 amino acid residues, at least or up to about 45 amino acid residues, at least or up to about 50 amino acid residues, at least or up to about 60 amino acid residues, at least or up to about 70 amino acid residues, at least or up to about 80 amino acid residues, at least or up to about 90 amino acid residues, at least or up to about 100 amino acid residues, at least or up to about 110 amino acid residues, at least or up to about 120 amino acid residues, at least or up to about 130 amino acid residues, at least or up to about 140 amino acid residues, at least or up to about 150 amino acid residues, or at least or up to about 200 amino acid residues.

In some embodiments, the polypeptide of the engineered effector as disclosed herein (e.g., engineered gene activator) can be heterologous to one or more members (e.g., at least 1, 2, 3, 4, 5, or more members, or any one of the members) selected from the group consisting of VP16, VP64, VP48, VP160, p65, EDLL, TAL, SET1A, SET1B, MLL, ASH1, SYMD2, NSD1, JHDM2a/b, UTX, JMJD3, GCN5, PCAF, CBP, p300, TAF1, TIP60/PLIP, MOZMYST3, MORFMYST4, SRC1, ACTR, PI60, CLOCK, TETDME, DML1, DML2, ROS1, Rta, and VPR. Such one or more members may be referred to as transcriptional activators.

In some embodiments, the polypeptide of the engineered effector as disclosed herein (e.g., engineered gene activator) can be heterologous to one or more members (e.g., 1, 2, 3, 4, or 5 members) selected from the group consisting of VP16, VP64, p65, Rta, and VPR. In some cases, the polypeptide can be heterologous to any one of the members (or all of the members) selected from the group consisting of VP16, VP64, p65, Rta, and VPR.

In some embodiments, the engineered gene effector as disclosed herein (e.g., the engineered gene activator) can be capable of (e.g., either alone or in conjunction with a heterologous endonuclease, such as Cas or dCas protein, e.g., deactivated variant of Un1Cas12f1 that comprises the polypeptide sequence of SEQ ID NO: 11) activating expression level of a target gene in a cell, as compared to a control. The control can be an expression level (e.g., a basal expression level) of the target gene in a control cell in absence of any external manipulation of the target gene (e.g., in absence of the engineered gene effector and/or the heterologous endonuclease). The control can be an expression level of the target gene in a control cell that is treated with the heterologous endonuclease (e.g., dCas) alone without any gene effector operatively coupled to the gene effector. The control can be an expression level of the target gene in a control cell that is treated with a control gene effector (e.g., either alone or in conjunction the heterologous endonuclease). Non-limiting examples of the control gene effector can include VP16, VP64, p65, Rta, and VPR.

In some embodiments, the expression level of the target gene that is activated via the engineered gene effector as disclosed herein (e.g., in conjunction with the heterologous endonuclease) can be at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100%, at least about 101%, at least about 102%, at least about 103%, at least about 104%, at least about 105%, at least about 106%, at least about 107%, at least about 108%, at least about 109%, at least about 110%, at least about 115%, at least about 120%, at least about 125%, at least about 130%, at least about 140%, at least about 150%, at least about 160%, at least about 170%, at least about 180%, at least about 190%, at least about 200%, or more, as compared to the expression level of the target gene in the control cell that is activated by VPR (e.g., in conjunction with the heterologous endonuclease).

In some embodiments, the expression level of the target gene that is activated via the engineered gene effector as disclosed herein (e.g., in conjunction with the heterologous endonuclease) can be at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100%, at least about 101%, at least about 102%, at least about 103%, at least about 104%, at least about 105%, at least about 106%, at least about 107%, at least about 108%, at least about 109%, at least about 110%, at least about 115%, at least about 120%, at least about 125%, at least about 130%, at least about 140%, at least about 150%, at least about 160%, at least about 170%, at least about 180%, at least about 190%, at least about 200%, or more, as compared to the expression level of the target gene in the control cell that is activated by Rta (e.g., in conjunction with the heterologous endonuclease).

In some embodiments, the expression level of the target gene that is activated via the engineered gene effector as disclosed herein (e.g., in conjunction with the heterologous endonuclease) can be at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100%, at least about 101%, at least about 102%, at least about 103%, at least about 104%, at least about 105%, at least about 106%, at least about 107%, at least about 108%, at least about 109%, at least about 110%, at least about 115%, at least about 120%, at least about 125%, at least about 130%, at least about 140%, at least about 150%, at least about 160%, at least about 170%, at least about 180%, at least about 190%, at least about 200%, or more, as compared to the expression level of the target gene in the control cell that is activated by p65 (e.g., in conjunction with the heterologous endonuclease).

In some embodiments, the expression level of the target gene that is activated via the engineered gene effector as disclosed herein (e.g., in conjunction with the heterologous endonuclease) can be at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100%, at least about 101%, at least about 102%, at least about 103%, at least about 104%, at least about 105%, at least about 106%, at least about 107%, at least about 108%, at least about 109%, at least about 110%, at least about 115%, at least about 120%, at least about 125%, at least about 130%, at least about 140%, at least about 150%, at least about 160%, at least about 170%, at least about 180%, at least about 190%, at least about 200%, or more, as compared to the expression level of the target gene in the control cell that is activated by VP64 (e.g., in conjunction with the heterologous endonuclease).

In some embodiments, the expression level of the target gene that is activated via the engineered gene effector as disclosed herein (e.g., in conjunction with the heterologous endonuclease) can be at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100%, at least about 101%, at least about 102%, at least about 103%, at least about 104%, at least about 105%, at least about 106%, at least about 107%, at least about 108%, at least about 109%, at least about 110%, at least about 115%, at least about 120%, at least about 125%, at least about 130%, at least about 140%, at least about 150%, at least about 160%, at least about 170%, at least about 180%, at least about 190%, at least about 200%, or more, as compared to the expression level of the target gene in the control cell that is activated by VP16 (e.g., in conjunction with the heterologous endonuclease).

In some embodiments, the expression level of the target gene that is activated via the engineered gene effector as disclosed herein (e.g., in conjunction with the heterologous endonuclease) can be at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, at least about 100%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, or more greater than the expression level of the target gene in the control cell that is activated by VPR (e.g., in conjunction with the heterologous endonuclease).

In some embodiments, the expression level of the target gene that is activated via the engineered gene effector as disclosed herein (e.g., in conjunction with the heterologous endonuclease) can be at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, at least about 100%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, or more greater than the expression level of the target gene in the control cell that is activated by Rta (e.g., in conjunction with the heterologous endonuclease).

In some embodiments, the expression level of the target gene that is activated via the engineered gene effector as disclosed herein (e.g., in conjunction with the heterologous endonuclease) can be at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, at least about 100%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, or more greater than the expression level of the target gene in the control cell that is activated by p65 (e.g., in conjunction with the heterologous endonuclease).

In some embodiments, the expression level of the target gene that is activated via the engineered gene effector as disclosed herein (e.g., in conjunction with the heterologous endonuclease) can be at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, at least about 100%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, or more greater than the expression level of the target gene in the control cell that is activated by VP64 (e.g., in conjunction with the heterologous endonuclease).

In some embodiments, the expression level of the target gene that is activated via the engineered gene effector as disclosed herein (e.g., in conjunction with the heterologous endonuclease) can be at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, at least about 100%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, or more greater than the expression level of the target gene in the control cell that is activated by VP16 (e.g., in conjunction with the heterologous endonuclease).

In some embodiments, the expression level of the target gene that is activated via the engineered gene effector as disclosed herein (e.g., in conjunction with the heterologous endonuclease) can be substantially sustained (e.g., persisting or maintaining at least about 50%, at least about 60%, at least about 70%, at least about 80%, at least about 90%, at least about 95%, at least about 99%, or about 100% of a peak value or that of the modulated level) for at least or up to about 1 hour, at least or up to about 2 hours, at least or up to about 6 hours, at least or up to about 12 hours, at least or up to about 18 hours, at least or up to about 24 hours, at least or up to about 2 days, at least or up to about 3 days, at least or up to about 4 days, at least or up to about 5 days, at least or up to about 6 days, at least or up to about 7 days, at least or up to about 8 days, at least or up to about 9 days, at least or up to about 10 days, at least or up to about 11 days, at least or up to about 12 days, at least or up to about 13 days, at least or up to about 14 days, at least or up to about 3 weeks, at least or up to about 4 weeks, at least or up to about 2 months, at least or up to about 4 months, or at least or up to about 6 months.

In some embodiments, the expression level of the target gene that is activated via the engineered gene effector as disclosed herein (e.g., in conjunction with the heterologous endonuclease) can be substantially sustained for at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or longer, as compared to the expression level of the target gene in the control cell that is activated by a control gene effector (e.g., VPR, Rta, p65, VP64, VP16, P300, etc. in conjunction with a comparable heterologous endonuclease).

In some embodiments, a population of cells contacted by the system as provided herein (e.g., at least the engineered gene effector) can result in a higher proportion of cells exhibiting the modulated expression level (e.g., activation or suppression) of the target gene compared to a population of cells contacted by the system comprising a control gene effector (e.g., VPR, Rta, p65, VP64, VP16, P300, etc.). In some embodiments, a proportion of cells exhibiting the modulated expression level can be at least about 10%, at least about 20%, at least about 30%, at least about 40%, at least about 50%, at least about 60%, at least about 70%, at least about 80%, at least about 90%, at least about 100% of the population of cells contacted by the systems as provided herein (e.g., at least the engineered gene effector). In some embodiments, a population of cells contacted by the system as provided herein (e.g., at least the engineered gene effector) can result in a higher proportion of cells exhibiting the modulated expression level, which can be at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or higher than that of a proportion of a population of cells exhibiting the modulated expression level contacted by the system comprising a control gene effector (e.g., VPR, Rta, p65, VP64, VP16, P300, etc.).

In some embodiments, the polypeptide of the engineered effector as disclosed herein (e.g., engineered gene repressor) can be heterologous to one or more members (e.g., at least 1, 2, 3, 4, 5, or more members, or any one of the members) selected from the group consisting of Kruppel associated box (KRAB or SKD); KOX1 repression domain; the Mad mSIN3 interaction domain (SID); the ERF repressor domain (ERD), the SRDX repression domain (e.g, for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like. Such one or more members may be referred to as transcriptional repressors.

In some embodiments, the polypeptide of the engineered effector as disclosed herein (e.g., engineered gene repressor) can be heterologous to one or more members (e.g., 1, 2, or 3 members) selected from the group consisting of KRAB, DNMT3A, and DNMT3L.

In some embodiments, the polypeptide of the engineered effector as disclosed herein (e.g., engineered gene repressor) may not and need not comprise KRAB, DNMT3A, and/or DNMT3L.

In some embodiments, the engineered gene effector as disclosed herein (e.g., the engineered gene repressor) can be capable of (e.g., either alone or in conjunction with a heterologous endonuclease, such as Cas or dCas protein, e.g., deactivated variant of Un1Cas12f1 that comprises the polypeptide sequence of SEQ ID NO: 11) reducing expression level of a target gene in a cell, as compared to a control. The control can be an expression level (e.g., a basal expression level) of the target gene in a control cell in absence of any external manipulation of the target gene (e.g., in absence of the engineered gene effector and/or the heterologous endonuclease). The control can be an expression level of the target gene in a control cell that is treated with the heterologous endonuclease (e.g., dCas) alone without any gene effector operatively coupled to the gene effector. The control can be an expression level of the target gene in a control cell that is treated with a control gene effector (e.g., either alone or in conjunction the heterologous endonuclease). Non-limiting examples of the control gene effector can include KRAB, DNMT3A, DNMT3L, and a fusion repressor comprising KRAB, DNMT3A, and DNMT3L (KAL).

In some embodiments, the expression level of the target gene that is reduced via the engineered gene effector as disclosed herein (e.g., in conjunction with the heterologous endonuclease) can be at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%, as compared to the expression level of the target gene in the control cell that is reduced by KRAB (e.g., in conjunction with the heterologous endonuclease).

In some embodiments, the expression level of the target gene that is reduced via the engineered gene effector as disclosed herein (e.g., in conjunction with the heterologous endonuclease) can be comparable (e.g., substantially the same as) to the expression level of the target gene in the control cell that is reduced by KRAB (e.g., in conjunction with the heterologous endonuclease).

In some embodiments, the expression level of the target gene that is reduced via the engineered gene effector as disclosed herein (e.g., in conjunction with the heterologous endonuclease) can be less than the expression level of the target gene in the control cell that is reduced by KRAB (e.g., in conjunction with the heterologous endonuclease), by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 40%, at least about 50%, at least about 60%, at least about 70%, at least about 80%, at least about 90%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

In some embodiments, the expression level of the target gene that is reduced via the engineered gene effector as disclosed herein (e.g., in conjunction with the heterologous endonuclease) can be at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%, as compared to the expression level of the target gene in the control cell that is reduced by DNMT3A (e.g., in conjunction with the heterologous endonuclease).

In some embodiments, the expression level of the target gene that is reduced via the engineered gene effector as disclosed herein (e.g., in conjunction with the heterologous endonuclease) can be comparable (e.g., substantially the same as) to the expression level of the target gene in the control cell that is reduced by DNMT3A (e.g., in conjunction with the heterologous endonuclease).

In some embodiments, the expression level of the target gene that is reduced via the engineered gene effector as disclosed herein (e.g., in conjunction with the heterologous endonuclease) can be less than the expression level of the target gene in the control cell that is reduced by DNMT3A (e.g., in conjunction with the heterologous endonuclease), by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 40%, at least about 50%, at least about 60%, at least about 70%, at least about 80%, at least about 90%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

In some embodiments, the expression level of the target gene that is reduced via the engineered gene effector as disclosed herein (e.g., in conjunction with the heterologous endonuclease) can be at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%, as compared to the expression level of the target gene in the control cell that is reduced by DNMT3L (e.g., in conjunction with the heterologous endonuclease).

In some embodiments, the expression level of the target gene that is reduced via the engineered gene effector as disclosed herein (e.g., in conjunction with the heterologous endonuclease) can be comparable (e.g., substantially the same as) to the expression level of the target gene in the control cell that is reduced by DNMT3L (e.g., in conjunction with the heterologous endonuclease).

In some embodiments, the expression level of the target gene that is reduced via the engineered gene effector as disclosed herein (e.g., in conjunction with the heterologous endonuclease) can be less than the expression level of the target gene in the control cell that is reduced by DNMT3L (e.g., in conjunction with the heterologous endonuclease), by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 40%, at least about 50%, at least about 60%, at least about 70%, at least about 80%, at least about 90%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

In some embodiments, the expression level of the target gene that is reduced via the engineered gene effector as disclosed herein (e.g., in conjunction with the heterologous endonuclease) can be at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, or at least or up to about 95%, as compared to the expression level of the target gene in the control cell that is reduced by KAL (e.g., in conjunction with the heterologous endonuclease).

In some embodiments, the expression level of the target gene that is reduced via the engineered gene effector as disclosed herein (e.g., in conjunction with the heterologous endonuclease) can be comparable (e.g., substantially the same as) to the expression level of the target gene in the control cell that is reduced by KAL (e.g., in conjunction with the heterologous endonuclease).

In some embodiments, the expression level of the target gene that is reduced via the engineered gene effector as disclosed herein (e.g., in conjunction with the heterologous endonuclease) can be less than the expression level of the target gene in the control cell that is reduced by KAL (e.g., in conjunction with the heterologous endonuclease), by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 40%, at least about 50%, at least about 60%, at least about 70%, at least about 80%, at least about 90%, at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

In some embodiments, the expression level of the target gene that is reduced via the engineered gene effector as disclosed herein (e.g., in conjunction with the heterologous endonuclease) can be substantially sustained for at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or longer, as compared to the expression level of the target gene in the control cell that is reduced by KRAB (e.g., in conjunction with the heterologous endonuclease).

In some embodiments, the expression level of the target gene that is reduced via the engineered gene effector as disclosed herein (e.g., in conjunction with the heterologous endonuclease) can be substantially sustained for at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or longer, as compared to the expression level of the target gene in the control cell that is reduced by DNMT3A (e.g., in conjunction with the heterologous endonuclease).

In some embodiments, the expression level of the target gene that is reduced via the engineered gene effector as disclosed herein (e.g., in conjunction with the heterologous endonuclease) can be substantially sustained for at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or longer, as compared to the expression level of the target gene in the control cell that is reduced by DNMT3L (e.g., in conjunction with the heterologous endonuclease).

In some embodiments, the expression level of the target gene that is reduced via the engineered gene effector as disclosed herein (e.g., in conjunction with the heterologous endonuclease) can be substantially sustained for at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or longer, as compared to the expression level of the target gene in the control cell that is reduced by KAL (e.g., in conjunction with the heterologous endonuclease).

In some embodiments, the engineered gene effector as disclosed herein can comprise the polypeptide and an additional polypeptide. The polypeptide and the additional polypeptide can be heterologous to each other. The polypeptide and the additional polypeptide can be coupled to each other, e.g., directly or indirectly (e.g., via a linker). For example, the polypeptide and the additional polypeptide can be fused to each other, e.g., directly or indirectly (e.g., via the linker). In another example, the polypeptide and the additional polypeptide can be non-covalently coupled to each other, e.g., via ionic bonds, hydrogen bonds, interactions mediated by oligomerization or dimerization domains, etc. In some embodiments, the engineered gene effector can comprise only one of the additional polypeptide as disclosed herein. In some embodiments, the engineered gene effector can comprise a plurality (e.g., at least 2, 3, 4, 5, 6, 7, 8, 9, 10, or more) of such additional polypeptide as disclosed herein. The plurality of such additional polypeptide may be substantially the same. Alternatively or in addition to, the plurality of such additional polypeptide may be different from one another.

In some embodiments, the additional polypeptide of the engineered gene effector may not comprise the polypeptide sequence of SEQ ID NO: 1.

Without wishing to be bound by theory, in some embodiments, (1) a first engineered gene effector can comprise at least a portion of the polypeptide sequence of SEQ ID NO: 1 and (2) a second engineered gene effector can comprise at least a portion of the polypeptide sequence of SEQ ID NO: 1 and being different than the first engineered gene effector, and the first engineered gene effector and the second engineered gene effector can exhibit different gene modulating activities. In some cases, even though (1) the at least the portion of the polypeptide sequence of SEQ ID NO: 1 of the first engineered gene effector may be substantially the same as (2) the at least the portion of the polypeptide sequence of SEQ ID NO: 1 of the second engineered gene effector, the first engineered gene effector and the second engineered gene effector can exhibit different (e.g., opposite) gene modulating activities. For example, the first engineered gene effector may be a gene activator, while the second engineered gene effector may be a gene repressor. The different activities of the first and second engineered gene effectors may be due to one or more differences between the first and the second engineered gene effector such as, for example, (i) the position of the at least the portion of the polypeptide sequence of SEQ ID NO: 1 within each of the engineered gene effectors, (ii) the overall size/length of the engineered gene effector, (iii) a presence, size, or amino acid residue composition of additional polypeptide sequence to the N-terminus of the at least the portion of the polypeptide sequence of SEQ ID NO: 1, (iv) a presence, size, or amino acid residue composition of additional polypeptide sequence to the C-terminus of the at least the portion of the polypeptide sequence of SEQ ID NO: 1, and/or (v) size or amino acid residue composition of the at least the portion of the polypeptide sequence of SEQ ID NO: 1.

In some embodiments, the additional polypeptide of the gene effector as disclosed herein can be disposed adjacent to the N-terminus of the polypeptide of the gene effector. In some embodiments, the additional polypeptide of the gene effector can be disposed adjacent to the C-terminus of the polypeptide of the gene effector. In some embodiments, a first type of the additional polypeptide (e.g., a first additional polypeptide) of the gene effector can be disposed adjacent to the N-terminus of the polypeptide of the gene effector, and a second type of the additional polypeptide (e.g., a second additional polypeptide) of the gene effector can be disposed adjacent to the C-terminus of the polypeptide of the gene effector (e.g., the first additional polypeptide—the polypeptide—the second additional polypeptide). The polypeptide can be flanked by the first additional polypeptide and the second additional polypeptide. The first additional polypeptide and the second additional polypeptide may be substantially the same. Alternatively, the first additional polypeptide and the second additional polypeptide may be different. In some embodiments, a first type of the polypeptide (e.g., a first polypeptide) of the gene effector can be disposed adjacent to the N-terminus of the additional polypeptide of the gene effector, and a second type of the polypeptide (e.g., a second polypeptide) of the gene effector can be disposed adjacent to the C-terminus of the additional polypeptide of the gene effector (e.g., the first polypeptide—the additional polypeptide—the second polypeptide). The additional polypeptide can be flanked by the first polypeptide and the second polypeptide. The first polypeptide and the second polypeptide may be substantially the same. Alternatively, the first polypeptide and the second polypeptide may be different.

In some embodiments, the additional polypeptide of the engineered gene effector, as disclosed herein, can be disposed within (e.g., inserted into) the polypeptide of the engineered gene effector, as disclosed herein. The additional polypeptide can be inserted within and adjacent to the N-terminus of the polypeptide. Alternatively or in addition to, the additional polypeptide can be inserted within and adjacent to the C-terminus of the polypeptide.

In some embodiments, a size of the polypeptide and a size of the additional polypeptide of the engineered gene effector as disclosed herein may be substantially the same. In some embodiments, the size of the polypeptide and the size of the additional polypeptide of the engineered gene effector may be different.

In some embodiments of the engineered gene effector as disclosed herein, the size of the polypeptide can be longer than the size of the additional polypeptide, e.g., by at least or up to about 1 amino acid residue, at least or up to about 2 amino acid residues, at least or up to about 3 amino acid residues, at least or up to about 4 amino acid residues, at least or up to about 5 amino acid residues, at least or up to about 10 amino acid residues, at least or up to about 15 amino acid residues, at least or up to about 20 amino acid residues, at least or up to about 25 amino acid residues, at least or up to about 30 amino acid residues, at least or up to about 35 amino acid residues, at least or up to about 40 amino acid residues, at least or up to about 45 amino acid residues, at least or up to about 50 amino acid residues, at least or up to about 60 amino acid residues, at least or up to about 70 amino acid residues, at least or up to about 80 amino acid residues, at least or up to about 90 amino acid residues, at least or up to about 100 amino acid residues, at least or up to about 110 amino acid residues, at least or up to about 120 amino acid residues, at least or up to about 130 amino acid residues, at least or up to about 140 amino acid residues, at least or up to about 150 amino acid residues, or at least or up to about 200 amino acid residues.

In some embodiments of the engineered gene effector as disclosed herein, the size of the additional polypeptide can be longer than the size of the polypeptide, e.g., by at least or up to about 1 amino acid residue, at least or up to about 2 amino acid residues, at least or up to about 3 amino acid residues, at least or up to about 4 amino acid residues, at least or up to about 5 amino acid residues, at least or up to about 10 amino acid residues, at least or up to about 15 amino acid residues, at least or up to about 20 amino acid residues, at least or up to about 25 amino acid residues, at least or up to about 30 amino acid residues, at least or up to about 35 amino acid residues, at least or up to about 40 amino acid residues, at least or up to about 45 amino acid residues, at least or up to about 50 amino acid residues, at least or up to about 60 amino acid residues, at least or up to about 70 amino acid residues, at least or up to about 80 amino acid residues, at least or up to about 90 amino acid residues, at least or up to about 100 amino acid residues, at least or up to about 110 amino acid residues, at least or up to about 120 amino acid residues, at least or up to about 130 amino acid residues, at least or up to about 140 amino acid residues, at least or up to about 150 amino acid residues, at least or up to about 200 amino acid residues, or more.

In some embodiments of the engineered gene effector as disclosed herein, the size of the polypeptide can be at least about 1%, at least about 2%, at least about 5%, at least about 10%, at least about 15%, at least about 20%, at least about 25%, at least about 26%, at least about 27%, at least about 28%, at least about 29%, at least about 30%, at least about 31%, at least about 32%, at least about 33%, at least about 34%, at least about 35%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 250%, at least about 300%, at least about 400%, at least about 500%, or more greater than the size of the additional polypeptide. In some embodiments of the engineered gene effector as disclosed herein, the size of the polypeptide can be at most about 1%, at most about 2%, at most about 5%, at most about 10%, at most about 15%, at most about 20%, at most about 25%, at most about 26%, at most about 27%, at most about 28%, at most about 29%, at most about 30%, at most about 31%, at most about 32%, at most about 33%, at most about 34%, at most about 35%, at most about 40%, at most about 50%, at most about 60%, at most about 70%, at most about 80%, at most about 90%, at most about 100%, at most about 110%, at most about 120%, at most about 130%, at most about 140%, at most about 150%, at most about 160%, at most about 170%, at most about 180%, at most about 190%, at most about 200%, at most about 250%, at most about 300%, at most about 400%, or at most about 500% greater than the size of the additional polypeptide.

In some embodiments of the engineered gene effector as disclosed herein, the size of the additional polypeptide can be at least about 1%, at least about 2%, at least about 5%, at least about 10%, at least about 15%, at least about 20%, at least about 25%, at least about 26%, at least about 27%, at least about 28%, at least about 29%, at least about 30%, at least about 31%, at least about 32%, at least about 33%, at least about 34%, at least about 35%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 250%, at least about 300%, at least about 400%, at least about 500%, or more greater than the size of the polypeptide. In some embodiments of the engineered gene effector as disclosed herein, the size of the additional polypeptide can be at most about 1%, at most about 2%, at most about 5%, at most about 10%, at most about 15%, at most about 20%, at most about 25%, at most about 26%, at most about 27%, at most about 28%, at most about 29%, at most about 30%, at most about 31%, at most about 32%, at most about 33%, at most about 34%, at most about 35%, at most about 40%, at most about 50%, at most about 60%, at most about 70%, at most about 80%, at most about 90%, at most about 100%, at most about 110%, at most about 120%, at most about 130%, at most about 140%, at most about 150%, at most about 160%, at most about 170%, at most about 180%, at most about 190%, at most about 200%, at most about 250%, at most about 300%, at most about 400%, or at most about 500% greater than the size of the polypeptide.

In some embodiments, the additional polypeptide of the engineered gene effector as disclosed herein can have a size of at least or up to about 500 amino acid residues, at least or up to about 480 amino acid residues, at least or up to about 460 amino acid residues, at least or up to about 450 amino acid residues, at least or up to about 440 amino acid residues, at least or up to about 420 amino acid residues, at least or up to about 400 amino acid residues, at least or up to about 380 amino acid residues, at least or up to about 360 amino acid residues, at least or up to about 350 amino acid residues, at least or up to about 340 amino acid residues, at least or up to about 320 amino acid residues, at least or up to about 300 amino acid residues, at least or up to about 290 amino acid residues, at least or up to about 280 amino acid residues, at least or up to about 270 amino acid residues, at least or up to about 260 amino acid residues, at least or up to about 250 amino acid residues, at least or up to about 240 amino acid residues, at least or up to about 230 amino acid residues, at least or up to about 220 amino acid residues, at least or up to about 210 amino acid residues, at least or up to about 200 amino acid residues, at least or up to about 190 amino acid residues, at least or up to about 180 amino acid residues, at least or up to about 170 amino acid residues, at least or up to about 160 amino acid residues, at least or up to about 150 amino acid residues, at least or up to about 140 amino acid residues, at least or up to about 130 amino acid residues, at least or up to about 120 amino acid residues, at least or up to about 110 amino acid residues, at least or up to about 100 amino acid residues, at least or up to about 95 amino acid residues, at least or up to about 90 amino acid residues, at least or up to about 85 amino acid residues, at least or up to about 80 amino acid residues, at least or up to about 70 amino acid residues, at least or up to about 60 amino acid residues, at least or up to about 50 amino acid residues, at least or up to about 45 amino acid residues, at least or up to about 40 amino acid residues, at least or up to about 35 amino acid residues, at least or up to about 30 amino acid residues, at least or up to about 25 amino acid residues, at least or up to about 20 amino acid residues, at least or up to about 15 amino acid residues, at least or up to about 10 amino acid residues, at least or up to about 9 amino acid residues, at least or up to about 8 amino acid residues, at least or up to about 7 amino acid residues, at least or up to about 6 amino acid residues, at least or up to about 5 amino acid residues, at least or up to about 4 amino acid residues, at least or up to about 3 amino acid residues, at least or up to about 2 amino acid residues, or at least or up to about 1 amino acid residues.

In some embodiments, the additional polypeptide of the engineered gene effector as disclosed herein can have a size of between about 2 and about 50, between about 2 and about 40, between about 2 and about 30, between about 2 and about 25, between about 2 and about 20, between about 2 and about 15, between about 2 and about 10, between about 2 and about 9, between about 2 and about 8, between about 2 and about 7, between about 2 and about 6, between about 2 and about 5, between about 2 and about 4, between about 3 and about 15, between about 3 and about 10, between about 3 and about 9, between about 3 and about 8, between about 3 and about 7, between about 3 and about 6, between about 3 and about 5, between about 1 and about 15, between about 1 and about 10, between about 1 and about 9, between about 1 and about 8, between about 1 and about 7, between about 1 and about 6, between about 1 and about 5, between about 1 and about 4, or between about 1 and about 3 amino acid residues.

In some embodiments, the additional polypeptide of the engineered gene effector as disclosed herein can have a size of less than or equal to about 50 amino acid residues, less than or equal to about 45 amino acid residues, less than or equal to about 44 amino acid residues, less than or equal to about 43 amino acid residues, less than or equal to about 42 amino acid residues, less than or equal to about 41 amino acid residues, less than or equal to about 40 amino acid residues, less than or equal to about 39 amino acid residues, less than or equal to about 38 amino acid residues, less than or equal to about 37 amino acid residues, less than or equal to about 36 amino acid residues, less than or equal to about 35 amino acid residues, less than or equal to about 34 amino acid residues, less than or equal to about 33 amino acid residues, less than or equal to about 32 amino acid residues, less than or equal to about 31 amino acid residues, less than or equal to about 30 amino acid residues, less than or equal to about 29 amino acid residues, less than or equal to about 28 amino acid residues, less than or equal to about 27 amino acid residues, less than or equal to about 26 amino acid residues, less than or equal to about 25 amino acid residues, less than or equal to about 24 amino acid residues, less than or equal to about 23 amino acid residues, less than or equal to about 22 amino acid residues, less than or equal to about 21 amino acid residues, less than or equal to about 20 amino acid residues, less than or equal to about 15 amino acid residues, less than or equal to about 10 amino acid residues, less than or equal to about 5 amino acid residues, less than or equal to about 4 amino acid residues, less than or equal to about 3 amino acid residues, less than or equal to about 2 amino acid residues, or about 1 amino acid residue.

In some embodiments, the additional polypeptide (of the engineered gene effector as disclosed herein) alone may be inert, e.g., not eliciting any desired biological activity in a cell on its own. In some embodiments, the additional polypeptide (of the engineered gene effector as disclosed herein) alone may not and need not encode (or comprise) any gene effector. Thus, the additional polypeptide may not be capable of regulating expression and/or activity of a target gene in a cell, in absence of the polypeptide of the engineered gene effector as disclosed herein.

In some embodiments, the additional polypeptide of the engineered gene effector as disclosed herein may comprise a filler polypeptide sequence. The filler polypeptide sequence can be used in conjunction with another polypeptide sequence (e.g., the polypeptide of the engineered gene effector) to increase the size of the whole polypeptide (e.g., the engineered gene effector). The filler polypeptide sequence alone may not be capable of exhibiting a biological activity of interest. For example, the filler polypeptide sequence alone may not be capable of regulating (e.g., activating, repressing, etc.) expression or activity level of a target gene in a cell, in absence of other portions of the engineered gene effector (e.g., in absence of the polypeptide of the engineered gene effector). The filler polypeptide sequence may not be found in a natural protein (e.g., in a natural mammalian protein). In some embodiments, the filler polypeptide sequence can comprise at least one (e.g., one of, or a plurality of) aliphatic amino acid selected from the group consisting of isoleucine (I), leucine (L), valine (V), alanine (A), and glycine (G). In some embodiments, the filler polypeptide sequence can comprise a plurality of I residues (e.g., II, III, etc.). In some embodiments, the filler polypeptide sequence can comprise a plurality of L residues (e.g., LL, LLL, etc.). In some embodiments, the filler polypeptide sequence can comprise a plurality of V residues (e.g., VV, VVV, etc.). In some embodiments, the filler polypeptide sequence can comprise a plurality of A residues (e.g., AA, AAA, etc.). In some embodiments, the filler polypeptide sequence can comprise a plurality of G residues (e.g., GG, GGG, etc.).

In some embodiments, the filler polypeptide sequence as disclosed herein can be a linker sequence. Any suitable linker can be used. A flexible linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains. Non-limiting examples of linkers include the sequences in Table 4, and repeats thereof, for example, 1, 2, 3, 4, 5, 6, 7, 8, 9, or 10 repeats. SEQ ID NOs: 65-70 provide flexible linkers or subunits thereof. SEQ ID NOs: 71-74 provide rigid linkers or subunits thereof.

TABLE 4

| SEQ ID NO: | Sequence |
|---|---|
| 65 | GGGGS |
| 66 | GGGS |
| 67 | GG |
| 68 | KESGSVSSEQLAQFRSLD |
| 69 | EGKSSGSGSESKST |
| 70 | GSAGSAAGSGEF |
| 71 | EAAAK |
| 72 | EAAAR |
| 73 | PAPAP |
| 74 | AEAAAKEAAAKA |

In some embodiments, a linker sequence as disclosed herein can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length.

In some embodiments, a linker sequence as disclosed herein can comprise at least 1, at least 2, at least 3, at least 5, at least 7, at least 9, at least 11, at least 13, at least 15, or at least 20 amino acids. In some embodiments, a linker sequence can comprise at most 5, at most 7, at most 9, at most 11, at most 13, at most 15, at most 20, at most 25, at most 30, at most 40, or at most 50 amino acids.

In some embodiments, non-peptide linkers are used. A non-peptide linker can be, for example a chemical linker. Two parts of a complex of the disclosure can be connected by a chemical linker. Each chemical linker of the disclosure can be alkylene, alkenylene, alkynylene, heteroalkylene, cycloalkylene, heterocycloalkylene, arylene, or heteroarylene, any of which is optionally substituted. In some embodiments, a chemical linker of the disclosure can be an ester, ether, amide, thioether, or polyethyleneglycol (PEG). In some embodiments, a linker can reverse the order of the amino acids sequence in a compound, for example, so that the amino acid sequences linked by the linked are head-to-head, rather than head-to-tail. Non-limiting examples of such linkers include diesters of dicarboxylic acids, such as oxalyl diester, malonyl diester, succinyl diester, glutaryl diester, adipyl diester, pimetyl diester, fumaryl diester, maleyl diester, phthalyl diester, isophthalyl diester, and terephthalyl diester. Non-limiting examples of such linkers include diamides of dicarboxylic acids, such as oxalyl diamide, malonyl diamide, succinyl diamide, glutaryl diamide, adipyl diamide, pimetyl diamide, fumaryl diamide, maleyl diamide, phthalyl diamide, isophthalyl diamide, and terephthalyl diamide. Non-limiting examples of such linkers include diamides of diamino linkers, such as ethylene diamine, 1,2-di(methylamino)ethane, 1,3-diaminopropane, 1,3-di(methylamino)propane, 1,4-di(methylamino)butane, 1,5-di(methylamino)pentane, 1,6-di(methylamino)hexane, and pipyrizine. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

In some embodiments, the additional polypeptide of the engineered gene effector as disclosed herein can comprise at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or about 1 glycine-serine (GS) linker(s). In some embodiments, the additional polypeptide of the engineered gene effector as disclosed herein (e.g., engineered gene repressor) can comprise at least about 1, at least about 2, at least about 3, at least about 4, at least about 5, at least about 6, at least about 7, at least about, at least about 9, at least about 10, at least about 11, at least about 12, at least about 13, at least about 14, at least about 15, at least about 20, or more GS linker(s).

In some embodiments, the additional polypeptide of the engineered gene effector as disclosed herein can comprise at most about 20, at most about 15, at most about 14, at most about 13, at most about 12, at most about 11, at most about 10, at most about 9, at most about 8, at most about 7, at most about 6, at most about 5, at most about 4, at most about 3, at most about 2, or about 1 glycine (G) linker(s). In some embodiments, the additional polypeptide of the engineered gene effector as disclosed herein (e.g., engineered gene repressor) can comprise at least about 1, at least about 2, at least about 3, at least about 4, at least about 5, at least about 6, at least about 7, at least about, at least about 9, at least about 10, at least about 11, at least about 12, at least about 13, at least about 14, at least about 15, at least about 20, or more G linker(s).

In some embodiments, the additional polypeptide (of the engineered gene effector as disclosed herein) alone can encode (or comprise) at least one gene effector. Thus, the at least one gene effector of the additional polypeptide can be capable of regulating expression and/or activity of a target gene in a cell, in absence of the polypeptide of the engineered gene effector. In some cases, the at least one gene effector can comprise a plurality of gene effectors (e.g., at least 2, 3, 4, 5, or more gene effectors) that are the same. Alternatively, the plurality of gene effectors may be different from one another. The plurality of gene effectors can comprise two or more gene activators. The plurality of gene effectors can comprise two or more gene repressors. The plurality of gene effectors can comprise a gene activator and a gene repressor.

In some embodiments, the additional polypeptide of the engineered gene effector as disclosed herein (e.g., engineered gene activator) can comprise at least a portion of one or more members selected from the group consisting of VP16, VP64, VP48, VP160, p65, EDLL, TAL, SET1A, SET1B, MLL, ASH1, SYMD2, NSD1, JHDM2a/b, UTX, JMJD3, GCN5, PCAF, CBP, p300, TAF1, TIP60/PLIP, MOZMYST3, MORFMYST4, SRC1, ACTR, PI60, CLOCK, TETDME, DML1, DML2, ROS1, Rta, and VPR. In some embodiments, the additional polypeptide of the engineered gene effector as disclosed herein (e.g., engineered gene activator) may not comprise two or more members selected from the group consisting of VP16, VP64, VP48, VP160, p65, EDLL, TAL, SET1A, SET1B, MLL, ASH1, SYMD2, NSD1, JHDM2a/b, UTX, JMJD3, GCN5, PCAF, CBP, p300, TAF1, TIP60/PLIP, MOZMYST3, MORFMYST4, SRC1, ACTR, PI60, CLOCK, TETDME, DML1, DML2, ROS1, and Rta.

In some embodiments, the additional polypeptide of the engineered gene effector as disclosed herein (e.g., engineered gene activator) can comprise at least a portion of one or more members selected from the group consisting of VP16, VP64, p65, Rta, and VPR.

In some embodiments, the additional polypeptide of the engineered gene effector as disclosed herein (e.g., engineered gene activator) may comprise two or more members selected from the group consisting of VP16, VP64, p65, and Rta. In some embodiments, the additional polypeptide of the engineered gene effectors as disclosed herein may comprise a plurality of VP16 domains. In some embodiments, the additional polypeptide of the engineered gene effectors as disclosed herein may comprise at least two VP16 domains, at least three VP16 domains, at least four VP16 domains, or at least five VP16 domains.

In some embodiments, the additional polypeptide of the engineered gene effector as disclosed herein (e.g., engineered gene activator) may not comprise two or more members selected from the group consisting of VP16, VP64, p65, and Rta. The additional polypeptide may comprise VP16 but not any of VP64, p65, and Rta. The additional polypeptide may comprise VP64, but not any of VP16, p65, and Rta. The additional polypeptide may comprise p65, but not any of VP16, VP64, and Rta. The additional polypeptide may comprise Rta, but not any of VP16, VP64, and p65. Alternatively, the additional polypeptide may not comprise any of VP16, VP64, p65, Rta, and VPR.

In some embodiments, the additional polypeptide of the engineered gene effector as disclosed herein (e.g., engineered gene activator) can comprise an amino acid sequence, and such amino acid sequence can be at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 9 (VPR).

In some embodiments, the engineered gene effector as disclosed herein does not comprise VPR.

In some embodiments, the additional polypeptide of the engineered gene effector as disclosed herein (e.g., engineered gene activator) can comprise an amino acid sequence, and such amino acid sequence can be at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 8 (Rta).

In some embodiments, the engineered gene effector as disclosed herein does not comprise Rta.

In some embodiments, the additional polypeptide of the engineered gene effector as disclosed herein (e.g., engineered gene activator) can comprise an amino acid sequence, and such amino acid sequence can be at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 7 (p65).

In some embodiments, the engineered gene effector as disclosed herein does not comprise p65.

In some embodiments, the additional polypeptide of the engineered gene effector as disclosed herein (e.g., engineered gene activator) can comprise an amino acid sequence, and such amino acid sequence can be at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 6 (VP64).

In some embodiments, the engineered gene effector as disclosed herein does not comprise VP64.

In some embodiments, the additional polypeptide of the engineered gene effector as disclosed herein (e.g., engineered gene activator) can comprise an amino acid sequence, and such amino acid sequence can be at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% identical to the polypeptide sequence of SEQ ID NO: 5 (VP16).

In some embodiments, the engineered gene effector as disclosed herein does not comprise VP16. In some embodiments, the engineered gene effector as disclosed herein (e.g., engineered gene activator) can comprise an amino acid sequence having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of a member from Table 2 (e.g., a member selected from SEQ ID NOs: 12-31).

TABLE 2

| SEQ ID | | |
|---|---|---|
| NO: | Amino Acid Sequence | Name |
| 75 | HVSGLDDCLPMVDHIEGCLLDLLSDVGQELPDLGDLGELLCETASPQ | EPIC-XV1.3 |
| | GPMQSEGGEEGSTESVSVLPATHPLESSAPGASVMGSG | |
| 76 | SPEGPVPSHVSGLDDCLPMVDHIEGCLLDLLSDVGQELPDLGDLGEL | EPIC-XV1.4 |
| | LCETASPQGPMQSEGGEEGSTESVSVLPATHPLESSAP | |
| 3 | PPAGQSQTPFSPEGPVPSHVSGLDDCLPMVDHIEGCLLDLLSDVGQEL | EPIC-XV1.5 |
| | PDLGDLGELLCETASPQGPMQSEGGEEGSTESVSVLP | |
| 77 | PHETQGLLAPPAGQSQTPFSPEGPVPSHVSGLDDCLPMVDHIEGCLLD | EPIC-XV1.6 |
| | LLSDVGQELPDLGDLGELLCETASPQGPMQSEGGEEG | |
| 78 | TGGLPSPVIFPHETQGLLAPPAGQSQTPFSPEGPVPSHVSGLDDCLPM | EPIC-XV1.7 |
| | VDHIEGCLLDLLSDVGQELPDLGDLGELLCETASPQG | |
| 2 | TPTDLCLPTGGLPSPVIFPHETQGLLAPPAGQSQTPFSPEGPVPSHVSG | EPIC-XV1.8 |
| | LDDCLPMVDHIEGCLLDLLSDVGQELPDLGDLGELL | |
| 12 | GGGSLDDCLPMVDHIEGCLLDLLSDVGQELPDLGDLGSAGSAAGSG | EPIC-XV1.9 |
| | EFGGSGSGSGGSGGSGSAGSAAGSGEFGGSGGSGSGGGS | |
| 13 | GSAGSAAGSGEFLDDCLPMVDHIEGCLLDLLSDVGQELPDLGDLGSA | EPIC-XV1.10 |
| | GSAAGSGEFGGSGSGSGGSGGSGSAGSAAGSGEFGSGS | |
| 14 | GSAGSAAGSGEFGSGSGSGSGSLDDCLPMVDHIEGCLLDLLSDVGQE | EPIC-XV1.11 |
| | LPDLGDLGSAGSAAGSGEFGGSGSGSAGSAAGSGEFGS | |
| 15 | GSAGSAAGSGEFGGSGSGSAGSAAGSGEFGSLDDCLPMVDHIEGCLL | EPIC-XV1.12 |
| | DLLSDVGQELPDLGDLGSAGSAAGSGEFGSGSGSGSGS | |
| 16 | GSAGSAAGSGEFGGSGSGSGGSGGSGSAGSAAGSGEFGSGSLDDCLP | EPIC-XV1.13 |
| | MVDHIEGCLLDLLSDVGQELPDLGDLGSAGSAAGSGEF | |
| 17 | GSAGSAAGSGEFGGSGSGSGGSGGSGSAGSAAGSGEFGGSGGSGSG | EPIC-XV1.14 |
| | GGSLDDCLPMVDHIEGCLLDLLSDVGQELPDLGDLGGGS | |
| 18 | GSAGSAAGSGEFLDDCLPMVDHIEGCLLDLLSDVGQELPDLGDLGGS | EPIC-XV1.27 |
| | GGSGGSLDDCLPMVDHIEGCLLDLLSDVGQELPDLGDL | |
| 19 | GSAGSAAGSGEFGGSGGGSLDDCLPMVDHIEGCLLDLLSDVGQELP | EPIC-XV1.28 |
| | DLGDLGSLDDCLPMVDHIEGCLLDLLSDVGQELPDLGDL | |
| 20 | LDDCLPMVDHIEGCLLDLLSDVGQELPDLGDLGSLDDCLPMVDHIEG | EPIC-XV1.29 |
| | CLLDLLSDVGQELPDLGDLGGGSGGSGSAGSAAGSGEF | |
| 21 | LDDCLPMVDHIEGCLLDLLSDVGQELPDLGDLGGSGGSGGSLDDCLP | EPIC-XV1.30 |
| | MVDHIEGCLLDLLSDVGQELPDLGDLGSAGSAAGSGEF | |
| 22 | LDDCLPMVDHIEGCLLDLLSDVGQELPDLGDLGSAGSAAGSGEFGSG | EPIC-XV1.31 |
| | SGSGGSLDDCLPMVDHIEGCLLDLLSDVGQELPDLGDL | |
| 23 | LDDCLPMVDHIEGCLLDLLSDVGQELPDLGDLGGSDALDDFDLDML | EPIC-XV1.32 |
| | GSDALDDFDLDMLGSDALDDFDLDMLGSDALDDFDLDML | |
| 24 | DALDDFDLDMLGSDALDDFDLDMLGSDALDDFDLDMLGSDALDDF | EPIC-XV1.33 |
| | DLDMLGGSLDDCLPMVDHIEGCLLDLLSDVGQELPDLGDL | |
| 25 | QGLLGESSNLPDLEVHMSGGPFWGGSQGLLGESSNLPDLEVHMSGG | EPIC-XV1.34 |
| | PFWGGGSLDDCLPMVDHIEGCLLDLLSDVGQELPDLGDL | |
| 26 | NVECFKKFACNQEYWEEFAASPIGSAGSAAGSGEFNVECFKKFACN | EPIC-XV1.35 |
| | QEYWEEFAASPILDDCLPMVDHIEGCLLDLLSDVGQELPDLGDL | |
| 27 | NVELYEEYFNFIVSSDTYFNGGSNVELYEEYFNFIVSSDTYFNGGGSG | EPIC-XV1.36 |
| | GGSGSLDDCLPMVDHIEGCLLDLLSDVGQELPDLGDL | |
| 28 | ANEKIIREDLPLSFYGSANEKIIREDLPLSFYGSANEKIIREDLPLSFYG | EPIC-XV1.37 |
| | GGSLDDCLPMVDHIEGCLLDLLSDVGQELPDLGDL | |
| 29 | GGGSANEKIIREDLPLSFYGGGSNVELYEEYFNFIVSSDTYFNGGGSN | EPIC-XV1.46 |
| | VECFKKFACNQEYWEEFAASPIGGGSQGLLGESSNLPDLEVHMSGGP | |
| | FWGGGSLDDCLPMVDHIEGCLLDLLSDVGQELPDLGDL | |
| 30 | GGGSLDDCLPMVDHIEGCLLDLLSDVGQELPDLGDLGGGSLDDCLP | EPIC-XV1.47 |
| | MVDHIEGCLLDLLSDVGQELPDLGDLGGGSLDDCLPMVDHIEGCLL | |
| | DLLSDVGQELPDLGDL | |
| 31 | GGSDALDDFDLDMLGGSLDDCLPMVDHIEGCLLDLLSDVGQELPDL | EPIC-XV1.48 |
| | GDLGGSDALDDFDLDMLGGSLDDCLPMVDHIEGCLLDLLSDVGQEL | |
| | PDLGDL | |
| 79 | LDDCLPMVDHIEGCLLDLLSDVGQELPDLGDLGGSDALDDFDLDML | CC.1 |
| | GSDALDDFDLDMLGSDALDDFDLDMLGSDALDDFDLDML | |
| 80 | LDDCLPMVDHIEGCLLDLLSDVGQELPDLGDLGSDALDDFDLDMLG | CC.2 |
| | SDALDDFDLDMLGSDALDDFDLDMLGSDALDDFDLDML | |
| 81 | LDDCLPMVDHIEGCLLDLLSDVGQELPDLGDLGDALDDFDLDMLGS | CC.3 |
| | DALDDFDLDMLGSDALDDFDLDMLGSDALDDFDLDML | |
| 82 | LDDCLPMVDHIEGCLLDLLSDVGQELPDLGDLDALDDFDLDMLGSD | CC.4 |
| | ALDDFDLDMLGSDALDDFDLDMLGSDALDDFDLDML | |
| 83 | LDDCLPMVDHIEGCLLDLLSDVGQELPDLGDLGSGSDALDDFDLDM | CC.5 |
| | LGSDALDDFDLDMLGSDALDDFDLDMLGSDALDDFDLDML | |
| 84 | LDDCLPMVDHIEGCLLDLLSDVGQELPDLGDLGGSGSDALDDFDLD | CC.6 |
| | MLGSDALDDFDLDMLGSDALDDFDLDMLGSDALDDFDLDML | |
| 85 | LDDCLPMVDHIEGCLLDLLSDVGQELPDLGDLGGSGGSDALDDFDL | CC.7 |
| | DMLGSDALDDFDLDMLGSDALDDFDLDMLGSDALDDFDLDML | |
| 86 | LDDCLPMVDHIEGCLLDLLSDVGQELPDLGDLGGSDALDDFDLDML | CC.8 |
| | GDALDDFDLDMLGDALDDFDLDMLGDALDDFDLDML | |
| 87 | LDDCLPMVDHIEGCLLDLLSDVGQELPDLGDLGGSDALDDFDLDML | CC.9 |
| | DALDDFDLDMLDALDDFDLDMLDALDDFDLDML | |
| 88 | LDDCLPMVDHIEGCLLDLLSDVGQELPDLGDLGGSDALDDFDLDML | CC.10 |
| | GGSDALDDFDLDMLGGSDALDDFDLDMLGGSDALDDFDLDML | |
| 89 | LDDCLPMVDHIEGCLLDLLSDVGQELPDLGDLGGSDALDDFDLDML | CC.11 |
| | GSGSDALDDFDLDMLGSGSDALDDFDLDMLGSGSDALDDFDLDML | |
| 90 | LDDCLPMVDHIEGCLLDLLSDVGQELPDLGDLGGSDALDDFDLDML | CC.12 |
| | GGSGSDALDDFDLDMLGGSGSDALDDFDLDMLGGSGSDALDDFDL | |
| | DML | |
| 91 | LDDCLPMVDHIEGCLLDLLSDVGQELPDLGDLGGSDALDDFDLDML | CC.13 |
| | GGSGGSDALDDFDLDMLGGSGGSDALDDFDLDMLGGSGGSDALDD | |
| | FDLDML | |
| 92 | LDDCLPMVDHIEGCLLDLLSDVGQELPDLGDLGGSDDFDLDLGSDD | CC.14 |
| | FDLDLGSDDFDLDLGSDDFDLDL | |
| 93 | LDDCLPMVDHIEGCLLDLLSDVGQELPDLGDLGGSDDFDLDLDDFD | CC.15 |
| | LDLDDFDLDLDDFDLDL | |
| 94 | LDDCLPMVDHIEGCLLDLLSDVGQELPDLGDLDLDLDDFDLDLGSD | CC.16 |
| | DFDLDLGSDDFDLDLGSDDFDLDL | |
| 95 | LDDCLPMVDHIEGCLLDLLSDVGQELPDLGDLDLDLDDFDLDLDDF | CC.17 |
| | DLDLDDFDLDLDDFDLDL | |
| 96 | LDDLLPMVDHIEGCLLDLLSDVGQELPDLGDLGGSDALDDFDLDML | CC.18 |
| | GSDALDDFDLDMLGSDALDDFDLDMLGSDALDDFDLDML | |
| 97 | LDDCDPMVDHIEGCLLDLLSDVGQELPDLGDLGGSDALDDFDLDML | CC.19 |
| | GSDALDDFDLDMLGSDALDDFDLDMLGSDALDDFDLDML | |
| 98 | LDDCLPDVDHIEGCLLDLLSDVGQELPDLGDLGGSDALDDFDLDML | CC.20 |
| | GSDALDDFDLDMLGSDALDDFDLDMLGSDALDDFDLDML | |
| 99 | LDDCLPMVDHIEGCLLDLDSDVGQELPDLGDLGGSDALDDFDLDML | CC.21 |
| | GSDALDDFDLDMLGSDALDDFDLDMLGSDALDDFDLDML | |
| 100 | LDDCLPMVDHIEGCLLDLLSDVGQLLPDLGDLGGSDALDDFDLDML | CC.22 |
| | GSDALDDFDLDMLGSDALDDFDLDMLGSDALDDFDLDML | |
| 101 | LDDCLPMVDHIEGCLLDLLSDVGQEDPDLGDLGGSDALDDFDLDML | CC.23 |
| | GSDALDDFDLDMLGSDALDDFDLDMLGSDALDDFDLDML | |
| 102 | LDDLDPDVDHIEGCLLDLDSDVGQLDPDLGDLGGSDALDDFDLDML | CC.24 |
| | GSDALDDFDLDMLGSDALDDFDLDMLGSDALDDFDLDML | |
| 103 | LDDLDPDVDHIEGCLLDLDSDVGQLDPDLGDLDLDLDDFDLDLDDF | CC.25 |
| | DLDLDDFDLDLDDFDLD | |
| 104 | LDDCLPMVDHIEGCLLDLLSDVGQELPDLGDLGGSGGSLDDCLPMV | CC.26 |
| | DHIEGCLLDLLSDVGQELPDLGDL | |
| 105 | GSAGSAAGSGEFLDDCLPMVDHIEGCLLDLLSDVGQELPDLGDLGGS | CC.27 |
| | GGSLDDCLPMVDHIEGCLLDLLSDVGQELPDLGDL | |
| 106 | GGSGGSLDDCLPMVDHIEGCLLDLLSDVGQELPDLGDLGGSGGSLD | CC.28 |
| | DCLPMVDHIEGCLLDLLSDVGQELPDLGDL | |
| 107 | LDDCLPMVDHIEGCLLDLLSDVGQELPDLGDLGGSGGSLDGLDPLEQ | CC.29 |
| | GVDSLLDLLCGEIHDVMPLCDDL | |
| 108 | GSAGSAAGSGEFLDDCLPMVDHIEGCLLDLLSDVGQELPDLGDLGGS | CC.30 |
| | GGSLDGLDPLEQGVDSLLDLLCGEIHDVMPLCDDL | |
| 109 | GGSGGSLDDCLPMVDHIEGCLLDLLSDVGQELPDLGDLGGSGGSLD | CC.31 |
| | GLDPLEQGVDSLLDLLCGEIHDVMPLCDDL | |
| 110 | LDDLDPDVDHIEGCLLDLDSDVGQLDPDLGDLGGSGGSLDDLDPDV | CC.32 |
| | DHIEGCLLDLDSDVGQLDPDLGDL | |
| 111 | GSAGSAAGSGEFLDDLDPDVDHIEGCLLDLDSDVGQLDPDLGDLGG | CC.33 |
| | SGGSLDDLDPDVDHIEGCLLDLDSDVGQLDPDLGDL | |
| 112 | GGSGGSLDDLDPDVDHIEGCLLDLDSDVGQLDPDLGDLGGSGGSLD | CC.34 |
| | DLDPDVDHIEGCLLDLDSDVGQLDPDLGDL | |
| 113 | LDDLDPDVDHIEGCLLDLDSDVGQLDPDLGDLGGSGGSLDGLDPDL | CC.35 |
| | QGVDSDLDLLCGEIHDVDPDLDDL | |
| 114 | GSAGSAAGSGEFLDDLDPDVDHIEGCLLDLDSDVGQLDPDLGDLGG | CC.36 |
| | SGGSLDGLDPDLQGVDSDLDLLCGEIHDVDPDLDDL | |
| 115 | GGSGGSLDDLDPDVDHIEGCLLDLDSDVGQLDPDLGDLGGSGGSLD | CC.37 |
| | GLDPDLQGVDSDLDLLCGEIHDVDPDLDDL | |
| 116 | LDDCLPMVDHIEGCLLDLLSDVGQELPDLGDLGGSGGSLDGLDPLEQ | CC.38 |
| | GVDSLLDLLCGEIHDVMPLCDDLGGSGGSLDDCLPMVDHIEGCLLD | |
| | LLSDVGQELPDLGDL | |
| 117 | LDDLDPDVDHIEGCLLDLDSDVGQLDPDLGDLGGSGGSLDDLDPDV | CC.39 |
| | DHIEGCLLDLDSDVGQLDPDLGDLGGSGGSLDDLDPDVDHIEGCLLD | |
| | LDSDVGQLDPDLGDL | |
| 118 | LDDLDPDVDHIEGCLLDLDSDVGQLDPDLGDLGGSGGSLDGLDPDL | CC.40 |
| | QGVDSDLDLLCGEIHDVDPDLDDLGGSGGSLDDLDPDVDHIEGCLL | |
| | DLDSDVGQLDPDLGDL | |
| 119 | LDDCLPMVDHIEGCLLDLLSDVGQELPDLGDLGGSDALDDFDLDML | CC.41 |
| | GSLMDLDFDDLADGGSGGSDALDDFDLDMLGSLMDLDFDDLAD | |
| 120 | LDDCLPMVDHIEGCLLDLLSDVGQELPDLGDLGGSDALDDFDLDML | CC.42 |
| | GSDALDDFDLDMLGGSGGSLMDLDFDDLADGSLMDLDFDDLAD | |
| 121 | LDDCLPMVDHIEGCLLDLLSDVGQELPDLGDLGGSDALDDFDLDML | CC.43 |
| | GSDALDDFDLDMLGGSGGSLMDLDFDDLADGSLMDLDFDDLADGS | |
| | LDGLDPLEQGVDSLLDLLCGEIHDVMPLCDDL | |
| 122 | GGSDALDDFDLDMLGSLDDCLPMVDHIEGCLLDLLSDVGQELPDLG | CC.44 |
| | DLGSDALDDFDLDMLGGSGGSDALDDFDLDMLGSLDDCLPMVDHI | |
| | EGCLLDLLSDVGQELPDLGDLGSDALDDFDLDML | |
| 123 | GGSDALDDFDLDMLGSLDDCLPMVDHIEGCLLDLLSDVGQELPDLG | CC.45 |
| | DLGSDALDDFDLDMLGGSGGSLMDLDFDDLADGSLDGLDPLEQGV | |
| | DSLLDLLCGEIHDVMPLCDDLGSLMDLDFDDLAD | |
| 124 | LDDCLPMVDHIEGCLLDLLSDVGQELPDLGDLGGSLDDFDLDGSLD | CC.46 |
| | DFDLDGGSGGSDLDFDDLGSDLDFDDLGSLDGLDPLEQGVDSLLDL | |
| | LCGEIHDVMPLCDDL | |
| 125 | GGSLDDFDLDGSLDDCLPMVDHIEGCLLDLLSDVGQELPDLGDLGSL | CC.47 |
| | DDFDLDGGSGGSLDDFDLDGSLDDCLPMVDHIEGCLLDLLSDVGQE | |
| | LPDLGDLGSLDDFDLD | |
| 126 | GGSLDDFDLDGSLDDCLPMVDHIEGCLLDLLSDVGQELPDLGDLGSL | CC.48 |
| | DDFDLDGGSGGSDLDFDDLGSLDGLDPLEQGVDSLLDLLCGEIHDV | |
| | MPLCDDLGSDLDFDDL | |
| 127 | LDDFDLDLDDFDLDLDDFDLDLDDFDLDGSAGSAAGSGEFLDDFDL | CC.49 |
| | DLDDFDLDLDDFDLDLDDFDLD | |
| 128 | GSAGSAAGSGEFLDDFDLDLDDFDLDLDDFDLDLDDFDLDGSAGSA | CC.50 |
| | AGSGEFLDDFDLDLDDFDLDLDDFDLDLDDFDLD | |
| 129 | GGSGGSLDDFDLDLDDFDLDLDDFDLDLDDFDLDGSAGSAAGSGEF | CC.51 |
| | LDDFDLDLDDFDLDLDDFDLDLDDFDLD | |
| 130 | LDDFDLDLDDFDLDLDDFDLDLDDFDLDGSAGSAAGSGEFDLDFDD | CC.52 |
| | LDLDFDDLDLDFDDLDLDFDDL | |
| 131 | GSAGSAAGSGEFLDDFDLDLDDFDLDLDDFDLDLDDFDLDGSAGSA | CC.53 |
| | AGSGEFDLDFDDLDLDFDDLDLDFDDLDLDFDDL | |
| 132 | GGSGGSLDDFDLDLDDFDLDLDDFDLDLDDFDLDGSAGSAAGSGEF | CC.54 |
| | DLDFDDLDLDFDDLDLDFDDLDLDFDDL | |
| 133 | LDDLDPDVDHIEGCLLDLDSDVGQLDPDLGDLDLDLDDFDLDLDDF | CC.55 |
| | DLDLDDFDLDLDDFDLDGGSGGSLDDLDPDVDHIEGCLLDLDSDVG | |
| | QLDPDLGDLDLDLDDFDLDLDDFDLDLDDFDLDLDDFDLD | |
| 134 | GSAGSAAGSGEFLDDLDPDVDHIEGCLLDLDSDVGQLDPDLGDLDL | CC.56 |
| | DLDDFDLDLDDFDLDLDDFDLDLDDFDLDGGSGGSLDDLDPDVDHI | |
| | EGCLLDLDSDVGQLDPDLGDLDLDLDDFDLDLDDFDLDLDDFDLDL | |
| | DDFDLD | |
| 135 | GGSGGSLDDLDPDVDHIEGCLLDLDSDVGQLDPDLGDLDLDLDDFD | CC.57 |
| | LDLDDFDLDLDDFDLDLDDFDLDGGSGGSLDDLDPDVDHIEGCLLD | |
| | LDSDVGQLDPDLGDLDLDLDDFDLDLDDFDLDLDDFDLDLDDFDLD | |
| 136 | LDDLDPDVDHIEGCLLDLDSDVGQLDPDLGDLDLDLDDFDLDLDDF | CC.58 |
| | DLDLDDFDLDLDDFDLDGGSGGSDLDFDDLDLDFDDLDLDFDDLDL | |
| | DFDDLLDLDLDGLDPDLQGVDSDLDLLCGEIHDVDPDLDDL | |
| 137 | GSAGSAAGSGEFLDDLDPDVDHIEGCLLDLDSDVGQLDPDLGDLDL | CC.59 |
| | DLDDFDLDLDDFDLDLDDFDLDLDDFDLDGGSGGSDLDFDDLDLDF | |
| | DDLDLDFDDLDLDFDDLLDLDLDGLDPDLQGVDSDLDLLCGEIHDV | |
| | DPDLDDL | |
| 138 | GGSGGSLDDLDPDVDHIEGCLLDLDSDVGQLDPDLGDLDLDLDDFD | CC.60 |
| | LDLDDFDLDLDDFDLDLDDFDLDGGSGGSDLDFDDLDLDFDDLDLD | |
| | FDDLDLDFDDLLDLDLDGLDPDLQGVDSDLDLLCGEIHDVDPDLDD | |
| | L | |

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of a member selected from SEQ ID NOs: 19, 20, 23, 24, 30, and 31 (e.g., a member selected from SEQ ID NOs: 23, 24, and 31).

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 19.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 20.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 23.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 24.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 30.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 31.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of a member selected from SEQ ID NOs: 79-84, 86-92, 94-103, 116, 119-120, 122, 123, and 125 (e.g., a member selected from SEQ ID NOs: 79-84, 88, 90, 91 and 95).

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 79.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 80.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 81.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 82.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 83.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 84.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 86.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 87.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 88.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 89.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 90.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 91.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 92.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 94.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 95.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 96.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 97.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 98.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 99.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 100.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 101.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 102.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 103.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 116.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 119.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 120.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 122.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 123.

In some embodiments, the engineered gene effector as disclosed herein (e.g., engineered gene activator) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 125.

In some embodiments, the engineered gene effector as disclosed herein (e.g., engineered gene repressor) can comprise at least a portion of the polypeptide sequence of SEQ ID NO: 1. In some embodiments, the engineered gene effector as disclosed herein (e.g., engineered gene repressor) may not and need not comprise the polypeptide sequence of SEQ ID NO: 1.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of a member from Table 3 (e.g., a member selected from SEQ ID NOs: 32-51).

TABLE 3

| SEQ ID | | |
|---|---|---|
| NO: | Amino Acid Sequence | Name |
| 32 | HVSGLDDCLPMVDHIEGCLLDLLSDVGQELPDLGDL | EPIC-XV1.15 |
| 33 | SPEGPVPSHVSGLDDCLPMVDHIEGCLLDLLSDVGQELPDLGDL | EPIC-XV1.16 |
| 34 | PPAGQSQTPFSPEGPVPSHVSGLDDCLPMVDHIEGCLLDLLSDVGQELP | EPIC-XV1.17 |
| | DLGDL | |
| 35 | PHETQGLLAPPAGQSQTPFSPEGPVPSHVSGLDDCLPMVDHIEGCLLDL | EPIC-XV1.18 |
| | LSDVGQELPDLGDL | |
| 36 | TGGLPSPVIFPHETQGLLAPPAGQSQTPFSPEGPVPSHVSGLDDCLPMV | EPIC-XV1.19 |
| | DHIEGCLLDLLSDVGQELPDLGDL | |
| 37 | TPTDLCLPTGGLPSPVIFPHETQGLLAPPAGQSQTPFSPEGPVPSHVSGL | EPIC-XV1.20 |
| | DDCLPMVDHIEGCLLDLLSDVGQELPDLGDL | |
| 38 | GGGSLDDCLPMVDHIEGCLLDLLSDVGQELPDLGDL | EPIC-XV1.21 |
| 39 | GSAGSAAGSGEFLDDCLPMVDHIEGCLLDLLSDVGQELPDLGDL | EPIC-XV1.22 |
| 40 | GSAGSAAGSGEFGGGSGSGGGSLDDCLPMVDHIEGCLLDLLSDVGQE | EPIC-XV1.23 |
| | LPDLGDL | |
| 41 | GSAGSAAGSGEFGGSGSGSAGSAAGSGEFGSLDDCLPMVDHIEGCLLD | EPIC-XV1.24 |
| | LLSDVGQELPDLGDL | |
| 42 | GSAGSAAGSGEFGGSGSGSGGSGSGSAGSAAGSGEFGSGSLDDCLPM | EPIC-XV1.25 |
| | VDHIEGCLLDLLSDVGQELPDLGDL | |
| 43 | GSAGSAAGSGEFGGSGSGSGGSGGSGSAGSAAGSGEFGGSGGSGSGG | EPIC-XV1.26 |
| | GSLDDCLPMVDHIEGCLLDLLSDVGQELPDLGDL | |
| 44 | RHDPVLRCDMFEKHHKPRPKRSRKRSIDHESCASSGDTVANESGPLCT | EPIC-XV1.38 |
| | NTFWTPGPVLQGLLGESSNLPDLEVHMSGGPFWKEVY | |
| 45 | PSKLRSYPKQHAYHAPSIRSAVPSPFQNTLQNVLAAATKRNCNVTQM | EPIC-XV1.39 |
| | RELPTLDSAVFNVECFKKFACNQEYWEEFAASPIRITT | |
| 46 | DKDSKEIVSILTKFVNVISNIHSKYKCKYMFVGIPAIILFNKLDSTDVQK | EPIC-XV1.40 |
| | LYSLFSTKLNTNVELYEEYFNFIVSSDTYFNKKKF | |
| 47 | GSAGSAAGSGEFSGGSGSGGSGSAGSAAGSGEFMGAYTSIMNINDFNK | EPIC-XV1.41 |
| | IYAYKQDYRCKYNVSYELANEKIIREDLPLSFYIESF | |
| 48 | GGGSQGLLGESSNLPDLEVHMSGGPFWGSGSQGLLGESSNLPDLEVH | EPIC-XV1.42 |
| | MSGGPFWGGGSQGLLGESSNLPDLEVHMSGGPFWGSGS | |
| 49 | GGGSNVECFKKFACNQEYWEEFAASPIGSGSNVECFKKFACNQEYWE | EPIC-XV1.43 |
| | EFAASPIGSGSNVECFKKFACNQEYWEEFAASPIGSGS | |
| 50 | GGGSGGSGSGGGSGSGSNVELYEEYFNFIVSSDTYFNGGGSNVELYEE | EPIC-XV1.44 |
| | YFNFIVSSDTYFNGGGSNVELYEEYFNFIVSSDTYFN | |
| 51 | GGGSGGSGSANEKIIREDLPLSFYGGGSANEKIIREDLPLSFYGGGSAN | EPIC-XV1.45 |
| | EKIIREDLPLSFYGGGSANEKIIREDLPLSFYGGGS | |

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of a member selected from SEQ ID NOs: 32-35, 38, and 39.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 32.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 33.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 34.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 35.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 38.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 39.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of a member selected from SEQ ID NOs: 44-51.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 44.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 45.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 46.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 47.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 48.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 49.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 50.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 51.

In some embodiments, the engineered gene effector as disclosed herein (e.g., engineered gene repressor) can comprise one or more members selected from the group consisting of (i) at least a portion of the polypeptide sequence of SEQ ID NO: 52, (ii) at least a portion of the polypeptide sequence of SEQ ID NO: 55, (iii) at least a portion of the polypeptide sequence of SEQ ID NO: 58, and (iv) at least a portion of the polypeptide sequence of SEQ ID NO: 61.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 52.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 53. For example, the amino acid sequence of the polypeptide of the engineered gene effector may not and need not be identical to the polypeptide sequence of SEQ ID NO: 53.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 54. For example, the amino acid sequence of the polypeptide of the engineered gene effector may not and need not be identical to the polypeptide sequence of SEQ ID NO: 54.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 55.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 56. For example, the amino acid sequence of the polypeptide of the engineered gene effector may not and need not be identical to the polypeptide sequence of SEQ ID NO: 56.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 57. For example, the amino acid sequence of the polypeptide of the engineered gene effector may not and need not be identical to the polypeptide sequence of SEQ ID NO: 57.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 58.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 59. For example, the amino acid sequence of the polypeptide of the engineered gene effector may not and need not be identical to the polypeptide sequence of SEQ ID NO: 59.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 60. For example, the amino acid sequence of the polypeptide of the engineered gene effector may not and need not be identical to the polypeptide sequence of SEQ ID NO: 60.

In some embodiments, the engineered gene effector as disclosed herein (e.g., engineered gene repressor) can comprise an amino acid sequence (e.g., a consecutive polypeptide sequence) having at least or up to about 50%, at least or up to about 55%, at least or up to about 60%, at least or up to about 62%, at least or up to about 64%, at least or up to about 65%, at least or up to about 66%, at least or up to about 68%, at least or up to about 70%, at least or up to about 72%, at least or up to about 74%, at least or up to about 75%, at least or up to about 76%, at least or up to about 78%, at least or up to about 80%, at least or up to about 82%, at least or up to about 84%, at least or up to about 85%, at least or up to about 86%, at least or up to about 88%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 61.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 62. For example, the amino acid sequence of the polypeptide of the engineered gene effector may not and need not be identical to the polypeptide sequence of SEQ ID NO: 62.

In some embodiments, the amino acid sequence of the polypeptide of the engineered gene effector as disclosed herein can be at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, or at most about 10% identical to the polypeptide sequence of SEQ ID NO: 63. For example, the amino acid sequence of the polypeptide of the engineered gene effector may not and need not be identical to the polypeptide sequence of SEQ ID NO: 63.

In some embodiments, the engineered gene effector can be enriched for hydrophobic amino acid residues (e.g., valine, leucine, isoleucine, methionine, proline) and/or acidic amino acid residues (e.g., aspartic acid, glutamic acid), as compared to other types of amino acid residues, such as aromatic amino acid residues (e.g., phenylalanine, tyrosine, tryptophan), neutral amino acid residues (e.g., asparagine, glutamine, glycine, alanine, etc.), and/or basic amino acid residues (e.g., histidine, lysine, arginine). Alternatively, in some embodiments, the engineered gene effector can be enriched for one or more of the aromatic amino acid residues, neutral amino acid residues, and/or basic amino acid residues, as compared to the hydrophobic amino acid residues and/or acidic amino acid residues.

In some embodiments, the polypeptide of the engineered gene effector can be enriched for hydrophobic amino acid residues (e.g., valine, leucine, isoleucine, methionine, proline) and/or acidic amino acid residues (e.g., aspartic acid, glutamic acid), as compared to other types of amino acid residues, such as aromatic amino acid residues (e.g., phenylalanine, tyrosine, tryptophan), neutral amino acid residues (e.g., asparagine, glutamine, glycine, alanine, etc.), and/or basic amino acid residues (e.g., histidine, lysine, arginine). Alternatively, in some embodiments, the polypeptide of the engineered gene effector can be enriched for one or more of the aromatic amino acid residues, neutral amino acid residues, and/or basic amino acid residues, as compared to the hydrophobic amino acid residues and/or acidic amino acid residues.

In some embodiments, the additional polypeptide of the engineered gene effector can be enriched for hydrophobic amino acid residues (e.g., valine, leucine, isoleucine, methionine, proline) and/or acidic amino acid residues (e.g., aspartic acid, glutamic acid), as compared to other types of amino acid residues, such as aromatic amino acid residues (e.g., phenylalanine, tyrosine, tryptophan), neutral amino acid residues (e.g., asparagine, glutamine, glycine, alanine, etc.), and/or basic amino acid residues (e.g., histidine, lysine, arginine). Alternatively, in some embodiments, the additional polypeptide of the engineered gene effector can be enriched for one or more of the aromatic amino acid residues, neutral amino acid residues, and/or basic amino acid residues, as compared to the hydrophobic amino acid residues and/or acidic amino acid residues.

Sequences

SEQ ID NO: 1 (vIRF2 domain 1)

| | |
|---|---|
| 1 | LDDCLPMVDH IEGCLLDLLS DVGQELPDLG DL |

SEQ ID NO: 2 (vIRF2 domain 2)

| | |
|---|---|
| 1 | TPTDLCLPTG GLPSPVIFPH ETQGLLAPPA GQSQTPFSPE GPVPSHVSGL |
| 51 | DDCLPMVDHI EGCLLDLLSD VGQELPDLGD LGELL |

SEQ ID NO: 3 (vIRF2 domain 3)

| | |
|---|---|
| 1 | PPAGQSQTPF SPEGPVPSHV SGLDDCLPMV DHIEGCLLDL LSDVGQELPD |
| 51 | LGDLGELLCE TASPQGPMQS EGGEEGSTES VSVLP |

SEQ ID NO: 4 (wild type viral IRF2 or “vIRF2”)

| | |
|---|---|
| 1 | MPRYTESEWL TDFIIDALDS GRFWGVGWLD EQKRIFTVPG RNRRERMPEG |
| 51 | FDDFYEAFLE ERRRHGLPEI PETETGLGCF GRLLRTANRA RQERPFTIYK |
| 101 | GKMKLNRWIM TPRPYKGCEG CLVYLTQEPA MKNMLKALFG IYPHDDKHRE |
| 151 | KALRRSLRKK AQREAARKQA AAVATPTTSS AAEVSSRSQS EDTESSDSEN |
| 201 | ELWVGAQGFV GRDMHSLFFE EPEPSGFGSS GQSSSLLAPD SPRPSTSQVQ |
| 251 | GPLHVHTPTD LCLPTGGLPS PVIFPHETQG LLAPPAGQSQ TPFSPEGPVP |
| 301 | SHVSGLDDCL PMVDHIEGCL LDLLSDVGQE LPDLGDLGEL LCETASPQGP |
| 351 | MQSEGGEEGS TESVSVLPAT HPLESSAPGA SVMGSGQELP DLGDLSELLC |
| 401 | ETASPQGPMQ SEGGEEGSTE SVSVLPATHP LESSAPGASV MGSSFQASDN |
| 451 | VDDFIDCIPP LCRDDRDVED QEKADQTFYW YGSDMRPKVL TATQSVAAYL |
| 501 | SKKQAIYKVG DKLVPLVVEV YYFGEKVKTH FDLTGGIVIC SQVPEASPEH |
| 551 | ICQTVPPYKC LLPRTAHCSV DANRTLEQTL DRFSMGVVAI GTNMGIFLKG |
| 601 | LLEYPAYFVG NASRRRIGKC RPLSHRHEIQ QAFDVERHNR EPEGSRYASL |
| 651 | FLGRRPSPEY DWDHYPVILH IYLAPFYHRD |

SEQ ID NO: 5 (VP16)

| | |
|---|---|
| 1 | DALDDFDLDM L |

SEQ ID NO: 6 (VP64)

| | |
|---|---|
| 1 | DALDDFDLDM LGSDALDDFD LDMLGSDALD DFDLDMLGSD ALDDFDLDML |

SEQ ID NO: 7 (p65)

| | |
|---|---|
| 1 | QYLPDTDDRH RIEEKRKRTY ETFKSIMKKS PFSGPTDPRP PPRRIAVPSR |
| 51 | SSASVPKPAP QPYPFTSSLS TINYDEFPTM VFPSGQISQA SALAPAPPQV |
| 101 | LPQAPAPAPA PAMVSALAQA PAPVPVLAPG PPQAVAPPAP KPTQAGEGTL |
| 151 | SEALLQLQFD DEDLGALLGN STDPAVFTDL ASVDNSEFQQ LLNQGIPVAP |
| 201 | HTTEPMLMEY PEAITRLVTG AQRPPDPAPA PLGAPGLPNG LLSGDEDFSS |
| 251 | IADMDFSALL SQISS |

SEQ ID NO: 8 (Rta)

| | |
|---|---|
| 1 | RDSREGMFLP KPEAGSAISD VFEGREVCQP KRIRPFHPPG SPWANRPLPA |
| 51 | SLAPTPTGPV HEPVGSLTPA PVPQPLDPAP AVTPEASHLL EDPDEETSQA |
| 101 | VKALREMADT VIPQKEEAAI CGQMDLSHPP PRGHLDELTT TLESMTEDLN |
| 151 | LDSPLTPELN EILDTFLNDE CLLHAMHIST GLSIFDTSLF |

SEQ ID NO: 9 (VP64-p65-Rta or “VPR” with linker sequences underlined)

| | |
|---|---|
| 1 | DALDDFDLDM LGSDALDDFD LDMLGSDALD DFDLDMLGSD ALDDFDLDML |
| 51 | GSGGSGSQYL PDTDDRHRIE EKRKRTYETF KSIMKKSPFS GPTDPRPPPR |
| 101 | RIAVPSRSSA SVPKPAPQPY PFTSSLSTIN YDFFPTMVFP SGQISQASAL |
| 151 | APAPPQVLPQ APAPAPAPAM VSALAQAPAP VPVLAPGPPQ AVAPPAPKPT |
| 201 | QAGFGTLSEA LLQLQFDDED LGALLGNSTD PAVFTDLASV DNSEFQQLLN |
| 251 | QGIPVAPHTT EPMLMEYPEA ITRLVTGAQR PPDPAPAPLG APGLPNGLLS |
| 301 | GDEDFSSIAD MDFSALLSQI SSGSGSGSRD SREGMFLPKP EAGSAISDVF |
| 351 | EGREVCQPKR IRPFHPPGSP WANRPLPASL APTPTGPVHE PVGSLTPAPV |
| 401 | PQPLDPAPAV TPEASHLLED PDEETSQAVK ALREMADTVI PQKEEAAICG |
| 451 | QMDLSHPPPR GHLDELTTTL ESMTEDLNLD SPLTPELNEI LDTFLNDECL |
| 501 | LHAMHISTGL SIFDTSLF |

SEQ ID NO: 10 (Un1Cas12fl or “CasMini”)

| | |
|---|---|
| 1 | MAKNTITKTL KLRIVRPYNS AEVEKIVADE KNNREKIALE KNKDKVKEAC |
| 51 | SKHLKVAAYC TTQVERNACL FCKARKLDDK FYQKLRGQFP DAVFWQEISE |
| 101 | IFRQLQKQAA EIYNQSLIEL YYEIFIKGKG IANASSVEHY LSDVCYTRAA |
| 151 | ELFKNAAIAS GLRSKIKSNF RLKELKNMKS GLPTTKSDNF PIPLVKQKGG |
| 201 | QYTGFEISNH NSDFIIKIPF GRWQVKKEID KYRPWEKFDF EQVQKSPKPI |
| 251 | SLLLSTQRRK RNKGWSKDEG TEAEIKKVMN GDYQTSYIEV KRGSKIGEKS |
| 301 | AWMLNLSIDV PKIDKGVDPS IIGGIDVGVK SPLVCAINNA FSRYSISDND |
| 351 | LFHFNKKMFA RRRILLKKNR HKRAGHGAKN KLKPITILTE KSERFRKKLI |
| 401 | ERWACEIADF FIKNKVGTVQ MENLESMKRK EDSYFNIRLR GFWPYAEMQN |
| 451 | KIEFKLKQYG IEIRKVAPNN TSKTCSKCGH LNNYFNFEYR KKNKFPHFKC |
| 501 | EKCNFKENAD YNAALNISNP KLKSTKEEP |

SEQ ID NO: 11 (deactivated nuclease variant of Un1Cas12flor “dCasMini”)

| | |
|---|---|
| 1 | MAKNTITKTL KLRIVRPYNS AEVEKIVADE KNNREKIALE KNKDKVKEAC |
| 51 | SKHLKVAAYC TTQVERNACL FCKARKLDDK FYQKLRGQFP DAVFWQEISE |
| 101 | IFRQLQKQAA EIYNQSLIEL YYEIFIKGKG IANASSVEHY LSRVCYRRAA |
| 151 | ELFKNAAIAS GLRSKIKSNF RLKELKNMKS GLPTTKSDNF PIPLVKQKGG |
| 201 | QYTGFEISNH NSDFIIKIPF GRWQVKKEID KYRPWEKFDF EQVQKSPKPI |
| 251 | SLLLSTQRRK RNKGWSKDEG TEAEIKKVMN GDYQTSYIEV KRGSKICEKS |
| 301 | AWMLNLSIDV PKIDKGVDPS IIGGIAVGVR SPLVCAINNA FSRYSISDND |
| 351 | LFHFNKKMFA RRRILLKKNR HKRAGHGAKN KLKPITILTE KSERFRKKLI |
| 401 | ERWACEIADF FIKNKVGTVQ MENLESMKRK EDSYFNIRLR GFWPYAEMQN |
| 451 | KIEFKLKQYG IEIRKVAPNN TSKTCSKCGH LNNYFNFEYR KKNKFPHFKC |
| 501 | EKCNFKENAA YNAALNISNP KLKSTKERP |

SEQ ID NO: 52 (Bel-1 domain 1)

| | |
|---|---|
| 1 | QGLLGESSNL PDLEVHMSGG PEW |

SEQ ID NO: 53 (Bel-1 domain 2)

| | |
|---|---|
| 1 | RHDPVLRCDM FEKHHKPRPK RSRKRSIDHE SCASSGDTVA NESGPLCTNT |
| 51 | FWTPGPVLQG LLGESSNLPD LEVHMSGGPF WKEVY |

SEQ ID NO: 54 (wild type Bel-1)

| | |
|---|---|
| 1 | MDSYQEEEPV ASTSGLQDLQ TLSELVGPEN AGEGDLVIAE EPEENPRRPR |
| 51 | RYTKRDVKCV SYHAYKELED KHPHHIKLQD WIPKPEEMSK SICKRLILCG |
| 101 | LYSGEKAREI LKKPFTVSWE QSETNPDCFI VSYTCIFCDA VIHDPMPVVW |
| 151 | DSEVEIWVKY KPLRGIVGSA VFIMEKHQKN CSLVKPSTSC PEGPKPRRRH |
| 201 | DPVLRCDMFE KHHKPRPKRS RKRSIDHESC ASSGDTVANE SGPLCTNTFW |
| 251 | TPGPVLQGLL GESSNLPDLE VHMSGGPFWK EVYGDSILGP PSGSGEHSVL |

SEQ ID NO: 55 (Chikungunya domain 1)

| | |
|---|---|
| 1 | NVECFKKFAC NQEYWEEFAA SPI |

SEQ ID NO: 56 (Chikungunya domain 2)

| | |
|---|---|
| 1 | PSKLRSYPKQ HAYHAPSIRS AVPSPFQNTL QNVLAAATKR NCNVTQMREL |
| 51 | PTLDSAVFNV ECFKKFACNQ EYWEEFAASP IRITT |

SEQ ID NO: 57 (wild type Chikungunya)

| | |
|---|---|
| 1 | MDPVYVDIDA DSAFLKALQR AYPMFEVEPR QVTPNDHANA RAFSHLAIKL |
| 51 | IEQEIDPDST ILDIGSAPAR RMMSDRKYHC VCPMRSAEDP ERLANYARKL |
| 101 | ASAAGKVLDR NISGKIGDLQ AVMAVPDTET PTFCLHTDVS CRQRADVAIY |
| 151 | QDVYAVHAPT SLYHQAIKGV RLAYWVGFDT TPFMYNAMAG AYPSYSTNWA |
| 201 | DEQVLKAKNI GLCSTDLTEG RRGKLSIMRG KKLEPCDRVL FSVGSTLYPE |
| 251 | SRKLLKSWHL PSVFHLKGKL SFTCRCDTVV SCEGYVVKRI TMSPGLYGKT |
| 301 | TGYAVTHHAD GFLMCKTTDT VDGERVSFSV CTYVPATICD QMTGILATEV |
| 351 | TPEDAQKLLV GLNQRIVVNG RTQRNTNTMK NYMIPVVAQA FSKWAKECRK |
| 401 | DMEDEKLLGV RERTLTCCCL WAFKKQKTHT VYKRPDTQSI QKVQAEFDSF |
| 451 | VVPSLWSSGL SIPLRTRIKW LLSKVPKTDL TPYSGDAQEA RDAEKEAEEE |
| 501 | REAELTLEAL PPLQAAQEDV QVEIDVEQLE DRAGAGIIET PRGAIKVTAQ |
| 551 | PTDHVVGEYL VLSPQTVLRS QKLSLIHALA EQVKTCTHSG RAGRYAVEAY |
| 601 | DGRVLVPSGY AISPEDFQSL SESATMVYNE REFVNRKLHH IAMHGPALNT |
| 651 | DEESYELVRA ERTEHEYVYD VDQRRCCKKE EAAGLVLVGD LTNPPYHEFA |
| 701 | YEGLKIRPAC PYKIAVIGVF GVPGSGKSAI IKNLVTRQDL VTSGKKENCQ |
| 751 | EITTDVMRQR GLEISARTVD SLLLNGCNRP VDVLYVDEAF ACHSGTLLAL |
| 801 | IALVRPRQKV VLCGDPKQCG FENMMQMKVN YNHNICTQVY HKSISRRCTL |
| 851 | PVTAIVSSLH YEGKMRTTNE YNKPIVVDTT GSTKPDPGDL VLTCFRGWVK |
| 901 | QLQIDYRGHE VMTAAASQGL TRKGVYAVRQ KVNENPLYAS TSEHVNVLLT |
| 951 | RTEGKLVWKT LSGDPWIKTL QNPPKGNFKA TIKEWEVEHA SIMAGICSHQ |
| 1001 | MTFDTFQNKA NVCWAKSLVP ILETAGIKLN DRQWSQIIQA FKEDKAYSPE |
| 1051 | VALNEICTRM YGVDLDSGLF SKPLVSVYYA DNHWDNRPGG KMFGFNPEAA |
| 1101 | SILERKYPFT KGKWNINKQI CVTTRRIEDF NPTTNIIPAN RALPHSLVAE |
| 1151 | HRPVKGERME WLVNKINGHH VLLVSGCSLA LPTKRVTWVA PLGVRGADYT |
| 1201 | YNLELGLPAT LGRYDLVVIN IHTPFRIHHY QQCVDHAMKL QMLGGDSLRL |
| 1251 | LKPGGSLLIR AYGYADRTSE RVICVLGRKF RSSRALKPPC VTSNTEMFFL |
| 1301 | FSNFDNGRRN FTTHVMNNQL NAAFVGQATR AGCAPSYRVK RMDIAKNDEE |
| 1351 | CVVNAANPRG LPGDGVCKAV YKKWPESFKN SATPVGTAKT VMCGTYPVIH |
| 1401 | AVGPNFSNYS ESEGDRELAA AYREVAKEVT RLGVNSVAIP LLSTGVYSGG |
| 1451 | KDRLTQSLNH LFTAMDSTDA DVVIYCRDKE WEKKISEAIQ MRTQVELLDE |
| 1501 | HISIDCDVVR VHPDSSLAGR KGYSTTEGAL YSYLEGTRFH QTAVDMAEIY |
| 1551 | TMWPKQTEAN EQVCLYALGE SIESIRQKCP VDDADASSPP KTVPCLCRYA |
| 1601 | MTPERVTRLR MNHVTSIIVC SSFPLPKYKI EGVQKVKCSK VMLFDHNVPS |
| 1651 | RVSPREYRPS QESVQEASTT TSLTHSQFDL SVDGKILPVP SDLDADAPAL |
| 1701 | EPALDDGAIH TLPSATGNLA AVSDWVMSTV PVAPPRRRRG RNLTVTCDER |
| 1751 | EGNITPMASV RFFRAELCPV VQETAETRDT AMSLQAPPST ATELSHPPIS |
| 1801 | FGAPSETFPI TFGDFNEGEI ESLSSELLTF GDFLPGEVDD LTDSDWSTCS |
| 1851 | DTDDELRLDR AGGYIFSSDT GPGHLQQKSV RQSVLPVNTL EEVHEEKCYP |
| 1901 | PKLDEAKEQL LLKKLQESAS MANRSRYQSR KVENMKATII QRLKRGCRLY |
| 1951 | LMSETPKVPT YRTTYPAPVY SPPINVRLSN PESAVAACNE FLARNYPTVS |
| 2001 | SYQITDEYDA YLDMVDGSES CLDRATFNPS KLRSYPKQHA YHAPSIRSAV |
| 2051 | PSPFQNTLQN VLAAATKRNC NVTQMRELPT LDSAVFNVEC FKKFACNQEY |
| 2101 | WEEFAASPIR ITTENLTTYV TKLKGPKAAA LFAKTHNLLP LQEVPMDRFT |
| 2151 | VDMKRDVKVT PGTKHTEERP KVQVIQAAEP LATAYLCGIH RELVRRLNAV |
| 2201 | LLPNVHTLFD MSAEDFDAII AAHFKPGDTV LETDIASFDK SQDDSLALTA |
| 2251 | LMLLEDLGVD HSLLDLIEAA FGEISSCHLP TGTRFKFGAM MKSGMFLTLF |
| 2301 | VNTLLNITIA SRVLEDRLTK SACAAFIGDD NIIHGVVSDE LMAARCATWM |
| 2351 | NMEVKIIDAV VSQKAPYFCG GFILHDIVTG TACRVADPLK RLFKLGKPLA |
| 2401 | AGDEQDEDRR RALADEVVRW QRTGLIDELE KAVYSRYEVQ GISVVVMSMA |
| 2451 | TFASSRSNFE KLRGPVVTLY GGPK |

SEQ ID NO: 58 (Eptv1 domain 1)

| | |
|---|---|
| 1 | NVELYEEYFN FIVSSDTYFN |

SEQ ID NO: 59 (Eptv1 domain 2)

| | |
|---|---|
| 1 | DKDSKEIVSI LTKFVNVISN IHSKYKCKYM FVGIPAIILF NKLDSTDVQK |
| 51 | LYSLFSTKLN TNVELYEEYF NFIVSSDTYF NKKKF |

SEQ ID NO: 60 (wild type Eptv1)

| | |
|---|---|
| 1 | MEDLFSITCD LHMNAFIEEV EKLWSSSLNN TSSLSRKSKN VIRNLFREVT |
| 51 | NSNISSSCYD ILLKKQLHGD SINAVYRSLY GSGSDIDKRV DTVGKYILFV |
| 101 | VVTYLAILID DKDSKEIVSI LTKFVNVISN IHSKYKCKYM FVGIPAIILF |
| 151 | NKLDSTDVQK LYSLFSTKLN TNVELYEEYF NFIVSSDTYF NKKKFVKFSY |
| 201 | GPVSFASSIS VPDFVMEGLT FRSCDRIEKS EDIDDVYVFI TVESDPKTYA |
| 251 | FSKLTKPLYE GGLVVETDDL DDATALVIFD AITTFDKFRN KALLLTLESI |
| 301 | VNKQVIDPTL NIDRYPTDEN ISDDTGENVI TPIKVGSSIG IVDYRLVINK |
| 351 | LTEWLNQCEE KCEGAVSPEV KELRERITDL EKQLDEATSG KTNCEYEKTK |
| 401 | IKDLESRLDS ERQRVTRLLD DLQKARDGKC DSDSNDKAII EELRKEIQNE |
| 451 | QNRRMELMKE LDKVRNGDGT SSCERELDLT RQWLHERDNE LREMSKQAKH |
| 501 | FERELERERI KNKQCDKYKK ELDEAKSKII RLETDLDKCL SQQNGSSDEV |
| 551 | RKLTSQIESL ERDLRECRAS SGGDEKLLAD IDLLKQQIGI LAQQLEQCET |
| 601 | RGDEKLLAEI EFLKEHINSL AQQLEQCEAS GGSGGSDTSK LQERITYLEN |
| 651 | ELDKYIKGEG NVNFTLLNEI NRLRDKNAEL QNQLDQAQSQ DKNNSYYKRA |
| 701 | LERERAKIIE LENELQKCFD NNSGSEYIIK MEQMERKIKS LEAELRLCKD |
| 751 | TDHDTEKIYK DKIAELQREL DKCKQSGGSS NSHTEIKVFY DVECRTESAR |
| 801 | LQQRINELND ELNRLRKEDK TDSYYKREVD RQRKKVIELE HELEKYFNDD |
| 851 | KIITYKKEMD AMQVVISEMR QELEKCKRDA SCSSSSDCSF EQKRIELLEL |
| 901 | ELRKTKEMVK QLEKFIEFSS AQKEYADKLE REKIARLDAE HALERERARK |
| 951 | DCGGNMCEQE LELERNKNKK LELYLETEKD KANFYKRELE KERFIKSSSQ |
| 1001 | EE |

SEQ ID NO: 61 (Eptv2 active domain 1)

| | |
|---|---|
| 1 | ANEKIIREDL PLSFY |

SEQ ID NO: 62 (Eptv2 active domain 2)

| | |
|---|---|
| 1 | VSYELANEKI IREDLPLSFY IESFMRWYAQ QRIQSVYTVN PVYKECLSCV |
| 51 | NYARAKIPLS KFNKAINHYN SDNNSVYLVD VRKYN |

SEQ ID NO: 63 (wild type Eptv2)

| | |
|---|---|
| 1 | MGAYTSIMNI NDFNKIYAYK QDYRCKYNVS YELANEKIIR EDLPLSFYIE |
| 51 | SFMRWYAQQR IQSVYTVNPV YKECLSCVNY ARAKIPLSKF NKAINHYNSD |
| 101 | NNSVYLVDVR KYNNMQKLQL YIGFYSYVTT KLKSEDAIKR CIHTCTYISD |
| 151 | EKTKLEFM |

SEQ ID NO: 64 (KRAB)

| | |
|---|---|
| 1 | DAKSLTAWSR TLVTFKDVFV DFTREEWKLL DTAQQILYRN VMLENYKNLV |
| 51 | SLGYQLTKPD VILRLEKGEE PWLVEREIHQ ETHPDSETAF EIKSSV |

### Additional Aspects of the Engineered Gene Effectors

In some embodiments, the engineered gene effector as disclosed herein can be used to effect regulation of expression and/or activity level of the target gene (e.g., one or more target genes of Table 1). In some cases, a heterologous polypeptide comprising at least the engineered gene effector can be introduced to a cell (e.g., a mammalian cell) to effect the regulation of the expression and/or activity level of the target gene. In some embodiments, the engineered gene effector as disclosed herein, or a protein comprising the engineered gene effector (e.g., a protein comprising the engineered gene effector coupled to the heterologous endonuclease) can be referred to as an actuator moiety.

In some embodiments, the engineered gene effector as disclosed herein can be coupled to a heterologous endonuclease (e.g., enzymatically active Cas protein, enzymatically deactivated Cas protein, etc.). The engineered gene effector and the heterologous endonuclease can be coupled to each other, e.g., directly or indirectly (e.g., via a linker). For example, the engineered gene effector and the heterologous endonuclease can be fused to each other, e.g., directly or indirectly (e.g., via the linker). In another example, the engineered gene effector and the heterologous endonuclease can be non-covalently coupled to each other, e.g., via ionic bonds, hydrogen bonds, interactions mediated by oligomerization or dimerization domains, etc. In some cases, the engineered gene effector and the heterologous endonuclease can be part of a single polypeptide molecule (e.g., a chimeric or fusion polypeptide).

Non-limiting examples of the heterologous endonuclease as disclosed herein can include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof; any variant thereof and any fragment thereof.

In some embodiments, the heterologous endonuclease as disclosed herein can have a length of at most about 1000 amino acids, at most about 950 amino acids, at most about 900 amino acids, at most about 850 amino acids, at most about 800 amino acids, at most about 750 amino acids, at most about 700 amino acids, at most about 650 amino acids, at most about 640 amino acids, at most about 630 amino acids, at most about 620 amino acids, at most about 610 amino acids, at most about 600 amino acids, at most about 590 amino acids, at most about 580 amino acids, at most about 570 amino acids, at most about 560 amino acids, at most about 550 amino acids, at most about 540 amino acids, at most about 530 amino acids, at most about 520 amino acids, at most about 510 amino acids, at most about 500 amino acids, at most about 490 amino acids, at most about 480 amino acids, at most about 470 amino acids, at most about 460 amino acids, at most about 450 amino acids, at most about 440 amino acids, at most about 430 amino acids, at most about 420 amino acids, at most about 410 amino acids, at most about 400 amino acids, at most about 350 amino acids, or at most about 300 amino acids.

In some embodiments, is the heterologous endonuclease as disclosed herein can be nuclease-deficient. In some embodiments, the heterologous endonuclease can be a nuclease-null DNA binding protein that does not induce transcriptional activation or repression of a target DNA sequence unless it is present in a complex with one or more heterologous gene effectors of the disclosure. In some embodiments, the heterologous endonuclease can be a nuclease-null DNA binding protein that can induce transcriptional activation or repression of a target DNA sequence (e.g., which can be altered or augmented by the presence of a heterologous gene effector as provided herein).

In some embodiments, the heterologous endonuclease as disclosed herein can be an RNA nuclease such as an engineered (e.g., programmable or targetable) RNA nuclease. In some embodiments, the heterologous endonuclease as disclosed herein can be a nuclease-null RNA binding protein that does not induce transcriptional activation or repression of a target RNA sequence unless it is present in a complex with one or more heterologous gene effectors of the disclosure. In some embodiments, the heterologous endonuclease as disclosed herein can be a nuclease-null RNA binding protein that can induce transcriptional activation or repression of a target RNA sequence (e.g., which can be altered or augmented by the presence of a heterologous gene effector as provided herein).

In some embodiments, the heterologous endonuclease can be a nucleic acid-guided targeting system. In some embodiments, the heterologous endonuclease can be a DNA-guided targeting system. In some embodiments, the heterologous endonuclease can be an RNA-guided targeting system. The nucleic acid-guided targeting system can comprise and utilize, for example, a guide nucleic acid sequence that facilitates specific binding of a CRISPR-Cas system (e.g., a nuclease deficient form thereof, such as dCas9 or dCas14) to a target gene (e.g., target endogenous gene) or target gene regulatory sequence. For example, the target gene may be any one of the genes listed in Table 1, and the target gene regulatory sequence may be operatively coupled to any one of the genes listed in Table 1. Binding specificity can be determined by use of a guide nucleic acid, such as a single guide RNA (sgRNA) or a part thereof. In some embodiments, the use of different sgRNAs allows the compositions and methods of the disclosure to be used with (e.g., targeted to) different target genes (e.g., target endogenous genes) or target gene regulatory sequences.

In some embodiments, the heterologous endonuclease can comprise an amino acid sequence having at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 10 (e.g., CasMini). In some embodiments, the heterologous endonuclease can comprise an amino acid sequence having at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100% sequence identity to the polypeptide sequence of SEQ ID NO: 11 (e.g., dCasMini).

In some embodiments, the amino acid sequence of the heterologous endonuclease as disclosed herein can be mutated and/or modified to yield a nuclease deficient protein or a protein with decreased nuclease activity relative to a wild-type Cas protein. A nuclease deficient protein can retain the ability to bind a target gene (e.g., DNA), but may lack or have reduced nucleic acid cleavage activity. In some embodiments, the heterologous endonuclease can exhibit reduced nuclease activity (e.g., nuclease deficient or nuclease null) as compared to wild type Un1Cas12fl. The reduced nuclease activity can be at most about 95%, at most about 90%, at most about 80%, at most about 70%, at most about 60%, at most about 50%, at most about 40%, at most about 30%, at most about 20%, at most about 10%, at most about 5%, at most about 1%, at most about 0.5%, at most about 0.1%, or less than that of the wild type Un1Cas12fl. In some cases, the heterologous endonuclease can comprise a substitution at D326 and/or D510, as compared to the polypeptide sequence of wild type Un1Cas12f1. For example, the D326 and/or the D510 substitution(s) can be alanine substitutions (e.g., D326A and/or D510A).

In some embodiments, the amino acid sequence of the heterologous endonuclease (e.g., a Cas protein) as disclosed herein can comprise one or more substitutions in the native amino acid sequence, where the positions of at least some of these substitutions follow one or more particular rules determined to have surprising advantages for the heterologous endonuclease. In some cases, the particular substitution rules have been selected for their ability to produce variants of the heterologous endonuclease, e.g., that can be capable of functioning within eukaryotic cells. According to these particular rules, all or some of the one or more substitutions in the native amino acid sequence are either (1) within or no more than 30 amino acids downstream of a (D/E/K/N)X(R/F)(E/K)N motif of the native amino acid sequence, (2) at or no more than 30 amino acids upstream or downstream of position 241 of the native amino acid sequence, (3) at or no more than 30 amino acids upstream or downstream of position 516 of the native amino acid sequence, and/or (4) having an electrically charged amino acid in the native amino acid sequence.

In some embodiments, the amino acid sequence of the heterologous endonuclease (e.g., a Cas protein) as disclosed herein can comprise one or more substitutions at amino acid positions within or no more than a threshold length (e.g., 30 amino acid residues) upstream and/or downstream of a (D/E/K/N)X(R/F)(E/K)N motif, e.g., as compared to the polypeptide sequence of wild type Un1Cas12fl. In some cases, at least one of the one or more substitutions can be, for example, within or no more than 28 amino acids, 26 amino acids, 24 amino acids, 22 amino acids, 20 amino acids, 18 amino acids, 16 amino acids, 14 amino acids, 12 amino acids, or 10 amino acids of the motif. In some cases, at least one of the one or more substitutions can be to an R, A, S, or G amino acid residue. In some cases, the one or more substitutions can include substitutions are at one or more positions selected from the group consisting of D143, T147, E151, and K154 (e.g., D143R, T147R, E151R, and/or K154R). In some cases, the one or more substitutions can include substitutions are at one or more positions selected from the group consisting of N504, E507, N516, N519, E527, and E528 (e.g., N504R, E507R, N516R, N519R, E527R, and/or E528R). In some cases, the one or more substitutions can include substitutions are at one or more positions selected from the group consisting of K11, K73, D143, T147, E151, K154, E241, D318, K330, K457, E425, E462, N504, E507, N516, N519, E527, and E528 (e.g., K1IR, K73R, D143R, T147R, E151R, K154R, E241R, D318R, K330R, E425N, K457R, E462R, N504R, E507R, N516R, N519R, E527R, and/or E528R).

In some embodiments, the amino acid sequence of the heterologous endonuclease comprising the one or more substitutions upstream and/or downstream of the (D/E/K/N)X(R/F)(E/K)N motif, as disclosed herein, can exhibit a cationic charge (e.g., a positive) that is greater than that of a wild type variant of the heterologous endonuclease, by at least or up to about 1 cationic charge, at least or up to about 2 cationic charges, at least or up to about 3 cationic charges, at least or up to about 4 cationic charges, at least or up to about 5 cationic charges, at least or up to about 6 cationic charges, at least or up to about 7 cationic charges, at least or up to about 8 cationic charges, at least or up to about 9 cationic charges, at least or up to about 10 cationic charges, at least or up to about 11 cationic charges, at least or up to about 12 cationic charges, at least or up to about 13 cationic charges, at least or up to about 14 cationic charges, at least or up to about 15 cationic charges, at least or up to about 16 cationic charges, at least or up to about 17 cationic charges, or at least or up to about 18 cationic charges.

In some embodiments, the heterologous endonuclease (e.g., and thus the engineered gene effector via being operatively coupled to the heterologous endonuclease) can form a complex with a guide nucleic acid, such as a guide RNA or a part thereof. In some embodiments, the heterologous endonuclease can form a complex with a single guide nucleic acid, such as a single guide RNA (sgRNA). In some embodiments, the heterologous endonuclease can be a RNA-binding protein (RBP) optionally complexed with a guide nucleic acid, such as a guide RNA (e.g., sgRNA), which is able to form a complex with a Cas protein. In some embodiments, the heterologous endonuclease can be a nuclease-null DNA binding protein that can induce transcriptional activation or repression of a target DNA sequence. In some embodiments, the heterologous endonuclease can be a nuclease-null RNA binding protein derived from a RNA.

A guide nucleic acid used in compositions and methods of the disclosure can be, for example, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 nucleotides.

In some embodiments, a guide nucleic acid used in compositions and methods of the disclosure is at most at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, or at most 40 nucleotides.

In some embodiments, a guide nucleic acid used in compositions and methods of the disclosure is between about 8 and about 40 nucleotides, between about 10 and about 40 nucleotides, between about 11 and about 40 nucleotides, between about 12 and about 40 nucleotides, between about 13 and about 40 nucleotides, between about 14 and about 40 nucleotides, between about 15 and about 40 nucleotides, between about 16 and about 40 nucleotides, between about 17 and about 40 nucleotides, between about 18 and about 40 nucleotides, between about 19 and about 40 nucleotides, between about 20 and about 40 nucleotides, between about 22 and about 40 nucleotides, between about 24 and about 40 nucleotides, between about 26 and about 40 nucleotides, between about 28 and about 40 nucleotides, between about 30 and about 40 nucleotides, between about 8 and about 30 nucleotides, between about 10 and about 30 nucleotides, between about 11 and about 30 nucleotides, between about 12 and about 30 nucleotides, between about 13 and about 30 nucleotides, between about 14 and about 30 nucleotides, between about 15 and about 30 nucleotides, between about 16 and about 30 nucleotides, between about 17 and about 30 nucleotides, between about 18 and about 30 nucleotides, between about 19 and about 30 nucleotides, between about 20 and about 30 nucleotides, between about 22 and about 30 nucleotides, between about 24 and about 30 nucleotides, between about 26 and about 30 nucleotides, between about 28 and about 30 nucleotides, between about 8 and about 25 nucleotides, between about 10 and about 25 nucleotides, between about 11 and about 25 nucleotides, between about 12 and about 25 nucleotides, between about 13 and about 25 nucleotides, between about 14 and about 25 nucleotides, between about 15 and about 25 nucleotides, between about 16 and about 25 nucleotides, between about 17 and about 25 nucleotides, between about 18 and about 25 nucleotides, between about 19 and about 25 nucleotides, between about 20 and about 25 nucleotides, between about 22 and about 25 nucleotides, between about 24 and about 25 nucleotides, between about 8 and about 20 nucleotides, between about 10 and about 20 nucleotides, between about 11 and about 20 nucleotides, between about 12 and about 20 nucleotides, between about 13 and about 20 nucleotides, between about 14 and about 20 nucleotides, between about 15 and about 20 nucleotides, between about 16 and about 20 nucleotides, between about 17 and about 20 nucleotides, between about 18 and about 20 nucleotides, between about 19 and about 20 nucleotides, between about 8 and about 18 nucleotides, between about 10 and about 18 nucleotides, between about 11 and about 18 nucleotides, between about 12 and about 18 nucleotides, between about 13 and about 18 nucleotides, between about 14 and about 18 nucleotides, between about 15 and about 18 nucleotides, between about 16 and about 18 nucleotides, between about 8 and about 16 nucleotides, between about 10 and about 16 nucleotides, between about 11 and about 16 nucleotides, between about 12 and about 16 nucleotides, between about 13 and about 16 nucleotides, between about 14 and about 16 nucleotides, or between about 15 and about 16 nucleotides. In some embodiments, a guide nucleic acid can be a guide RNA or a part thereof.

The heterologous endonuclease as disclosed herein can be modified to enhance regulation of gene expression by compositions and methods of the disclosure, e.g., as part of a complex disclosed herein. The heterologous endonuclease can be modified to increase or decrease nucleic acid binding affinity, nucleic acid binding specificity, enzymatic activity, and/or binding to other factors, such as heterodimerization or oligomerization domains and induce ligands. The heterologous endonuclease can also be modified to change any other activity or property of the protein, such as stability. For example, one or more nuclease domains of the heterologous endonuclease can be modified, deleted, or inactivated, or at least a portion of the heterologous endonuclease can be truncated to remove domains that are not essential for the desired function of the protein or complex. The heterologous endonuclease can be modified to modulate (e.g., enhance or reduce) the activity of the heterologous endonuclease for regulating gene expression by a complex of the disclosure that comprises a heterologous gene effector.

For example, the heterologous endonuclease can be coupled (e.g., fused, covalently coupled, or non-covalently coupled) to a heterologous gene effector (e.g., an epigenetic modification domain, a transcriptional activation domain, and/or a transcriptional repressor domain). The heterologous endonuclease can be coupled (e.g., fused, covalently coupled, or non-covalently coupled) to an oligomerization or dimerization domain as disclosed herein (e.g., a heterodimerization domain). The heterologous endonuclease can be coupled (e.g., fused, covalently coupled, or non-covalently coupled) to a heterologous polypeptide that provides increased or decreased stability. The heterologous endonuclease can be coupled (e.g., fused, covalently coupled, or non-covalently coupled) to a sequence that can facilitate degradation of the heterologous endonuclease or a complex containing the heterologous endonuclease.

The heterologous endonuclease can be coupled (e.g., fused, covalently coupled, or non-covalently coupled) to any suitable number of partners, for example, at least one, at least two, at least three, at least four, or at least five, at least six, at least seven, or at least 8 partners. In some embodiments, the heterologous endonuclease of the disclosure is coupled (e.g., fused, covalently coupled, or non-covalently coupled) to at most two, at most three, at most four, at most five, at most six, at most seven, at most eight, or at most ten partners. In some embodiments, the heterologous endonuclease of the disclosure is coupled (e.g., fused, covalently coupled, or non-covalently coupled) to 1-5, 1-4, 1-3, 1-2, 2-5, 2-4, 2-3, 3-5, 3-4, or 4-5 partners. In some embodiments, the heterologous endonuclease of the disclosure is coupled (e.g., fused, covalently coupled, or non-covalently coupled) to one partner. In some embodiments, the heterologous endonuclease of the disclosure is coupled (e.g., fused, covalently coupled, or non-covalently coupled) to two partners. In some embodiments, the heterologous endonuclease of the disclosure is coupled (e.g., fused, covalently coupled, or non-covalently coupled) to three partners. In some embodiments, the heterologous endonuclease of the disclosure is coupled (e.g., fused, covalently coupled, or non-covalently coupled) to four partners. In some embodiments, the heterologous endonuclease of the disclosure is coupled (e.g., fused, covalently coupled, or non-covalently coupled) to five partners. In some embodiments, the heterologous endonuclease of the disclosure is coupled (e.g., fused, covalently coupled, or non-covalently coupled) to six partners.

The heterologous endonuclease as disclosed herein can be coupled to (e.g., covalently or non-covalently attached to) one or more of the partners as disclosed herein. The one or more partners can comprise (i) any one of the engineered gene effectors as disclosed herein and (ii) one or more additional partner(s) provided in the present disclosure.

The heterologous endonuclease as disclosed herein can be a fusion protein, e.g., a fusion comprising the heterologous endonuclease and one or more of the partners as disclosed herein. The fused domain can be located at the N-terminus, the C-terminus, or internally within the heterologous endonuclease.

A partner of the heterologous endonuclease (e.g., covalently or non-covalently coupled to a nuclease deficient or null variant of the heterologous endonuclease as disclosed herein) can be a transcriptional effector (e.g., a transcriptional activator or a transcriptional repressor). The transcriptional effector can be heterologous to the cell as provided herein.

In some embodiments, the transcriptional effector can be a histone epigenetic modifier (or a histone modifier). In some cases, the histone epigenetic modifier can modulate histones through methylation (e.g., a histone methylation modifier, such as an amino acid methyltransferase, e.g., KRAB).

In some cases, the histone epigenetic modifier can modulate histones through acetylation. In some cases, the histone epigenetic modifier can modulate histones through phosphorylation. In some cases, the histone epigenetic modifier can modulate histones through ADP-ribosylation. In some cases, the histone epigenetic modifier can modulate histones through glycosylation. In some cases, the histone epigenetic modifier can modulate histones through SUMOylation. In some cases, the histone epigenetic modifier can modulate histones through ubiquitination. In some cases, the histone epigenetic modifier can modulate histones by remodeling histone structure, e.g., via an ATP hydrolysis-dependent process.

In some embodiments, the transcriptional effector can be a gene epigenetic modifier (or a gene modifier). In some cases, a gene modifier can modulate genes through methylation (e.g., a gene methylation modifier, such as a DNA methyltransferase or DNMT). In some cases, a gene modifier can modulate genes through acetylation.

In some embodiments, the transcriptional effector is from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, the transcriptional effector can comprise an epigenetic modifier. In some embodiments, the transcriptional effector comprises a histone epigenetic modifier (e.g., a histone lysine methyltransferase, a histone lysine demethylase, or a DNA methylase). Non-limiting examples of an epigenetic modifier can include EZH subfamily, Non-SET subfamily, Other SET subfamily, PRDM subfamily, SET1 subfamily, SET2 subfamily, SUV39 subfamily, SYMD subfamily, ASH1L, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SYMD1, SYMD2, SYMD3, SYMD4, and SYMD5.

Examples of proteins (or fragments thereof) that can be used as a fusion partner to increase transcription include but are not limited to: transcriptional activators such as VP16, VP64, VP48, VP160, p65 subdomain (e.g, from NFkB), and activation domain of EDLL and/or TAL activation domain (e.g, for activity in plants), SET1A, SET1B, MLL1 to 5, ASH1, SYMD2, NSD1, JHDM2a/b, UTX, JMJD3, GCN5, PCAF, CBP, p300, TAF1, TIP60/PLIP, MOZMYST3, MORFMYST4, SRC1, ACTR, PI 60, CLOCK, Ten-Eleven Translocation (TET) dioxygenase 1 (TET1CD), TET1, DME, DML1, DML2, ROS1, etc.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g, for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

The heterologous endonuclease as disclosed herein can be provided in any form. For example, the heterologous endonuclease can be provided in the form of a protein, such as the heterologous endonuclease alone or complexed with a guide nucleic acid as a ribonucleoprotein. The heterologous endonuclease can be provided in a complex, for example, complexed with a guide nucleic acid and/or one or more heterologous gene effectors of the disclosure. The heterologous endonuclease can be provided in the form of a nucleic acid encoding at least the heterologous endonuclease, such as an RNA (e.g., messenger RNA (mRNA)), or DNA. The nucleic acid encoding at least the heterologous endonuclease can be codon optimized for efficient translation into protein in a particular cell or organism (e.g., human codon optimized).

Nucleic acids encoding at least the heterologous endonuclease as disclosed herein, fragments, or derivatives thereof can be stably integrated in the genome of a cell. Nucleic acids encoding at least the heterologous endonuclease can be operably linked to a promoter, for example, a promoter that is constitutively or inducibly active in the cell. Nucleic acids encoding at least the heterologous endonuclease can be operably linked to a promoter in an expression construct. Expression constructs can include any nucleic acid constructs capable of directing expression of a gene or other nucleic acid sequence of interest (e.g., at least the heterologous endonuclease) and which can transfer such a nucleic acid sequence of interest to a target cell.

In some embodiments, the heterologous endonuclease as disclosed herein can associate with a single guide RNA (sgRNA) to activate or repress transcription of a target gene (e.g., target endogenous gene), for example, in combination with heterologous gene effector(s) disclosed herein. sgRNAs can be introduced into cells expressing the heterologous endonuclease or variant thereof, as provided herein. In some cases, such cells can contain one or more different sgRNAs that target the same target gene (e.g., target endogenous gene) or target gene regulatory sequence. In other cases, the sgRNAs target different nucleic acids in the cell (e.g., different target genes, different target gene regulatory sequences, or different sequences within the same target gene or target gene regulatory sequence).

Enzymatically inactive (e.g., nuclease deficient) can refer to a nuclease that can bind to a nucleic acid sequence in a polynucleotide in a sequence-specific manner, but may not cleave a target polynucleotide or will cleave it at a substantially reduced frequency. An enzymatically inactive guide moiety can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a comparable wild-type activity (e.g., nucleic acid cleaving activity, wild-type Cas activity).

In some embodiments, the target nucleic acid of the heterologous endonuclease as disclosed herein can be dsDNA. In such embodiments, dsDNA-targeting specificity is determined, at least in part, by two parameters: the gRNA spacer targeting a protospacer in the target dsDNA (the sequence in the target dsDNA corresponding to the gRNA spacer on the non-complementary DNA strand) and a short sequence, the protospacer-adjacent motif (PAM), located immediately 5′ (upstream) of the protospacer on the non-complementary DNA strand. In some embodiments, the PAM is 5′-TTTG-3′ or 5′-TTTA-3′. In some embodiments, the PAM is 5′-TTTG-3′. In some embodiments, the PAM is 5′-TTTA-3′.

In some embodiments, the target nucleic acid of the heterologous endonuclease as disclosed herein can be RNA. In such embodiments, RNA-targeting specificity is determined, at least in part, by the gRNA spacer targeting a protospacer-like sequence in the target RNA (the sequence in the target RNA complementary to the gRNA spacer), and is independent of the sequence located immediately 5′ (upstream) of the protospacer-like sequence. In some embodiments, the heterologous endonuclease can be further capable of targeting a dsDNA molecule, wherein the gRNA spacer is selected such that it targets a protospacer in the target dsDNA molecule having a PAM selected from 5′-TTTG-3′ and 5′-TTTA-3′. In other embodiments, the heterologous endonuclease is incapable of targeting a dsDNA molecule, wherein the gRNA spacer is selected such that any protospacers in the dsDNA molecule targeted by the gRNA spacer do not have a PAM selected from 5′-TTTG-3′ and 5′-TTTA-3′.

In some embodiments, the heterologous polypeptide comprising the engineered gene effector and the heterologous endonuclease (e.g., and/or a complex comprising the heterologous polypeptide) can regulate expression and/or activity of a target gene (e.g., target endogenous gene). In some embodiments, the heterologous polypeptide and/or a complex thereof can edit the sequence of a nucleic acid (e.g., a gene and/or gene product). A nuclease-active variant of the heterologous endonuclease can edit a nucleic acid sequence by generating a double-stranded break or single-stranded break in a target polynucleotide.

In some embodiments, the heterologous polypeptide comprising the engineered gene effector and the heterologous endonuclease (e.g., and/or a complex comprising the heterologous polypeptide) can generate a double-strand break in a target polynucleotide, such as DNA. A double-strand break in DNA can result in DNA break repair which allows for the introduction of gene modification(s) (e.g., nucleic acid editing). In some embodiments, a nuclease induces site-specific single-strand DNA breaks or nicks, thus resulting in HDR.

A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided.

In some embodiments, the heterologous polypeptide comprising the engineered gene effector and the heterologous endonuclease (e.g., and/or a complex comprising the heterologous polypeptide) does not generate a double-strand break in a target polynucleotide, such as DNA. Binding of the heterologous polypeptide or the complex comprising the heterologous polypeptide (e.g., a complex comprising a nuclease deficient variant of the heterologous endonuclease and a guide RNA) without a nucleic acid break can be sufficient to regulate expression (e.g., enhance or suppress) of a target gene (e.g., endogenous target gene).

### Target Gene

The disclosure provides compositions, methods, and systems for modulating expression of one or more target genes. The target gene(s) can be one or more heterologous target genes. The target gene(s) can be one or more endogenous target genes, such as (i) a disease causing allele, e.g., a mutant allele, and/or (ii) a non-disease causing allele, e.g., a wild type allele. For example, disclosed herein are complexes that comprise a guide moiety and one or more heterologous polypeptides comprising the engineered gene effector and the heterologous endonuclease that can modulate (e.g., increase or decrease) an activity or expression level of a target gene (e.g., in a cell).

In some embodiments, a target gene or regulatory sequence thereof is endogenous to a cell, for example, present in the cell's genome, or endogenous to a subject, for example, present in the subject's genome. In some embodiments, a target gene or regulatory sequence thereof is not part of an engineered reporter system.

In some embodiments, a target gene is exogenous to a host subject, for example, a pathogen target gene or an exogenous gene expressed as a result of a therapeutic intervention, such as a gene therapy and/or cell therapy. In some embodiments, a target gene is an exogenous reporter gene. In some embodiments, a target gene is an exogenous synthetic gene.

In some embodiments, the systems and methods as disclosed herein can modulate (e.g., increase or decrease) expression of a target gene (e.g., upon introducing a complex comprising the engineered gene effector and the heterologous polypeptide into a cell or population of cells). In some embodiments, an expression level is an RNA expression level can be measured by, for example, RNAseq, qPCR, microarray, gene array, FISH, etc. In some embodiments, an expression level is a protein expression level can be measured by, for example, Western Blot, ELISA, multiplex immunoassay, mass spectrometry, NMR, proteomics, flow cytometry, mass cytometry, etc.

In some embodiments, the systems and methods as disclosed herein can modulate (e.g., increase or decrease) expression of a target gene (e.g., upon introducing a complex comprising the engineered gene effector and the heterologous polypeptide into a cell or population of cells) by at least about 10%, at least about 20%, at least about 30%, at least about 40%, at least about 50%, at least about 60%, at least about 70%, at least about 80%, at least about 90%, at least about 2-fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14, at least fold about 15 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 250 fold, at least about 300 fold, at least about 350 fold, at least about 400 fold, at least about 500 fold, at least about 600 fold, at least about 700 fold, at least about 800 fold, at least about 900 fold, at least about 1000 fold, at least about 1500 fold, at least about 2000 fold, or at least about 3000 fold.

In some embodiments, the systems and methods as disclosed herein can modulate (e.g., increase or decrease) expression of a target gene (e.g., upon introducing a complex comprising the engineered gene effector and the heterologous polypeptide into a cell or population of cells) by at most about 50%, at most about 60%, at most about 70%, at most about 80%, at most about 90%, at most about 2-fold, at most about 3 fold, at most about 4 fold, at most about 5 fold, at most about 6 fold, at most about 7 fold, at most about 8 fold, at most about 9 fold, at most about 10 fold, at most about 11 fold, at most about 12 fold, at most about 13 fold, at most about 14, at most fold about 15 fold, at most about 20 fold, at most about 30 fold, at most about 40 fold, at most about 50 fold, at most about 60 fold, at most about 70 fold, at most about 80 fold, at most about 90 fold, at most about 100 fold, at most about 150 fold, at most about 200 fold, at most about 250 fold, at most about 300 fold, at most about 350 fold, at most about 400 fold, at most about 500 fold, at most about 600 fold, at most about 700 fold, at most about 800 fold, at most about 900 fold, at most about 1000 fold, at most about 1500 fold, at most about 2000 fold, at most about 3000 fold, at most about 5000 fold, or at most about 10000 fold.

In some embodiments, the systems and methods as disclosed herein can modulate (e.g., increase or decrease) expression of a target gene (e.g., upon introducing a complex comprising the engineered gene effector and the heterologous polypeptide into a cell or population of cells) by about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, the systems and methods as disclosed herein can modulate (e.g., increase or decrease) expression of a target gene (e.g., upon introducing a complex comprising the engineered gene effector and the heterologous polypeptide into a cell or population of cells) from below a limit of detection to a detectable level.

In some embodiments, the degree in change of expression is relative to before introducing the system of the present disclosure (e.g., a complex comprising the engineered gene effector and the heterologous polypeptide) into the cell or population of cells. In some embodiments, the degree in change of expression is relative to a corresponding control cell or population of cells that are not treated with the system of the present disclosure. In some embodiments, the degree in change of expression is relative to a corresponding control cell or population of cells that are treated with an alternative to the system of the present disclosure.

In some embodiments, the systems and methods as disclosed herein can modulate (e.g., increase or decrease) an activity level of a target gene (e.g., upon introducing a complex comprising the engineered gene effector and the heterologous polypeptide comprising the heterologous endonuclease as disclosed herein into a cell or population of cells). An activity level can be determined by a suitable functional assay for the target gene in question depending on the functional characteristics of the target gene. For example, an activity level of a target gene that is a mitogen could be determined by measuring cell proliferation; an activity level of a target gene that induces apoptosis could be measured by an annexin V assay or other suitable cell death assay; an activity level of an anti-inflammatory cytokine could be measured by an LPS-induced cytokine release assay.

The systems and methods of the present disclosure can, in some cases, elicit changes in expression and/or activity level of a target gene (e.g., target endogenous gene) that persists for longer than can be achieved with alternative compositions and methods (e.g., suppression via RNAi, e.g., using siRNA). In some embodiments, persistent modulation of gene expression (e.g., durable gene activation or durable gene suppression) is advantageous as compared to transient modulation.

In some embodiments, the systems and methods as disclosed herein can modulate (e.g., increase or decrease) expression and/or activity level of a target gene for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 18 hours, at least about 20 hours, at least about 1 day, at least about 2 days, at least about 3 days, at least about 4 days, at least about 5 days, at least about 6 days, at least about 7 days, at least about 8 days, at least about 9 days, at least about 10 days, at least about 14 days, at least about 21 days, at least about 28 days, at least about 5 weeks, at least about 6 weeks, at least about 7 weeks, at least about 8 weeks, at least about 9 weeks, at least about 10 weeks, at least about 12 weeks, at least about 14 weeks, at least about 18 weeks, at least about 20 weeks, at least about 26 weeks, or at least about 5 months, at least about 6 months, at least about 9 months, at least about 12 months, or more.

In some embodiments the systems and methods as disclosed herein can modulate (e.g., increase or decrease) expression and/or activity level of a target gene (e.g., target endogenous gene) to above a certain threshold for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 18 hours, at most about 20 hours, at most about 1 day, at most about 2 days, at most about 3 days, at most about 4 days, at most about 5 days, at most about 6 days, at most about 7 days, at most about 8 days, at most about 9 days, at most about 10 days, at most about 14 days, at most about 21 days, at most about 28 days, at most about 5 weeks, at most about 6 weeks, at most about 7 weeks, at most about 8 weeks, at most about 9 weeks, at most about 10 weeks, at most about 12 weeks, at most about 14 weeks, at most about 18 weeks, at most about 20 weeks, at most about 26 weeks, or at most about 5 months, at most about 6 months, at most about 9 months, at most about 12 months, or more.

In some embodiments, the systems and methods as disclosed herein can modulate (e.g., increase or decrease) expression and/or activity level of a target gene (e.g., target endogenous gene) to above a certain threshold for about 1 hour, about 2 hours, about 3 hours, about 4 hours, about 5 hours, about 6 hours, about 7 hours, about 8 hours, about 9 hours, about 10 hours, about 12 hours, about 14 hours, about 18 hours, about 20 hours, about 1 day, about 2 days, about 3 days, about 4 days, about 5 days, about 6 days, about 7 days, about 8 days, about 9 days, about 10 days, about 14 days, about 21 days, about 28 days, about 5 weeks, about 6 weeks, about 7 weeks, about 8 weeks, about 9 weeks, about 10 weeks, about 12 weeks, about 14 weeks, about 18 weeks, about 20 weeks, about 26 weeks, about 5 months, about 6 months, about 9 months, or about 12 months.

In some embodiments, the target gene (e.g., endogenous target gene) can be a disease-causing allele, such as a mutant variant of a wild type allele. The disease can be a genetic disease, such as a hereditary disorder. Non-limiting examples of the genetic disorder can include Duchenne muscular dystrophy (DMD), hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease. In some cases, the target gene can be a gene encoding a protein. In some cases, the target gene can be a gene regulatory sequence (e.g., promoters, enhancers, repressors, silencers, insulators, cis-regulatory elements, trans-regulatory elements, epigenetic modification (e.g., DNA methylation) sites, etc.) that can influence expression of a gene encoding a protein of interest as provided herein. For example, target gene regulatory sequences can be physically located outside of the transcriptional unit or open reading frame that encodes a product of the target gene.

In some embodiments, a target gene regulatory sequence does not contain a nucleotide sequence that is exogenous to the subject or host cell. In some embodiments, a target gene regulatory sequence does not contain an engineered or artificially generated or introduced nucleotide sequence.

In some embodiments, a target gene (e.g., target endogenous gene) is a gene that is over-expressed or under-expressed in a disease or condition. In some embodiments, a target gene is a gene that is over-expressed or under-expressed in a heritable genetic disease.

In some embodiments, a target gene (e.g., target endogenous gene) is a gene that is over-expressed or under-expressed in a cancer, for example, acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or a combination thereof.

Non-limiting examples of a target gene or a gene encoding a protein of interest, as disclosed herein, are included in Table 1.

TABLE 1
List of examples of target genes (e.g., encoding a protein of interest)
List of target genes/proteins of interest

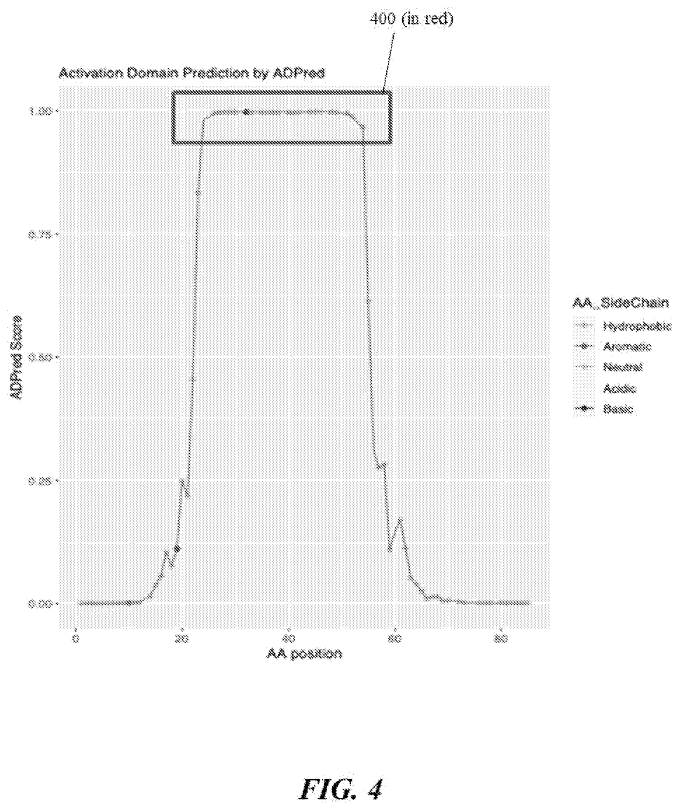

A1BG, CCND3, FAM163A, KCNK10, NRG4, REM1, TECTB, A1CF, CCNDBP1, FAM163B,
KCNK12, NRGN, REM2, TEDDM1, A2M, CCNE1, FAM166A, KCNK13, NRIP1, REN, TEF, A2ML1,
CCNE2, FAM166B, KCNK15, NRIP2, RENBP, TEFM, A3GALT2, CCNF, FAM167A, KCNK16,
NRIP3, REP15, TEK, A4GALT, CCNG1, FAM167B, KCNK17, NRK, REPIN1, TEKT1, A4GNT,
CCNG2, FAM168A, KCNK18, NRL, REPS1, TEKT2, AAAS, CCNH, FAM168B, KCNK2, NRM,
REPS2, TEKT3, AACS, CCNI, FAM169A, KCNK3, NRN1, RER1, TEKT4, AADAC, CCNI2,
FAM169B, KCNK4, NRN1L, RERE, TEKT5, AADACL2, CCNJ, FAM170A, KCNK5, NRP1, RERG,
TELO2, AADACL3, CCNJL, FAM170B, KCNK6, NRP2, RERGL, TEN1, AADACL4, CCNK,
FAM171A1, KCNK7, NRROS, RESP18, TENC1, AADAT, CCNL1, FAM171A2, KCNK9, NRSN1,
REST, TENM1, AAED1, CCNL2, FAM171B, KCNMA1, NRSN2, RET, TENM2, AAGAB, CCNO,
FAM172A, KCNMB1, NRTN, RETN, TENM3, AAK1, CCNT1, FAM173A, KCNMB2, NRXN1,
RETNLB, TENM4, AAMDC, CCNT2, FAM173B, KCNMB3, NRXN2, RETSAT, TEP1, AAMP,
CCNY, FAM174A, KCNMB4, NRXN3, REV1, TEPP, AANAT, CCNYL1, FAM174B, KCNN1, NSA2,
REV3L, TERF1, AAR2, CCP110, FAM175A, KCNN2, NSD1, REXO1, TERF2, AARD, CCPG1,
FAM175B, KCNN3, NSDHL, REXO2, TERF2IP, AARS, CCR1, FAM177A1, KCNN4, NSF, REXO4,
TERT, AARS2, CCR10, FAM177B, KCNQ1, NSFL1C, RFC1, TES, AARSD1, CCR2, FAM178A,
KCNQ2, NSG1, RFC2, TESC, AASDH, CCR3, FAM178B, KCNQ3, NSL1, RFC3, TESK1,
AASDHPPT, CCR4, FAM179A, KCNQ4, NSMAF, RFC4, TESK2, AASS, CCR5, FAM179B, KCNQ5,
NSMCE1, RFC5, TESPA1, AATF, CCR6, FAM180A, KCNRG, NSMCE2, RFESD, TET1, AATK,
CCR7, FAM180B, KCNS1, NSMCE4A, RFFL, TET2, ABAT, CCR8, FAM181A, KCNS2, NSMF, RFK,
TET3, ABCA1, CCR9, FAM181B, KCNS3, NSRP1, RFNG, TEX10, ABCA10, CCRL2, FAM183A,
KCNT1, NSUN2, RFPL1, TEX101, ABCA12, CCRN4L, FAM184A, KCNT2, NSUN3, RFPL2, TEX11,
ABCA13, CCS, FAM184B, KCNU1, NSUN4, RFPL3, TEX12, ABCA2, CCSAP, FAM185A, KCNV1,
NSUN5, RFPL4A, TEX13A, ABCA3, CCSER1, FAM186A, KCNV2, NSUN6, RFPL4AL1, TEX13B,
ABCA4, CCSER2, FAM186B, KCP, NSUN7, RFPL4B, TEX14, ABCA5, CCT2, FAM187B, KCTD1,
NT5C, RFT1, TEX15, ABCA6, CCT3, FAM188A, KCTD10, NT5C1A, RFTN1, TEX19, ABCA7,
CCT4, FAM188B, KCTD11, NT5C1B, RFTN2, TEX2, ABCA8, CCT5, FAM189A1, KCTD12,
NT5C1B-RDH14, RFWD2, TEX22, ABCA9, CCT6A, FAM189A2, KCTD13, NT5C2, RFWD3, TEX26,
ABCB1, CCT6B, FAM189B, KCTD14, NT5C3A, RFX1, TEX261, ABCB10, CCT7, FAM192A,
KCTD15, NT5C3B, RFX2, TEX264, ABCB11, CCT8, FAM193A, KCTD16, NT5DC1, RFX3, TEX28,
ABCB4, CCT8L2, FAM193B, KCTD17, NT5DC2, RFX4, TEX29, ABCB5, CCZ1, FAM194A,
KCTD18, NT5DC3, RFX5, TEX30, ABCB6, CCZ1B, FAM194B, KCTD19, NT5E, RFX6, TEX33,
ABCB7, CD101, FAM195A, KCTD2, NT5M, RFX7, TEX35, ABCB8, CD109, FAM195B, KCTD20,
NTAN1, RFX8, TEX36, ABCB9, CD14, FAM196A, KCTD21, NTF3, RFXANK, TEX37, ABCC1,
CD151, FAM196B, KCTD3, NTF4, RFXAP, TEX38, ABCC10, CD160, FAM198A, KCTD4, NTHL1,
RGAG1, TEX40, ABCC11, CD163, FAM198B, KCTD5, NTM, RGAG4, TEX9, ABCC12, CD163L1,
FAM199X, KCTD6, NTMT1, RGCC, TF, ABCC2, CD164, FAM19A1, KCTD7, NTN1, RGL1, TFAM,
ABCC3, CD164L2, FAM19A2, KCTD8, NTN3, RGL2, TFAP2A, ABCC4, CD177, FAM19A3, KCTD9,
NTN4, RGL3, TFAP2B, ABCC5, CD180, FAM19A4, KDELC1, NTN5, RGL4, TFAP2C, ABCC6,
CD19, FAM19A5, KDELC2, NTNG1, RGMA, TFAP2D, ABCC8, CD1A, FAM200A, KDELR1,
NTNG2, RGMB, TFAP2E, ABCC9, CD1B, FAM203A, KDELR2, NTPCR, RGN, TFAP4, ABCD1,
CD1C, FAM203B, KDELR3, NTRK1, RGP1, TFB1M, ABCD2, CD1D, FAM204A, KDM1A, NTRK2,
RGPD1, TFB2M, ABCD3, CD1E, FAM205A, KDM1B, NTRK3, RGPD2, TFCP2, ABCD4, CD2,
FAM206A, KDM2A, NTS, RGPD3, TFCP2L1, ABCE1, CD200, FAM207A, KDM2B, NTSR1, RGPD4,
TFDP1, ABCF1, CD200R1, FAM208A, KDM3A, NTSR2, RGPD5, TFDP2, ABCF2, CD200R1L,
FAM208B, KDM3B, NUAK1, RGPD6, TFDP3, ABCF3, CD207, FAM209A, KDM4A, NUAK2,
RGPD8, TFE3, ABCG1, CD209, FAM209B, KDM4B, NUB1, RGR, TFEB, ABCG2, CD22, FAM20A,
KDM4C, NUBP1, RGS1, TFEC, ABCG4, CD226, FAM20B, KDM4D, NUBP2, RGS10, TFF1, ABCG5,
CD24, FAM20C, KDM4E, NUBPL, RGS11, TFF2, ABCG8, CD244, FAM210A, KDM5A, NUCB1,
RGS12, TFF3, ABHD1, CD247, FAM210B, KDM5B, NUCB2, RGS13, TFG, ABHD10, CD248,
FAM211A, KDM5C, NUCKS1, RGS14, TFIP11, ABHD11, CD27, FAM211B, KDM5D, NUDC,
RGS16, TFPI, ABHD12, CD274, FAM212A, KDM6A, NUDCD1, RGS17, TFPI2, ABHD12B, CD276,
FAM212B, KDM6B, NUDCD2, RGS18, TFPT, ABHD13, CD28, FAM213A, KDM8, NUDCD3,
RGS19, TFR2, ABHD14A, CD2AP, FAM213B, KDR, NUDT1, RGS2, TFRC, ABHD14B, CD2BP2,
FAM214A, KDSR, NUDT10, RGS20, TG, ABHD15, CD300A, FAM214B, KEAP1, NUDT11, RGS21,
TGDS, ABHD16A, CD300C, FAM216A, KEL, NUDT12, RGS22, TGFA, ABHD16B, CD300E,
FAM216B, KERA, NUDT13, RGS3, TGFB1, ABHD17A, CD300LB, FAM217A, KHDC1, NUDT14,
RGS4, TGFB1I1, ABHD17B, CD300LD, FAM217B, KHDC1L, NUDT15, RGS5, TGFB2, ABHD17C,
CD300LF, FAM218A, KHDC3L, NUDT16, RGS6, TGFB3, ABHD2, CD300LG, FAM219A,
KHDRBS1, NUDT16L1, RGS7, TGFBI, ABHD3, CD302, FAM219B, KHDRBS2, NUDT17, RGS7BP,
TGFBR1, ABHD4, CD320, FAM21A, KHDRBS3, NUDT18, RGS8, TGFBR2, ABHD5, CD33,
FAM21B, KHK, NUDT19, RGS9, TGFBR3, ABHD6, CD34, FAM21C, KHNYN, NUDT2, RGS9BP,
TGFBR3L, ABHD8, CD36, FAM220A, KHSRP, NUDT21, RGSL1, TGFBRAP1, ABI1, CD37,
FAM221A, KIAA0020, NUDT22, RHAG, TGIF1, ABI2, CD38, FAM221B, KIAA0040, NUDT3,
RHBDD1, TGIF2, ABI3, CD3D, FAM222A, KIAA0100, NUDT4, RHBDD2, TGIF2-C20orf24,
ABI3BP, CD3E, FAM222B, KIAA0101, NUDT5, RHBDD3, TGIF2LX, ABL1, CD3EAP, FAM227A,
KIAA0141, NUDT6, RHBDF1, TGIF2LY, ABL2, CD3G, FAM227B, KIAA0195, NUDT7, RHBDF2,
TGM1, ABLIM1, CD4, FAM228A, KIAA0196, NUDT8, RHBDL1, TGM2, ABLIM2, CD40,
FAM228B, KIAA0226, NUDT9, RHBDL2, TGM3, ABLIM3, CD40LG, FAM229A, KIAA0226L,
NUF2, RHBDL3, TGM4, ABO, CD44, FAM229B, KIAA0232, NUFIP1, RHBG, TGM5, ABR, CD46,
FAM230A, KIAA0247, NUFIP2, RHCE, TGM6, ABRA, CD47, FAM24A, KIAA0319, NUGGC,
RHCG, TGM7, ABRACL, CD48, FAM24B, KIAA0319L, NUMA1, RHD, TGOLN2, ABT1, CD5,
FAM25A, KIAA0355, NUMB, RHEB, TGS1, ABTB1, CD52, FAM25C, KIAA0368, NUMBL,
RHEBL1, TH, ABTB2, CD53, FAM25G, KIAA0391, NUP107, RHNO1, THADA, ACAA1, CD55,
FAM26D, KIAA0408, NUP133, RHO, THAP1, ACAA2, CD58, FAM26E, KIAA0430, NUP153,
RHOA, THAP10, ACACA, CD59, FAM26F, KIAA0513, NUP155, RHOB, THAP11, ACACB, CD5L,
FAM32A, KIAA0556, NUP160, RHOBTB1, THAP2, ACAD10, CD6, FAM35A, KIAA0586, NUP188,
RHOBTB2, THAP3, ACAD11, CD63, FAM3A, KIAA0753, NUP205, RHOBTB3, THAP4, ACAD8,
CD68, FAM3B, KIAA0754, NUP210, RHOC, THAP5, ACAD9, CD69, FAM3C, KIAA0825, NUP210L,
RHOD, THAP6, ACADL, CD7, FAM3D, KIAA0895, NUP214, RHOF, THAP7, ACADM, CD70,
FAM43A, KIAA0895L, NUP35, RHOG, THAP8, ACADS, CD72, FAM43B, KIAA0907, NUP37,
RHOH, THAP9, ACADSB, CD74, FAM45A, KIAA0922, NUP43, RHOJ, THBD, ACADVL, CD79A,
FAM46A, KIAA0930, NUP50, RHOQ, THBS1, ACAN, CD79B, FAM46B, KIAA0947, NUP54,
RHOT1, THBS2, ACAP1, CD80, FAM46C, KIAA1009, NUP62, RHOT2, THBS3, ACAP2, CD81,
FAM46D, KIAA1024, NUP62CL, RHOU, THBS4, ACAP3, CD82, FAM47A, KIAA1024L, NUP85,
RHOV, THEG, ACAT1, CD83, FAM47B, KIAA1033, NUP88, RHOXF1, THEG5, ACAT2, CD84,
FAM47C, KIAA1045, NUP93, RHOXF2, THEGL, ACBD3, CD86, FAM47E, KIAA1107, NUP98,
RHOXF2B, THEM4, ACBD4, CD8A, FAM47E-STBD1, KIAA1109, NUPL1, RHPN1, THEM5,
ACBD5, CD8B, FAM49A, KIAA1143, NUPL2, RHPN2, THEM6, ACBD6, CD9, FAM49B, KIAA1147,
NUPR1, RIBC1, THEMIS, ACBD7, CD93, FAM50A, KIAA1161, NUPR1L, RIBC2, THEMIS2, ACCS,
CD96, FAM50B, KIAA1191, NUS1, RIC3, THG1L, ACCSL, CD97, FAM53A, KIAA1199, NUSAP1,
RIC8A, THNSL1, ACD, CD99, FAM53B, KIAA1210, NUTF2, RIC8B, THNSL2, ACE, CD99L2,
FAM53C, KIAA1211, NUTM1, RICTOR, THOC1, ACE2, CDA, FAM57A, KIAA1211L, NUTM2A,
RIF1, THOC2, ACER1, CDADC1, FAM57B, KIAA1217, NUTM2B, RIIAD1, THOC3, ACER2,
CDAN1, FAM58A, KIAA1239, NUTM2F, RILP, THOC5, ACER3, CDC123, FAM60A, KIAA1244,
NUTM2G, RILPL1, THOC6, ACHE, CDC14A, FAM63A, KIAA1257, NVL, RILPL2, THOC7, ACIN1,
CDC14B, FAM63B, KIAA1279, NWD1, RIMBP2, THOP1, ACKR1, CDC16, FAM64A, KIAA1324,
NXF1, RIMBP3, THPO, ACKR2, CDC20, FAM65A, KIAA1324L, NXF2, RIMBP3B, THRA, ACKR3,
CDC20B, FAM65B, KIAA1328, NXF2B, RIMBP3C, THRAP3, ACKR4, CDC23, FAM65C,
KIAA1377, NXF3, RIMKLA, THRB, ACLY, CDC25A, FAM69A, KIAA1407, NXF5, RIMKLB,
THRSP, ACMSD, CDC25B, FAM69B, KIAA1429, NXN, RIMS1, THSD1, ACN9, CDC25C, FAM69C,
KIAA1430, NXNL1, RIMS2, THSD4, ACO1, CDC26, FAM71A, KIAA1432, NXNL2, RIMS3,
THSD7A, ACO2, CDC27, FAM71B, KIAA1456, NXPE1, RIMS4, THSD7B, ACOT1, CDC34,
FAM71C, KIAA1462, NXPE2, RIN1, THTPA, ACOT11, CDC37, FAM71D, KIAA1467, NXPE3, RIN2,
THUMPD1, ACOT12, CDC37L1, FAM71E1, KIAA1468, NXPE4, RIN3, THUMPD2, ACOT13,
CDC40, FAM71E2, KIAA1522, NXPH1, RING1, THUMPD3, ACOT2, CDC42, FAM71F1, KIAA1524,
NXPH2, RINL, THY1, ACOT4, CDC42BPA, FAM71F2, KIAA1549, NXPH3, RINT1, THYN1,
ACOT6, CDC42BPB, FAM72A, KIAA1549L, NXPH4, RIOK1, TIA1, ACOT7, CDC42BPG, FAM72B,
KIAA1551, NXT1, RIOK2, TIAF1, ACOT8, CDC42EP1, FAM72D, KIAA1586, NXT2, RIOK3, TIAL1,
ACOT9, CDC42EP2, FAM73A, KIAA1598, NYAP1, RIPK1, TIAM1, ACOX1, CDC42EP3, FAM73B,
KIAA1614, NYAP2, RIPK2, TIAM2, ACOX2, CDC42EP4, FAM76A, KIAA1644, NYNRIN, RIPK3,
TICAM1, ACOX3, CDC42EP5, FAM76B, KIAA1671, NYX, RIPK4, TICAM2, ACOXL, CDC42SE1,
FAM78A, KIAA1683, OAF, RIPPLY1, TICRR, ACP1, CDC42SE2, FAM78B, KIAA1715, OARD1,
RIPPLY2, TIE1, ACP2, CDC45, FAM81A, KIAA1731, OAS1, RIPPLY3, TIFA, ACP5, CDC5L,
FAM81B, KIAA1737, OAS2, RIT1, TIFAB, ACP6, CDC6, FAM83A, KIAA1751, OAS3, RIT2, TIGD2,
ACPL2, CDC7, FAM83B, KIAA1755, OASL, RLBP1, TIGD3, ACPP, CDC73, FAM83C, KIAA1804,
OAT, RLF, TIGD4, ACPT, CDCA2, FAM83D, KIAA1841, OAZ1, RLIM, TIGD5, ACR, CDCA3,
FAM83E, KIAA1919, OAZ2, RLN1, TIGD6, ACRBP, CDCA4, FAM83F, KIAA1958, OAZ3, RLN2,
TIGD7, ACRC, CDCA5, FAM83G, KIAA1984, OBFC1, RLN3, TIGIT, ACRV1, CDCA7, FAM83H,
KIAA2013, OBP2A, RLTPR, TIMD4, ACSBG1, CDCA7L, FAM84A, KIAA2018, OBP2B, RMDN1,
TIMELESS, ACSBG2, CDCA8, FAM84B, KIAA2022, OBSCN, RMDN2, TIMM10, ACSF2, CDCP1,
FAM86A, KIAA2026, OBSL1, RMDN3, TIMM10B, ACSF3, CDCP2, FAM86B1, KIDINS220, OC90,
RMI1, TIMM13, ACSL1, CDH1, FAM86B2, KIF11, OCA2, RMI2, TIMM17A, ACSL3, CDH10,
FAM86C1, KIF12, OCEL1, RMND1, TIMM17B, ACSL4, CDH11, FAM86KP, KIF13A, OCIAD1,
RMND5A, TIMM21, ACSL5, CDH12, FAM89A, KIF13B, OCIAD2, RMND5B, TIMM22, ACSL6,
CDH13, FAM89B, KIF14, OCLM, RNASE1, TIMM23, ACSM1, CDH15, FAM8A1, KIF15, OCLN,
RNASE10, TIMM23B, ACSM2A, CDH16, FAM90A1, KIF16B, OCM, RNASE11, TIMM44, ACSM2B,
CDH17, FAM91A1, KIF17, OCM2, RNASE12, TIMM50, ACSM3, CDH18, FAM92A1, KIF18A,
OCRL, RNASE13, TIMM8A, ACSM4, CDH19, FAM92B, KIF18B, OCSTAMP, RNASE2, TIMM8B,
ACSM5, CDH2, FAM96A, KIF19, ODAM, RNASE3, TIMM9, ACSS1, CDH20, FAM96B, KIF1A,
ODC1, RNASE4, TIMMDC1, ACSS2, CDH22, FAM98A, KIF1B, ODF1, RNASE6, TIMP1, ACSS3,
CDH23, FAM98B, KIF1C, ODF2, RNASE7, TIMP2, ACTA1, CDH24, FAM98C, KIF20A, ODF2L,
RNASE8, TIMP3, ACTA2, CDH26, FAM9A, KIF20B, ODF3, RNASE9, TIMP4, ACTB, CDH3,
FAM9B, KIF21A, ODF3B, RNASEH1, TINAG, ACTBL2, CDH4, FAM9C, KIF21B, ODF3L1,
RNASEH2A, TINAGL1, ACTC1, CDH5, FAN1, KIF22, ODF3L2, RNASEH2B, TINF2, ACTG1,
CDH6, FANCA, KIF23, ODF4, RNASEH2C, TIPARP, ACTG2, CDH7, FANCB, KIF24, OFCC1,
RNASEK, TIPIN, ACTL10, CDH8, FANCC, KIF25, OFD1, RNASEL, TIPRL, ACTL6A, CDH9,
FANCD2, KIF26A, OGDH, RNASET2, TIRAP, ACTL6B, CDHR1, FANCD2OS, KIF26B, OGDHL,
RND1, TJAP1, ACTL7A, CDHR2, FANCE, KIF27, OGFOD1, RND2, TJP1, ACTL7B, CDHR3,
FANCF, KIF28P, OGFOD2, RND3, TJP2, ACTL8, CDHR4, FANCG, KIF2A, OGFOD3, RNF10, TJP3,
ACTL9, CDHR5, FANCI, KIF2B, OGFR, RNF103, TK1, ACTN1, CDIP1, FANCL, KIF2C, OGFRL1,
RNF103-CHMP3, TK2, ACTN2, CDIPT, FANCM, KIF3A, OGG1, RNF11, TKT, ACTN3, CDK1,
FANK1, KIF3B, OGN, RNF111, TKTL1, ACTN4, CDK10, FAP, KIF3C, OGT, RNF112, TKTL2,
ACTR10, CDK11A, FAR1, KIF4A, OIP5, RNF113A, TLCD1, ACTR1A, CDK11B, FAR2, KIF4B,
OIT3, RNF113B, TLCD2, ACTR1B, CDK12, FARP1, KIF5A, OLA1, RNF114, TLDC1, ACTR2,
CDK13, FARP2, KIF5B, OLAH, RNF115, TLDC2, ACTR3, CDK14, FARS2, KIF5C, OLFM1,
RNF121, TLE1, ACTR3B, CDK15, FARSA, KIF6, OLFM2, RNF122, TLE2, ACTR3C, CDK16,
FARSB, KIF7, OLFM3, RNF123, TLE3, ACTR5, CDK17, FAS, KIF9, OLFM4, RNF125, TLE4,
ACTR6, CDK18, FASLG, KIFAP3, OLFML1, RNF126, TLE6, ACTR8, CDK19, FASN, KIFC1,
OLFML2A, RNF128, TLK1, ACTRT1, CDK2, FASTK, KIFC2, OLFML2B, RNF13, TLK2, ACTRT2,
CDK20, FASTKD1, KIFC3, OLFML3, RNF130, TLL1, ACTRT3, CDK2AP1, FASTKD2, KIN, OLIG1,
RNF133, TLL2, ACVR1, CDK2AP2, FASTKD3, KIR2DL1, OLIG2, RNF135, TLN1, ACVR1B, CDK3,
FASTKD5, KIR2DL3, OLIG3, RNF138, TLN2, ACVR1C, CDK4, FAT1, KIR2DL4, OLR1, RNF139,
TLR1, ACVR2A, CDK5, FAT2, KIR2DS4, OMA1, RNF14, TLR10, ACVR2B, CDK5R1, FAT3,
KIR3DL1, OMD, RNF141, TLR2, ACVRL1, CDK5R2, FAT4, KIR3DL2, OMG, RNF144A, TLR3,
ACY1, CDK5RAP1, FATE1, KIR3DL3, OMP, RNF144B, TLR4, ACY3, CDK5RAP2, FAU, KIRREL,
ONECUT1, RNF145, TLR5, ACYP1, CDK5RAP3, FAXC, KIRREL2, ONECUT2, RNF146, TLR6,
ACYP2, CDK6, FAXDC2, KIRREL3, ONECUT3, RNF148, TLR7, ADA, CDK7, FBF1, KISS1, OOEP,
RNF149, TLR8, ADAD1, CDK8, FBL, KISS1R, OOSP2, RNF150, TLR9, ADAD2, CDK9, FBLIM1,
KIT, OPA1, RNF151, TLX1, ADAL, CDKAL1, FBLN1, KITLG, OPA3, RNF152, TLX1NB, ADAM10,
CDKL1, FBLN2, KL, OPALIN, RNF157, TLX2, ADAM11, CDKL2, FBLN5, KLB, OPCML, RNF165,
TLX3, ADAM12, CDKL3, FBLN7, KLC1, OPHN1, RNF166, TM2D1, ADAM15, CDKL4, FBN1,
KLC2, OPLAH, RNF167, TM2D2, ADAM17, CDKL5, FBN2, KLC3, OPN1LW, RNF168, TM2D3,
ADAM18, CDKN1A, FBN3, KLC4, OPN1MW, RNF169, TM4SF1, ADAM19, CDKN1B, FBP1, KLF1,
OPN1MW2, RNF17, TM4SF18, ADAM2, CDKN1C, FBP2, KLF10, OPN1SW, RNF170, TM4SF19,
ADAM20, CDKN2A, FBRS, KLF11, OPN3, RNF175, TM4SF20, ADAM21, CDKN2AIP, FBRSL1,
KLF12, OPN4, RNF180, TM4SF4, ADAM22, CDKN2AIPNL, FBXL12, KLF13, OPN5, RNF181,
TM4SF5, ADAM23, CDKN2B, FBXL13, KLF14, OPRD1, RNF182, TM6SF1, ADAM28, CDKN2C,
FBXL14, KLF15, OPRK1, RNF183, TM6SF2, ADAM29, CDKN2D, FBXL15, KLF16, OPRL1,
RNF185, TM7SF2, ADAM30, CDKN3, FBXL16, KLF17, OPRM1, RNF186, TM7SF3, ADAM32,
CDNF, FBXL17, KLF2, OPTC, RNF187, TM9SF1, ADAM33, CDO1, FBXL18, KLF3, OPTN,
RNF19A, TM9SF2, ADAM7, CDON, FBXL19, KLF4, OR10A2, RNF19B, TM9SF3, ADAM8, CDPF1,
FBXL2, KLF5, OR10A3, RNF2, TM9SF4, ADAM9, CDR1, FBXL20, KLF6, OR10A4, RNF20,
TMA16, ADAMDEC1, CDR2, FBXL21, KLF7, OR10A5, RNF207, TMA7, ADAMTS1, CDR2L,
FBXL22, KLF8, OR10A6, RNF208, TMBIM1, ADAMTS10, CDRT1, FBXL3, KLF9, OR10A7,
RNF212, TMBIM4, ADAMTS12, CDRT15, FBXL4, KLHDC1, OR10AG1, RNF213, TMBIM6,
ADAMTS13, CDRT15L2, FBXL5, KLHDC10, OR10C1, RNF214, TMC1, ADAMTS14, CDRT4,
FBXL6, KLHDC2, OR10G2, RNF215, TMC2, ADAMTS15, CDS1, FBXL7, KLHDC3, OR10G3,
RNF216, TMC3, ADAMTS16, CDS2, FBXL8, KLHDC4, OR10G4, RNF217, TMC4, ADAMTS17,
CDSN, FBXO10, KLHDC7A, OR10G7, RNF219, TMC5, ADAMTS18, CDT1, FBXO11, KLHDC7B,
OR10G8, RNF220, TMC6, ADAMTS19, CDV3, FBXO15, KLHDC8A, OR10G9, RNF222, TMC7,
ADAMTS2, CDX1, FBXO16, KLHDC8B, OR10H1, RNF223, TMC8, ADAMTS20, CDX2, FBXO17,
KLHDC9, OR10H2, RNF224, TMCC1, ADAMTS3, CDX4, FBXO18, KLHL1, OR10H3, RNF24,
TMCC2, ADAMTS4, CDY1, FBXO2, KLHL10, OR10H4, RNF25, TMCC3, ADAMTS5, CDY1B,
FBXO21, KLHL11, OR10H5, RNF26, TMCO1, ADAMTS6, CDY2A, FBXO22, KLHL12, OR10J1,
RNF31, TMCO2, ADAMTS7, CDY2B, FBXO24, KLHL13, OR10J3, RNF32, TMCO3, ADAMTS8,
CDYL, FBXO25, KLHL14, OR10J5, RNF34, TMCO4, ADAMTS9, CDYL2, FBXO27, KLHL15,
OR10K1, RNF38, TMCOSA, ADAMTSLI, CEACAM1, FBXO28, KLHL17, OR10K2, RNF39,
TMCO6, ADAMTSL2, CEACAM16, FBXO3, KLHL18, OR10P1, RNF4, TMED1, ADAMTSL3,
CEACAM18, FBXO30, KLHL2, OR10Q1, RNF40, TMED10, ADAMTSL4, CEACAM19, FBXO31,
KLHL20, OR10R2, RNF41, TMED2, ADAMTSL5, CEACAM20, FBXO32, KLHL21, OR10S1, RNF43,
TMED3, ADAP1, CEACAM21, FBXO33, KLHL22, OR10T2, RNF44, TMED4, ADAP2, CEACAM3,
FBXO34, KLHL23, OR10V1, RNF5, TMED5, ADAR, CEACAM4, FBXO36, KLHL24, OR10W1,
RNF6, TMED6, ADARB1, CEACAM5, FBXO38, KLHL25, OR10X1, RNF7, TMED7, ADARB2,
CEACAM6, FBXO39, KLHL26, OR10Z1, RNF8, TMED7-TICAM2, ADAT1, CEACAM7, FBXO4,
KLHL28, OR11A1, RNFT1, TMED8, ADAT2, CEACAM8, FBXO40, KLHL29, OR11G2, RNFT2,
TMED9, ADAT3, CEBPA, FBXO41, KLHL3, OR11H1, RNGTT, TMEFF1, ADC, CEBPB, FBXO42,
KLHL30, OR11H12, RNH1, TMEFF2, ADCK1, CEBPD, FBXO43, KLHL31, OR11H2, RNLS,
TMEM100, ADCK2, CEBPE, FBXO44, KLHL32, OR11H4, RNMT, TMEM101, ADCK3, CEBPG,
FBXO45, KLHL33, OR11H6, RNMTL1, TMEM102, ADCK4, CEBPZ, FBXO46, KLHL34, OR11L1,
RNPC3, TMEM104, ADCK5, CECR1, FBXO47, KLHL35, OR12D2, RNPEP, TMEM105, ADCY1,
CECR2, FBXO48, KLHL36, OR12D3, RNPEPL1, TMEM106A, ADCY10, CECR5, FBXO5, KLHL38,
OR13A1, RNPS1, TMEM106B, ADCY2, CECR6, FBXO6, KLHL4, OR13C2, ROBO1, TMEM106C,
ADCY3, CEL, FBXO7, KLHL40, OR13C3, ROBO2, TMEM107, ADCY4, CELA1, FBXO8, KLHL41,
OR13C4, ROBO3, TMEM108, ADCY5, CELA2A, FBXO9, KLHL42, OR13C5, ROBO4, TMEM109,
ADCY6, CELA2B, FBXW10, KLHL5, OR13C8, ROCK1, TMEM11, ADCY7, CELA3A, FBXW11,
KLHL6, OR13C9, ROCK2, TMEM110, ADCY8, CELA3B, FBXW12, KLHL7, OR13D1, ROGDI,
TMEM110-MUSTN1, ADCY9, CELF1, FBXW2, KLHL8, OR13F1, ROM1, TMEM114, ADCYAP1,
CELF2, FBXW4, KLHL9, OR13G1, ROMO1, TMEM115, ADCYAP1R1, CELF3, FBXW5, KLK1,
OR13H1, ROPN1, TMEM116, ADD1, CELF4, FBXW7, KLK10, OR13J1, ROPN1B, TMEM117,
ADD2, CELF5, FBXW8, KLK11, OR14A16, ROPN1L, TMEM119, ADD3, CELF6, FBXW9, KLK12,
OR14C36, ROR1, TMEM120A, ADGB, CELSR1, FCAMR, KLK13, OR14J1, ROR2, TMEM120B,
ADH1A, CELSR2, FCAR, KLK14, OR1A1, RORA, TMEM121, ADH1B, CELSR3, FCER1A, KLK15,
OR1A2, RORB, TMEM123, ADH1C, CEMP1, FCER1G, KLK2, OR1B1, RORC, TMEM125, ADH4,
CEND1, FCER2, KLK3, OR1C1, ROS1, TMEM126A, ADH5, CENPA, FCF1, KLK4, OR1D2, RP1,
TMEM126B, ADH6, CENPB, FCGBP, KLK5, OR1D5, RP1L1, TMEM127, ADH7, CENPBD1,
FCGR1A, KLK6, OR1E1, RP2, TMEM128, ADHFE1, CENPC, FCGR1B, KLK7, OR1E2, RP9,
TMEM129, ADI1, CENPE, FCGR2A, KLK8, ORIF1, RPA1, TMEM130, ADIG, CENPF, FCGR2B,
KLK9, OR1G1, RPA2, TMEM131, ADIPOQ, CENPH, FCGR2C, KLKB1, OR1I1, RPA3, TMEM132A,
ADIPOR1, CENPI, FCGR3A, KLLN, OR1J1, RPA4, TMEM132B, ADIPOR2, CENPJ, FCGR3B,
KLRB1, OR1J4, RPAIN, TMEM132C, ADIRF, CENPK, FCGRT, KLRC1, OR1K1, RPAP1,
TMEM132D, ADK, CENPL, FCHO1, KLRC2, OR1L1, RPAP2, TMEM132E, ADM, CENPM, FCHO2,
KLRC3, OR1L3, RPAP3, TMEM133, ADM2, CENPN, FCHSD1, KLRC4, OR1L4, RPE, TMEM134,
ADM5, CENPO, FCHSD2, KLRC4-KLRK1, OR1L6, RPE65, TMEM135, ADNP, CENPP, FCN1,
KLRD1, OR1L8, RPF1, TMEM136, ADNP2, CENPQ, FCN2, KLRF1, OR1M1, RPF2, TMEM138,
ADO, CENPT, FCN3, KLRF2, OR1N1, RPGR, TMEM139, ADORA1, CENPU, FCRL1, KLRG1,
OR1N2, RPGRIP1, TMEM140, ADORA2A, CENPV, FCRL2, KLRG2, OR1Q1, RPGRIP1L,
TMEM141, ADORA2B, CENPW, FCRL3, KLRK1, OR1S1, RPH3A, TMEM143, ADORA3, CEP104,
FCRL4, KMO, OR1S2, RPH3AL, TMEM144, ADPGK, CEP112, FCRL5, KMT2A, OR2A12, RPIA,
TMEM145, ADPRH, CEP120, FCRL6, KMT2B, OR2A14, RPL10, TMEM147, ADPRHL1, CEP128,
FCRLA, KMT2C, OR2A2, RPL10A, TMEM14A, ADPRHL2, CEP135, FCRLB, KMT2D, OR2A25,
RPL10L, TMEM14B, ADPRM, CEP152, FDCSP, KMT2E, OR2A4, RPL11, TMEM14C, ADRA1A,
CEP164, FDFT1, KNCN, OR2A5, RPL12, TMEM14E, ADRA1B, CEP170, FDPS, KNDC1, OR2A7,
RPL13, TMEM150A, ADRA1D, CEP170B, FDX1, KNG1, OR2AE1, RPL13A, TMEM150B, ADRA2A,
CEP19, FDX1L, KNOP1, OR2AG1, RPL14, TMEM150C, ADRA2B, CEP192, FDXACB1, KNSTRN,
OR2AG2, RPL15, TMEM151A, ADRA2C, CEP250, FDXR, KNTC1, OR2AK2, RPL17, TMEM151B,
ADRB1, CEP290, FECH, KPNA1, OR2AP1, RPL17-C18orf32, TMEM154, ADRB2, CEP350, FEM1A,
KPNA2, OR2AT4, RPL18, TMEM155, ADRB3, CEP41, FEM1B, KPNA3, OR2B11, RPL18A,
TMEM156, ADRBK1, CEP44, FEM1C, KPNA4, OR2B2, RPL19, TMEM158, ADRBK2, CEP55, FEN1,
KPNA5, OR2B3, RPL21, TMEM159, ADRM1, CEP57, FER, KPNA6, OR2B6, RPL22, TMEM160,
ADSL, CEP57L1, FER1L5, KPNA7, OR2C1, RPL22L1, TMEM161A, ADSS, CEP63, FER1L6,
KPNB1, OR2C3, RPL23, TMEM161B, ADSSL1, CEP68, FERD3L, KPRP, OR2D2, RPL23A,
TMEM163, ADTRP, CEP70, FERMT1, KPTN, OR2D3, RPL24, TMEM164, AEBP1, CEP72, FERMT2,
KRAS, OR2F1, RPL26, TMEM165, AEBP2, CEP76, FERMT3, KRBA1, OR2F2, RPL26L1,
TMEM167A, AEN, CEP78, FES, KRBA2, OR2G2, RPL27, TMEM167B, AES, CEP85, FETUB,
KRBOX1, OR2G3, RPL27A, TMEM168, AFAP1, CEP85L, FEV, KRBOX4, OR2G6, RPL28,
TMEM169, AFAP1L1, CEP89, FEZ1, KRCC1, OR2H1, RPL29, TMEM17, AFAP1L2, CEP95, FEZ2,
KREMEN1, OR2J2, RPL3, TMEM170A, AFF1, CEP97, FEZF1, KREMEN2, OR2J3, RPL30,
TMEM170B, AFF2, CEPT1, FEZF2, KRI1, OR2K2, RPL31, TMEM171, AFF3, CER1, FFAR1, KRIT1,
OR2L13, RPL32, TMEM173, AFF4, CERCAM, FFAR2, KRR1, OR2L2, RPL34, TMEM174, AFG3L2,
CERK, FFAR3, KRT1, OR2L3, RPL35, TMEM175, AFM, CERKL, FFAR4, KRT10, OR2L5, RPL35A,
TMEM176A, AFMID, CERS1, FGA, KRT12, OR2L8, RPL36, TMEM176B, AFP, CERS2, FGB,
KRT13, OR2M2, RPL36A, TMEM177, AFTPH, CERS3, FGD1, KRT14, OR2M3, RPL36A-HNRNPH2,
TMEM178A, AGA, CERS4, FGD2, KRT15, OR2M4, RPL36AL, TMEM178B, AGAP1, CERS5, FGD3,
KRT16, OR2M5, RPL37, TMEM179, AGAP10, CERS6, FGD4, KRT17, OR2M7, RPL37A,
TMEM179B, AGAP11, CES1, FGD5, KRT18, OR2S2, RPL38, TMEM18, AGAP2, CES2, FGD6,
KRT19, OR2T1, RPL39, TMEM180, AGAP3, CES3, FGF1, KRT2, OR2T10, RPL39L, TMEM181,
AGAP4, CES4A, FGF10, KRT20, OR2T11, RPL3L, TMEM182, AGAP5, CES5A, FGF11, KRT222,
OR2T12, RPL4, TMEM183A, AGAP6, CETN1, FGF12, KRT23, OR2T2, RPL41, TMEM183B,
AGAP7, CETN2, FGF13, KRT24, OR2T27, RPL5, TMEM184A, AGAP8, CETN3, FGF14, KRT25,
OR2T29, RPL6, TMEM184B, AGAP9, CETP, FGF16, KRT26, OR2T3, RPL7, TMEM184C, AGBL1,
CFB, FGF17, KRT27, OR2T33, RPL7A, TMEM185A, AGBL2, CFC1, FGF18, KRT28, OR2T34,
RPL7L1, TMEM185B, AGBL3, CFC1B, FGF19, KRT3, OR2T35, RPL8, TMEM186, AGBL4, CFD,
FGF2, KRT31, OR2T4, RPL9, TMEM187, AGBL5, CFDP1, FGF20, KRT32, OR2T5, RPLP0,
TMEM189, AGER, CFH, FGF21, KRT33A, OR2T6, RPLP1, TMEM189-UBE2V1, AGFG1, CFHR1,
FGF22, KRT33B, OR2T8, RPLP2, TMEM19, AGFG2, CFHR2, FGF23, KRT34, OR2V1, RPN1,
TMEM190, AGGF1, CFHR3, FGF3, KRT35, OR2V2, RPN2, TMEM191B, AGK, CFHR4, FGF4,
KRT36, OR2W1, RPP14, TMEM191C, AGL, CFHR5, FGF5, KRT37, OR2W3, RPP21, TMEM192,
AGMAT, CFI, FGF6, KRT38, OR2W5, RPP25, TMEM194A, AGMO, CFL1, FGF7, KRT39, OR2Y1,
RPP25L, TMEM194B, AGO1, CFL2, FGF8, KRT4, OR2Z1, RPP30, TMEM196, AGO2, CFLAR, FGF9,
KRT40, OR3A1, RPP38, TMEM198, AGO3, CFP, FGFBP1, KRT5, OR3A2, RPP40, TMEM199,
AGO4, CFTR, FGFBP2, KRT6A, OR3A3, RPRD1A, TMEM2, AGPAT1, CGA, FGFBP3, KRT6B,
OR4A15, RPRD1B, TMEM200A, AGPAT2, CGB, FGFR1, KRT6C, OR4A16, RPRD2, TMEM200B,
AGPAT3, CGB1, FGFR1OP, KRT7, OR4A47, RPRM, TMEM201, AGPAT4, CGB2, FGFR1OP2,
KRT71, OR4A5, RPRML, TMEM202, AGPAT5, CGB5, FGFR2, KRT72, OR4B1, RPS10, TMEM203,
AGPAT6, CGB7, FGFR3, KRT73, OR4C11, RPS10-NUDT3, TMEM204, AGPAT9, CGB8, FGFR4,
KRT74, OR4C12, RPS11, TMEM205, AGPS, CGGBP1, FGFRL1, KRT75, OR4C13, RPS12,
TMEM206, AGR2, CGN, FGG, KRT76, OR4C15, RPS13, TMEM207, AGR3, CGNL1, FGGY, KRT77,
OR4C16, RPS14, TMEM208, AGRN, CGREF1, FGL1, KRT78, OR4C3, RPS15, TMEM209, AGRP,
CGRRF1, FGL2, KRT79, OR4C45, RPS15A, TMEM210, AGT, CH25H, FGR, KRT8, OR4C46, RPS16,
TMEM211, AGTPBP1, CHAC1, FH, KRT80, OR4C6, RPS17, TMEM212, AGTR1, CHAC2, FHAD1,
KRT81, OR4D1, RPS17L, TMEM213, AGTR2, CHAD, FHDC1, KRT82, OR4D10, RPS18, TMEM214,
AGTRAP, CHADL, FHIT, KRT83, OR4D11, RPS19, TMEM215, AGXT, CHAF1A, FHL1, KRT84,
OR4D2, RPS19BP1, TMEM216, AGXT2, CHAF1B, FHL2, KRT85, OR4D5, RPS2, TMEM217,
AHCTF1, CHAMP1, FHL3, KRT86, OR4D6, RPS20, TMEM218, AHCY, CHAT, FHL5, KRT9,
OR4D9, RPS21, TMEM219, AHCYL1, CHCHD1, FHOD1, KRTAP10-1, OR4E2, RPS23, TMEM220,
AHCYL2, CHCHD10, FHOD3, KRTAP10-10, OR4F15, RPS24, TMEM221, AHDC1, CHCHD2,
FIBCD1, KRTAP10-11, OR4F16, RPS25, TMEM222, AHI1, CHCHD3, FIBIN, KRTAP10-12, OR4F21,
RPS26, TMEM223, AHNAK, CHCHD4, FIBP, KRTAP10-2, OR4F29, RPS27, TMEM225, AHNAK2,
CHCHD5, FICD, KRTAP10-3, OR4F3, RPS27A, TMEM229A, AHR, CHCHD6, FIG. 4, KRTAP10-4,
OR4F4, RPS27L, TMEM229B, AHRR, CHCHD7, FIGF, KRTAP10-5, OR4F5, RPS28, TMEM230,
AHSA1, CHD1, FIGLA, KRTAP10-6, OR4F6, RPS29, TMEM231, AHSA2, CHD1L, FIGN, KRTAP10-
7, OR4K1, RPS3, TMEM232, AHSG, CHD2, FIGNL1, KRTAP10-8, OR4K13, RPS3A, TMEM233,
AHSP, CHD3, FIGNL2, KRTAP10-9, OR4K14, RPS4X, TMEM234, AICDA, CHD4, FILIP1, KRTAP1-
1, OR4K15, RPS4Y1, TMEM235, AIDA, CHD5, FILIP1L, KRTAP11-1, OR4K17, RPS4Y2,
TMEM236, AIF1, CHD6, FIPIL1, KRTAP12-1, OR4K2, RPS5, TMEM237, AIF1L, CHD7, FIS1,
KRTAP12-2, OR4K5, RPS6, TMEM238, AIFM1, CHD8, FITM1, KRTAP12-3, OR4L1, RPS6KA1,
TMEM239, AIFM2, CHD9, FITM2, KRTAP12-4, OR4M1, RPS6KA2, TMEM240, AIFM3, CHDC2,
FIZ1, KRTAP1-3, OR4M2, RPS6KA3, TMEM241, AIG1, CHDH, FJX1, KRTAP13-1, OR4N2,
RPS6KA4, TMEM242, AIM1, CHEK1, FKBP10, KRTAP13-2, OR4N4, RPS6KA5, TMEM243, AIM1L,
CHEK2, FKBP11, KRTAP13-3, OR4N5, RPS6KA6, TMEM244, AIM2, CHERP, FKBP14, KRTAP13-
4, OR4P4, RPS6KB1, TMEM245, AIMP1, CHFR, FKBP15, KRTAP1-4, OR4Q3, RPS6KB2,
TMEM246, AIMP2, CHGA, FKBP1A, KRTAP1-5, OR4S1, RPS6KC1, TMEM247, AIP, CHGB,
FKBP1B, KRTAP15-1, OR4S2, RPS6KL1, TMEM248, AIPL1, CHI3L1, FKBP2, KRTAP16-1, OR4X1,
RPS7, TMEM249, AIRE, CHI3L2, FKBP3, KRTAP17-1, OR4X2, RPS8, TMEM25, AJAP1, CHIA,
FKBP4, KRTAP19-1, OR51A2, RPS9, TMEM251, AJUBA, CHIC1, FKBP5, KRTAP19-2, OR51A4,
RPSA, TMEM252, AK1, CHIC2, FKBP6, KRTAP19-3, OR51A7, RPTN, TMEM253, AK2, CHID1,
FKBP7, KRTAP19-4, OR51B2, RPTOR, TMEM254, AK3, CHIT1, FKBP8, KRTAP19-5, OR51B4,
RPUSD1, TMEM255A, AK4, CHKA, FKBP9, KRTAP19-6, OR51B5, RPUSD2, TMEM255B, AK5,
CHKB, FKBPL, KRTAP19-7, OR51B6, RPUSD3, TMEM256, AK6, CHL1, FKRP, KRTAP19-8,
OR51D1, RPUSD4, TMEM257, AK7, CHM, FKTN, KRTAP20-1, OR51E1, RQCD1, TMEM258, AK8,
CHML, FLAD1, KRTAP20-2, OR51E2, RRAD, TMEM259, AK9, CHMP1A, FLCN, KRTAP20-3,
OR51F1, RRAGA, TMEM26, AKAP1, CHMP1B, FLG, KRTAP2-1, OR51F2, RRAGB, TMEM260,
AKAP10, CHMP2A, FLG2, KRTAP21-1, OR51G1, RRAGC, TMEM261, AKAP11, CHMP2B, FLI1,
KRTAP21-2, OR51G2, RRAGD, TMEM27, AKAP12, CHMP3, FLII, KRTAP21-3, OR51I1, RRAS,
TMEM30A, AKAP13, CHMP4A, FLJ22184, KRTAP2-2, OR51I2, RRAS2, TMEM30B, AKAP14,
CHMP4B, FLJ25363, KRTAP22-1, OR51L1, RRBP1, TMEM31, AKAP17A, CHMP4C, FLJ44313,
KRTAP22-2, OR51M1, RREB1, TMEM33, AKAP2, CHMP5, FLJ44635, KRTAP2-3, OR51Q1, RRH,
TMEM35, AKAP3, CHMP6, FLJ45513, KRTAP23-1, OR51S1, RRM1, TMEM37, AKAP4, CHMP7,
FLNA, KRTAP2-4, OR51T1, RRM2, TMEM38A, AKAP5, CHN1, FLNB, KRTAP24-1, OR51V1,
RRM2B, TMEM38B, AKAP6, CHN2, FLNC, KRTAP25-1, OR52A1, RRN3, TMEM39A, AKAP7,
CHODL, FLOT1, KRTAP26-1, OR52A5, RRNAD1, TMEM39B, AKAP8, CHORDC1, FLOT2,
KRTAP27-1, OR52B2, RRP1, TMEM40, AKAP8L, CHP1, FLRT1, KRTAP29-1, OR52B4, RRP12,
TMEM41A, AKAP9, CHP2, FLRT2, KRTAP3-1, OR52B6, RRP15, TMEM41B, AKIP1, CHPF, FLRT3,
KRTAP3-2, OR52D1, RRP1B, TMEM42, AKIRIN1, CHPF2, FLT1, KRTAP3-3, OR52E2, RRP36,
TMEM43, AKIRIN2, CHPT1, FLT3, KRTAP4-1, OR52E4, RRP7A, TMEM44, AKNA, CHRAC1,
FLT3LG, KRTAP4-11, OR52E6, RRP8, TMEM45A, AKNAD1, CHRD, FLT4, KRTAP4-12, OR52E8,
RRP9, TMEM45B, AKR1A1, CHRDL1, FLVCR1, KRTAP4-2, OR52H1, RRS1, TMEM47, AKR1B1,
CHRDL2, FLVCR2, KRTAP4-3, OR52I1, RS1, TMEM5, AKR1B10, CHRFAM7A, FLYWCH1,
KRTAP4-4, OR52I2, RSAD1, TMEM50A, AKR1B15, CHRM1, FLYWCH2, KRTAP4-5, OR52J3,
RSAD2, TMEM50B, AKR1C1, CHRM2, FMN1, KRTAP4-6, OR52K1, RSBN1, TMEM51, AKR1C2,
CHRM3, FMN2, KRTAP4-7, OR52K2, RSBN1L, TMEM52, AKR1C3, CHRM4, FMNL1, KRTAP4-8,
OR52L1, RSC1A1, TMEM52B, AKR1C4, CHRM5, FMNL2, KRTAP4-9, OR52M1, RSF1, TMEM53,
AKR1D1, CHRNA1, FMNL3, KRTAP5-1, OR52N1, RSG1, TMEM54, AKR1E2, CHRNA10, FMO1,
KRTAP5-10, OR52N4, RSL1D1, TMEM55A, AKR7A2, CHRNA2, FMO2, KRTAP5-11, OR52N5,
RSL24D1, TMEM55B, AKR7A3, CHRNA3, FMO3, KRTAP5-2, OR52R1, RSPH1, TMEM56, AKT1,
CHRNA4, FMO4, KRTAP5-3, OR52W1, RSPH10B, TMEM56-RWDD3, AKT1S1, CHRNA5, FMO5,
KRTAP5-4, OR56A1, RSPH10B2, TMEM57, AKT2, CHRNA6, FMOD, KRTAP5-5, OR56A3, RSPH3,
TMEM59, AKT3, CHRNA7, FMR1, KRTAP5- 6, OR56A4, RSPH4A, TMEM59L, AKTIP, CHRNA9,
FMR1NB, KRTAP5-7, OR56A5, RSPH6A, TMEM60, ALAD, CHRNB1, FN1, KRTAP5-8, OR56B1,
RSPH9, TMEM61, ALAS1, CHRNB2, FN3K, KRTAP5-9, OR56B4, RSPO1, TMEM62, ALAS2,
CHRNB3, FN3KRP, KRTAP6-1, OR5A1, RSPO2, TMEM63A, ALB, CHRNB4, FNBP1, KRTAP6-2,
OR5A2, RSPO3, TMEM63B, ALCAM, CHRND, FNBP1L, KRTAP6-3, OR5AC2, RSPO4, TMEM63C,
ALDH16A1, CHRNE, FNBP4, KRTAP7-1, OR5AK2, RSPRY1, TMEM64, ALDH18A1, CHRNG,
FNDC1, KRTAP8-1, OR5AN1, RSRC1, TMEM65, ALDH1A1, CHST1, FNDC3A, KRTAP9-1,
OR5AP2, RSRC2, TMEM66, ALDH1A2, CHST10, FNDC3B, KRTAP9-2, OR5AR1, RSU1, TMEM67,
ALDH1A3, CHST11, FNDC4, KRTAP9-3, OR5AS1, RTBDN, TMEM68, ALDH1B1, CHST12,
FNDC5, KRTAP9-4, OR5AU1, RTCA, TMEM69, ALDH1L1, CHST13, FNDC7, KRTAP9-6, OR5B12,
RTCB, TMEM70, ALDH1L2, CHST14, FNDC8, KRTAP9-7, OR5B17, RTDR1, TMEM71, ALDH2,
CHST15, FNDC9, KRTAP9- 8, OR5B2, RTEL1, TMEM72, ALDH3A1, CHST2, FNIP1, KRTAP9-9,
OR5B21, RTF1, TMEM74, ALDH3A2, CHST3, FNIP2, KRTCAP2, OR5B3, RTFDC1, TMEM74B,
ALDH3B1, CHST4, FNTA, KRTCAP3, OR5C1, RTKN, TMEM79, ALDH3B2, CHST5, FNTB,
KRTDAP, OR5D13, RTKN2, TMEM80, ALDH4A1, CHST6, FOCAD, KSR1, OR5D14, RTL1,
TMEM81, ALDH5A1, CHST7, FOLH1, KSR2, OR5D16, RTN1, TMEM82, ALDH6A1, CHST8,
FOLH1B, KTI12, OR5D18, RTN2, TMEM86A, ALDH7A1, CHST9, FOLR1, KTN1, OR5F1, RTN3,
TMEM86B, ALDH8A1, CHSY1, FOLR2, KXD1, OR5H1, RTN4, TMEM87A, ALDH9A1, CHSY3,
FOLR3, KY, OR5H14, RTN4IP1, TMEM87B, ALDOA, CHTF18, FOLR4, KYNU, OR5H15, RTN4R,
TMEM88, ALDOB, CHTF8, FOPNL, L1CAM, OR5H2, RTN4RL1, TMEM88B, ALDOC, CHTOP,
FOS, L1TD1, OR5H6, RTN4RL2, TMEM89, ALG1, CHUK, FOSB, L2HGDH, OR5I1, RTP1,
TMEM8A, ALG10, CHURC1, FOSL1, L3HYPDH, OR5J2, RTP2, TMEM8B, ALG10B, CHURC1-
FNTB, FOSL2, L3MBTL1, OR5K1, RTP3, TMEM8C, ALG11, CIAO1, FOXA1, L3MBTL2, OR5K2,
RTP4, TMEM9, ALG12, CIAPIN1, FOXA2, L3MBTL3, OR5K3, RTTN, TMEM91, ALG13, CIB1,
FOXA3, L3MBTL4, OR5K4, RUFY1, TMEM92, ALG14, CIB2, FOXB1, LACC1, OR5L1, RUFY2,
TMEM95, ALG1L, CIB3, FOXB2, LACE1, OR5L2, RUFY3, TMEM97, ALG1L2, CIB4, FOXC1,
LACRT, OR5M1, RUFY4, TMEM98, ALG2, CIC, FOXC2, LACTB, OR5M10, RUNDC1, TMEM99,
ALG3, CIDEA, FOXD1, LACTB2, OR5M11, RUNDC3A, TMEM9B, ALG5, CIDEB, FOXD2,
LACTBL1, OR5M3, RUNDC3B, TMF1, ALG6, CIDEC, FOXD3, LAD1, OR5M8, RUNX1, TMIE,
ALG8, CIITA, FOXD4, LAG3, OR5M9, RUNX1T1, TMIGD1, ALG9, CILP, FOXD4L1, LAGE3,
OR5P2, RUNX2, TMIGD2, ALK, CILP2, FOXD4L2, LAIR1, OR5P3, RUNX3, TMLHE, ALKBH1,
CINP, FOXD4L3, LAIR2, OR5R1, RUSC1, TMOD1, ALKBH2, CIR1, FOXD4L4, LALBA, OR5T1,
RUSC1-AS1, TMOD2, ALKBH3, CIRBP, FOXD4L5, LAMA1, OR5T2, RUSC2, TMOD3, ALKBH4,
CIRH1A, FOXD4L6, LAMA2, OR5T3, RUVBL1, TMOD4, ALKBH5, CISD1, FOXE1, LAMA3,
OR5V1, RUVBL2, TMPO, ALKBH6, CISD2, FOXE3, LAMA4, OR5W2, RWDD1, TMPPE, ALKBH7,
CISD3, FOXF1, LAMA5, OR6A2, RWDD2A, TMPRSS11A, ALKBH8, CISH, FOXF2, LAMB1,
OR6B1, RWDD2B, TMPRSS11B, ALLC, CIT, FOXG1, LAMB2, OR6B3, RWDD3, TMPRSS11D,
ALMS1, CITED1, FOXH1, LAMB3, OR6C1, RWDD4, TMPRSS11E, ALOX12, CITED2, FOXI1,
LAMB4, OR6C2, RXFP1, TMPRSS11F, ALOX12B, CITED4, FOXI2, LAMC1, OR6C3, RXFP2,
TMPRSS12, ALOX15, CIZ1, FOXI3, LAMC2, OR6C4, RXFP3, TMPRSS13, ALOX15B, CKAP2,
FOXJ1, LAMC3, OR6C6, RXFP4, TMPRSS15, ALOX5, CKAP2L, FOXJ2, LAMP1, OR6C65, RXRA,
TMPRSS2, ALOX5AP, CKAP4, FOXJ3, LAMP2, OR6C68, RXRB, TMPRSS3, ALOXE3, CKAP5,
FOXK1, LAMP3, OR6C70, RXRG, TMPRSS4, ALPI, CKB, FOXK2, LAMP5, OR6C74, RYBP,
TMPRSS5, ALPK1, CKLF, FOXL1, LAMTOR1, OR6C75, RYK, TMPRSS6, ALPK2, CKLF-CMTM1,
FOXL2, LAMTOR2, OR6C76, RYR1, TMPRSS7, ALPK3, CKM, FOXM1, LAMTOR3, OR6F1, RYR2,
TMPRSS9, ALPL, CKMT1A, FOXN1, LAMTOR4, OR6K2, RYR3, TMSB10, ALPP, CKMT1B,
FOXN2, LAMTOR5, OR6K3, S100A1, TMSB15A, ALPPL2, CKMT2, FOXN3, LANCLI, OR6K6,
S100A10, TMSB15B, ALS2, CKS1B, FOXN4, LANCL2, OR6M1, S100A11, TMSB4X, ALS2CL,
CKS2, FOXO1, LANCL3, OR6N1, S100A12, TMSB4Y, ALS2CR11, CLASP1, FOX03, LAP3, OR6N2,
S100A13, TMTC1, ALS2CR12, CLASP2, FOXO4, LAPTM4A, OR6P1, S100A14, TMTC2, ALX1,
CLASRP, FOXO6, LAPTM4B, OR6Q1, S100A16, TMTC3, ALX3, CLC, FOXP1, LAPTM5, OR6S1,
S100A2, TMTC4, ALX4, CLCA1, FOXP2, LARGE, OR6T1, S100A3, TMUB1, ALYREF, CLCA2,
FOXP3, LARP1, OR6V1, S100A4, TMUB2, AMACR, CLCA4, FOXP4, LARP1B, OR6X1, S100A5,
TMX1, AMBN, CLCC1, FOXQ1, LARP4, OR6Y1, S100A6, TMX2, AMBP, CLCF1, FOXR1,
LARP4B, OR7A10, S100A7, TMX3, AMBRA1, CLCN1, FOXR2, LARP6, OR7A17, S100A7A, TMX4,
AMD1, CLCN2, FOXRED1, LARP7, OR7A5, S100A7L2, TNC, AMDHD1, CLCN3, FOXRED2,
LARS, OR7C1, S100A8, TNF, AMDHD2, CLCN4, FOXS1, LARS2, OR7C2, S100A9, TNFAIP1,
AMELX, CLCN5, FPGS, LAS1L, OR7D2, S100B, TNFAIP2, AMELY, CLCN6, FPGT, LASP1,
OR7D4, S100G, TNFAIP3, AMER1, CLCN7, FPGT-TNNI3K, LAT, OR7E24, S100P, TNFAIP6,
AMER2, CLCNKA, FPR1, LAT2, OR7G1, S100PBP, TNFAIP8, AMER3, CLCNKB, FPR2, LATS1,
OR7G2, S100Z, TNFAIP8L1, AMFR, CLDN1, FPR3, LATS2, OR7G3, S1PR1, TNFAIP8L2, AMH,
CLDN10, FRA10AC1, LAX1, OR8A1, S1PR2, TNFAIP8L2-SCNM1, AMHR2, CLDN11, FRAS1,
LAYN, OR8B12, SIPR3, TNFAIP8L3, AMICA1, CLDN12, FRAT1, LBH, OR8B2, S1PR4,
TNFRSF10A, AMIGO1, CLDN14, FRAT2, LBP, OR8B3, S1PR5, TNFRSF10B, AMIGO2, CLDN15,
FREM1, LBR, OR8B4, SAA1, TNFRSF10C, AMIGO3, CLDN16, FREM2, LBX1, OR8B8, SAA2,
TNFRSF10D, AMMECR1, CLDN17, FREM3, LBX2, OR8D1, SAA2-SAA4, TNFRSF11A,
AMMECR1L, CLDN18, FRG1, LCA5, OR8D2, SAA4, TNFRSF11B, AMN, CLDN19, FRG2, LCA5L,
OR8D4, SAAL1, TNFRSF12A, AMN1, CLDN2, FRG2B, LCAT, OR8G1, SAC3D1, TNFRSF13B,
AMOT, CLDN20, FRG2C, LCE1A, OR8G2, SACM1L, TNFRSF13C, AMOTL1, CLDN22, FRK,
LCE1B, OR8G5, SACS, TNFRSF14, AMOTL2, CLDN23, FRMD1, LCE1C, OR8H1, SAE1,
TNFRSF17, AMPD1, CLDN24, FRMD3, LCE1D, OR8H2, SAFB, TNFRSF18, AMPD2, CLDN25,
FRMD4A, LCE1E, OR8H3, SAFB2, TNFRSF19, AMPD3, CLDN3, FRMD4B, LCE1F, OR8I2, SAG,
TNFRSF1A, AMPH, CLDN4, FRMD5, LCE2A, OR8J1, SAGE1, TNFRSF1B, AMT, CLDN5, FRMD6,
LCE2B, OR8J3, SALL1, TNFRSF21, AMTN, CLDN6, FRMD7, LCE2C, OR8K1, SALL2, TNFRSF25,
AMY1A, CLDN7, FRMD8, LCE2D, OR8K3, SALL3, TNFRSF4, AMY1B, CLDN8, FRMPD1, LCE3A,
OR8K5, SALL4, TNFRSF6B, AMY1C, CLDN9, FRMPD2, LCE3B, OR8S1, SAMD1, TNFRSF8,
AMY2A, CLDND1, FRMPD3, LCE3C, OR8U1, SAMD10, TNFRSF9, AMY2B, CLDND2, FRMPD4,
LCE3D, OR8U8, SAMD11, TNFSF10, AMZ1, CLEC10A, FRRS1, LCE3E, OR9A2, SAMD12,
TNFSF11, AMZ2, CLEC11A, FRRS1L, LCE4A, OR9A4, SAMD13, TNFSF12, ANAPC1, CLEC12A,
FRS2, LCE5A, OR9G1, SAMD14, TNFSF12-TNFSF13, ANAPC10, CLEC12B, FRS3, LCE6A, OR9G4,
SAMD15, TNFSF13, ANAPC11, CLEC14A, FRY, LCK, OR9G9, SAMD3, TNFSF13B, ANAPC13,
CLEC16A, FRYL, LCLAT1, OR9I1, SAMD4A, TNFSF14, ANAPC15, CLEC17A, FRZB, LCMT1,
OR9K2, SAMD4B, TNFSF15, ANAPC16, CLEC18A, FSBP, LCMT2, OR9Q1, SAMD5, TNFSF18,
ANAPC2, CLEC18B, FSCB, LCN1, OR9Q2, SAMD7, TNFSF4, ANAPC4, CLEC18C, FSCN1, LCN10,
ORAI1, SAMD8, TNFSF8, ANAPC5, CLEC19A, FSCN2, LCN12, ORAI2, SAMD9, TNFSF9,
ANAPC7, CLEC1A, FSCN3, LCN15, ORAI3, SAMD9L, TNIK, ANG, CLEC1B, FSD1, LCN2,
ORAOV1, SAMHD1, TNIP1, ANGEL1, CLEC2A, FSD1L, LCN6, ORC1, SAMM50, TNIP2, ANGEL2,
CLEC2B, FSD2, LCN8, ORC2, SAMSN1, TNIP3, ANGPT1, CLEC2D, FSHB, LCN9, ORC3, SAP130,
TNK1, ANGPT2, CLEC2L, FSHR, LCNL1, ORC4, SAP18, TNK2, ANGPT4, CLEC3A, FSIP1, LCOR,
ORC5, SAP25, TNKS, ANGPTL1, CLEC3B, FSIP2, LCORL, ORC6, SAP30, TNKS1BP1, ANGPTL2,
CLEC4A, FST, LCP1, ORM1, SAP30BP, TNKS2, ANGPTL3, CLEC4C, FSTL1, LCP2, ORM2,
SAP30L, TNMD, ANGPTL4, CLEC4D, FSTL3, LCT, ORMDL1, SAPCD1, TNN, ANGPTL5, CLEC4E,
FSTL4, LCTL, ORMDL2, SAPCD2, TNNC1, ANGPTL6, CLEC4F, FSTL5, LDB1, ORMDL3, SAR1A,
TNNC2, ANGPTL7, CLEC4G, FTCD, LDB2, OS9, SAR1B, TNNI1, ANHX, CLEC4M, FTH1, LDB3,
OSBP, SARDH, TNNI2, ANK1, CLEC5A, FTH1P18, LDHA, OSBP2, SARM1, TNNI3, ANK2,
CLEC6A, FTHL17, LDHAL6A, OSBPL10, SARNP, TNNI3K, ANK3, CLEC7A, FTL, LDHAL6B,
OSBPL11, SARS, TNNT1, ANKAR, CLEC9A, FTMT, LDHB, OSBPL1A, SARS2, TNNT2,
ANKDD1A, CLECL1, FTO, LDHC, OSBPL2, SART1, TNNT3, ANKDD1B, CLGN, FTSJ1, LDHD,
OSBPL3, SART3, TNP1, ANKEF1, CLHC1, FTSJ2, LDLR, OSBPL5, SASH1, TNP2, ANKFN1,
CLIC1, FTSJ3, LDLRAD1, OSBPL6, SASH3, TNPO1, ANKFY1, CLIC2, FUBP1, LDLRAD2,
OSBPL7, SASS6, TNPO2, ANKH, CLIC3, FUBP3, LDLRAD3, OSBPL8, SAT1, TNPO3, ANKHD1,
CLIC4, FUCA1, LDLRAD4, OSBPL9, SAT2, TNR, ANKHD1-EIF4EBP3, CLIC5, FUCA2, LDLRAP1,
OSCAR, SATB1, TNRC18, ANKIB1, CLIC6, FUK, LDOC1, OSCP1, SATB2, TNRC6A, ANKK1,
CLINT1, FUNDC1, LDOC1L, OSER1, SATL1, TNRC6B, ANKLE1, CLIP1, FUNDC2, LEAP2,
OSGEP, SAV1, TNRC6C, ANKLE2, CLIP2, FUOM, LECT1, OSGEPL1, SAYSD1, TNS1, ANKMY1,
CLIP3, FURIN, LECT2, OSGIN1, SBDS, TNS3, ANKMY2, CLIP4, FUS, LEF1, OSGIN2, SBF1, TNS4,
ANKRA2, CLK1, FUT1, LEFTY1, OSM, SBF2, TNXB, ANKRD1, CLK2, FUT10, LEFTY2, OSMR,
SBK1, TOB1, ANKRD10, CLK3, FUT11, LEKR1, OSR1, SBK2, TOB2, ANKRD11, CLK4, FUT2,
LELP1, OSR2, SBK3, TOE1, ANKRD12, CLLU1, FUT3, LEMD1, OST4, SBNO1, TOLLIP,
ANKRD13A, CLLU1OS, FUT4, LEMD2, OSTC, SBNO2, TOM1, ANKRD13B, CLMN, FUT5,
LEMD3, OSTF1, SBSN, TOM1L1, ANKRD13C, CLMP, FUT6, LENEP, OSTM1, SBSPON, TOM1L2,
ANKRD13D, CLN3, FUT7, LENG1, OSTN, SC5D, TOMM20, ANKRD16, CLN5, FUT8, LENG8,
OTC, SCAF1, TOMM20L, ANKRD17, CLN6, FUT9, LENG9, OTOA, SCAF11, TOMM22,
ANKRD18A, CLN8, FUZ, LEO1, OTOF, SCAF4, TOMM34, ANKRD18B, CLNK, FXN, LEP, OTOG,
SCAF8, TOMM40, ANKRD2, CLNS1A, FXR1, LEPR, OTOGL, SCAI, TOMM40L, ANKRD20A1,
CLOCK, FXR2, LEPRE1, OTOL1, SCAMP1, TOMM5, ANKRD20A2, CLP1, FXYD1, LEPREL1,
OTOP1, SCAMP2, TOMM6, ANKRD20A3, CLPB, FXYD2, LEPREL2, OTOP2, SCAMP3, TOMM7,
ANKRD20A4, CLPP, FXYD3, LEPREL4, OTOP3, SCAMP4, TOMM70A, ANKRD22, CLPS, FXYD4,
LEPROT, OTOR, SCAMP5, TONSL, ANKRD23, CLPSL1, FXYD5, LEPROTL1, OTOS, SCAND1,
TOP1, ANKRD24, CLPSL2, FXYD6, LETM1, OTP, SCAND3, TOP1MT, ANKRD26, CLPTM1,
FXYD6-FXYD2, LETM2, OTUB1, SCAP, TOP2A, ANKRD27, CLPTM1L, FXYD7, LETMD1,
OTUB2, SCAPER, TOP2B, ANKRD28, CLPX, FYB, LEUTX, OTUD1, SCARA3, TOP3A, ANKRD29,
CLRN1, FYCO1, LFNG, OTUD3, SCARA5, TOP3B, ANKRD30A, CLRN2, FYN, LGALS1, OTUD4,
SCARB1, TOPAZ1, ANKRD30B, CLRN3, FYTTD1, LGALS12, OTUD5, SCARB2, TOPBP1,
ANKRD31, CLSPN, FZD1, LGALS13, OTUD6A, SCARF1, TOPORS, ANKRD32, CLSTN1, FZD10,
LGALS14, OTUD6B, SCARF2, TOR1A, ANKRD33, CLSTN2, FZD2, LGALS16, OTUD7A, SCCPDH,
TOR1AIP1, ANKRD33B, CLSTN3, FZD3, LGALS2, OTUD7B, SCD, TOR1AIP2, ANKRD34A,
CLTA, FZD4, LGALS3, OTX1, SCD5, TOR1B, ANKRD34B, CLTB, FZD5, LGALS3BP, OTX2,
SCEL, TOR2A, ANKRD34C, CLTC, FZD6, LGALS4, OVCA2, SCFD1, TOR3A, ANKRD35, CLTCL1,
FZD7, LGALS7, OVCH1, SCFD2, TOR4A, ANKRD36, CLU, FZD8, LGALS7B, OVCH2, SCG2, TOX,
ANKRD36B, CLUAP1, FZD9, LGALS8, OVGP1, SCG3, TOX2, ANKRD36C, CLUH, FZR1, LGALS9,
OVOL1, SCG5, TOX3, ANKRD37, CLUL1, G0S2, LGALS9B, OVOL2, SCGB1A1, TOX4, ANKRD39,
CLVS1, G2E3, LGALS9C, OVOL3, SCGB1C1, TP53, ANKRD40, CLVS2, G3BP1, LGALSL, OVOS,
SCGB1D1, TP53AIP1, ANKRD42, CLYBL, G3BP2, LGI1, OVOS2, SCGB1D2, TP53BP1, ANKRD44,
CMA1, G6PC, LGI2, OXA1L, SCGB1D4, TP53BP2, ANKRD45, CMAS, G6PC2, LGI3, OXCT1,
SCGB2A1, TP53I11, ANKRD46, CMBL, G6PC3, LGI4, OXCT2, SCGB2A2, TP53I13, ANKRD49,
CMC1, G6PD, LGMN, OXER1, SCGB2B2, TP53I3, ANKRD50, CMC2, GAA, LGR4, OXGR1,
SCGB3A1, TP53INP1, ANKRD52, CMC4, GAB1, LGR5, OXLD1, SCGB3A2, TP53INP2, ANKRD53,
CMIP, GAB2, LGR6, OXNAD1, SCGN, TP53RK, ANKRD54, CMKLR1, GAB3, LGSN, OXR1,
SCHIP1, TP53TG3, ANKRD55, CMPK1, GAB4, LHB, OXSM, SCIMP, TP53TG3B, ANKRD6,
CMPK2, GABARAP, LHCGR, OXSR1, SCIN, TP53TG3C, ANKRD60, CMSS1, GABARAPL1, LHFP,
OXT, SCLT1, TP53TG3D, ANKRD61, CMTM1, GABARAPL2, LHFPL1, OXTR, SCLY, TP53TG5,
ANKRD62, CMTM2, GABBR1, LHFPL2, P2RX1, SCMH1, TP63, ANKRD63, CMTM3, GABBR2,
LHFPL3, P2RX2, SCML1, TP73, ANKRD65, CMTM4, GABPA, LHFPL4, P2RX3, SCML2, TPBG,
ANKRD66, CMTM5, GABPB1, LHFPL5, P2RX4, SCML4, TPBGL, ANKRD7, CMTM6, GABPB2,
LHPP, P2RX5, SCN10A, TPCN1, ANKRD9, CMTM7, GABRA1, LHX1, P2RX6, SCN11A, TPCN2,
ANKS1A, CMTM8, GABRA2, LHX2, P2RX7, SCN1A, TPD52, ANKS1B, CMTR1, GABRA3, LHX3,
P2RY1, SCN1B, TPD52L1, ANKS3, CMTR2, GABRA4, LHX4, P2RY10, SCN2A, TPD52L2,
ANKS4B, CMYA5, GABRA5, LHX5, P2RY11, SCN2B, TPD52L3, ANKS6, CNBD1, GABRA6,
LHX6, P2RY12, SCN3A, TPGS1, ANKUB1, CNBD2, GABRB1, LHX8, P2RY13, SCN3B, TPGS2,
ANKZF1, CNBP, GABRB2, LHX9, P2RY14, SCN4A, TPH1, ANLN, CNDP1, GABRB3, LIAS,
P2RY2, SCN4B, TPH2, ANO1, CNDP2, GABRD, LIF, P2RY4, SCN5A, TPI1, ANO10, CNEP1R1,
GABRE, LIFR, P2RY6, SCN7A, TPK1, ANO2, CNFN, GABRG1, LIG1, P2RY8, SCN8A, TPM1,
ANO3, CNGA1, GABRG2, LIG3, P4HA1, SCN9A, TPM2, ANO4, CNGA2, GABRG3, LIG4, P4HA2,
SCNM1, TPM3, ANO5, CNGA3, GABRP, LILRA1, P4HA3, SCNN1A, TPM4, ANO6, CNGA4,
GABRQ, LILRA2, P4HB, SCNN1B, TPMT, ANO7, CNGB1, GABRR1, LILRA3, P4HTM, SCNN1D,
TPO, ANO8, CNGB3, GABRR2, LILRA4, PA2G4, SCNN1G, TPP1, ANO9, CNIH1, GABRR3,
LILRA5, PAAF1, SCO1, TPP2, ANP32A, CNIH2, GAD1, LILRA6, PABPC1, SCO2, TPPP, ANP32B,
CNIH3, GAD2, LILRB1, PABPC1L, SCOC, TPPP2, ANP32C, CNIH4, GADD45A, LILRB2,
PABPC1L2A, SCP2, TPPP3, ANP32D, CNKSR1, GADD45B, LILRB3, PABPC1L2B, SCP2D1, TPR,
ANP32E, CNKSR2, GADD45G, LILRB4, PABPC3, SCPEP1, TPRA1, ANPEP, CNKSR3,
GADD45GIP1, LILRB5, PABPC4, SCRG1, TPRG1, ANTXR1, CNN1, GADL1, LIM2, PABPC4L,
SCRIB, TPRG1L, ANTXR2, CNN2, GAGE1, LIMA1, PABPC5, SCRN1, TPRKB, ANTXRL, CNN3,
GAGE10, LIMCH1, PABPN1, SCRN2, TPRN, ANXA1, CNNM1, GAGE12B, LIMD1, PABPN1L,
SCRN3, TPRX1, ANXA10, CNNM2, GAGE12C, LIMD2, PACRG, SCRT1, TPSAB1, ANXA11,
CNNM3, GAGE12D, LIME1, PACRGL, SCRT2, TPSB2, ANXA13, CNNM4, GAGE12E, LIMK1,
PACS1, SCT, TPSD1, ANXA2, CNOT1, GAGE12F, LIMK2, PACS2, SCTR, TPSG1, ANXA2R,
CNOT10, GAGE12G, LIMS1, PACSIN1, SCUBE1, TPST1, ANXA3, CNOT11, GAGE12H, LIMS2,
PACSIN2, SCUBE2, TPST2, ANXA4, CNOT2, GAGE12I, LIMS3, PACSIN3, SCUBE3, TPT1,
ANXA5, CNOT3, GAGE12J, LIN28A, PADI1, SCXA, TPTE, ANXA6, CNOT4, GAGE13, LIN28B,
PADI2, SCXB, TPTE2, ANXA7, CNOT6, GAGE2A, LIN37, PADI3, SCYL1, TPX2, ANXA8,
CNOT6L, GAGE2B, LIN52, PADI4, SCYL2, TRA2A, ANXA8L1, CNOT7, GAGE2C, LIN54, PADI6,
SCYL3, TRA2B, ANXA8L2, CNOT8, GAGE2D, LIN7A, PAEP, SDAD1, TRABD, ANXA9, CNP,
GAGE2E, LIN7B, PAF1, SDC1, TRABD2A, AOAH, CNPPD1, GAGE4, LIN7C, PAFAH1B1, SDC2,
TRABD2B, AOC1, CNPY1, GAGE5, LIN9, PAFAH1B2, SDC3, TRADD, AOC2, CNPY2, GAGE6,
LINC00452, PAFAH1B3, SDC4, TRAF1, AOC3, CNPY3, GAGE7, LINC00984, PAFAH2, SDCBP,
TRAF2, AOX1, CNPY4, GAGE8, LINGO1, PAG1, SDCBP2, TRAF3, AP1AR, CNR1, GAK, LINGO2,
PAGE1, SDCCAG3, TRAF3IP1, AP1B1, CNR2, GAL, LINGO3, PAGE2, SDCCAG8, TRAF3IP2,
APIG1, CNRIP1, GAL3ST1, LINGO4, PAGE2B, SDE2, TRAF3IP3, AP1G2, CNST, GAL3ST2, LINS,
PAGE4, SDF2, TRAF4, AP1M1, CNTD1, GAL3ST3, LIPA, PAGE5, SDF2L1, TRAF5, AP1M2,
CNTD2, GAL3ST4, LIPC, PAGR1, SDF4, TRAF6, AP1S1, CNTF, GALC, LIPE, PAH, SDHA, TRAF7,
AP1S2, CNTFR, GALE, LIPF, PAICS, SDHAF1, TRAFD1, APIS3, CNTLN, GALK1, LIPG, PAIP1,
SDHAF2, TRAIP, AP2A1, CNTN1, GALK2, LIPH, PAIP2, SDHB, TRAK1, AP2A2, CNTN2, GALM,
LIPI, PAIP2B, SDHC, TRAK2, AP2B1, CNTN3, GALNS, LIPJ, PAK1, SDHD, TRAM1, AP2M1,
CNTN4, GALNT1, LIPK, PAK1IP1, SDK1, TRAM1L1, AP2S1, CNTN5, GALNT10, LIPM, PAK2,
SDK2, TRAM2, AP3B1, CNTN6, GALNT11, LIPN, PAK3, SDPR, TRANK1, AP3B2, CNTNAP1,
GALNT12, LIPT1, PAK4, SDR16C5, TRAP1, AP3D1, CNTNAP2, GALNT13, LIPT2, PAK6,
SDR39U1, TRAPPC1, AP3M1, CNTNAP3, GALNT14, LITAF, PAK7, SDR42E1, TRAPPC10, AP3M2,
CNTNAP3B, GALNT15, LIX1, PALB2, SDR9C7, TRAPPC11, AP3S1, CNTNAP4, GALNT16, LIX1L,
PALD1, SDS, TRAPPC12, AP3S2, CNTNAP5, GALNT18, LLGL1, PALLD, SDSL, TRAPPC13,
AP4B1, CNTRL, GALNT2, LLGL2, PALM, SEBOX, TRAPPC2, AP4E1, CNTROB, GALNT3, LLPH,
PALM2, SEC11A, TRAPPC2L, AP4M1, COA1, GALNT4, LMAN1, PALM2-AKAP2, SEC11C,
TRAPPC3, AP4S1, COA3, GALNT5, LMAN1L, PALM3, SEC13, TRAPPC3L, AP5B1, COA4,
GALNT6, LMAN2, PALMD, SEC14L1, TRAPPC4, AP5M1, COA5, GALNT7, LMAN2L, PAM,
SEC14L2, TRAPPC5, AP5S1, COA6, GALNT8, LMBR1, PAM16, SEC14L3, TRAPPC6A, AP5Z1,
COASY, GALNT9, LMBR1L, PAMR1, SEC14L4, TRAPPC6B, APAF1, COBL, GALNTL5, LMBRD1,
PAN2, SEC14L5, TRAPPC8, APBA1, COBLL1, GALNTL6, LMBRD2, PAN3, SEC14L6, TRAPPC9,
APBA2, COCH, GALP, LMCD1, PANK1, SEC16A, TRAT1, APBA3, COG1, GALR1, LMF1, PANK2,
SEC16B, TRDMT1, APBB1, COG2, GALR2, LMF2, PANK3, SEC22A, TRDN, APBB1IP, COG3,
GALR3, LMLN, PANK4, SEC22B, TREH, APBB2, COG4, GALT, LMNA, PANX1, SEC22C, TREM1,
APBB3, COG5, GAMT, LMNB1, PANX2, SEC23A, TREM2, APC, COG6, GAN, LMNB2, PANX3,
SEC23B, TREML1, APC2, COG7, GANAB, LMO1, PAOX, SEC23IP, TREML2, APCDD1, COG8,
GANC, LMO2, PAPD4, SEC24A, TREML4, APCDD1L, COIL, GAP43, LMO3, PAPD5, SEC24B,
TRERF1, APCS, COL10A1, GAPDH, LMO4, PAPD7, SEC24C, TREX1, APEH, COL11A1, GAPDHS,
LMO7, PAPL, SEC24D, TREX2, APEX1, COL11A2, GAPT, LMOD1, PAPLN, SEC31A, TRH,
APEX2, COL12A1, GAPVD1, LMOD2, PAPOLA, SEC31B, TRHDE, APH1A, COL13A1, GAR1,
LMOD3, PAPOLB, SEC61A1, TRHR, APH1B, COL14A1, GAREM, LMTK2, PAPOLG, SEC61A2,
TRIAP1, API5, COL15A1, GAREML, LMTK3, PAPPA, SEC61B, TRIB1, APIP, COL16A1, GARNL3,
LMX1A, PAPPA2, SEC61G, TRIB2, APITD1, COL17A1, GARS, LMX1B, PAPSS1, SEC62, TRIB3,
APITD1-CORT, COL18A1, GART, LNP1, PAPSS2, SEC63, TRIL, APLF, COL19A1, GAS1, LNPEP,
PAQR3, SECISBP2, TRIM10, APLN, COL1A1, GAS2, LNX1, PAQR4, SECISBP2L, TRIM11,
APLNR, COL1A2, GAS2L1, LNX2, PAQR5, SECTM1, TRIM13, APLP1, COL20A1, GAS2L2,
LOH12CR1, PAQR6, SEH1L, TRIM14, APLP2, COL21A1, GAS2L3, LONP1, PAQR7, SEL1L,
TRIM15, APMAP, COL22A1, GAS6, LONP2, PAQR8, SEL1L2, TRIM16, APOA1, COL23A1, GAS7,
LONRF1, PAQR9, SEL1L3, TRIM16L, APOA1BP, COL24A1, GAS8, LONRF2, PARD3, SELE,
TRIM17, APOA2, COL25A1, GAST, LONRF3, PARD3B, SELENBP1, TRIM2, APOA4, COL26A1,
GATA1, LOR, PARD6A, SELK, TRIM21, APOA5, COL27A1, GATA2, LOX, PARD6B, SELL,
TRIM22, APOB, COL28A1, GATA3, LOXHD1, PARD6G, SELM, TRIM23, APOBEC1, COL2A1,
GATA4, LOXL1, PARG, SELO, TRIM24, APOBEC2, COL3A1, GATA5, LOXL2, PARK2, SELP,
TRIM25, APOBEC3A, COL4A1, GATA6, LOXL3, PARK7, SELPLG, TRIM26, APOBEC3B,
COL4A2, GATAD1, LOXL4, PARL, SELRC1, TRIM27, APOBEC3C, COL4A3, GATAD2A, LPA,
PARM1, SELT, TRIM28, APOBEC3D, COL4A3BP, GATAD2B, LPAR1, PARN, SELV, TRIM29,
APOBEC3F, COL4A4, GATC, LPAR2, PARP1, SEMA3A, TRIM3, APOBEC3G, COL4A5, GATM,
LPAR3, PARP10, SEMA3B, TRIM31, APOBEC3H, COL4A6, GATS, LPAR4, PARP11, SEMA3C,
TRIM32, APOBEC4, COL5A1, GATSL1, LPAR5, PARP12, SEMA3D, TRIM33, APOBR, COL5A2,
GATSL2, LPAR6, PARP14, SEMA3E, TRIM34, APOC1, COL5A3, GATSL3, LPCAT1, PARP15,
SEMA3F, TRIM35, APOC2, COL6A1, GBA, LPCAT2, PARP16, SEMA3G, TRIM36, APOC3,
COL6A2, GBA2, LPCAT3, PARP2, SEMA4A, TRIM37, APOC4, COL6A3, GBA3, LPCAT4, PARP3,
SEMA4B, TRIM38, APOD, COL6A5, GBAS, LPGAT1, PARP4, SEMA4C, TRIM39, APOE, COL6A6,
GBE1, LPHN1, PARP6, SEMA4D, TRIM39-RPP21, APOF, COL7A1, GBF1, LPHN2, PARP8,
SEMA4F, TRIM4, APOH, COL8A1, GBGT1, LPHN3, PARP9, SEMA4G, TRIM40, APOL1, COL8A2,
GBP1, LPIN1, PARPBP, SEMA5A, TRIM41, APOL2, COL9A1, GBP2, LPIN2, PARS2, SEMA5B,
TRIM42, APOL3, COL9A2, GBP3, LPIN3, PARVA, SEMA6A, TRIM43, APOL4, COL9A3, GBP4,
LPL, PARVB, SEMA6B, TRIM43B, APOL5, COLCA2, GBP5, LPO, PARVG, SEMA6C, TRIM44,
APOL6, COLEC10, GBP6, LPP, PASD1, SEMA6D, TRIM45, APOLD1, COLEC11, GBP7, LPPR1,
PASK, SEMA7A, TRIM46, APOM, COLEC12, GBX1, LPPR2, PATE1, SEMG1, TRIM47, APOO,
COLGALT1, GBX2, LPPR3, PATE2, SEMG2, TRIM48, APOOL, COLGALT2, GC, LPPR4, PATE3,
SENP1, TRIM49, APOPT1, COLQ, GCA, LPPR5, PATE4, SENP2, TRIM49B, APP, COMMD1, GCAT,
LPXN, PATL1, SENP3, TRIM49C, APPBP2, COMMD10, GCC1, LRAT, PATL2, SENP5, TRIM49D1,
APPL1, COMMD2, GCC2, LRBA, PATZ1, SENP6, TRIM49D2P, APPL2, COMMD3, GCDH, LRCH1,
PAWR, SENP7, TRIM5, APRT, COMMD3-BMI1, GCFC2, LRCH2, PAX1, SENP8, TRIM50, APTX,
COMMD4, GCG, LRCH3, PAX2, SEPHS1, TRIM51, AQP1, COMMD5, GCGR, LRCH4, PAX3,
SEPHS2, TRIM52, AQP10, COMMD6, GCH1, LRCOL1, PAX4, SEPN1, TRIM54, AQP11, COMMD7,
GCHFR, LRFN1, PAX5, SEPP1, TRIM55, AQP12A, COMMD8, GCK, LRFN2, PAX6, SEPSECS,
TRIM56, AQP12B, COMMD9, GCKR, LRFN3, PAX7, SEPT1, TRIM58, AQP2, COMP, GCLC,
LRFN4, PAX8, SEPT10, TRIM59, AQP3, COMT, GCLM, LRFN5, PAX9, SEPT11, TRIM6, AQP4,
COMTD1, GCM1, LRG1, PAXBP1, SEPT12, TRIM60, AQP5, COPA, GCM2, LRGUK, PAXIP1,
SEPT14, TRIM61, AQP6, COPB1, GCN1L1, LRIF1, PBDC1, SEPT15, TRIM62, AQP7, COPB2,
GCNT1, LRIG1, PBK, SEPT2, TRIM63, AQP8, COPE, GCNT2, LRIG2, PBLD, SEPT3, TRIM64,
AQP9, COPG1, GCNT3, LRIG3, PBOV1, SEPT4, TRIM64B, AQPEP, COPG2, GCNT4, LRIT1,
PBRM1, SEPT5, TRIM64C, AQR, COPRS, GCNT7, LRIT2, PBX1, SEPT6, TRIM65, AR, COPS2,
GCOM1, LRIT3, PBX2, SEPT7, TRIM66, ARAF, COPS3, GCSAM, LRMP, PBX3, SEPT8, TRIM67,
ARAP1, COPS4, GCSAML, LRP1, PBX4, SEPT9, TRIM68, ARAP2, COPS5, GCSH, LRP10, PBXIP1,
SEPW1, TRIM69, ARAP3, COPS6, GDA, LRP11, PC, SERAC1, TRIM6-TRIM34, ARC, COPS7A,
GDAP1, LRP12, PCBD1, SERBP1, TRIM7, ARCN1, COPS7B, GDAP1L1, LRP1B, PCBD2, SERF1A,
TRIM71, AREG, COPS8, GDAP2, LRP2, PCBP1, SERF1B, TRIM72, AREGB, COPZ1, GDE1,
LRP2BP, PCBP2, SERF2, TRIM73, AREL1, COPZ2, GDF1, LRP3, PCBP3, SERGEF, TRIM74, ARF1,
COQ10A, GDF10, LRP4, PCBP4, SERHL2, TRIM77, ARF3, COQ10B, GDF11, LRP5, PCCA,
SERINC1, TRIM8, ARF4, COQ2, GDF15, LRP5L, PCCB, SERINC2, TRIM9, ARF5, COQ3, GDF2,
LRP6, PCDH1, SERINC3, TRIML1, ARF6, COQ4, GDF3, LRP8, PCDH10, SERINC4, TRIML2,
ARFGAP1, COQ5, GDF5, LRPAP1, PCDH11X, SERINC5, TRIO, ARFGAP2, COQ6, GDF6, LRPPRC,
PCDH11Y, SERP1, TRIOBP, ARFGAP3, COQ7, GDF7, LRR1, PCDH12, SERP2, TRIP10, ARFGEF1,
COQ9, GDF9, LRRC1, PCDH15, SERPINA1, TRIP11, ARFGEF2, CORIN, GDI1, LRRC10, PCDH17,
SERPINA10, TRIP12, ARFIP1, CORO1A, GDI2, LRRC10B, PCDH18, SERPINA11, TRIP13, ARFIP2,
CORO1B, GDNF, LRRC14, PCDH19, SERPINA12, TRIP4, ARFRP1, CORO1C, GDNF-AS1,
LRRC14B, PCDH20, SERPINA3, TRIP6, ARG1, CORO2A, GDPD1, LRRC15, PCDH7, SERPINA4,
TRIQK, ARG2, CORO2B, GDPD2, LRRC16A, PCDH8, SERPINA5, TRIT1, ARGFX, CORO6,
GDPD3, LRRC16B, PCDH9, SERPINA6, TRMT1, ARGLU1, CORO7, GDPD4, LRRC17, PCDHA1,
SERPINA7, TRMT10A, ARHGAP1, CORO7-PAM16, GDPD5, LRRC18, PCDHA10, SERPINA9,
TRMT10B, ARHGAP10, CORT, GDPGP1, LRRC19, PCDHA11, SERPINB1, TRMT10C,
ARHGAP11A, COTL1, GEM, LRRC2, PCDHA12, SERPINB10, TRMT11, ARHGAP11B, COX10,
GEMIN2, LRRC20, PCDHA13, SERPINB11, TRMT112, ARHGAP12, COX11, GEMIN4, LRRC23,
PCDHA2, SERPINB12, TRMT12, ARHGAP15, COX14, GEMIN5, LRRC24, PCDHA3, SERPINB13,
TRMT13, ARHGAP17, COX15, GEMIN6, LRRC25, PCDHA4, SERPINB2, TRMT1L, ARHGAP18,
COX16, GEMIN7, LRRC26, PCDHA5, SERPINB3, TRMT2A, ARHGAP19, COX17, GEMIN8,
LRRC27, PCDHA6, SERPINB4, TRMT2B, ARHGAP20, COX18, GEN1, LRRC28, PCDHA7,
SERPINB5, TRMT44, ARHGAP21, COX19, GET4, LRRC29, PCDHA8, SERPINB6, TRMT5,
ARHGAP22, COX20, GFAP, LRRC3, PCDHA9, SERPINB7, TRMT6, ARHGAP23, COX4I1, GFER,
LRRC30, PCDHAC1, SERPINB8, TRMT61A, ARHGAP24, COX4I2, GFI1, LRRC31, PCDHAC2,
SERPINB9, TRMT61B, ARHGAP25, COX5A, GFI1B, LRRC32, PCDHB1, SERPINC1, TRMU,
ARHGAP26, COX5B, GFM1, LRRC34, PCDHB10, SERPIND1, TRNAU1AP, ARHGAP27, COX6A1,
GFM2, LRRC36, PCDHB11, SERPINE1, TRNP1, ARHGAP28, COX6A2, GFOD1, LRRC37A2,
PCDHB12, SERPINE2, TRNT1, ARHGAP29, COX6B1, GFOD2, LRRC37A3, PCDHB13, SERPINE3,
TRO, ARHGAP30, COX6B2, GFPT1, LRRC37B, PCDHB14, SERPINF1, TROAP, ARHGAP31,
COX6C, GFPT2, LRRC38, PCDHB15, SERPINF2, TROVE2, ARHGAP32, COX7A1, GFRA1,
LRRC39, PCDHB16, SERPING1, TRPA1, ARHGAP33, COX7A2, GFRA2, LRRC3B, PCDHB2,
SERPINH1, TRPC1, ARHGAP35, COX7A2L, GFRA3, LRRC3C, PCDHB3, SERPINI1, TRPC3,
ARHGAP36, COX7B, GFRA4, LRRC4, PCDHB4, SERPINI2, TRPC4, ARHGAP39, COX7B2,
GFRAL, LRRC40, PCDHB5, SERTAD1, TRPC4AP, ARHGAP4, COX7C, GGA1, LRRC41, PCDHB6,
SERTAD2, TRPC5, ARHGAP40, COX8A, GGA2, LRRC42, PCDHB7, SERTAD3, TRPC5OS,
ARHGAP42, COX8C, GGA3, LRRC43, PCDHB8, SERTAD4, TRPC6, ARHGAP44, CP, GGACT,
LRRC45, PCDHB9, SERTM1, TRPC7, ARHGAP5, CPA1, GGCT, LRRC46, PCDHGA1, SESN1,
TRPM1, ARHGAP6, CPA2, GGCX, LRRC47, PCDHGA10, SESN2, TRPM2, ARHGAP8, CPA3, GGH,
LRRC48, PCDHGA11, SESN3, TRPM3, ARHGAP9, CPA4, GGN, LRRC49, PCDHGA12, SESTD1,
TRPM4, ARHGDIA, CPA5, GGNBP2, LRRC4B, PCDHGA2, SET, TRPM5, ARHGDIB, CPA6,
GGPS1, LRRC4C, PCDHGA3, SETBP1, TRPM6, ARHGDIG, CPAMD8, GGT1, LRRC52, PCDHGA4,
SETD1A, TRPM7, ARHGEF1, CPB1, GGT2, LRRC55, PCDHGA5, SETD1B, TRPM8, ARHGEF10,
CPB2, GGT5, LRRC56, PCDHGA6, SETD2, TRPS1, ARHGEF10L, CPD, GGT6, LRRC57, PCDHGA7,
SETD3, TRPT1, ARHGEF11, CPE, GGT7, LRRC58, PCDHGA8, SETD4, TRPV1, ARHGEF12,
CPEB1, GGTLC1, LRRC59, PCDHGA9, SETD5, TRPV2, ARHGEF15, CPEB2, GGTLC2, LRRC6,
PCDHGB1, SETD6, TRPV3, ARHGEF16, CPEB3, GH1, LRRC61, PCDHGB2, SETD7, TRPV4,
ARHGEF17, CPEB4, GH2, LRRC63, PCDHGB3, SETD8, TRPV5, ARHGEF18, CPED1, GHDC,
LRRC66, PCDHGB4, SETD9, TRPV6, ARHGEF19, CPLX1, GHITM, LRRC69, PCDHGB5, SETDB1,
TRRAP, ARHGEF2, CPLX2, GHR, LRRC7, PCDHGB6, SETDB2, TRUB1, ARHGEF25, CPLX3,
GHRH, LRRC70, PCDHGB7, SETMAR, TRUB2, ARHGEF26, CPLX4, GHRHR, LRRC71,
PCDHGC3, SETSIP, TSACC, ARHGEF28, CPM, GHRL, LRRC72, PCDHGC4, SETX, TSC1,
ARHGEF3, CPN1, GHSR, LRRC73, PCDHGC5, SEZ6, TSC2, ARHGEF33, CPN2, GID4, LRRC8A,
PCDP1, SEZ6L, TSC22D1, ARHGEF35, CPNE1, GID8, LRRC8B, PCED1A, SEZ6L2, TSC22D2,
ARHGEF37, CPNE2, GIF, LRRC8C, PCED1B, SF1, TSC22D3, ARHGEF38, CPNE3, GIGYF1,
LRRC8D, PCF11, SF3A1, TSC22D4, ARHGEF39, CPNE4, GIGYF2, LRRC8E, PCGF1, SF3A2,
TSEN15, ARHGEF4, CPNE5, GIMAP1, LRRCC1, PCGF2, SF3A3, TSEN2, ARHGEF40, CPNE6,
GIMAP1-GIMAP5, LRRD1, PCGF3, SF3B1, TSEN34, ARHGEF5, CPNE7, GIMAP2, LRRFIP1,
PCGF5, SF3B14, TSEN54, ARHGEF6, CPNE8, GIMAP4, LRRFIP2, PCGF6, SF3B2, TSFM,
ARHGEF7, CPNE9, GIMAP5, LRRIQ1, PCID2, SF3B3, TSG101, ARHGEF9, CPO, GIMAP6, LRRIQ3,
PCIF1, SF3B4, TSGA10, ARID1A, CPOX, GIMAP7, LRRIQ4, PCK1, SF3B5, TSGA10IP, ARID1B,
CPPED1, GIMAP8, LRRK1, PCK2, SFI1, TSGA13, ARID2, CPQ, GIMD1, LRRK2, PCLO, SFMBT1,
TSHB, ARID3A, CPS1, GIN1, LRRN1, PCM1, SFMBT2, TSHR, ARID3B, CPSF1, GINM1, LRRN2,
PCMT1, SFN, TSHZ1, ARID3C, CPSF2, GINS1, LRRN3, PCMTD1, SFPQ, TSHZ2, ARID4A, CPSF3,
GINS2, LRRN4, PCMTD2, SFR1, TSHZ3, ARID4B, CPSF3L, GINS3, LRRN4CL, PCNA, SFRP1,
TSKS, ARID5A, CPSF4, GINS4, LRRTM1, PCNP, SFRP2, TSKU, ARID5B, CPSF4L, GIP, LRRTM2,
PCNT, SFRP4, TSLP, ARIH1, CPSF6, GIPC1, LRRTM3, PCNX, SFRP5, TSN, ARIH2, CPSF7, GIPC2,
LRRTM4, PCNXL2, SFSWAP, TSNARE1, ARIH2OS, CPT1A, GIPC3, LRSAM1, PCNXL3, SFT2D1,
TSNAX, ARL1, CPT1B, GIPR, LRTM1, PCNXL4, SFT2D2, TSNAXIP1, ARL10, CPT1C, GIT1,
LRTM2, PCOLCE, SFT2D3, TSPAN1, ARL11, CPT2, GIT2, LRTOMT, PCOLCE2, SFTA2, TSPAN10,
ARL13A, CPVL, GJA1, LRWD1, PCP2, SFTA3, TSPAN11, ARL13B, CPXCR1, GJA10, LSAMP,
PCP4, SFTPA1, TSPAN12, ARL14, CPXM1, GJA3, LSG1, PCP4L1, SFTPA2, TSPAN13, ARL14EP,
CPXM2, GJA4, LSM1, PCSK1, SFTPB, TSPAN14, ARL14EPL, CPZ, GJA5, LSM10, PCSK1N,
SFTPC, TSPAN15, ARL15, CR1, GJA8, LSM11, PCSK2, SFTPD, TSPAN16, ARL16, CR1L, GJA9,
LSM12, PCSK4, SFXN1, TSPAN17, ARL17A, CR2, GJB1, LSM14A, PCSK5, SFXN2, TSPAN18,
ARL17B, CRABP1, GJB2, LSM14B, PCSK6, SFXN3, TSPAN19, ARL2, CRABP2, GJB3, LSM2,
PCSK7, SFXN4, TSPAN2, ARL2BP, CRADD, GJB4, LSM3, PCSK9, SFXN5, TSPAN3, ARL3,
CRAMP1L, GJB5, LSM4, PCTP, SGCA, TSPAN31, ARL4A, CRAT, GJB6, LSM5, PCYOX1, SGCB,
TSPAN32, ARL4C, CRB1, GJB7, LSM6, PCYOX1L, SGCD, TSPAN33, ARL4D, CRB2, GJC1, LSM7,
PCYT1A, SGCE, TSPAN4, ARL5A, CRB3, GJC2, LSMD1, PCYT1B, SGCG, TSPAN5, ARL5B,
CRBN, GJC3, LSMEM1, PCYT2, SGCZ, TSPAN6, ARL5C, CRCP, GJD2, LSMEM2, PDAP1, SGIP1,
TSPAN7, ARL6, CRCT1, GJD3, LSP1, PDC, SGK1, TSPAN8, ARL6IP1, CREB1, GJD4, LSR, PDCD1,
SGK2, TSPAN9, ARL6IP4, CREB3, GK, LSS, PDCD10, SGK223, TSPEAR, ARL6IP5, CREB3L1,
GK2, LST1, PDCD11, SGK3, TSPO, ARL6IP6, CREB3L2, GK5, LTA, PDCD1LG2, SGK494, TSPO2,
ARL8A, CREB3L3, GKAP1, LTA4H, PDCD2, SGMS1, TSPY1, ARL8B, CREB3L4, GKN1, LTB,
PDCD2L, SGMS2, TSPY10, ARL9, CREB5, GKN2, LTB4R, PDCD4, SGOL1, TSPY2, ARMC1,
CREBBP, GLA, LTB4R2, PDCD5, SGOL2, TSPY3, ARMC10, CREBL2, GLB1, LTBP1, PDCD6,
SGPL1, TSPY4, ARMC12, CREBRF, GLB1L, LTBP2, PDCD6IP, SGPP1, TSPY8, ARMC2, CREBZF,
GLB1L2, LTBP3, PDCD7, SGPP2, TSPYL1, ARMC3, CREG1, GLB1L3, LTBP4, PDCL, SGSH,
TSPYL2, ARMC4, CREG2, GLCCI1, LTBR, PDCL2, SGSM1, TSPYL4, ARMC5, CRELD1, GLCE,
LTC4S, PDCL3, SGSM2, TSPYL5, ARMC6, CRELD2, GLDC, LTF, PDDC1, SGSM3, TSPYL6,
ARMC7, CREM, GLDN, LTK, PDE10A, SGTA, TSR1, ARMC8, CRH, GLE1, LTN1, PDE11A, SGTB,
TSR2, ARMC9, CRHBP, GLG1, LTV1, PDE12, SH2B1, TSR3, ARMCX1, CRHR1, GLI1, LUC7L,
PDE1A, SH2B2, TSSC1, ARMCX2, CRHR2, GLI2, LUC7L2, PDE1B, SH2B3, TSSC4, ARMCX3,
CRIM1, GLI3, LUC7L3, PDE1C, SH2D1A, TSSK1B, ARMCX4, CRIP1, GLI4, LUM, PDE2A,
SH2D1B, TSSK2, ARMCX5, CRIP2, GLIPR1, LURAP1, PDE3A, SH2D2A, TSSK3, ARMCX5-
GPRASP2, CRIP3, GLIPR1L1, LURAP1L, PDE3B, SH2D3A, TSSK4, ARMCX6, CRIPAK,
GLIPR1L2, LUZP1, PDE4A, SH2D3C, TSSK6, ARMS2, CRIPT, GLIPR2, LUZP2, PDE4B, SH2D4A,
TST, ARNT, CRISP1, GLIS1, LUZP4, PDE4C, SH2D4B, TSTA3, ARNT2, CRISP2, GLIS2, LUZP6,
PDE4D, SH2D5, TSTD1, ARNTL, CRISP3, GLIS3, LXN, PDE4DIP, SH2D6, TSTD2, ARNTL2,
CRISPLD1, GLMN, LY6D, PDE5A, SH2D7, TSTD3, ARPC1A, CRISPLD2, GLO1, LY6E, PDE6A,
SH3BGR, TTBK1, ARPC1B, CRK, GLOD4, LY6G5B, PDE6B, SH3BGRL, TTBK2, ARPC2, CRKL,
GLOD5, LY6G5C, PDE6C, SH3BGRL2, TTC1, ARPC3, CRLF1, GLP1R, LY6G6C, PDE6D,
SH3BGRL3, TTC12, ARPC4, CRLF2, GLP2R, LY6G6D, PDE6G, SH3BP1, TTC13, ARPC4-TTLL3,
CRLF3, GLRA1, LY6G6F, PDE6H, SH3BP2, TTC14, ARPC5, CRLS1, GLRA2, LY6H, PDE7A,
SH3BP4, TTC16, ARPC5L, CRMP1, GLRA3, LY6K, PDE7B, SH3BP5, TTC17, ARPP19, CRNKL1,
GLRA4, LY75, PDE8A, SH3BP5L, TTC18, ARPP21, CRNN, GLRB, LY75-CD302, PDE8B, SH3D19,
TTC19, ARR3, CROCC, GLRX, LY86, PDE9A, SH3D21, TTC21A, ARRB1, CROT, GLRX2, LY9,
PDF, SH3GL1, TTC21B, ARRB2, CRP, GLRX3, LY96, PDGFA, SH3GL2, TTC22, ARRDC1,
CRTAC1, GLRX5, LYAR, PDGFB, SH3GL3, TTC23, ARRDC2, CRTAM, GLS, LYG1, PDGFC,
SH3GLB1, TTC23L, ARRDC3, CRTAP, GLS2, LYG2, PDGFD, SH3GLB2, TTC24, ARRDC4, CRTC1,
GLT1D1, LYL1, PDGFRA, SH3KBP1, TTC25, ARRDC5, CRTC2, GLT6D1, LYN, PDGFRB,
SH3PXD2A, TTC26, ARSA, CRTC3, GLT8D1, LYNX1, PDGFRL, SH3PXD2B, TTC27, ARSB, CRX,
GLT8D2, LYPD1, PDHA1, SH3RF1, TTC28, ARSD, CRY1, GLTP, LYPD2, PDHA2, SH3RF2, TTC29,
ARSE, CRY2, GLTPD1, LYPD3, PDHB, SH3RF3, TTC3, ARSF, CRYAA, GLTPD2, LYPD4, PDHX,
SH3TC1, TTC30A, ARSG, CRYAB, GLTSCR1, LYPD5, PDIA2, SH3TC2, TTC30B, ARSH, CRYBA1,
GLTSCR1L, LYPD6, PDIA3, SH3YL1, TTC31, ARSI, CRYBA2, GLTSCR2, LYPD6B, PDIA4,
SHANK1, TTC32, ARSJ, CRYBA4, GLUD1, LYPD8, PDIA5, SHANK2, TTC33, ARSK, CRYBB1,
GLUD2, LYPLA1, PDIA6, SHANK3, TTC34, ARTI, CRYBB2, GLUL, LYPLA2, PDIK1L, SHARPIN,
TTC36, ART3, CRYBB3, GLYAT, LYPLAL1, PDILT, SHB, TTC37, ART4, CRYBG3, GLYATL1,
LYRM1, PDK1, SHBG, TTC38, ART5, CRYGA, GLYATL2, LYRM2, PDK2, SHC1, TTC39A, ARTN,
CRYGB, GLYATL3, LYRM4, PDK3, SHC2, TTC39B, ARV1, CRYGC, GLYCTK, LYRM5, PDK4,
SHC3, TTC39C, ARVCF, CRYGD, GLYR1, LYRM7, PDLIM1, SHC4, TTC4, ARX, CRYGN, GM2A,
LYRM9, PDLIM2, SHCBP1, TTC40, AS3MT, CRYGS, GMCL1, LYSMD1, PDLIM3, SHCBP1L,
TTC5, ASAH1, CRYL1, GMDS, LYSMD2, PDLIM4, SHD, TTC6, ASAH2, CRYM, GMEB1,
LYSMD3, PDLIM5, SHE, TTC7A, ASAH2B, CRYZ, GMEB2, LYSMD4, PDLIM7, SHF, TTC7B,
ASAP1, CRYZL1, GMFB, LYST, PDP1, SHFM1, TTC8, ASAP2, CS, GMFG, LYVE1, PDP2, SHH,
TTC9, ASAP3, CSAD, GMIP, LYZ, PDPK1, SHISA2, TTC9B, ASB1, CSAG1, GML, LYZL1, PDPN,
SHISA3, TTC9C, ASB10, CSAG2, GMNC, LYZL2, PDPR, SHISA4, TTF1, ASB11, CSAG3, GMNN,
LYZL4, PDRG1, SHISA5, TTF2, ASB12, CSDC2, GMPPA, LYZL6, PDS5A, SHISA6, TTI1, ASB13,
CSDE1, GMPPB, LZIC, PDS5B, SHISA7, TTI2, ASB14, CSE1L, GMPR, LZTFL1, PDSS1, SHISA8,
TTK, ASB15, CSF1, GMPR2, LZTR1, PDSS2, SHISA9, TTL, ASB16, CSF1R, GMPS, LZTS1, PDX1,
SHKBP1, TTLL1, ASB17, CSF2, GNA11, LZTS2, PDXDC1, SHMT1, TTLL10, ASB18, CSF2RA,
GNA12, LZTS3, PDXK, SHMT2, TTLL11, ASB2, CSF2RB, GNA13, M1AP, PDXP, SHOC2, TTLL12,
ASB3, CSF3, GNA14, M6PR, PDYN, SHOX, TTLL13, ASB4, CSF3R, GNA15, MAATS1, PDZD11,
SHOX2, TTLL2, ASB5, CSGALNACT1, GNAI1, MAB21L1, PDZD2, SHPK, TTLL3, ASB6,
CSGALNACT2, GNAI2, MAB21L2, PDZD3, SHPRH, TTLL4, ASB7, CSH1, GNAI3, MAB21L3,
PDZD4, SHQ1, TTLL5, ASB8, CSH2, GNAL, MACC1, PDZD7, SHROOM1, TTLL6, ASB9, CSHL1,
GNAO1, MACF1, PDZD8, SHROOM2, TTLL7, ASCC1, CSK, GNAQ, MACROD1, PDZD9,
SHROOM3, TTLL8, ASCC2, CSMD1, GNAS, MACROD2, PDZK1, SHROOM4, TTLL9, ASCC3,
CSMD2, GNAT1, MAD1L1, PDZK1IP1, SI, TTN, ASCL1, CSMD3, GNAT2, MAD2L1, PDZRN3,
SIAE, TTPA, ASCL2, CSN1S1, GNAT3, MAD2L1BP, PDZRN4, SIAH1, TTPAL, ASCL3, CSN2,
GNAZ, MAD2L2, PEA15, SIAH2, TTR, ASCL4, CSN3, GNB1, MADCAM1, PEAK1, SIAH3, TTYH1,
ASCL5, CSNK1A1, GNB1L, MADD, PEAR1, SIDT1, TTYH2, ASF1A, CSNK1A1L, GNB2, MAEA,
PEBP1, SIDT2, TTYH3, ASF1B, CSNK1D, GNB2L1, MAEL, PEBP4, SIGIRR, TUB, ASGR1,
CSNK1E, GNB3, MAF, PECAM1, SIGLEC1, TUBA1A, ASGR2, CSNK1G1, GNB4, MAF1, PECR,
SIGLEC10, TUBA1B, ASH1L, CSNK1G2, GNB5, MAFA, PEF1, SIGLEC11, TUBA1C, ASH2L,
CSNK1G3, GNE, MAFB, PEG10, SIGLEC12, TUBA3C, ASIC1, CSNK2A1, GNG10, MAFF, PEG3,
SIGLEC14, TUBA3D, ASIC2, CSNK2A2, GNG11, MAFG, PELI1, SIGLEC15, TUBA3E, ASIC3,
CSNK2A3, GNG12, MAFK, PELI2, SIGLEC5, TUBA4A, ASIC4, CSNK2B, GNG13, MAG, PELI3,
SIGLEC6, TUBA8, ASIC5, CSPG4, GNG2, MAGEA1, PELO, SIGLEC7, TUBAL3, ASIP, CSPG5,
GNG3, MAGEA10, PELP1, SIGLEC8, TUBB, ASL, CSPP1, GNG4, MAGEA10-MAGEA5, PEMT,
SIGLEC9, TUBB1, ASMT, CSRNP1, GNG5, MAGEA11, PENK, SIGLECL1, TUBB2A, ASMTL,
CSRNP2, GNG7, MAGEA12, PEPD, SIGMAR1, TUBB2B, ASNA1, CSRNP3, GNG8, MAGEA3,
PER1, SIK1, TUBB3, ASNS, CSRP1, GNGT1, MAGEA4, PER2, SIK2, TUBB4A, ASNSD1, CSRP2,
GNGT2, MAGEA5, PER3, SIK3, TUBB4B, ASPA, CSRP2BP, GNL1, MAGEA6, PERM1, SIKE1,
TUBB6, ASPDH, CSRP3, GNL2, MAGEA8, PERP, SIL1, TUBB8, ASPG, CST1, GNL3, MAGEB1,
PES1, SIM1, TUBD1, ASPH, CST11, GNL3L, MAGEB10, PET100, SIM2, TUBE1, ASPHD1, CST2,
GNLY, MAGEB16, PET112, SIMC1, TUBG1, ASPHD2, CST3, GNMT, MAGEB17, PET117, SIN3A,
TUBG2, ASPM, CST4, GNPAT, MAGEB18, PEX1, SIN3B, TUBGCP2, ASPN, CST5, GNPDA1,
MAGEB2, PEX10, SIPA1, TUBGCP3, ASPRV1, CST6, GNPDA2, MAGEB3, PEX11A, SIPA1L1,
TUBGCP4, ASPSCR1, CST7, GNPNAT1, MAGEB4, PEX11B, SIPA1L2, TUBGCP5, ASRGL1, CST8,
GNPTAB, MAGEB5, PEX11G, SIPAIL3, TUBGCP6, ASS1, CST9, GNPTG, MAGEB6, PEX12,
SIRPA, TUFM, ASTE1, CST9L, GNRH1, MAGEC1, PEX13, SIRPB1, TUFT1, ASTL, CSTA, GNRH2,
MAGEC2, PEX14, SIRPB2, TULP1, ASTN1, CSTB, GNRHR, MAGEC3, PEX16, SIRPD, TULP2,
ASTN2, CSTF1, GNS, MAGED1, PEX19, SIRPG, TULP3, ASUN, CSTF2, GOLGA1, MAGED2,
PEX2, SIRT1, TULP4, ASXL1, CSTF2T, GOLGA2, MAGEE1, PEX26, SIRT2, TUSC1, ASXL2,
CSTF3, GOLGA3, MAGEE2, PEX3, SIRT3, TUSC2, ASXL3, CSTL1, GOLGA4, MAGEF1, PEX5,
SIRT4, TUSC3, ASZ1, CT45A1, GOLGA5, MAGEH1, PEX5L, SIRT5, TUSC5, ATAD1, CT45A2,
GOLGA6A, MAGEL2, PEX6, SIRT6, TUT1, ATAD2, CT45A3, GOLGA6B, MAGI1, PEX7, SIRT7,
TVP23A, ATAD2B, CT45A4, GOLGA6C, MAGI2, PF4, SIT1, TVP23B, ATAD3A, CT45A5,
GOLGA6D, MAGI3, PF4V1, SIVA1, TVP23C, ATAD3B, CT45A6, GOLGA6L1, MAGIX, PFAS,
SIX1, TVP23C-CDRT4, ATAD3C, CT47A1, GOLGA6L10, MAGOH, PFDN1, SIX2, TWF1, ATAD5,
CT47A10, GOLGA6L2, MAGOHB, PFDN2, SIX3, TWF2, ATAT1, CT47A11, GOLGA6L3, MAGT1,
PFDN4, SIX4, TWIST1, ATCAY, CT47A12, GOLGA6L4, MAK, PFDN5, SIX5, TWIST2, ATE1,
CT47A2, GOLGA6L6, MAK16, PFDN6, SIX6, TWISTNB, ATF1, CT47A3, GOLGA6L9, MAL,
PFKFB1, SKA1, TWSG1, ATF2, CT47A4, GOLGA7, MAL2, PFKFB2, SKA2, TXK, ATF3, CT47A5,
GOLGA7B, MALL, PFKFB3, SKA3, TXLNA, ATF4, CT47A6, GOLGA8A, MALSU1, PFKFB4,
SKAP1, TXLNB, ATF5, CT47A7, GOLGA8B, MALT1, PFKL, SKAP2, TXLNG, ATF6, CT47A8,
GOLGA8H, MAMDC2, PFKM, SKI, TXN, ATF6B, CT47A9, GOLGA8J, MAMDC4, PFKP, SKIDA1,
TXN2, ATF7, CT47B1, GOLGA8K, MAML1, PFN1, SKIL, TXNDC11, ATF7IP, CT62, GOLGA8M,
MAML2, PFN2, SKIV2L, TXNDC12, ATF7IP2, CTAG1A, GOLGA80, MAML3, PFN3, SKIV2L2,
TXNDC15, ATG10, CTAG1B, GOLGA8R, MAMLD1, PFN4, SKOR1, TXNDC16, ATG12, CTAG2,
GOLGB1, MAMSTR, PGA3, SKOR2, TXNDC17, ATG13, CTAGE1, GOLIM4, MAN1A1, PGA4,
SKP1, TXNDC2, ATG14, CTAGE15, GOLM1, MAN1A2, PGA5, SKP2, TXNDC5, ATG16L1,
CTAGE4, GOLPH3, MAN1B1, PGAM1, SLA, TXNDC8, ATG16L2, CTAGE5, GOLPH3L, MAN1C1,
PGAM2, SLA2, TXNDC9, ATG2A, CTAGE6, GOLT1A, MAN2A1, PGAM4, SLAIN1, TXNIP,
ATG2B, CTAGE8, GOLT1B, MAN2A2, PGAM5, SLAIN2, TXNL1, ATG3, CTAGE9, GON4L,
MAN2B1, PGAP1, SLAMF1, TXNL4A, ATG4A, CTBP1, GOPC, MAN2B2, PGAP2, SLAMF6,
TXNL4B, ATG4B, CTBP2, GORAB, MAN2C1, PGAP3, SLAMF7, TXNRD1, ATG4C, CTBS,
GORASP1, MANBA, PGBD1, SLAMF8, TXNRD2, ATG4D, CTC1, GORASP2, MANBAL, PGBD2,
SLAMF9, TXNRD3, ATG5, CTCF, GOSR1, MANEA, PGBD3, SLBP, TXNRD3NB, ATG7, CTCFL,
GOSR2, MANEAL, PGBD4, SLC10A1, TYK2, ATG9A, CTDNEP1, GOT1, MANF, PGBD5,
SLC10A2, TYMP, ATG9B, CTDP1, GOT1L1, MANSC1, PGC, SLC10A3, TYMS, ATHL1, CTDSP1,
GOT2, MANSC4, PGD, SLC10A4, TYR, ATIC, CTDSP2, GP1BA, MAOA, PGF, SLC10A5, TYRO3,
ATL1, CTDSPL, GP1BB, MAOB, PGGT1B, SLC10A6, TYROBP, ATL2, CTDSPL2, GP2, MAP10,
PGK1, SLC10A7, TYRP1, ATL3, CTF1, GP5, MAP1A, PGK2, SLC11A1, TYSND1, ATM, CTGF,
GP6, MAP1B, PGLS, SLC11A2, TYW1, ATMIN, CTH, GP9, MAP1LC3A, PGLYRP1, SLC12A1,
TYW1B, ATN1, CTHRC1, GPA33, MAP1LC3B, PGLYRP2, SLC12A2, TYW3, ATOH1, CTIF,
GPAA1, MAP1LC3B2, PGLYRP3, SLC12A3, TYW5, ATOH7, CTLA4, GPALPP1, MAP1LC3C,
PGLYRP4, SLC12A4, U2AF1, ATOH8, CTNNA1, GPAM, MAP1S, PGM1, SLC12A5, U2AF1L4,
ATOX1, CTNNA2, GPANK1, MAP2, PGM2, SLC12A6, U2AF2, ATP10A, CTNNA3, GPAT2,
MAP2K1, PGM2L1, SLC12A7, U2SURP, ATP10B, CTNNAL1, GPATCH1, MAP2K2, PGM3,
SLC12A8, UACA, ATP10D, CTNNB1, GPATCH11, MAP2K3, PGM5, SLC12A9, UAP1, ATP11A,
CTNNBIP1, GPATCH2, MAP2K4, PGP, SLC13A1, UAP1L1, ATP11B, CTNNBL1, GPATCH2L,
MAP2K5, PGPEP1, SLC13A2, UBA1, ATP11C, CTNND1, GPATCH3, MAP2K6, PGPEP1L,
SLC13A3, UBA2, ATP12A, CTNND2, GPATCH4, MAP2K7, PGR, SLC13A4, UBA3, ATP13A1,
CTNS, GPATCH8, MAP3K1, PGRMC1, SLC13A5, UBA5, ATP13A2, CTPS1, GPBAR1, MAP3K10,
PGRMC2, SLC14A1, UBA52, ATP13A3, CTPS2, GPBP1, MAP3K11, PGS1, SLC14A2, UBA6,
ATP13A4, CTR9, GPBP1L1, MAP3K12, PHACTR1, SLC15A1, UBA7, ATP13A5, CTRB1, GPC1,
MAP3K13, PHACTR2, SLC15A2, UBAC1, ATP1A1, CTRB2, GPC2, MAP3K14, PHACTR3,
SLC15A3, UBAC2, ATP1A2, CTRC, GPC3, MAP3K15, PHACTR4, SLC15A4, UBALD1, ATP1A3,
CTRL, GPC4, MAP3K19, PHAX, SLC15A5, UBALD2, ATP1A4, CTSA, GPC5, MAP3K2, PHB,
SLC16A1, UBAP1, ATP1B1, CTSB, GPC6, MAP3K3, PHB2, SLC16A10, UBAP1L, ATP1B2, CTSC,
GPCPD1, MAP3K4, PHC1, SLC16A11, UBAP2, ATP1B3, CTSD, GPD1, MAP3K5, PHC2, SLC16A12,
UBAP2L, ATP1B4, CTSE, GPD1L, MAP3K6, PHC3, SLC16A13, UBASH3A, ATP2A1, CTSF, GPD2,
MAP3K7, PHEX, SLC16A14, UBASH3B, ATP2A2, CTSG, GPER1, MAP3K7CL, PHF1, SLC16A2,
UBB, ATP2A3, CTSH, GPHA2, MAP3K8, PHF10, SLC16A3, UBC, ATP2B1, CTSK, GPHB5,
MAP3K9, PHF11, SLC16A4, UBD, ATP2B2, CTSL, GPHN, MAP4, PHF12, SLC16A5, UBE2A,
ATP2B3, CTSO, GPI, MAP4K1, PHF13, SLC16A6, UBE2B, ATP2B4, CTSS, GPIHBP1, MAP4K2,
PHF14, SLC16A7, UBE2C, ATP2C1, CTSV, GPKOW, MAP4K3, PHF19, SLC16A8, UBE2D1,
ATP2C2, CTSW, GPLD1, MAP4K4, PHF2, SLC16A9, UBE2D2, ATP4A, CTSZ, GPM6A, MAP4K5,
PHF20, SLC17A1, UBE2D3, ATP4B, CTTN, GPM6B, MAP6, PHF20L1, SLC17A2, UBE2D4,
ATP5A1, CTTNBP2, GPN1, MAP6D1, PHF21A, SLC17A3, UBE2E1, ATP5B, CTTNBP2NL, GPN2,
MAP7, PHF21B, SLC17A4, UBE2E2, ATP5C1, CTU1, GPN3, MAP7D1, PHF23, SLC17A5, UBE2E2-
AS1, ATP5D, CTU2, GPNMB, MAP7D2, PHF3, SLC17A6, UBE2E3, ATP5E, CTXN1, GPR1,
MAP7D3, PHF5A, SLC17A7, UBE2F, ATP5F1, CTXN2, GPR101, MAP9, PHF6, SLC17A8, UBE2G1,
ATP5G1, CTXN3, GPR107, MAPK1, PHF7, SLC17A9, UBE2G2, ATP5G2, CUBN, GPR108,
MAPK10, PHF8, SLC18A1, UBE2H, ATP5G3, CUEDC1, GPR110, MAPK11, PHGDH, SLC18A2,
UBE2I, ATP5H, CUEDC2, GPR111, MAPK12, PHGR1, SLC18A3, UBE2J1, ATP5I, CUL1, GPR112,
MAPK13, PHIP, SLC18B1, UBE2J2, ATP5J, CUL2, GPR113, MAPK14, PHKA1, SLC19A1, UBE2K,
ATP5J2, CUL3, GPR114, MAPK15, PHKA2, SLC19A2, UBE2L3, ATP5J2-PTCD1, CUL4A, GPR115,
MAPK1IP1L, PHKB, SLC19A3, UBE2L6, ATP5L, CUL4B, GPR116, MAPK3, PHKG1, SLCIA1,
UBE2M, ATP5L2, CUL5, GPR119, MAPK4, PHKG2, SLC1A2, UBE2N, ATP5O, CUL7, GPR12,
MAPK6, PHLDA1, SLC1A3, UBE2NL, ATP5S, CUL9, GPR123, MAPK7, PHLDA2, SLC1A4,
UBE2O, ATP5SL, CUTA, GPR124, MAPK8, PHLDA3, SLC1A5, UBE2Q1, ATP6AP1, CUTC,
GPR125, MAPK8IP1, PHLDB1, SLC1A6, UBE2Q2, ATP6AP1L, CUX1, GPR126, MAPK8IP2,
PHLDB2, SLC1A7, UBE2QL1, ATP6AP2, CUX2, GPR128, MAPK8IP3, PHLDB3, SLC20A1,
UBE2R2, ATP6V0A1, CUZD1, GPR132, MAPK9, PHLPP1, SLC20A2, UBE2S, ATP6V0A2, CWC15,
GPR133, MAPKAP1, PHLPP2, SLC22A1, UBE2T, ATP6V0A4, CWC22, GPR135, MAPKAPK2,
PHOSPHO1, SLC22A10, UBE2U, ATP6V0B, CWC25, GPR137, MAPKAPK3, PHOSPHO2,
SLC22A11, UBE2V1, ATP6V0C, CWC27, GPR137B, MAPKAPK5, PHOSPHO2-KLHL23,
SLC22A12, UBE2V2, ATP6V0D1, CWF19L1, GPR137C, MAPKBP1, PHOX2A, SLC22A13, UBE2W,
ATP6V0D2, CWF19L2, GPR139, MAPRE1, PHOX2B, SLC22A14, UBE2Z, ATP6V0E1, CWH43,
GPR142, MAPRE2, PHPT1, SLC22A15, UBE3A, ATP6V0E2, CX3CL1, GPR143, MAPRE3, PHRF1,
SLC22A16, UBE3B, ATP6V1A, CX3CR1, GPR148, MAPT, PHTF1, SLC22A17, UBE3C, ATP6V1B1,
CXADR, GPR149, 1-Mar, PHTF2, SLC22A18, UBE3D, ATP6V1B2, CXCL1, GPR15, 10-Mar, PHYH,
SLC22A18AS, UBE4A, ATP6V1C1, CXCL10, GPR150, 11- Mar, PHYHD1, SLC22A2, UBE4B,
ATP6V1C2, CXCL11, GPR151, 2-Mar, PHYHIP, SLC22A20, UBFD1, ATP6V1D, CXCL12, GPR152,
3-Mar, PHYHIPL, SLC22A23, UBIAD1, ATP6V1E1, CXCL13, GPR153, 4- Mar, PHYKPL,
SLC22A24, UBL3, ATP6V1E2, CXCL14, GPR155, 5-Mar, PI15, SLC22A25, UBL4A, ATP6V1F,
CXCL16, GPR156, 6-Mar, PI16, SLC22A3, UBL4B, ATP6V1G1, CXCL17, GPR157, 7-Mar, PI3,
SLC22A31, UBL5, ATP6V1G2, CXCL2, GPR158, 8-Mar, PI4K2A, SLC22A4, UBL7, ATP6V1G3,
CXCL3, GPR160, 9-Mar, PI4K2B, SLC22A5, UBLCP1, ATP6V1H, CXCL5, GPR161, MARCKS,
PI4KA, SLC22A6, UBN1, ATP7A, CXCL6, GPR162, MARCKSL1, PI4KB, SLC22A7, UBN2, ATP7B,
CXCL9, GPR17, MARCO, PIANP, SLC22A8, UBOX5, ATP8A1, CXCR1, GPR171, MARK1, PIAS1,
SLC22A9, UBP1, ATP8A2, CXCR2, GPR173, MARK2, PIAS2, SLC23A1, UBQLN1, ATP8B1,
CXCR3, GPR174, MARK3, PIAS3, SLC23A2, UBQLN2, ATP8B2, CXCR4, GPR176, MARK4, PIAS4,
SLC23A3, UBQLN3, ATP8B3, CXCR5, GPR179, MARS, PIBF1, SLC24A1, UBQLN4, ATP8B4,
CXCR6, GPR18, MARS2, PICALM, SLC24A2, UBQLNL, ATP9A, CXorf21, GPR180, MARVELD1,
PICK1, SLC24A3, UBR1, ATP9B, CXorf22, GPR182, MARVELD2, PID1, SLC24A4, UBR2, ATPAF1,
CXorf23, GPR183, MARVELD3, PIDD, SLC24A5, UBR3, ATPAF2, CXorf27, GPR19, MAS1,
PIEZO1, SLC25A1, UBR4, ATPIF1, CXorf30, GPR20, MAS1L, PIEZO2, SLC25A10, UBR5, ATR,
CXorf36, GPR21, MASP1, PIF1, SLC25A11, UBR7, ATRAID, CXorf38, GPR22, MASP2, PIFO,
SLC25A12, UBTD1, ATRIP, CXorf40A, GPR25, MAST1, PIGA, SLC25A13, UBTD2, ATRN,
CXorf40B, GPR26, MAST2, PIGB, SLC25A14, UBTF, ATRNL1, CXorf48, GPR27, MAST3, PIGC,
SLC25A15, UBTFL1, ATRX, CXorf49, GPR3, MAST4, PIGF, SLC25A16, UBXN1, ATXN1,
CXorf49B, GPR31, MASTL, PIGG, SLC25A17, UBXN10, ATXN10, CXorf51A, GPR32, MAT1A,
PIGH, SLC25A18, UBXN11, ATXN1L, CXorf51B, GPR33, MAT2A, PIGK, SLC25A19, UBXN2A,
ATXN2, CXorf56, GPR34, MAT2B, PIGL, SLC25A2, UBXN2B, ATXN2L, CXorf57, GPR35, MATK,
PIGM, SLC25A20, UBXN4, ATXN3, CXorf58, GPR37, MATN1, PIGN, SLC25A21, UBXN6,
ATXN3L, CXorf61, GPR37L1, MATN2, PIGO, SLC25A22, UBXN7, ATXN7, CXorf64, GPR39,
MATN3, PIGP, SLC25A23, UBXN8, ATXN7L1, CXorf65, GPR4, MATN4, PIGQ, SLC25A24,
UCHL1, ATXN7L2, CXorf66, GPR45, MATR3, PIGR, SLC25A25, UCHL3, ATXN7L3, CXXC1,
GPR50, MAU2, PIGS, SLC25A26, UCHL5, ATXN7L3B, CXXC11, GPR52, MAVS, PIGT, SLC25A27,
UCK1, AUH, CXXC4, GPR55, MAX, PIGU, SLC25A28, UCK2, AUNIP, CXXC5, GPR56, MAZ,
PIGV, SLC25A29, UCKL1, AUP1, CYB561, GPR6, MB, PIGW, SLC25A3, UCMA, AURKA,
CYB561A3, GPR61, MB21D1, PIGX, SLC25A30, UCN, AURKAIP1, CYB561D1, GPR62, MB21D2,
PIGY, SLC25A31, UCN2, AURKB, CYB561D2, GPR63, MBD1, PIGZ, SLC25A32, UCN3, AURKC,
CYB5A, GPR64, MBD2, PIH1D1, SLC25A33, UCP1, AUTS2, CYB5B, GPR65, MBD3, PIH1D2,
SLC25A34, UCP2, AVEN, CYB5D1, GPR68, MBD3L1, PIH1D3, SLC25A35, UCP3, AVIL, CYB5D2,
GPR75, MBD4, PIK3AP1, SLC25A36, UEVLD, AVL9, CYB5R1, GPR75-ASB3, MBD5, PIK3C2A,
SLC25A37, UFC1, AVP, CYB5R2, GPR78, MBD6, PIK3C2B, SLC25A38, UFD1L, AVPI1, CYB5R3,
GPR82, MBIP, PIK3C2G, SLC25A39, UFL1, AVPR1A, CYB5R4, GPR83, MBL2, PIK3C3, SLC25A4,
UFM1, AVPR1B, CYB5RL, GPR84, MBLAC1, PIK3CA, SLC25A40, UFSP1, AVPR2, CYBA, GPR85,
MBLAC2, PIK3CB, SLC25A41, UFSP2, AWAT1, CYBB, GPR87, MBNL1, PIK3CD, SLC25A42,
UGCG, AWAT2, CYBRD1, GPR88, MBNL2, PIK3CG, SLC25A43, UGDH, AXDND1, CYC1,
GPR89A, MBNL3, PIK3IP1, SLC25A44, UGGT1, AXIN1, CYCS, GPR89B, MBOAT1, PIK3R1,
SLC25A45, UGGT2, AXIN2, CYFIP1, GPR89C, MBOAT2, PIK3R2, SLC25A46, UGP2, AXL,
CYFIP2, GPR97, MBOAT4, PIK3R3, SLC25A47, UGT1A1, AZGP1, CYGB, GPR98, MBOAT7,
PIK3R4, SLC25A48, UGT1A10, AZI1, CYHR1, GPRASP1, MBP, PIK3R5, SLC25A5, UGT1A3, AZI2,
CYLC1, GPRASP2, MBTD1, PIK3R6, SLC25A51, UGT1A4, AZIN1, CYLC2, GPRC5A, MBTPS1,
PIKFYVE, SLC25A52, UGT1A5, AZU1, CYLD, GPRC5B, MBTPS2, PILRA, SLC25A53, UGT1A6,
B2M, CYorf17, GPRC5C, MC1R, PILRB, SLC25A6, UGT1A7, B3GALNT1, CYP11A1, GPRC5D,
MC2R, PIM1, SLC26A1, UGT1A8, B3GALNT2, CYP11B1, GPRC6A, MC3R, PIM2, SLC26A10,
UGT1A9, B3GALT1, CYP11B2, GPRIN1, MC4R, PIM3, SLC26A11, UGT2A1, B3GALT2, CYP17A1,
GPRIN2, MC5R, PIN1, SLC26A2, UGT2A2, B3GALT4, CYP19A1, GPRIN3, MCAM, PIN4,
SLC26A3, UGT2A3, B3GALT5, CYP1A1, GPS1, MCAT, PINK1, SLC26A4, UGT2B10, B3GALT6,
CYP1A2, GPS2, MCC, PINLYP, SLC26A5, UGT2B11, B3GALTL, CYP1B1, GPSM1, MCCC1,
PINX1, SLC26A6, UGT2B15, B3GAT1, CYP20A1, GPSM2, MCCC2, PIP, SLC26A7, UGT2B17,
B3GAT2, CYP21A2, GPSM3, MCCD1, PIP4K2A, SLC26A8, UGT2B28, B3GAT3, CYP24A1, GPT,
MCEE, PIP4K2B, SLC26A9, UGT2B4, B3GNT1, CYP26A1, GPT2, MCF2, PIP4K2C, SLC27A1,
UGT2B7, B3GNT2, CYP26B1, GPX1, MCF2L, PIP5K1A, SLC27A2, UGT3A1, B3GNT3, CYP26C1,
GPX2, MCF2L2, PIP5K1B, SLC27A3, UGT3A2, B3GNT4, CYP27A1, GPX3, MCFD2, PIP5K1C,
SLC27A4, UGT8, B3GNT5, CYP27B1, GPX4, MCHR1, PIP5KL1, SLC27A5, UHMK1, B3GNT6,
CYP27C1, GPX5, MCHR2, PIPOX, SLC27A6, UHRF1, B3GNT7, CYP2A13, GPX6, MCIDAS, PIR,
SLC28A1, UHRF1BP1, B3GNT8, CYP2A6, GPX7, MCL1, PIRT, SLC28A2, UHRF1BP1L, B3GNT9,
CYP2A7, GPX8, MCM10, PISD, SLC28A3, UHRF2, B3GNTL1, CYP2B6, GRAMD1A, MCM2,
PITHD1, SLC29A1, UIMC1, B4GALNT1, CYP2C18, GRAMD1B, MCM3, PITPNA, SLC29A2,
ULBP1, B4GALNT2, CYP2C19, GRAMD1C, MCM3AP, PITPNB, SLC29A3, ULBP2, B4GALNT3,
CYP2C8, GRAMD2, MCM4, PITPNC1, SLC29A4, ULBP3, B4GALNT4, CYP2C9, GRAMD3, MCM5,
PITPNM1, SLC2A1, ULK1, B4GALT1, CYP2D6, GRAMD4, MCM6, PITPNM2, SLC2A10, ULK2,
B4GALT2, CYP2E1, GRAP, MCM7, PITPNM3, SLC2A11, ULK3, B4GALT3, CYP2F1, GRAP2,
MCM8, PITRM1, SLC2A12, ULK4, B4GALT4, CYP2J2, GRAPL, MCM9, PITX1, SLC2A13, UMOD,
B4GALT5, CYP2R1, GRASP, MCMBP, PITX2, SLC2A14, UMODL1, B4GALT6, CYP2S1, GRB10,
MCMDC2, PITX3, SLC2A2, UMPS, B4GALT7, CYP2U1, GRB14, MCOLN1, PIWIL1, SLC2A3,
UNC119, B9D1, CYP2W1, GRB2, MCOLN2, PIWIL2, SLC2A4, UNC119B, B9D2, CYP39A1, GRB7,
MCOLN3, PIWIL3, SLC2A4RG, UNC13A, BAALC, CYP3A4, GREB1, MCPH1, PIWIL4, SLC2A5,
UNC13B, BAAT, CYP3A43, GREM1, MCRS1, PJA1, SLC2A6, UNC13C, BABAM1, CYP3A5,
GREM2, MCTP1, PJA2, SLC2A7, UNC13D, BACE1, CYP3A7, GRHL1, MCTP2, PKD1, SLC2A8,
UNC45A, BACE2, CYP3A7- CYP3AP1, GRHL2, MCTS1, PKD1L1, SLC2A9, UNC45B, BACH1,
CYP46A1, GRHL3, MCU, PKD1L2, SLC30A1, UNC50, BACH2, CYP4A11, GRHPR, MCUR1,
PKD1L3, SLC30A10, UNC5A, BAD, CYP4A22, GRIA1, MDC1, PKD2, SLC30A2, UNC5B, BAG1,
CYP4B1, GRIA2, MDFI, PKD2L1, SLC30A3, UNC5C, BAG2, CYP4F11, GRIA3, MDFIC, PKD2L2,
SLC30A4, UNC5CL, BAG3, CYP4F12, GRIA4, MDGA1, PKDCC, SLC30A5, UNC5D, BAG4,
CYP4F2, GRID1, MDGA2, PKDREJ, SLC30A6, UNC79, BAG5, CYP4F22, GRID2, MDH1, PKHD1,
SLC30A7, UNC80, BAG6, CYP4F3, GRID2IP, MDH1B, PKHD1L1, SLC30A8, UNC93A, BAGE,
CYP4F8, GRIFIN, MDH2, PKIA, SLC30A9, UNC93B1, BAGE2, CYP4V2, GRIK1, MDK, PKIB,
SLC31A1, UNCX, BAGE3, CYP4X1, GRIK2, MDM1, PKIG, SLC31A2, UNG, BAHCC1, CYP4Z1,
GRIK3, MDM2, PKLR, SLC32A1, UNK, BAHD1, CYP51A1, GRIK4, MDM4, PKM, SLC33A1,
UNKL, BAI1, CYP7A1, GRIK5, MDN1, PKMYT1, SLC34A1, UPB1, BAI2, CYP7B1, GRIN1, MDP1,
PKN1, SLC34A2, UPF1, BAI3, CYP8B1, GRIN2A, ME1, PKN2, SLC34A3, UPF2, BAIAP2, CYR61,
GRIN2B, ME2, PKN3, SLC35A1, UPF3A, BAIAP2L1, CYS1, GRIN2C, ME3, PKNOX1, SLC35A2,
UPF3B, BAIAP2L2, CYSLTR1, GRIN2D, MEA1, PKNOX2, SLC35A3, UPK1A, BAIAP3, CYSLTR2,
GRIN3A, MEAF6, PKP1, SLC35A4, UPK1B, BAK1, CYSTM1, GRIN3B, MECOM, PKP2, SLC35A5,
UPK2, BAMBI, CYTH1, GRINA, MECP2, PKP3, SLC35B1, UPK3A, BANF1, CYTH2, GRIP1,
MECR, PKP4, SLC35B2, UPK3B, BANF2, CYTH3, GRIP2, MED1, PLA1A, SLC35B3, UPK3BL,
BANK1, CYTH4, GRIPAP1, MED10, PLA2G10, SLC35B4, UPP1, BANP, CYTIP, GRK1, MED11,
PLA2G12A, SLC35C1, UPP2, BAP1, CYTL1, GRK4, MED12, PLA2G12B, SLC35C2, UPRT, BARD1,
CYYR1, GRK5, MED12L, PLA2G15, SLC35D1, UQCC1, BARHL1, D2HGDH, GRK6, MED13,
PLA2G16, SLC35D2, UQCC2, BARHL2, DAAM1, GRK7, MED13L, PLA2G1B, SLC35D3, UQCR10,
BARX1, DAAM2, GRM1, MED14, PLA2G2A, SLC35E1, UQCR11, BARX2, DAB1, GRM2, MED15,
PLA2G2C, SLC35E2, UQCRB, BASP1, DAB2, GRM3, MED16, PLA2G2D, SLC35E2B, UQCRC1,
BATF, DAB2IP, GRM4, MED17, PLA2G2E, SLC35E3, UQCRC2, BATF2, DACH1, GRM5, MED18,
PLA2G2F, SLC35E4, UQCRFS1, BATF3, DACH2, GRM6, MED19, PLA2G3, SLC35F1, UQCRH,
BAX, DACT1, GRM7, MED20, PLA2G4A, SLC35F2, UQCRHL, BAZ1A, DACT2, GRM8, MED21,
PLA2G4B, SLC35F3, UQCRQ, BAZ1B, DACT3, GRN, MED22, PLA2G4C, SLC35F4, URAD,
BAZ2A, DAD1, GRP, MED23, PLA2G4D, SLC35F5, URB1, BAZ2B, DAG1, GRPEL1, MED24,
PLA2G4E, SLC35F6, URB2, BBC3, DAGLA, GRPEL2, MED25, PLA2G4F, SLC35G1, URGCP,
BBIP1, DAGLB, GRPR, MED26, PLA2G5, SLC35G2, URGCP-MRPS24, BBOX1, DAK, GRSF1,
MED27, PLA2G6, SLC35G3, URI1, BBS1, DALRD3, GRTP1, MED28, PLA2G7, SLC35G4, URM1,
BBS10, DAND5, GRWD1, MED29, PLA2R1, SLC35G5, UROC1, BBS12, DAO, GRXCR1, MED30,
PLAA, SLC35G6, UROD, BBS2, DAOA, GRXCR2, MED31, PLAC1, SLC36A1, UROS, BBS4, DAP,
GSAP, MED4, PLAC4, SLC36A2, USB1, BBS5, DAP3, GSC, MED6, PLAC8, SLC36A3, USE1, BBS7,
DAPK1, GSC2, MED7, PLAC8L1, SLC36A4, USF1, BBS9, DAPK2, GSDMA, MED8, PLAC9,
SLC37A1, USF2, BBX, DAPK3, GSDMB, MED9, PLAG1, SLC37A2, USH1C, BCAM, DAPL1,
GSDMC, MEDAG, PLAGL1, SLC37A3, USH1G, BCAN, DAPP1, GSDMD, MEF2A, PLAGL2,
SLC37A4, USH2A, BCAP29, DARS, GSE1, MEF2B, PLAT, SLC38A1, USHBP1, BCAP31, DARS2,
GSG1, MEF2BNB, PLAU, SLC38A10, USMG5, BCAR1, DAW1, GSG1L, MEF2BNB-MEF2B,
PLAUR, SLC38A11, USO1, BCAR3, DAXX, GSG2, MEF2C, PLB1, SLC38A2, USP1, BCAS1, DAZ1,
GSK3A, MEF2D, PLBD1, SLC38A3, USP10, BCAS2, DAZ2, GSK3B, MEFV, PLBD2, SLC38A4,
USP11, BCAS3, DAZ3, GSKIP, MEGF10, PLCB1, SLC38A5, USP12, BCAS4, DAZ4, GSN, MEGF11,
PLCB2, SLC38A6, USP13, BCAT1, DAZAP1, GSPT1, MEGF6, PLCB3, SLC38A7, USP14, BCAT2,
DAZAP2, GSPT2, MEGF8, PLCB4, SLC38A8, USP15, BCCIP, DAZL, GSR, MEGF9, PLCD1,
SLC38A9, USP16, BCDIN3D, DBF4, GSS, MEI1, PLCD3, SLC39A1, USP17L24, BCHE, DBF4B,
GSTA1, MEI4, PLCD4, SLC39A10, USP17L25, BCKDHA, DBH, GSTA2, MEIG1, PLCE1,
SLC39A11, USP17L26, BCKDHB, DBI, GSTA3, MEIOB, PLCG1, SLC39A12, USP17L28, BCKDK,
DBN1, GSTA4, MEIS1, PLCG2, SLC39A13, USP17L29, BCL10, DBNDD1, GSTA5, MEIS2, PLCH1,
SLC39A14, USP18, BCL11A, DBNDD2, GSTCD, MEIS3, PLCH2, SLC39A2, USP19, BCL11B,
DBNL, GSTK1, MELK, PLCL1, SLC39A3, USP2, BCL2, DBP, GSTM1, MEMO1, PLCL2, SLC39A4,
USP20, BCL2A1, DBR1, GSTM2, MEN1, PLCXD1, SLC39A5, USP21, BCL2L1, DBT, GSTM3,
MEOX1, PLCXD2, SLC39A6, USP22, BCL2L10, DBX1, GSTM4, MEOX2, PLCXD3, SLC39A7,
USP24, BCL2L11, DBX2, GSTM5, MEP1A, PLCZ1, SLC39A8, USP25, BCL2L12, DCAF10, GSTO1,
MEP1B, PLD1, SLC39A9, USP26, BCL2L13, DCAF11, GSTO2, MEPCE, PLD2, SLC3A1, USP27X,
BCL2L14, DCAF12, GSTP1, MEPE, PLD3, SLC3A2, USP28, BCL2L15, DCAF12L1, GSTT1,
MERTK, PLD4, SLC40A1, USP29, BCL2L2, DCAF12L2, GSTT2, MESDC1, PLD5, SLC41A1, USP3,
BCL2L2-PABPN1, DCAF13, GSTT2B, MESDC2, PLD6, SLC41A2, USP30, BCL3, DCAF15, GSTZ1,
MESP1, PLEC, SLC41A3, USP31, BCL6, DCAF16, GSX1, MESP2, PLEK, SLC43A1, USP32, BCL6B,
DCAF17, GSX2, MEST, PLEK2, SLC43A2, USP33, BCL7A, DCAF4, GTDC1, MET, PLEKHA1,
SLC43A3, USP34, BCL7B, DCAF4L1, GTF2A1, METAP1, PLEKHA2, SLC44A1, USP35, BCL7C,
DCAF4L2, GTF2A1L, METAP1D, PLEKHA3, SLC44A2, USP36, BCL9, DCAF5, GTF2A2, METAP2,
PLEKHA4, SLC44A3, USP37, BCL9L, DCAF6, GTF2B, METRN, PLEKHA5, SLC44A4, USP38,
BCLAF1, DCAF7, GTF2E1, METRNL, PLEKHA6, SLC44A5, USP39, BCMO1, DCAF8, GTF2E2,
METTL1, PLEKHA7, SLC45A1, USP4, BCO2, DCAF8L1, GTF2F1, METTL10, PLEKHA8, SLC45A2,
USP40, BCOR, DCAKD, GTF2F2, METTL11B, PLEKHB1, SLC45A3, USP41, BCORL1, DCBLD1,
GTF2H1, METTL12, PLEKHB2, SLC45A4, USP42, BCR, DCBLD2, GTF2H2, METTL13, PLEKHD1,
SLC46A1, USP43, BCS1L, DCC, GTF2H2C, METTL14, PLEKHF1, SLC46A2, USP44, BDH1, DCD,
GTF2H3, METTL15, PLEKHF2, SLC46A3, USP45, BDH2, DCDC1, GTF2H4, METTL16, PLEKHG1,
SLC47A1, USP46, BDKRB1, DCDC2, GTF2H5, METTL17, PLEKHG2, SLC47A2, USP47, BDKRB2,
DCDC2B, GTF2I, METTL18, PLEKHG3, SLC48A1, USP48, BDNF, DCDC2C, GTF2IRD1, METTL20,
PLEKHG4, SLC4A1, USP49, BDP1, DCDC5, GTF2IRD2, METTL21A, PLEKHG4B, SLC4A10, USP5,
BEAN1, DCHS1, GTF2IRD2B, METTL21B, PLEKHG5, SLC4A11, USP50, BECN1, DCHS2, GTF3A,
METTL21C, PLEKHG6, SLC4A1AP, USP51, BECN1P1, DCK, GTF3C1, METTL22, PLEKHG7,
SLC4A2, USP53, BEGAIN, DCLK1, GTF3C2, METTL23, PLEKHH1, SLC4A3, USP54, BEND2,
DCLK2, GTF3C3, METTL24, PLEKHH2, SLC4A4, USP6, BEND3, DCLK3, GTF3C4, METTL25,
PLEKHH3, SLC4A5, USP6NL, BEND4, DCLRE1A, GTF3C5, METTL2A, PLEKHJ1, SLC4A7, USP7,
BEND5, DCLRE1B, GTF3C6, METTL2B, PLEKHM1, SLC4A8, USP8, BEND6, DCLRE1C, GTPBP1,
METTL3, PLEKHM2, SLC4A9, USP9X, BEND7, DCN, GTPBP10, METTL4, PLEKHM3, SLC50A1,
USP9Y, BEST1, DCP1A, GTPBP2, METTL5, PLEKHN1, SLC51A, USPL1, BEST2, DCP1B, GTPBP3,
METTL6, PLEKHO1, SLC51B, UST, BEST3, DCP2, GTPBP4, METTL7A, PLEKHO2, SLC52A1,
UTF1, BEST4, DCPS, GTPBP6, METTL7B, PLEKHS1, SLC52A2, UTP11L, BET1, DCST1, GTPBP8,
METTL8, PLET1, SLC52A3, UTP14A, BET1L, DCST2, GTSCR1, METTL9, PLG, SLC5A1, UTP14C,
BEX1, DCSTAMP, GTSE1, MEX3A, PLGLB1, SLC5A10, UTP15, BEX2, DCT, GTSF1, MEX3B,
PLGLB2, SLC5A11, UTP18, BEX4, DCTD, GTSF1L, MEX3C, PLGRKT, SLC5A12, UTP20, BEX5,
DCTN1, GUCA1A, MEX3D, PLIN1, SLC5A2, UTP23, BFAR, DCTN2, GUCA1B, MFAP1, PLIN2,
SLC5A3, UTP3, BFSP1, DCTN3, GUCA1C, MFAP2, PLIN3, SLC5A4, UTP6, BFSP2, DCTN4,
GUCA2A, MFAP3, PLIN4, SLC5A5, UTRN, BGLAP, DCTN5, GUCA2B, MFAP3L, PLIN5, SLC5A6,
UTS2, BGN, DCTN6, GUCD1, MFAP4, PLK1, SLC5A7, UTS2B, BHLHA15, DCTPP1, GUCY1A2,
MFAP5, PLK1S1, SLC5A8, UTY, BHLHA9, DCUN1D1, GUCY1A3, MFF, PLK2, SLC5A9, UVRAG,
BHLHB9, DCUN1D2, GUCY1B3, MFGE8, PLK3, SLC6A1, UVSSA, BHLHE22, DCUN1D3,
GUCY2C, MFHAS1, PLK4, SLC6A11, UXS1, BHLHE23, DCUN1D4, GUCY2D, MFI2, PLK5,
SLC6A12, UXT, BHLHE40, DCUN1D5, GUCY2F, MFN1, PLLP, SLC6A13, VAC14, BHLHE41,
DCX, GUF1, MFN2, PLN, SLC6A14, VAMP1, BHMT, DCXR, GUK1, MFNG, PLOD1, SLC6A15,
VAMP2, BHMT2, DDA1, GULP1, MFRP, PLOD2, SLC6A16, VAMP3, BICC1, DDAH1, GUSB,
MFSD1, PLOD3, SLC6A17, VAMP4, BICD1, DDAH2, GXYLT1, MFSD10, PLP1, SLC6A18, VAMP5,
BICD2, DDB1, GXYLT2, MFSD11, PLP2, SLC6A19, VAMP7, BID, DDB2, GYG1, MFSD12, PLRG1,
SLC6A2, VAMP8, BIK, DDC, GYG2, MFSD2A, PLS1, SLC6A20, VANGL1, BIN1, DDHD1,
GYLTL1B, MFSD2B, PLS3, SLC6A3, VANGL2, BIN2, DDHD2, GYPA, MFSD3, PLSCR1, SLC6A4,
VAPA, BIN3, DDI1, GYPB, MFSD4, PLSCR2, SLC6A5, VAPB, BIRC2, DDI2, GYPC, MFSD5,
PLSCR3, SLC6A6, VARS, BIRC3, DDIT3, GYPE, MFSD6, PLSCR4, SLC6A7, VARS2, BIRC5,
DDIT4, GYS1, MFSD6L, PLSCR5, SLC6A8, VASH1, BIRC6, DDIT4L, GYS2, MFSD7, PLTP,
SLC6A9, VASH2, BIRC7, DDN, GZF1, MFSD8, PLVAP, SLC7A1, VASN, BIRC8, DDO, GZMA,
MFSD9, PLXDC1, SLC7A10, VASP, BIVM, DDOST, GZMB, MGA, PLXDC2, SLC7A11, VAT1,
BIVM-ERCC5, DDR1, GZMH, MGAM, PLXNA1, SLC7A13, VAT1L, BLCAP, DDR2, GZMK,
MGARP, PLXNA2, SLC7A14, VAV1, BLID, DDRGK1, GZMM, MGAT1, PLXNA3, SLC7A2, VAV2,
BLK, DDT, H1F0, MGAT2, PLXNA4, SLC7A3, VAV3, BLM, DDTL, H1FNT, MGAT3, PLXNB1,
SLC7A4, VAX1, BLMH, DDX1, H1FOO, MGAT4A, PLXNB2, SLC7A5, VAX2, BLNK, DDX10,
H1FX, MGAT4B, PLXNB3, SLC7A6, VBP1, BLOC1S1, DDX11, H2AFB1, MGAT4C, PLXNC1,
SLC7A6OS, VCAM1, BLOC1S2, DDX17, H2AFB2, MGAT5, PLXND1, SLC7A7, VCAN, BLOC1S3,
DDX18, H2AFB3, MGAT5B, PM20D1, SLC7A8, VCL, BLOCIS4, DDX19A, H2AFJ, MGEA5,
PM20D2, SLC7A9, VCP, BLOC1S5, DDX19B, H2AFV, MGLL, PMAIP1, SLC8A1, VCPIP1,
BLOC1S6, DDX20, H2AFX, MGME1, PMCH, SLC8A2, VCPKMT, BLVRA, DDX21, H2AFY,
MGMT, PMEL, SLC8A3, VCX, BLVRB, DDX23, H2AFY2, MGP, PMEPA1, SLC8B1, VCX2, BLZF1,
DDX24, H2AFZ, MGRN1, PMF1, SLC9A1, VCX3A, BMF, DDX25, H2BFM, MGST1, PMF1-BGLAP,
SLC9A2, VCX3B, BMI1, DDX26B, H2BFWT, MGST2, PMFBP1, SLC9A3, VCY, BMP1, DDX27,
H3F3A, MGST3, PML, SLC9A3R1, VCY1B, BMP10, DDX28, H3F3B, MIA, PMM1, SLC9A3R2,
VDAC1, BMP15, DDX31, H3F3C, MIA2, PMM2, SLC9A4, VDAC2, BMP2, DDX39A, H6PD, MIA3,
PMP2, SLC9A5, VDAC3, BMP2K, DDX39B, HAAO, MIB1, PMP22, SLC9A6, VDR, BMP3, DDX3X,
HABP2, MIB2, PMPCA, SLC9A7, VEGFA, BMP4, DDX3Y, HABP4, MICA, PMPCB, SLC9A8,
VEGFB, BMP5, DDX4, HACE1, MICAL1, PMS1, SLC9A9, VEGFC, BMP6, DDX41, HACL1,
MICAL2, PMS2, SLC9B1, VENTX, BMP7, DDX42, HADH, MICAL3, PMVK, SLC9B2, VEPH1,
BMP8A, DDX43, HADHA, MICALCL, PNCK, SLC9C1, VEZF1, BMP8B, DDX46, HADHB,
MICALL1, PNISR, SLC9C2, VEZT, BMPER, DDX47, HAGH, MICALL2, PNKD, SLCO1A2, VGF,
BMPR1A, DDX49, HAGHL, MICB, PNKP, SLCO1B1, VGLL1, BMPR1B, DDX5, HAL, MICU1,
PNLDC1, SLCO1B3, VGLL2, BMPR2, DDX50, HAMP, MICU2, PNLIP, SLCO1B7, VGLL3, BMS1,
DDX51, HAND1, MICU3, PNLIPRP1, SLCO1C1, VGLL4, BMX, DDX52, HAND2, MID1, PNLIPRP2,
SLCO2A1, VHL, BNC1, DDX53, HAO1, MID1IP1, PNLIPRP3, SLCO2B1, VHLL, BNC2, DDX54,
HAO2, MID2, PNMA2, SLCO3A1, VIL1, BNIP1, DDX55, HAP1, MIDN, PNMA3, SLCO4A1, VILL,
BNIP2, DDX56, HAPLN1, MIEF1, PNMA5, SLCO4C1, VIM, BNIP3, DDX58, HAPLN2, MIEF2,
PNMA6A, SLCO5A1, VIMP, BNIP3L, DDX59, HAPLN3, MIEN1, PNMA6C, SLCO6A1, VIP, BNIPL,
DDX6, HAPLN4, MIER1, PNMAL1, SLFN11, VIPAS39, BOC, DDX60, HARBI1, MIER2, PNMAL2,
SLFN12, VIPR1, BOD1, DDX60L, HARS, MIER3, PNMT, SLFN12L, VIPR2, BOD1L1, DEAF1,
HARS2, MIF, PNN, SLFN13, VIT, BOD1L2, 1-Dec, HAS1, MIF4GD, PNO1, SLFN14, VKORC1,
BOK, DECR1, HAS2, MIIP, PNOC, SLFN5, VKORC1L1, BOLA1, DECR2, HAS3, MILR1, PNP,
SLFNL1, VLDLR, BOLA2, DEDD, HAT1, MINA, PNPLA1, SLIRP, VMA21, BOLA2B, DEDD2,
HAUS1, MINK1, PNPLA2, SLIT1, VMAC, BOLA3, DEF6, HAUS2, MINOS1, PNPLA3, SLIT2,
VMO1, BOLL, DEF8, HAUS3, MINOS1-NBL1, PNPLA4, SLIT3, VMP1, BOP1, DEFA1, HAUS4,
MINPP1, PNPLA5, SLITRK1, VN1R1, BORA, DEFA1B, HAUS5, MIOS, PNPLA6, SLITRK2, VN1R2,
BPGM, DEFA3, HAUS6, MIOX, PNPLA7, SLITRK3, VN1R4, BPHL, DEFA4, HAUS7, MIP,
PNPLA8, SLITRK4, VN1R5, BPI, DEFA5, HAUS8, MIPEP, PNPO, SLITRK5, VNN1, BPIFA1,
DEFA6, HAVCR1, MIPOL1, PNPT1, SLITRK6, VNN2, BPIFA2, DEFB1, HAVCR2, MIR205HG,
PNRC1, SLK, VOPP1, BPIFA3, DEFB103A, HAX1, MIS12, PNRC2, SLMAP, VPRBP, BPIFB1,
DEFB103B, HBA1, MIS18A, POC1A, SLMO1, VPREB1, BPIFB2, DEFB104A, HBA2, MIS18BP1,
POC1B, SLMO2, VPREB3, BPIFB3, DEFB104B, HBB, MISP, POC1B-GALNT4, SLN, VPS11,
BPIFB4, DEFB105A, HBD, MITD1, POC5, SLPI, VPS13A, BPIFB6, DEFB105B, HBE1, MITF, PODN,
SLTM, VPS13B, BPIFC, DEFB106A, HBEGF, MIXL1, PODNL1, SLU7, VPS13C, BPNT1,
DEFB106B, HBG1, MKI67, PODXL, SLURP1, VPS13D, BPTF, DEFB107A, HBG2, MKKS, PODXL2,
SLX1A, VPS16, BPY2, DEFB107B, HBM, MKL1, POF1B, SLX1B, VPS18, BPY2B, DEFB108B,
HBP1, MKL2, POFUT1, SLX4, VPS25, BPY2C, DEFB110, HBQ1, MKLN1, POFUT2, SLX4IP,
VPS26A, BRAF, DEFB112, HBS1L, MKNK1, POGK, SMAD1, VPS26B, BRAP, DEFB113, HBZ,
MKNK2, POGLUT1, SMAD2, VPS28, BRAT1, DEFB114, HCAR1, MKRN1, POGZ, SMAD3, VPS29,
BRCA1, DEFB115, HCAR2, MKRN2, POLA1, SMAD4, VPS33A, BRCA2, DEFB116, HCAR3,
MKRN3, POLA2, SMAD5, VPS33B, BRCC3, DEFB118, HCCS, MKS1, POLB, SMAD6, VPS35,
BRD1, DEFB119, HCFC1, MKX, POLD1, SMAD7, VPS36, BRD2, DEFB121, HCFC1R1, MLANA,
POLD2, SMAD9, VPS37A, BRD3, DEFB123, HCFC2, MLC1, POLD3, SMAGP, VPS37B, BRD4,
DEFB124, HCK, MLEC, POLD4, SMAP1, VPS37C, BRD7, DEFB125, HCLS1, MLF1, POLDIP2,
SMAP2, VPS37D, BRD8, DEFB126, HCN1, MLF2, POLDIP3, SMARCA1, VPS39, BRD9, DEFB127,
HCN2, MLH1, POLE, SMARCA2, VPS41, BRDT, DEFB128, HCN3, MLH3, POLE2, SMARCA4,
VPS45, BRE, DEFB129, HCN4, MLIP, POLE3, SMARCA5, VPS4A, BRF1, DEFB130, HCRT, MLKL,
POLE4, SMARCAD1, VPS4B, BRF2, DEFB131, HCRTR1, MLLT1, POLG, SMARCAL1, VPS51,
BRI3, DEFB132, HCRTR2, MLLT10, POLG2, SMARCB1, VPS52, BRI3BP, DEFB133, HCST,
MLLT11, POLH, SMARCC1, VPS53, BRICD5, DEFB134, HDAC1, MLLT3, POLI, SMARCC2,
VPS54, BRINP1, DEFB135, HDAC10, MLLT4, POLK, SMARCD1, VPS72, BRINP2, DEFB136,
HDAC11, MLLT6, POLL, SMARCD2, VPS8, BRINP3, DEFB4A, HDAC2, MLN, POLM, SMARCD3,
VPS9D1, BRIP1, DEFB4B, HDAC3, MLNR, POLN, SMARCE1, VRK1, BRIX1, DEGS1, HDAC4,
MLPH, POLQ, SMC1A, VRK2, BRK1, DEGS2, HDAC5, MLST8, POLR1A, SMC1B, VRK3, BRMS1,
DEK, HDAC6, MLX, POLR1B, SMC2, VRTN, BRMS1L, DENND1A, HDAC7, MLXIP, POLR1C,
SMC3, VSIG1, BROX, DENND1B, HDAC8, MLXIPL, POLR1D, SMC4, VSIG10, BRPF1, DENND1C,
HDAC9, MLYCD, POLR1E, SMC5, VSIG10L, BRPF3, DENND2A, HDC, MMAA, POLR2A, SMC6,
VSIG2, BRS3, DENND2C, HDDC2, MMAB, POLR2B, SMCHD1, VSIG4, BRSK1, DENND2D,
HDDC3, MMACHC, POLR2C, SMCO2, VSIG8, BRSK2, DENND3, HDGF, MMADHC, POLR2D,
SMCO3, VSNL1, BRWD1, DENND4A, HDGFL1, MMD, POLR2E, SMCO4, VSTM1, BRWD3,
DENND4B, HDGFRP2, MMD2, POLR2F, SMCP, VSTM2A, BSCL2, DENND4C, HDGFRP3, MME,
POLR2G, SMCR8, VSTM2B, BSDC1, DENND5A, HDHD1, MMEL1, POLR2H, SMCR9, VSTM2L,
BSG, DENND5B, HDHD2, MMGT1, POLR2I, SMDT1, VSTM4, BSN, DENND6A, HDHD3, MMP1,
POLR2J, SMEK1, VSTM5, BSND, DENND6B, HDLBP, MMP10, POLR2J2, SMEK2, VSX1, BSPH1,
DENR, HDX, MMP11, POLR2J3, SMG1, VSX2, BSPRY, DEPDC1, HEATR1, MMP12, POLR2K,
SMG5, VTA1, BST1, DEPDC1B, HEATR2, MMP13, POLR2L, SMG6, VTCN1, BST2, DEPDC4,
HEATR3, MMP14, POLR2M, SMG7, VTI1A, BSX, DEPDC5, HEATR4, MMP15, POLR3A, SMG8,
VTI1B, BTAF1, DEPDC7, HEATR5A, MMP16, POLR3B, SMG9, VTN, BTBD1, DEPTOR,
HEATR5B, MMP17, POLR3C, SMIM1, VWA1, BTBD10, DERA, HEATR6, MMP19, POLR3D,
SMIM10, VWA2, BTBD11, DERL1, HEBP1, MMP2, POLR3E, SMIM11, VWA3A, BTBD16, DERL2,
HEBP2, MMP20, POLR3F, SMIM12, VWA3B, BTBD17, DERL3, HECA, MMP21, POLR3G,
SMIM13, VWA5A, BTBD18, DES, HECTD1, MMP23B, POLR3GL, SMIM14, VWA5B1, BTBD19,
DESI1, HECTD2, MMP24, POLR3H, SMIM15, VWA5B2, BTBD2, DESI2, HECTD3, MMP25,
POLR3K, SMIM17, VWA7, BTBD3, DET1, HECTD4, MMP26, POLRMT, SMIM18, VWA8, BTBD6,
DEXI, HECW1, MMP27, POM121, SMIM19, VWA9, BTBD7, DFFA, HECW2, MMP28, POM121C,
SMIM2, VWC2, BTBD8, DFFB, HEG1, MMP3, POM121L12, SMIM20, VWC2L, BTBD9, DFNA5,
HELB, MMP7, POM121L2, SMIM21, VWCE, BTC, DFNB31, HELLS, MMP8, POMC, SMIM22,
VWDE, BTD, DFNB59, HELQ, MMP9, POMGNT1, SMIM3, VWF, BTF3, DGAT1, HELT, MMRN1,
POMGNT2, SMIM4, WAC, BTF3L4, DGAT2, HELZ, MMRN2, POMK, SMIM5, WAPAL, BTG1,
DGAT2L6, HELZ2, MMS19, POMP, SMIM6, WARS, BTG2, DGCR14, HEMGN, MMS22L, POMT1,
SMIM7, WARS2, BTG3, DGCR2, HEMK1, MN1, POMT2, SMIM8, WAS, BTG4, DGCR6, HENMT1,
MNAT1, POMZP3, SMIM9, WASF1, BTK, DGCR6L, HEPACAM, MND1, PON1, SMKR1, WASF2,
BTLA, DGCR8, HEPACAM2, MNDA, PON2, SMLR1, WASF3, BTN1A1, DGKA, HEPH, MNS1,
PON3, SMN1, WASH1, BTN2A1, DGKB, HEPHL1, MNT, POP1, SMN2, WASL, BTN2A2, DGKD,
HEPN1, MNX1, POP4, SMNDC1, WBP1, BTN3A1, DGKE, HERC1, MOAP1, POP5, SMO, WBP11,
BTN3A2, DGKG, HERC2, MOB1A, POP7, SMOC1, WBP1L, BTN3A3, DGKH, HERC3, MOB1B,
POPDC2, SMOC2, WBP2, BTNL10, DGKI, HERC4, MOB2, POPDC3, SMOX, WBP2NL, BTNL2,
DGKK, HERC5, MOB3A, POR, SMPD1, WBP4, BTNL3, DGKQ, HERC6, MOB3B, PORCN, SMPD2,
WBP5, BTNL8, DGKZ, HERPUD1, MOB3C, POSTN, SMPD3, WBSCR16, BTNL9, DGUOK,
HERPUD2, MOB4, POT1, SMPD4, WBSCR17, BTRC, DHCR24, HES1, MOBP, POTEA, SMPD5,
WBSCR22, BUB1, DHCR7, HES2, MOCOS, POTEB, SMPDL3A, WBSCR27, BUB1B, DHDDS,
HES3, MOCS1, POTEB2, SMPDL3B, WBSCR28, BUB3, DHDH, HES4, MOCS2, POTEC, SMPX,
WDFY1, BUD13, DHFR, HES5, MOCS3, POTED, SMR3A, WDFY2, BUD31, DHFRL1, HES6, MOG,
POTEE, SMR3B, WDFY3, BVES, DHH, HES7, MOGAT1, POTEF, SMS, WDFY4, BYSL, DHODH,
HESX1, MOGAT2, POTEG, SMTN, WDHD1, BZRAP1, DHPS, HEXA, MOGAT3, POTEH, SMTNL1,
WDPCP, BZW1, DHRS1, HEXB, MOGS, POTEI, SMTNL2, WDR1, BZW2, DHRS11, HEXDC, MOK,
POTEJ, SMU1, WDR11, C10orf10, DHRS12, HEXIM1, MON1A, POTEM, SMUG1, WDR12,
C10orf105, DHRS13, HEXIM2, MON1B, POU1F1, SMURF1, WDR13, C10orf107, DHRS2, HEY1,
MON2, POU2AF1, SMURF2, WDR16, C10orf11, DHRS3, HEY2, MORC1, POU2F1, SMYD1,
WDR17, C10orf111, DHRS4, HEYL, MORC2, POU2F2, SMYD2, WDR18, C10orf112, DHRS4L1,
HFE, MORC3, POU2F3, SMYD3, WDR19, C10orf113, DHRS4L2, HFE2, MORC4, POU3F1, SMYD4,
WDR20, C10orf118, DHRS7, HFM1, MORF4L1, POU3F2, SMYD5, WDR24, C10orf12, DHRS7B,
HGC6.3, MORF4L2, POU3F3, SNAI1, WDR25, C10orf120, DHRS7C, HGD, MORN1, POU3F4,
SNAI2, WDR26, C10orf126, DHRS9, HGF, MORN2, POU4F1, SNAI3, WDR27, C10orf128, DHRSX,
HGFAC, MORN3, POU4F2, SNAP23, WDR3, C10orf129, DHTKD1, HGS, MORN4, POU4F3,
SNAP25, WDR31, C10orf131, DHX15, HGSNAT, MORN5, POU5F1, SNAP29, WDR33, C10orf137,
DHX16, HHAT, MOS, POU5F1B, SNAP47, WDR34, C10orf2, DHX29, HHATL, MOSPD1, POU5F2,
SNAP91, WDR35, C10orf25, DHX30, HHEX, MOSPD2, POU6F1, SNAPC1, WDR36, C10orf32,
DHX32, HHIP, MOSPD3, POU6F2, SNAPC2, WDR37, C10orf35, DHX33, HHIPL1, MOV10, PP2D1,
SNAPC3, WDR38, C10orf53, DHX34, HHIPL2, MOV10L1, PPA1, SNAPC4, WDR4, C10orf54,
DHX35, HHLA1, MOXD1, PPA2, SNAPC5, WDR41, C10orf55, DHX36, HHLA2, MPC1, PPAN,
SNAPIN, WDR43, C10orf62, DHX37, HHLA3, MPC1L, PPAN-P2RY11, SNCA, WDR44, C10orf67,
DHX38, HIAT1, MPC2, PPAP2A, SNCAIP, WDR45, C10orf68, DHX40, HIATL1, MPDU1, PPAP2B,
SNCB, WDR45B, C10orf71, DHX57, HIBADH, MPDZ, PPAP2C, SNCG, WDR46, C10orf76, DHX58,
HIBCH, MPEG1, PPAPDC1A, SND1, WDR47, C10orf82, DHX8, HIC1, MPG, PPAPDC1B, SNED1,
WDR48, C10orf88, DHX9, HIC2, MPHOSPH10, PPAPDC2, SNF8, WDR49, C10orf90, DIABLO,
HID1, MPHOSPH6, PPAPDC3, SNIP1, WDR5, C10orf91, DIAPH1, HIF1A, MPHOSPH8, PPARA,
SNN, WDR52, C10orf95, DIAPH2, HIF1AN, MPHOSPH9, PPARD, SNPH, WDR53, C10orf99,
DIAPH3, HIF3A, MPI, PPARG, SNRK, WDR54, C11orf1, DICER1, HIGD1A, MPL, PPARGC1A,
SNRNP200, WDR55, C11orf16, DIDO1, HIGD1B, MPLKIP, PPARGC1B, SNRNP25, WDR59,
C11orf21, DIEXF, HIGDIC, MPND, PPAT, SNRNP27, WDR5B, C11orf24, DIMT1, HIGD2A, MPO,
PPBP, SNRNP35, WDR6, C11orf30, DIO1, HILPDA, MPP1, PPCDC, SNRNP40, WDR60, C11orf31,
DIO2, HINFP, MPP2, PPCS, SNRNP48, WDR61, C11orf35, DIO3, HINT1, MPP3, PPDPF, SNRNP70,
WDR62, C11orf40, DIP2A, HINT2, MPP4, PPEF1, SNRPA, WDR63, C11orf42, DIP2B, HINT3, MPP5,
PPEF2, SNRPA1, WDR64, C11orf44, DIP2C, HIP1, MPP6, PPFIA1, SNRPB, WDR65, C11orf45,
DIRAS1, HIP1R, MPP7, PPFIA2, SNRPB2, WDR66, C11orf48, DIRAS2, HIPK1, MPPE1, PPFIA3,
SNRPC, WDR7, C11orf49, DIRAS3, HIPK2, MPPED1, PPFIA4, SNRPD1, WDR70, C11orf52, DIRC1,
HIPK3, MPPED2, PPFIBP1, SNRPD2, WDR72, C11orf53, DIRC2, HIPK4, MPRIP, PPFIBP2,
SNRPD3, WDR73, C11orf54, DIS3, HIRA, MPST, PPHLN1, SNRPE, WDR74, C11orf57, DIS3L,
HIRIP3, MPV17, PPIA, SNRPF, WDR75, C11orf58, DIS3L2, HIST1H1A, MPV17L, PPIAL4A,
SNRPG, WDR76, C11orf63, DISC1, HIST1H1B, MPV17L2, PPIAL4B, SNRPN, WDR77, C11orf65,
DISP1, HIST1H1C, MPZ, PPIAL4C, SNTA1, WDR78, C11orf68, DISP2, HIST1H1D, MPZL1,
PPIAL4D, SNTB1, WDR81, C11orf70, DIXDC1, HIST1H1E, MPZL2, PPIAL4E, SNTB2, WDR82,
C11orf71, DKC1, HIST1H1T, MPZL3, PPIAL4F, SNTG1, WDR83, C11orf73, DKK1, HIST1H2AA,
MR1, PPIAL4G, SNTG2, WDR83OS, C11orf74, DKK2, HIST1H2AB, MRAP, PPIB, SNTN, WDR86,
C11orf80, DKK3, HIST1H2AC, MRAP2, PPIC, SNUPN, WDR87, C11orf82, DKK4, HIST1H2AD,
MRAS, PPID, SNURF, WDR88, C11orf83, DKKL1, HIST1H2AE, MRC2, PPIE, SNW1, WDR89,
C11orf84, DLAT, HIST1H2AG, MRE11A, PPIF, SNX1, WDR90, C11orf85, DLC1, HIST1H2AH,
MREG, PPIG, SNX10, WDR91, C11orf86, DLD, HIST1H2AI, MRFAP1, PPIH, SNX11, WDR92,
C11orf87, DLEC1, HIST1H2AJ, MRFAP1L1, PPIL1, SNX12, WDR93, C11orf88, DLEU7,
HIST1H2AK, MRGBP, PPIL2, SNX13, WDR96, C11orf91, DLG1, HIST1H2AL, MRGPRD, PPIL3,
SNX14, WDSUB1, C11orf94, DLG2, HIST1H2AM, MRGPRE, PPIL4, SNX15, WDTC1, C11orf95,
DLG3, HIST1H2BA, MRGPRF, PPIL6, SNX16, WDYHV1, C11orf96, DLG4, HIST1H2BB, MRGPRG,
PPIP5K1, SNX17, WEE1, C12orf10, DLG5, HIST1H2BC, MRGPRX1, PPIP5K2, SNX18, WEE2,
C12orf23, DLGAP1, HIST1H2BD, MRGPRX2, PPL, SNX19, WFDC1, C12orf29, DLGAP2,
HIST1H2BE, MRGPRX3, PPM1A, SNX2, WFDC10A, C12orf39, DLGAP4, HIST1H2BF, MRGPRX4,
PPM1B, SNX20, WFDC10B, C12orf4, DLGAP5, HIST1H2BG, MRI1, PPM1D, SNX21, WFDC11,
C12orf40, DLK1, HIST1H2BH, MRM1, PPM1E, SNX22, WFDC12, C12orf42, DLK2, HIST1H2BI,
MRO, PPM1F, SNX24, WFDC13, C12orf43, DLL1, HIST1H2BJ, MROH1, PPM1G, SNX25, WFDC2,
C12orf44, DLL3, HIST1H2BK, MROH2A, PPM1H, SNX27, WFDC3, C12orf45, DLL4, HIST1H2BL,
MROH2B, PPM1J, SNX29, WFDC5, C12orf49, DLST, HIST1H2BM, MROH5, PPM1K, SNX3,
WFDC6, C12orf5, DLX1, HIST1H2BN, MROH6, PPM1L, SNX30, WFDC8, C12orf50, DLX2,
HIST1H2BO, MROH7, PPM1M, SNX31, WFDC9, C12orf52, DLX3, HIST1H3A, MROH8, PPM1N,
SNX32, WFIKKN1, C12orf54, DLX4, HIST1H3B, MROH9, PPME1, SNX33, WFIKKN2, C12orf55,
DLX5, HIST1H3C, MRP63, PPOX, SNX4, WFS1, C12orf56, DLX6, HIST1H3D, MRPL1, PPP1CA,
SNX5, WHAMM, C12orf57, DMAP1, HIST1H3E, MRPL10, PPP1CB, SNX6, WHSC1, C12orf60,
DMBT1, HIST1H3F, MRPL11, PPP1CC, SNX7, WHSC1L1, C12orf61, DMBX1, HIST1H3G, MRPL12,
PPP1R10, SNX8, WIBG, C12orf65, DMC1, HIST1H3H, MRPL13, PPP1R11, SNX9, WIF1, C12orf66,
DMD, HIST1H3I, MRPL14, PPP1R12A, SOAT1, WIPF1, C12orf68, DMGDH, HIST1H3J, MRPL15,
PPP1R12B, SOAT2, WIPF2, C12orf71, DMKN, HIST1H4A, MRPL16, PPP1R12C, SOBP, WIPF3,
C12orf73, DMP1, HIST1H4B, MRPL17, PPP1R13B, SOCS1, WIPI1, C12orf74, DMPK, HIST1H4C,
MRPL18, PPP1R13L, SOCS2, WIPI2, C12orf75, DMRT1, HIST1H4D, MRPL19, PPP1R14A, SOCS3,
WISP1, C12orf76, DMRT2, HIST1H4E, MRPL2, PPP1R14B, SOCS4, WISP2, C12orf77, DMRT3,
HIST1H4F, MRPL20, PPP1R14C, SOCS5, WISP3, C12orf79, DMRTA1, HIST1H4G, MRPL21,
PPP1R14D, SOCS6, WIZ, C12orf80, DMRTA2, HIST1H4H, MRPL22, PPP1R15A, SOCS7, WLS,
C13orf35, DMRTB1, HIST1H4I, MRPL23, PPP1R15B, SOD1, WNK1, C13orf45, DMRTC1,
HIST1H4J, MRPL24, PPP1R16A, SOD2, WNK2, C14orf1, DMRTC1B, HIST1H4K, MRPL27,
PPP1R16B, SOD3, WNK3, C14orf105, DMRTC2, HIST1H4L, MRPL28, PPP1R17, SOGA1, WNK4,
C14orf119, DMTF1, HIST2H2AA3, MRPL3, PPP1R18, SOGA2, WNT1, C14orf132, DMTN,
HIST2H2AA4, MRPL30, PPP1R1A, SOGA3, WNT10A, C14orf142, DMWD, HIST2H2AB, MRPL32,
PPP1R1B, SOHLH1, WNT10B, C14orf159, DMXL1, HIST2H2AC, MRPL33, PPP1R1C, SOHLH2,
WNT11, C14orf164, DMXL2, HIST2H2BE, MRPL34, PPP1R2, SON, WNT16, C14orf166, DNA2,
HIST2H2BF, MRPL35, PPP1R21, SORBS1, WNT2, C14orf166B, DNAAF1, HIST2H3A, MRPL36,
PPP1R26, SORBS2, WNT2B, C14orf169, DNAAF2, HIST2H3C, MRPL37, PPP1R27, SORBS3, WNT3,
C14orf177, DNAAF3, HIST2H3D, MRPL38, PPP1R32, SORCS1, WNT3A, C14orf178, DNAH1,
HIST2H4A, MRPL39, PPP1R35, SORCS2, WNT4, C14orf180, DNAH10, HIST2H4B, MRPL4,
PPP1R36, SORCS3, WNT5A, C14orf182, DNAH11, HIST3H2A, MRPL40, PPP1R37, SORD, WNT5B,
C14orf183, DNAH12, HIST3H2BB, MRPL41, PPP1R3A, SORL1, WNT6, C14orf2, DNAH14,
HIST3H3, MRPL42, PPP1R3B, SORT1, WNT7A, C14orf28, DNAH17, HIST4H4, MRPL43, PPP1R3C,
SOS1, WNT7B, C14orf37, DNAH2, HIVEP1, MRPL44, PPP1R3D, SOS2, WNT8A, C14orf39, DNAH3,
HIVEP2, MRPL45, PPP1R3E, SOST, WNT8B, C14orf79, DNAH5, HIVEP3, MRPL46, PPP1R3F,
SOSTDC1, WNT9A, C14orf80, DNAH6, HJURP, MRPL47, PPP1R3G, SOWAHA, WNT9B, C14orf93,
DNAH7, HK1, MRPL48, PPP1R42, SOWAHB, WRAP53, C15orf26, DNAH8, HK2, MRPL49, PPP1R7,
SOWAHC, WRAP73, C15orf27, DNAH9, HK3, MRPL50, PPP1R8, SOWAHD, WRB, C15orf32,
DNAI1, HKDC1, MRPL51, PPP1R9A, SOX1, WRN, C15orf38, DNAI2, HKR1, MRPL52, PPP1R9B,
SOX10, WRNIP1, C15orf38-AP3S2, DNAJA1, HLA-A, MRPL53, PPP2CA, SOX11, WSB1, C15orf39,
DNAJA2, HLA-B, MRPL54, PPP2CB, SOX12, WSB2, C15orf40, DNAJA3, HLA-C, MRPL55,
PPP2R1A, SOX13, WSCD1, C15orf41, DNAJA4, HLA-DMA, MRPL9, PPP2R1B, SOX14, WSCD2,
C15orf43, DNAJB1, HLA-DMB, MRPS10, PPP2R2A, SOX15, WT1, C15orf48, DNAJB11, HLA-DOA,
MRPS11, PPP2R2B, SOX17, WTAP, C15orf52, DNAJB12, HLA-DOB, MRPS12, PPP2R2C, SOX18,
WTH3DI, C15orf53, DNAJB13, HLA-DPA1, MRPS14, PPP2R2D, SOX2, WTIP, C15orf54, DNAJB14,
HLA-DPB1, MRPS15, PPP2R3A, SOX21, WWC1, C15orf56, DNAJB2, HLA- DQA1, MRPS16,
PPP2R3B, SOX3, WWC2, C15orf57, DNAJB3, HLA-DQA2, MRPS17, PPP2R3C, SOX30, WWC3,
C15orf59, DNAJB4, HLA-DQB1, MRPS18A, PPP2R4, SOX4, WWOX, C15orf60, DNAJB5, HLA-
DQB2, MRPS18B, PPP2R5A, SOX5, WWP1, C15orf61, DNAJB6, HLA-DRA, MRPS18C, PPP2R5B,
SOX6, WWP2, C15orf62, DNAJB7, HLA-DRB1, MRPS2, PPP2R5C, SOX7, WWTR1, C15orf65,
DNAJB8, HLA- DRB5, MRPS21, PPP2R5D, SOX8, XAB2, C16orf11, DNAJB9, HLA-E, MRPS22,
PPP2R5E, SOX9, XAF1, C16orf13, DNAJC1, HLA-F, MRPS23, PPP3CA, SP1, XAGE1D, C16orf3,
DNAJC10, HLA-G, MRPS24, PPP3CB, SP100, XAGE2, C16orf45, DNAJC11, HLCS, MRPS25,
PPP3CC, SP110, XAGE3, C16orf46, DNAJC12, HLF, MRPS26, PPP3R1, SP140, XAGE5, C16orf47,
DNAJC13, HLTF, MRPS27, PPP3R2, SP140L, XBP1, C16orf52, DNAJC14, HLX, MRPS28, PPP4C,
SP2, XCL1, C16orf54, DNAJC15, HM13, MRPS30, PPP4R1, SP3, XCL2, C16orf58, DNAJC16,
HMBOX1, MRPS31, PPP4R2, SP4, XCR1, C16orf59, DNAJC17, HMBS, MRPS33, PPP4R4, SP5,
XDH, C16orf62, DNAJC18, HMCES, MRPS34, PPP5C, SP6, XG, C16orf70, DNAJC19, HMCN1,
MRPS35, PPP5D1, SP7, XIAP, C16orf71, DNAJC2, HMCN2, MRPS36, PPP6C, SP8, XIRP1, C16orf72,
DNAJC21, HMG20A, MRPS5, PPP6R1, SP9, XIRP2, C16orf74, DNAJC22, HMG20B, MRPS6,
PPP6R2, SPA17, XK, C16orf78, DNAJC24, HMGA1, MRPS7, PPP6R3, SPACA1, XKR3, C16orf80,
DNAJC25, HMGA2, MRPS9, PPRC1, SPACA3, XKR4, C16orf82, DNAJC25-GNG10, HMGB1,
MRRF, PPT1, SPACA4, XKR5, C16orf86, DNAJC27, HMGB2, MRS2, PPT2, SPACA5, XKR6,
C16orf87, DNAJC28, HMGB3, MRTO4, PPTC7, SPACA5B, XKR7, C16orf89, DNAJC3, HMGB4,
MRVI1, PPWD1, SPACA7, XKR8, C16orf90, DNAJC30, HMGCL, MS4A1, PPY, SPAG1, XKR9,
C16orf91, DNAJC4, HMGCLL1, MS4A10, PQBP1, SPAG11A, XKRX, C16orf92, DNAJC5, HMGCR,
MS4A12, PQLC1, SPAG11B, XKRY, C16orf93, DNAJC5B, HMGCS1, MS4A13, PQLC2, SPAG16,
XKRY2, C16orf95, DNAJC5G, HMGCS2, MS4A14, PQLC3, SPAG17, XPA, C16orf96, DNAJC6,
HMGN1, MS4A15, PRAC1, SPAG4, XPC, C16orf97, DNAJC7, HMGN2, MS4A2, PRAC2, SPAG5,
XPNPEP1, C17orf100, DNAJC8, HMGN3, MS4A3, PRADC1, SPAG6, XPNPEP2, C17orf102,
DNAJC9, HMGN4, MS4A4A, PRAF2, SPAG7, XPNPEP3, C17orf103, DNAL1, HMGN5, MS4A4E,
PRAM1, SPAG8, XPO1, C17orf104, DNAL4, HMGXB3, MS4A5, PRAME, SPAG9, XPO4, C17orf105,
DNALI1, HMGXB4, MS4A6A, PRAMEF1, SPAM1, XPO5, C17orf107, DNASE1, HMHA1, MS4A6E,
PRAMEF10, SPANXA1, XPO6, C17orf112, DNASE1L1, HMHB1, MS4A7, PRAMEF11, SPANXA2,
XPO7, C17orf47, DNASE1L2, HMMR, MS4A8, PRAMEF12, SPANXB1, XPOT, C17orf49,
DNASE1L3, HMOX1, MSANTD1, PRAMEF13, SPANXB2, XPR1, C17orf50, DNASE2, HMOX2,
MSANTD2, PRAMEF14, SPANXC, XRCC1, C17orf51, DNASE2B, HMP19, MSANTD3, PRAMEF15,
SPANXD, XRCC2, C17orf53, DND1, HMSD, MSANTD3-TMEFF1, PRAMEF16, SPANXN1, XRCC3,
C17orf58, DNER, HMX1, MSANTD4, PRAMEF17, SPANXN2, XRCC4, C17orf59, DNHD1, HMX2,
MSC, PRAMEF18, SPANXN3, XRCC5, C17orf62, DNLZ, HMX3, MSGN1, PRAMEF19, SPANXN4,
XRCC6, C17orf64, DNM1, HN1, MSH2, PRAMEF2, SPANXN5, XRCC6BP1, C17orf66, DNM1L,
HN1L, MSH3, PRAMEF20, SPARC, XRN1, C17orf67, DNM2, HNF1A, MSH4, PRAMEF21,
SPARCL1, XRN2, C17orf70, DNM3, HNF1B, MSH5, PRAMEF22, SPAST, XRRA1, C17orf72,
DNMBP, HNF4A, MSH6, PRAMEF23, SPATA1, XXYLT1, C17orf74, DNMT1, HNF4G, MSI1,
PRAMEF25, SPATA12, XYLB, C17orf75, DNMT3A, HNMT, MSI2, PRAMEF3, SPATA13, XYLT1,
C17orf77, DNMT3B, HNRNPA0, MSL1, PRAMEF4, SPATA16, XYLT2, C17orf78, DNMT3L,
HNRNPA1, MSL2, PRAMEF5, SPATA17, YAE1D1, C17orf80, DNPEP, HNRNPA1L2, MSL3,
PRAMEF6, SPATA18, YAF2, C17orf82, DNPH1, HNRNPA2B1, MSLN, PRAMEF7, SPATA19,
YAP1, C17orf85, DNTT, HNRNPA3, MSMB, PRAMEF8, SPATA2, YARS, C17orf89, DNTTIP1,
HNRNPAB, MSMO1, PRAMEF9, SPATA20, YARS2, C17orf96, DNTTIP2, HNRNPC, MSMP,
PRAP1, SPATA21, YBEY, C17orf97, DOC2A, HNRNPCL1, MSN, PRB1, SPATA22, YBX1, C17orf98,
DOC2B, HNRNPCP5, MSR1, PRB2, SPATA24, YBX2, C17orf99, DOCK1, HNRNPD, MSRA, PRB3,
SPATA25, YBX3, C18orf21, DOCK10, HNRNPDL, MSRB1, PRB4, SPATA2L, YDJC, C18orf25,
DOCK11, HNRNPF, MSRB2, PRC1, SPATA3, YEATS2, C18orf32, DOCK2, HNRNPH1, MSRB3,
PRCC, SPATA31A1, YEATS4, C18orf42, DOCK3, HNRNPH2, MSS51, PRCD, SPATA31A2, YES1,
C18orf54, DOCK4, HNRNPH3, MST1, PRCP, SPATA31A3, YIF1A, C18orf56, DOCK5, HNRNPK,
MST1L, PRDM1, SPATA31A4, YIF1B, C18orf63, DOCK6, HNRNPL, MST1R, PRDM10,
SPATA31A5, YIPF1, C18orf8, DOCK7, HNRNPLL, MST4, PRDM11, SPATA31A6, YIPF2, C19orf10,
DOCK8, HNRNPM, MSTN, PRDM12, SPATA31A7, YIPF3, C19orf12, DOCK9, HNRNPR, MSTO1,
PRDM13, SPATA31C1, YIPF4, C19orf18, DOHH, HNRNPU, MSX1, PRDM14, SPATA31C2, YIPF5,
C19orf24, DOK1, HNRNPUL1, MSX2, PRDM15, SPATA31D1, YIPF6, C19orf25, DOK2,
HNRNPUL2, MT1A, PRDM16, SPATA31D3, YIPF7, C19orf26, DOK3, HOGA1, MT1B, PRDM2,
SPATA31D4, YJEFN3, C19orf33, DOK4, HOMER1, MT1E, PRDM4, SPATA31E1, YKT6, C19orf35,
DOK5, HOMER2, MT1F, PRDM5, SPATA32, YLPM1, C19orf38, DOK6, HOMER3, MT1G, PRDM6,
SPATA33, YME1L1, C19orf40, DOK7, HOMEZ, MT1H, PRDM7, SPATA4, YOD1, C19orf43, DOLK,
HOOK1, MT1HL1, PRDM8, SPATA5, YPEL1, C19orf44, DOLPP1, HOOK2, MT1M, PRDM9,
SPATA5L1, YPEL2, C19orf45, DONSON, HOOK3, MT1X, PRDX1, SPATA6, YPEL3, C19orf47,
DOPEY1, HOPX, MT2A, PRDX2, SPATA6L, YPEL4, C19orf48, DOPEY2, HORMAD1, MT3,
PRDX3, SPATA7, YPEL5, C19orf52, DOT1L, HORMAD2, MT4, PRDX4, SPATA8, YRDC, C19orf53,
DPAGT1, HOXA1, MTA1, PRDX5, SPATA9, YTHDC1, C19orf54, DPCD, HOXA10, MTA2, PRDX6,
SPATC1, YTHDC2, C19orf55, DPCR1, HOXA11, MTA3, PREB, SPATC1L, YTHDF1, C19orf57,
DPEP1, HOXA13, MTAP, PRELID1, SPATS1, YTHDF2, C19orf59, DPEP2, HOXA2, MTBP,
PRELID2, SPATS2, YTHDF3, C19orf60, DPEP3, HOXA3, MTCH1, PRELP, SPATS2L, YWHAB,
C19orf66, DPF1, HOXA4, MTCH2, PREP, SPC24, YWHAE, C19orf67, DPF2, HOXA5, MTCP1,
PREPL, SPC25, YWHAG, C19orf68, DPF3, HOXA6, MTDH, PREX1, SPCS1, YWHAH, C19orf69,
DPH1, HOXA7, MTERF, PREX2, SPCS2, YWHAQ, C19orf70, DPH2, HOXA9, MTERFD1, PRF1,
SPCS3, YWHAZ, C19orf71, DPH3, HOXB1, MTERFD2, PRG2, SPDEF, YY1, C19orf73, DPH3P1,
HOXB13, MTERFD3, PRG3, SPDL1, YY1AP1, C19orf77, DPH5, HOXB2, MTF1, PRG4, SPDYA,
YY2, C19orf80, DPH6, HOXB3, MTF2, PRH1, SPDYC, ZACN, C19orf81, DPH7, HOXB4, MTFMT,
PRH2, SPDYE1, ZADH2, C19orf82, DPM1, HOXB5, MTFP1, PRICKLE1, SPDYE2, ZAK, C1D,
DPM2, HOXB6, MTFR1, PRICKLE2, SPDYE2B, ZAN, C1GALT1, DPM3, HOXB7, MTFR1L,
PRICKLE3, SPDYE3, ZAP70, C1GALT1C1, DPP10, HOXB8, MTFR2, PRICKLE4, SPDYE4, ZAR1,
C1orf100, DPP3, HOXB9, MTG1, PRIM1, SPDYE5, ZAR1L, C1orf101, DPP4, HOXC10, MTG2,
PRIM2, SPDYE6, ZBBX, C1orf105, DPP6, HOXC11, MTHFD1, PRIMA1, SPECC1, ZBED1,
C1orf106, DPP7, HOXC12, MTHFD1L, PRIMPOL, SPECC1L, ZBED2, C1orf109, DPP8, HOXC13,
MTHFD2, PRKAA1, SPEF1, ZBED3, C1orf110, DPP9, HOXC4, MTHFD2L, PRKAA2, SPEF2,
ZBED4, C1orf111, DPPA2, HOXC5, MTHFR, PRKAB1, SPEG, ZBED5, C1orf112, DPPA3, HOXC6,
MTHFS, PRKAB2, SPEMI, ZBED6, C1orf115, DPPA4, HOXC8, MTHFSD, PRKACA, SPEN,
ZBED6CL, C1orf116, DPPA5, HOXC9, MTIF2, PRKACB, SPERT, ZBP1, C1orf122, DPRX, HOXD1,
MTIF3, PRKACG, SPESP1, ZBTB1, C1orf123, DPT, HOXD10, MTL5, PRKAG1, SPG11, ZBTB10,
C1orf127, DPY19L1, HOXD11, MTM1, PRKAG2, SPG20, ZBTB11, C1orf131, DPY19L2, HOXD12,
MTMR1, PRKAG3, SPG21, ZBTB12, C1orf137, DPY19L3, HOXD13, MTMR10, PRKAR1A, SPG7,
ZBTB14, C1orf141, DPY19L4, HOXD3, MTMR11, PRKAR1B, SPHAR, ZBTB16, C1orf146, DPY30,
HOXD4, MTMR12, PRKAR2A, SPHK1, ZBTB17, C1orf158, DPYD, HOXD8, MTMR14, PRKAR2B,
SPHK2, ZBTB18, C1orf159, DPYS, HOXD9, MTMR2, PRKCA, SPHKAP, ZBTB2, C1orf162,
DPYSL2, HP, MTMR3, PRKCB, SPI1, ZBTB20, C1orf167, DPYSL3, HP1BP3, MTMR4, PRKCD,
SPIB, ZBTB21, C1orf168, DPYSL4, HPCA, MTMR6, PRKCDBP, SPIC, ZBTB22, C1orf172, DPYSL5,
HPCAL1, MTMR7, PRKCE, SPICE1, ZBTB24, C1orf173, DQX1, HPCAL4, MTMR8, PRKCG, SPIDR,
ZBTB25, C1orf174, DR1, HPD, MTMR9, PRKCH, SPIN1, ZBTB26, C1orf177, DRAM1, HPDL,
MTNR1A, PRKCI, SPIN2A, ZBTB3, C1orf185, DRAM2, HPGD, MTNR1B, PRKCQ, SPIN2B,
ZBTB32, C1orf186, DRAP1, HPGDS, MTO1, PRKCSH, SPIN3, ZBTB33, C1orf189, DRAXIN, HPN,
MTOR, PRKCZ, SPIN4, ZBTB34, C1orf192, DRC1, HPR, MTPAP, PRKD1, SPINK1, ZBTB37,
C1orf194, DRD1, HPRT1, MTPN, PRKD2, SPINK13, ZBTB38, C1orf195, DRD2, HPS1, MTR,
PRKD3, SPINK14, ZBTB39, C1orf198, DRD3, HPS3, MTRF1, PRKDC, SPINK2, ZBTB4, C1orf204,
DRD4, HPS4, MTRF1L, PRKG1, SPINK4, ZBTB40, C1orf21, DRD5, HPS5, MTRNR2L10, PRKG2,
SPINK5, ZBTB41, C1orf210, DRG1, HPS6, MTRNR2L2, PRKRA, SPINK6, ZBTB42, C1orf216,
DRG2, HPSE, MTRNR2L3, PRKRIP1, SPINK7, ZBTB43, C1orf226, DRGX, HPSE2, MTRNR2L4,
PRKRIR, SPINK8, ZBTB44, C1orf227, DROSHA, HPX, MTRNR2L5, PRKX, SPINK9, ZBTB45,
C1orf228, DRP2, HR, MTRNR2L6, PRL, SPINTI, ZBTB46, C1orf229, DSC1, HRAS, MTRNR2L7,
PRLH, SPINT2, ZBTB47, C1orf233, DSC2, HRASLS, MTRNR2L9, PRLHR, SPINT3, ZBTB48,
C1orf234, DSC3, HRASLS2, MTRR, PRLR, SPINT4, ZBTB49, C1orf27, DSCAM, HRASLS5, MTSS1,
PRM1, SPIRE1, ZBTB5, C1orf35, DSCAML1, HRC, MTSS1L, PRM2, SPIRE2, ZBTB6, C1orf43,
DSCC1, HRCT1, MTTP, PRM3, SPN, ZBTB7A, C1orf50, DSCR3, HRG, MTURN, PRMT1, SPNS1,
ZBTB7B, C1orf51, DSCR4, HRH1, MTUS1, PRMT10, SPNS2, ZBTB7C, C1orf52, DSE, HRH2,
MTUS2, PRMT2, SPNS3, ZBTB8A, C1orf53, DSEL, HRH3, MTX1, PRMT3, SPO11, ZBTB8B,
C1orf54, DSG1, HRH4, MTX2, PRMT5, SPOCD1, ZBTB8OS, C1orf56, DSG2, HRK, MTX3, PRMT6,
SPOCK1, ZBTB9, C1orf61, DSG3, HRNR, MUC1, PRMT7, SPOCK2, ZC2HC1A, C1orf63, DSG4,
HRSP12, MUC12, PRMT8, SPOCK3, ZC2HC1B, C1orf64, DSN1, HS1BP3, MUC13, PRND, SPON1,
ZC2HC1C, C1orf65, DSP, HS2ST1, MUC15, PRNP, SPON2, ZC3H10, C1orf68, DSPP, HS3ST1,
MUC16, PROB1, SPOP, ZC3H11A, C1orf74, DST, HS3ST2, MUC17, PROC, SPOPL, ZC3H12A,
C1orf85, DSTN, HS3ST3A1, MUC19, PROCA1, SPP1, ZC3H12B, C1orf86, DSTNP4, HS3ST3B1,
MUC2, PROCR, SPP2, ZC3H12C, C1orf87, DSTYK, HS3ST4, MUC20, PRODH, SPPL2A, ZC3H12D,
C1orf94, DTD1, HS3ST5, MUC21, PRODH2, SPPL2B, ZC3H13, C1orf95, DTD2, HS3ST6, MUC22,
PROK1, SPPL2C, ZC3H14, C1QA, DTHD1, HS6ST1, MUC3A, PROK2, SPPL3, ZC3H15, C1QB, DTL,
HS6ST2, MUC4, PROKR1, SPR, ZC3H18, C1QBP, DTNA, HS6ST3, MUC5AC, PROKR2, SPRED1,
ZC3H3, C1QC, DTNB, HSBP1, MUC5B, PROL1, SPRED2, ZC3H4, C1QL1, DTNBP1, HSBP1L1,
MUC6, PROM1, SPRED3, ZC3H6, C1QL2, DTWD1, HSCB, MUC7, PROM2, SPRN, ZC3H7A,
C1QL3, DTWD2, HSD11B1, MUC8, PROP1, SPRR1A, ZC3H7B, C1QL4, DTX1, HSD11B1L,
MUCL1, PROS1, SPRR1B, ZC3H8, C1QTNF1, DTX2, HSD11B2, MUL1, PROSC, SPRR2A,
ZC3HAV1, C1QTNF2, DTX3, HSD17B1, MUM1, PROSER1, SPRR2B, ZC3HAV1L, C1QTNF3,
DTX3L, HSD17B10, MUM1L1, PROSER2, SPRR2D, ZC3HC1, C1QTNF4, DTX4, HSD17B11,
MURC, PROX1, SPRR2E, ZC4H2, C1QTNF5, DTYMK, HSD17B12, MUS81, PROX2, SPRR2F,
ZCCHC10, C1QTNF6, DUOX1, HSD17B13, MUSK, PROZ, SPRR2G, ZCCHC11, C1QTNF7, DUOX2,
HSD17B14, MUSTN1, PRPF18, SPRR3, ZCCHC12, C1QTNF8, DUOXA1, HSD17B2, MUT, PRPF19,
SPRR4, ZCCHC13, C1QTNF9, DUOXA2, HSD17B3, MUTYH, PRPF3, SPRTN, ZCCHC14,
C1QTNF9B, DUPD1, HSD17B4, MVB12A, PRPF31, SPRY1, ZCCHC16, C1QTNF9B-AS1, DUS1L,
HSD17B6, MVB12B, PRPF38A, SPRY2, ZCCHC17, C1R, DUS2, HSD17B7, MVD, PRPF38B, SPRY3,
ZCCHC18, C1RL, DUS3L, HSD17B8, MVK, PRPF39, SPRY4, ZCCHC2, C1S, DUS4L, HSD3B1,
MVP, PRPF4, SPRYD3, ZCCHC24, C2, DUSP1, HSD3B2, MX1, PRPF40A, SPRYD4, ZCCHC3,
C20orf112, DUSP10, HSD3B7, MX2, PRPF40B, SPRYD7, ZCCHC4, C20orf141, DUSP11, HSDL1,
MXD1, PRPF4B, SPSB1, ZCCHC5, C20orf144, DUSP12, HSDL2, MXD3, PRPF6, SPSB2, ZCCHC6,
C20orf166, DUSP13, HSF1, MXD4, PRPF8, SPSB3, ZCCHC7, C20orf173, DUSP14, HSF2, MXI1,
PRPH, SPSB4, ZCCHC8, C20orf194, DUSP15, HSF2BP, MXRA5, PRPH2, SPTA1, ZCCHC9,
C20orf195, DUSP16, HSF4, MXRA7, PRPS1, SPTAN1, ZCRB1, C20orf196, DUSP18, HSF5, MXRA8,
PRPS1L1, SPTB, ZCWPW1, C20orf197, DUSP19, HSFX1, MYADM, PRPS2, SPTBN1, ZCWPW2,
C20orf201, DUSP2, HSFX2, MYADML2, PRPSAP1, SPTBN2, ZDBF2, C20orf202, DUSP21, HSFY1,
MYB, PRPSAP2, SPTBN4, ZDHHC1, C20orf203, DUSP22, HSFY2, MYBBP1A, PRR11, SPTBN5,
ZDHHC11, C20orf24, DUSP23, HSH2D, MYBL1, PRR12, SPTLC1, ZDHHC11B, C20orf26, DUSP26,
HSP90AA1, MYBL2, PRR13, SPTLC2, ZDHHC12, C20orf27, DUSP27, HSP90AB1, MYBPC1,
PRR14, SPTLC3, ZDHHC13, C20orf62, DUSP28, HSP90B1, MYBPC2, PRR14L, SPTSSA, ZDHHC14,
C20orf78, DUSP3, HSPA12A, MYBPC3, PRR15, SPTSSB, ZDHHC15, C20orf85, DUSP4, HSPA12B,
MYBPH, PRR15L, SPTY2D1, ZDHHC16, C20orf96, DUSP5, HSPA13, MYBPHL, PRR16, SPZ1,
ZDHHC17, C21orf140, DUSP6, HSPA14, MYC, PRR18, SQLE, ZDHHC18, C21orf2, DUSP7,
HSPA1A, MYCBP, PRR19, SQRDL, ZDHHC19, C21orf33, DUSP8, HSPA1B, MYCBP2, PRR20A,
SQSTM1, ZDHHC2, C21orf58, DUSP9, HSPA1L, MYCBPAP, PRR20B, SRA1, ZDHHC20, C21orf59,
DUT, HSPA2, MYCL, PRR20C, SRBD1, ZDHHC21, C21orf62, DUXA, HSPA4, MYCN, PRR20D,
SRC, ZDHHC22, C21orf91, DVL1, HSPA4L, MYCT1, PRR20E, SRCAP, ZDHHC23, C22orf15, DVL2,
HSPA5, MYD88, PRR21, SRCIN1, ZDHHC24, C22orf23, DVL3, HSPA6, MYEF2, PRR22, SRCRB4D,
ZDHHC3, C22orf24, DXO, HSPA8, MYEOV, PRR23A, SRD5A1, ZDHHC4, C22orf26, DYDC1,
HSPA9, MYEOV2, PRR23B, SRD5A2, ZDHHC5, C22orf29, DYDC2, HSPB1, MYF5, PRR23C,
SRD5A3, ZDHHC6, C22orf31, DYM, HSPB11, MYF6, PRR24, SREBF1, ZDHHC7, C22orf39,
DYNAP, HSPB2, MYH1, PRR25, SREBF2, ZDHHC8, C22orf42, DYNC1H1, HSPB3, MYH10, PRR3,
SREK1, ZDHHC9, C22orf43, DYNC1I1, HSPB6, MYH11, PRR30, SREK1IP1, ZEB1, C22orf46,
DYNC1I2, HSPB7, MYH13, PRR4, SRF, ZEB2, C2CD2, DYNC1LI1, HSPB8, MYH14, PRR5,
SRFBP1, ZER1, C2CD2L, DYNC1LI2, HSPB9, MYH15, PRR5-ARHGAP8, SRGAP1, ZFAND1,
C2CD3, DYNC2H1, HSPBAP1, MYH2, PRR5L, SRGAP2, ZFAND2A, C2CD4A, DYNC2LI1,
HSPBP1, MYH3, PRR7, SRGAP2B, ZFAND2B, C2CD4B, DYNLL1, HSPD1, MYH4, PRR9,
SRGAP2C, ZFAND3, C2CD4C, DYNLL2, HSPE1, MYH6, PRRC1, SRGAP3, ZFAND4, C2CD4D,
DYNLRB1, HSPE1-MOB4, MYH7, PRRC2A, SRGN, ZFAND5, C2CD5, DYNLRB2, HSPG2,
MYH7B, PRRC2B, SRI, ZFAND6, C2orf15, DYNLT1, HSPH1, MYH8, PRRC2C, SRL, ZFAT,
C2orf16, DYNLT3, HTATIP2, MYH9, PRRG1, SRM, ZFC3H1, C2orf27A, DYRK1A, HTATSF1,
MYL1, PRRG2, SRMS, ZFHX2, C2orf27B, DYRK1B, HTN1, MYL10, PRRG3, SRP14, ZFHX3,
C2orf40, DYRK2, HTN3, MYL12A, PRRG4, SRP19, ZFHX4, C2orf42, DYRK3, HTR1A, MYL12B,
PRRT1, SRP54, ZFP1, C2orf43, DYRK4, HTR1B, MYL2, PRRT2, SRP68, ZFP14, C2orf44, DYSF,
HTR1D, MYL3, PRRT3, SRP72, ZFP2, C2orf47, DYTN, HTR1E, MYL4, PRRT4, SRP9, ZFP28,
C2orf48, DYX1C1, HTR1F, MYL5, PRRX1, SRPK1, ZFP3, C2orf49, DZANK1, HTR2A, MYL6,
PRRX2, SRPK2, ZFP30, C2orf50, DZIP1, HTR2B, MYL6B, PRSS1, SRPK3, ZFP36, C2orf53, DZIP1L,
HTR2C, MYL7, PRSS12, SRPR, ZFP36L1, C2orf54, DZIP3, HTR3A, MYL9, PRSS16, SRPRB,
ZFP36L2, C2orf57, E2F1, HTR3B, MYLIP, PRSS2, SRPX, ZFP37, C2orf61, E2F2, HTR3C, MYLK,
PRSS21, SRPX2, ZFP41, C2orf62, E2F3, HTR3D, MYLK2, PRSS22, SRR, ZFP42, C2orf66, E2F4,
HTR3E, MYLK3, PRSS23, SRRD, ZFP57, C2orf68, E2F5, HTR4, MYLK4, PRSS27, SRRM1, ZFP62,
C2orf69, E2F6, HTR5A, MYLPF, PRSS3, SRRM2, ZFP64, C2orf70, E2F7, HTR6, MYNN, PRSS33,
SRRM3, ZFP69, C2orf71, E2F8, HTR7, MYO10, PRSS35, SRRM4, ZFP69B, C2orf72, E4F1, HTRA1,
MYO15A, PRSS36, SRRM5, ZFP82, C2orf73, EAF1, HTRA2, MYO16, PRSS37, SRRT, ZFP90,
C2orf74, EAF2, HTRA3, MYO18A, PRSS38, SRSF1, ZFP91, C2orf76, EAPP, HTRA4, MYO18B,
PRSS41, SRSF10, ZFP92, C2orf78, EARS2, HTT, MYO19, PRSS42, SRSF11, ZFPL1, C2orf80,
EBAG9, HUNK, MYO1A, PRSS44, SRSF12, ZFPM1, C2orf81, EBF1, HUS1, MYO1B, PRSS45,
SRSF2, ZFPM2, C2orf82, EBF2, HUS1B, MYO1C, PRSS46, SRSF3, ZFR, C2orf83, EBF3, HUWE1,
MYO1D, PRSS48, SRSF4, ZFR2, C2orf88, EBF4, HVCN1, MYO1E, PRSS50, SRSF5, ZFX, C2orf91,
EBI3, HYAL1, MYO1F, PRSS53, SRSF6, ZFY, C3, EBLN1, HYAL2, MYO1G, PRSS54, SRSF7,
ZFYVE1, C3AR1, EBLN2, HYAL3, MYO1H, PRSS55, SRSF8, ZFYVE16, C3orf14, EBNA1BP2,
HYAL4, MYO3A, PRSS56, SRSF9, ZFYVE19, C3orf17, EBP, HYDIN, MYO3B, PRSS57, SRXN1,
ZFYVE20, C3orf18, EBPL, HYI, MYO5A, PRSS58, SRY, ZFYVE21, C3orf20, ECD, HYKK, MYO5B,
PRSS8, SS18, ZFYVE26, C3orf22, ECE1, HYLS1, MYO5C, PRTFDC1, SS18L1, ZFYVE27, C3orf27,
ECE2, HYOU1, MYO6, PRTG, SS18L2, ZFYVE28, C3orf30, ECEL1, HYPK, MYO7A, PRTN3, SSB,
ZFYVE9, C3orf33, ECH1, IAH1, MYO7B, PRUNE, SSBP1, ZG16, C3orf35, ECHDC1, IAPP, MYO9A,
PRUNE2, SSBP2, ZG16B, C3orf36, ECHDC2, IARS, MYO9B, PRX, SSBP3, ZGLP1, C3orf38,
ECHDC3, IARS2, MYOC, PRY, SSBP4, ZGPAT, C3orf43, ECHS1, IBA57, MYOCD, PRY2, SSC5D,
ZHX1, C3orf52, ECI1, IBSP, MYOD1, PSAP, SSFA2, ZHX1-C8ORF76, C3orf55, ECI2, IBTK, MYOF,
PSAPL1, SSH1, ZHX2, C3orf56, ECM1, ICA1, MYOG, PSAT1, SSH2, ZHX3, C3orf58, ECM2, ICA1L,
MYOM1, PSCA, SSH3, ZIC1, C3orf62, ECSCR, ICAM1, MYOM2, PSD, SSMEM1, ZIC2, C3orf67,
ECSIT, ICAM2, MYOM3, PSD2, SSNA1, ZIC3, C3orf70, ECT2, ICAM3, MYOT, PSD3, SSPN, ZIC4,
C3orf72, ECT2L, ICAM4, MYOZ1, PSD4, SSPO, ZIC5, C3orf79, EDA, ICAM5, MYOZ2, PSEN1,
SSR1, ZIK1, C3orf80, EDA2R, ICK, MYOZ3, PSEN2, SSR2, ZIM2, C3orf83, EDAR, ICMT, MYPN,
PSENEN, SSR3, ZIM3, C3orf84, EDARADD, ICOS, MYPOP, PSG1, SSR4, ZKSCAN1, C4A, EDC3,
ICOSLG, MYRF, PSG11, SSRP1, ZKSCAN2, C4B, EDC4, ICT1, MYRFL, PSG2, SSSCA1, ZKSCAN3,
C4B_2, EDDM3A, ID1, MYRIP, PSG3, SST, ZKSCAN4, C4BPA, EDDM3B, ID2, MYSM1, PSG4,
SSTR1, ZKSCAN5, C4BPB, EDEM1, ID3, MYT1, PSG5, SSTR2, ZKSCAN7, C4orf17, EDEM2, ID4,
MYT1L, PSG6, SSTR3, ZKSCAN8, C4orf19, EDEM3, IDE, MYZAP, PSG7, SSTR4, ZMAT1, C4orf21,
EDF1, IDH1, MZB1, PSG8, SSTR5, ZMAT2, C4orf22, EDIL3, IDH2, MZF1, PSG9, SSU72, ZMAT3,
C4orf26, EDN1, IDH3A, MZT1, PSIP1, SSUH2, ZMAT4, C4orf27, EDN2, IDH3B, MZT2A, PSKH1,
SSX1, ZMAT5, C4orf29, EDN3, IDH3G, MZT2B, PSKH2, SSX2, ZMIZ1, C4orf3, EDNRA, IDI1,
N4BP1, PSMA1, SSX2B, ZMIZ2, C4orf32, EDNRB, IDI2, N4BP2, PSMA2, SSX2IP, ZMPSTE24,
C4orf33, EEA1, IDNK, N4BP2L1, PSMA3, SSX3, ZMYM1, C4orf36, EED, IDO1, N4BP2L2, PSMA4,
SSX4, ZMYM2, C4orf40, EEF1A1, IDO2, N4BP3, PSMA5, SSX4B, ZMYM3, C4orf45, EEF1A2, IDS,
N6AMT1, PSMA6, SSX5, ZMYM4, C4orf46, EEF1B2, IDUA, N6AMT2, PSMA7, SSX7, ZMYM5,
C4orf47, EEF1D, IER2, NAA10, PSMA8, ST13, ZMYM6, C4orf48, EEF1E1, IER3, NAA11, PSMB1,
ST14, ZMYM6NB, C4orf50, EEF1G, IER3IP1, NAA15, PSMB10, ST18, ZMYND10, C4orf51, EEF2,
IER5, NAA16, PSMB11, ST20, ZMYND11, C4orf6, EEF2K, IER5L, NAA20, PSMB2, ST20-MTHFS,
ZMYND12, C5, EEFSEC, IFFO1, NAA25, PSMB3, ST3GAL1, ZMYND15, C5AR1, EEPD1, IFFO2,
NAA30, PSMB4, ST3GAL2, ZMYND19, C5AR2, EFCAB1, IFI16, NAA35, PSMB5, ST3GAL3,
ZMYND8, C5orf15, EFCAB11, IFI27, NAA38, PSMB6, ST3GAL4, ZNF10, C5orf20, EFCAB12,
IFI27L1, NAA40, PSMB7, ST3GAL5, ZNF100, C5orf22, EFCAB13, IFI27L2, NAA50, PSMB8,
ST3GAL6, ZNF101, C5orf24, EFCAB14, IFI30, NAA60, PSMB9, ST5, ZNF106, C5orf28, EFCAB2,
IFI35, NAAA, PSMC1, ST6GAL1, ZNF107, C5orf30, EFCAB3, IFI44, NAALAD2, PSMC2, ST6GAL2,
ZNF112, C5orf34, EFCAB4A, IFI44L, NAALADL1, PSMC3, ST6GALNAC1, ZNF114, C5orf38,
EFCAB4B, IFI6, NAALADL2, PSMC3IP, ST6GALNAC2, ZNF117, C5orf42, EFCAB5, IFIH1, NAB1,
PSMC4, ST6GALNAC3, ZNF12, C5orf45, EFCAB6, IFIT1, NAB2, PSMC5, ST6GALNAC4, ZNF121,
C5orf46, EFCAB7, IFIT1B, NABP1, PSMC6, ST6GALNAC5, ZNF124, C5orf47, EFCAB8, IFIT2,
NABP2, PSMD1, ST6GALNAC6, ZNF131, C5orf48, EFCAB9, IFIT3, NACA, PSMD10, ST7, ZNF132,
C5orf49, EFCC1, IFIT5, NACA2, PSMD11, ST7L, ZNF133, C5orf50, EFEMP1, IFITM1, NACAD,
PSMD12, ST8SIA1, ZNF134, C5orf51, EFEMP2, IFITM10, NACC1, PSMD13, ST8SIA2, ZNF135,
C5orf52, EFHB, IFITM2, NACC2, PSMD14, ST8SIA3, ZNF136, C5orf55, EFHC1, IFITM3, NADK,
PSMD2, ST8SIA4, ZNF138, C5orf58, EFHC2, IFITM5, NADK2, PSMD3, ST8SIA5, ZNF14, C5orf60,
EFHD1, IFLTD1, NADSYN1, PSMD4, ST8SIA6, ZNF140, C5orf63, EFHD2, IFNA1, NAE1, PSMD5,
STAB1, ZNF141, C5orf64, EFNA1, IFNA10, NAF1, PSMD6, STAB2, ZNF142, C6, EFNA2, IFNA13,
NAGA, PSMD7, STAC, ZNF143, C6orf1, EFNA3, IFNA14, NAGK, PSMD8, STAC2, ZNF146,
C6orf10, EFNA4, IFNA16, NAGLU, PSMD9, STAC3, ZNF148, C6orf106, EFNA5, IFNA17, NAGPA,
PSME1, STAG1, ZNF154, C6orf118, EFNB1, IFNA2, NAGS, PSME2, STAG2, ZNF155, C6orf132,
EFNB2, IFNA21, NAIF1, PSME3, STAG3, ZNF157, C6orf136, EFNB3, IFNA4, NAIP, PSME4, STAM,
ZNF16, C6orf141, EFR3A, IFNA5, NALCN, PSMF1, STAM2, ZNF160, C6orf15, EFR3B, IFNA6,
NAMPT, PSMG1, STAMBP, ZNF165, C6orf163, EFS, IFNA7, NANOG, PSMG2, STAMBPL1,
ZNF169, C6orf165, EFTUD1, IFNA8, NANOGNB, PSMG3, STAP1, ZNF17, C6orf183, EFTUD2,
IFNAR1, NANOS1, PSMG4, STAP2, ZNF174, C6orf195, EGF, IFNAR2, NANOS2, PSORS1C1,
STAR, ZNF175, C6orf201, EGFL6, IFNB1, NANOS3, PSORS1C2, STARD10, ZNF177, C6orf203,
EGFL7, IFNE, NANP, PSPC1, STARD13, ZNF18, C6orf211, EGFL8, IFNG, NANS, PSPH, STARD3,
ZNF180, C6orf222, EGFLAM, IFNGR1, NAP1L1, PSPN, STARD3NL, ZNF181, C6orf223, EGFR,
IFNGR2, NAP1L2, PSRC1, STARD4, ZNF182, C6orf226, EGLN1, IFNK, NAP1L3, PSTK, STARD5,
ZNF184, C6orf25, EGLN2, IFNL1, NAP1L4, PSTPIP1, STARD6, ZNF185, C6orf47, EGLN3, IFNL2,
NAP1L5, PSTPIP2, STARD7, ZNF189, C6orf48, EGR1, IFNL3, NAPA, PTAFR, STARD8, ZNF19,
C6orf52, EGR2, IFNL4, NAPB, PTAR1, STARD9, ZNF195, C6orf57, EGR3, IFNLR1, NAPEPLD,
PTBP1, STAT1, ZNF197, C6orf58, EGR4, IFNW1, NAPG, PTBP2, STAT2, ZNF2, C6orf62, EHBP1,
IFRD1, NAPRT1, PTBP3, STAT3, ZNF20, C6orf7, EHBP1L1, IFRD2, NAPSA, PTCD1, STAT4,
ZNF200, C6orf89, EHD1, IFT122, NARF, PTCD2, STAT5A, ZNF202, C6orf99, EHD2, IFT140,
NARFL, PTCD3, STAT5B, ZNF205, C7, EHD3, IFT172, NARG2, PTCH1, STAT6, ZNF207, C7orf10,
EHD4, IFT20, NARR, PTCH2, STATH, ZNF208, C7orf25, EHF, IFT27, NARS, PTCHD1, STAU1,
ZNF211, C7orf26, EHHADH, IFT43, NARS2, PTCHD2, STAU2, ZNF212, C7orf31, EHMT1, IFT46,
NASP, PTCHD3, STBD1, ZNF213, C7orf33, EHMT2, IFT52, NAT1, PTCHD4, STC1, ZNF214,
C7orf34, EI24, IFT57, NAT10, PTCRA, STC2, ZNF215, C7orf43, EID1, IFT74, NAT14, PTDSS1,
STEAP1, ZNF217, C7orf49, EID2, IFT80, NAT16, PTDSS2, STEAP1B, ZNF219, C7orf50, EID2B,
IFT81, NAT2, PTEN, STEAP2, ZNF22, C7orf55, EID3, IFT88, NAT6, PTER, STEAP3, ZNF221,
C7orf55- LUC7L2, EIF1, IGBP1, NAT8, PTF1A, STEAP4, ZNF222, C7orf57, EIF1AD, IGDCC3,
NAT8B, PTGDR, STH, ZNF223, C7orf60, EIF1AX, IGDCC4, NAT8L, PTGDR2, STIL, ZNF224,
C7orf61, EIF1AY, IGF1, NAT9, PTGDS, STIM1, ZNF225, C7orf62, EIF1B, IGF1R, NAV1, PTGER1,
STIM2, ZNF226, C7orf63, EIF2A, IGF2, NAV2, PTGER2, STIP1, ZNF227, C7orf65, EIF2AK1,
IGF2BP1, NAV3, PTGER3, STK10, ZNF229, C7orf66, EIF2AK2, IGF2BP2, NBAS, PTGER4, STK11,
ZNF23, C7orf69, EIF2AK3, IGF2BP3, NBEA, PTGES, STK11IP, ZNF230, C7orf71, EIF2AK4, IGF2R,
NBEAL1, PTGES2, STK16, ZNF232, C7orf72, EIF2B1, IGFALS, NBEAL2, PTGES3, STK17A,
ZNF233, C7orf73, EIF2B2, IGFBP1, NBL1, PTGES3L, STK17B, ZNF234, C7orf76, EIF2B3, IGFBP2,
NBN, PTGES3L-AARSD1, STK19, ZNF235, C8A, EIF2B4, IGFBP3, NBPF14, PTGFR, STK24,
ZNF236, C8B, EIF2B5, IGFBP4, NBPF3, PTGFRN, STK25, ZNF239, C8G, EIF2D, IGFBP5, NBPF4,
PTGIR, STK3, ZNF24, C8orf22, EIF2S1, IGFBP6, NBPF6, PTGIS, STK31, ZNF248, C8orf31, EIF2S2,
IGFBP7, NBPF7, PTGR1, STK32A, ZNF25, C8orf33, EIF2S3, IGFBPL1, NBR1, PTGR2, STK32B,
ZNF250, C8orf34, EIF3A, IGFL1, NCALD, PTGS1, STK32C, ZNF251, C8orf37, EIF3B, IGFL2,
NCAM1, PTGS2, STK33, ZNF253, C8orf4, EIF3C, IGFL3, NCAM2, PTH, STK35, ZNF254, C8orf44,
EIF3CL, IGFL4, NCAN, PTH1R, STK36, ZNF256, C8orf44-SGK3, EIF3D, IGFLR1, NCAPD2, PTH2,
STK38, ZNF257, C8orf46, EIF3E, IGFN1, NCAPD3, PTH2R, STK38L, ZNF259, C8orf47, EIF3F,
IGHMBP2, NCAPG, PTHLH, STK39, ZNF26, C8orf48, EIF3G, IGIP, NCAPG2, PTK2, STK4, ZNF260,
C8orf58, EIF3H, IGJ, NCAPH, PTK2B, STK40, ZNF263, C8orf59, EIF3I, IGLL1, NCAPH2, PTK6,
STMN1, ZNF264, C8orf74, EIF3J, IGLL5, NCBP1, PTK7, STMN2, ZNF266, C8orf76, EIF3K,
IGLON5, NCBP2, PTMA, STMN3, ZNF267, C8orf82, EIF3L, IGSF1, NCCRP1, PTMS, STMN4,
ZNF268, C8orf86, EIF3M, IGSF10, NCDN, PTN, STMND1, ZNF273, C8orf87, EIF4A1, IGSF11,
NCEH1, PTOV1, STOM, ZNF274, C9, EIF4A2, IGSF21, NCF1, PTP4A1, STOML1, ZNF275,
C9orf106, EIF4A3, IGSF22, NCF2, PTP4A2, STOML2, ZNF276, C9orf114, EIF4B, IGSF23, NCF4,
PTP4A3, STOML3, ZNF277, C9orf116, EIF4E, IGSF3, NCK1, PTPDC1, STON1, ZNF28, C9orf117,
EIF4E1B, IGSF5, NCK2, PTPLA, STON1-GTF2A1L, ZNF280A, C9orf129, EIF4E2, IGSF6, NCKAP1,
PTPLAD1, STON2, ZNF280B, C9orf131, EIF4E3, IGSF8, NCKAP1L, PTPLAD2, STOX1, ZNF280C,
C9orf135, EIF4EBP1, IGSF9, NCKAP5, PTPLB, STOX2, ZNF280D, C9orf139, EIF4EBP2, IGSF9B,
NCKAP5L, PTPMT1, STPG1, ZNF281, C9orf142, EIF4EBP3, IHH, NCKIPSD, PTPN1, STPG2,
ZNF282, C9orf152, EIF4ENIF1, IK, NCL, PTPN11, STRA13, ZNF283, C9orf153, EIF4G1, IKBIP,
NCLN, PTPN12, STRA6, ZNF284, C9orf156, EIF4G2, IKBKAP, NCMAP, PTPN13, STRA8, ZNF285,
C9orf16, EIF4G3, IKBKB, NCOA1, PTPN14, STRADA, ZNF286A, C9orf163, EIF4H, IKBKE,
NCOA2, PTPN18, STRADB, ZNF286B, C9orf169, EIF5, IKBKG, NCOA3, PTPN2, STRAP, ZNF287,
C9orf170, EIF5A, IKZF1, NCOA4, PTPN20A, STRBP, ZNF292, C9orf171, EIF5A2, IKZF2, NCOA5,
PTPN20B, STRC, ZNF296, C9orf173, EIF5AL1, IKZF3, NCOA6, PTPN21, STRIP1, ZNF3, C9orf24,
EIF5B, IKZF4, NCOA7, PTPN22, STRIP2, ZNF30, C9orf3, EIF6, IKZF5, NCOR1, PTPN23, STRN,
ZNF300, C9orf37, ELAC1, INTERLEUKIN, IL10, NCOR2, PTPN3, STRN3, ZNF302, C9orf40,
ELAC2, IL10RA, NCR1, PTPN4, STRN4, ZNF304, C9orf41, ELANE, IL10RB, NCR2, PTPN5, STS,
ZNF311, C9orf43, ELAVL1, IL11, NCR3, PTPN6, STT3A, ZNF316, C9orf47, ELAVL2, IL11RA,
NCR3LG1, PTPN7, STT3B, ZNF317, C9orf50, ELAVL3, IL12A, NCS1, PTPN9, STUB1, ZNF318,
C9orf57, ELAVL4, IL12B, NCSTN, PTPRA, STX10, ZNF319, C9orf62, ELF1, IL12RB1, NDC1,
PTPRB, STX11, ZNF32, C9orf64, ELF2, IL12RB2, NDC80, PTPRC, STX12, ZNF320, C9orf66, ELF3,
IL13, NDE1, PTPRCAP, STX16, ZNF322, C9orf69, ELF4, IL13RA1, NDEL1, PTPRD, STX17,
ZNF324, C9orf72, ELF5, IL13RA2, NDFIP1, PTPRE, STX18, ZNF324B, C9orf78, ELFN1, IL15,
NDFIP2, PTPRF, STX19, ZNF326, C9orf84, ELFN2, IL15RA, NDN, PTPRG, STX1A, ZNF329,
C9orf85, ELK1, IL16, NDNF, PTPRH, STX1B, ZNF330, C9orf89, ELK3, IL17A, NDNL2, PTPRJ,
STX2, ZNF331, C9orf9, ELK4, IL17B, NDOR1, PTPRK, STX3, ZNF333, C9orf91, ELL, IL17C, NDP,
PTPRM, STX4, ZNF334, C9orf92, ELL2, IL17D, NDRG1, PTPRN, STX5, ZNF335, C9orf96, ELL3,
IL17F, NDRG2, PTPRN2, STX6, ZNF337, CA1, ELMO1, IL17RA, NDRG3, PTPRO, STX7, ZNF33A,
CA10, ELMO2, IL17RB, NDRG4, PTPRQ, STX8, ZNF33B, CA11, ELMO3, IL17RC, NDST1, PTPRR,
STXBP1, ZNF34, CA12, ELMOD1, IL17RD, NDST2, PTPRS, STXBP2, ZNF341, CA13, ELMOD2,
IL17RE, NDST3, PTPRT, STXBP3, ZNF343, CA14, ELMOD3, IL17REL, NDST4, PTPRU, STXBP4,
ZNF345, CA2, ELMSAN1, IL18, NDUFA1, PTPRZ1, STXBP5, ZNF346, CA3, ELN, IL18BP,
NDUFA10, PTRF, STXBP5L, ZNF347, CA4, ELOF1, IL18R1, NDUFA11, PTRH1, STXBP6, ZNF35,
CASA, ELOVL1, IL18RAP, NDUFA12, PTRH2, STYK1, ZNF350, CA5B, ELOVL2, IL19, NDUFA13,
PTRHD1, STYX, ZNF354A, CA6, ELOVL3, IL1A, NDUFA2, PTS, STYXL1, ZNF354B, CA7,
ELOVL4, IL1B, NDUFA3, PTTG1, SUB1, ZNF354C, CA8, ELOVL5, IL1F10, NDUFA4, PTTG1IP,
SUCLA2, ZNF358, CA9, ELOVL6, IL1R1, NDUFA4L2, PTTG2, SUCLG1, ZNF362, CAAP1,
ELOVL7, IL1R2, NDUFA5, PTX3, SUCLG2, ZNF365, CAB39, ELP2, IL1RAP, NDUFA6, PTX4,
SUCNR1, ZNF366, CAB39L, ELP3, IL1RAPL1, NDUFA7, PUF60, SUCO, ZNF367, CABIN1, ELP4,
IL1RAPL2, NDUFA8, PUM1, SUDS3, ZNF37A, CABLES1, ELP5, IL1RL1, NDUFA9, PUM2, SUFU,
ZNF382, CABLES2, ELP6, IL1RL2, NDUFAB1, PURA, SUGP1, ZNF383, CABP1, ELSPBP1, IL1RN,
NDUFAF1, PURB, SUGP2, ZNF384, CABP2, ELTD1, IL2, NDUFAF2, PURG, SUGT1, ZNF385A,
CABP4, EMB, IL20, NDUFAF3, PUS1, SULF1, ZNF385B, CABP5, EMC1, IL20RA, NDUFAF4,
PUS10, SULF2, ZNF385C, CABP7, EMC10, IL20RB, NDUFAF5, PUS3, SULT1A1, ZNF385D,
CABS1, EMC2, IL21, NDUFAF6, PUS7, SULT1A2, ZNF391, CABYR, EMC3, IL21R, NDUFAF7,
PUS7L, SULT1A3, ZNF394, CACFD1, EMC4, IL22, NDUFB1, PUSL1, SULT1A4, ZNF395,
CACHD1, EMC6, IL22RA1, NDUFB10, PVALB, SULT1B1, ZNF396, CACNA1A, EMC7, IL22RA2,
NDUFB11, PVR, SULT1C2, ZNF397, CACNA1B, EMC8, IL23A, NDUFB2, PVRIG, SULT1C3,
ZNF398, CACNA1C, EMC9, IL23R, NDUFB3, PVRL1, SULT1C4, ZNF404, CACNA1D, EMCN, IL24,
NDUFB4, PVRL2, SULT1E1, ZNF407, CACNA1E, EMD, IL25, NDUFB5, PVRL3, SULT2A1,
ZNF408, CACNA1F, EME1, IL26, NDUFB6, PVRL4, SULT2B1, ZNF41, CACNA1G, EME2, IL27,
NDUFB7, PWP1, SULT4A1, ZNF410, CACNA1H, EMG1, IL27RA, NDUFB8, PWP2, SULT6B1,
ZNF414, CACNA1I, EMID1, IL2RA, NDUFB9, PWWP2A, SUMF1, ZNF415, CACNA1S, EMILIN1,
IL2RB, NDUFC1, PWWP2B, SUMF2, ZNF416, CACNA2D1, EMILIN2, IL2RG, NDUFC2, PXDC1,
SUMO1, ZNF417, CACNA2D2, EMILIN3, IL3, NDUFC2-KCTD14, PXDN, SUMO2, ZNF418,
CACNA2D3, EML1, IL31, NDUFS1, PXDNL, SUMO3, ZNF419, CACNA2D4, EML2, IL31RA,
NDUFS2, PXK, SUMO4, ZNF420, CACNB1, EML3, IL32, NDUFS3, PXMP2, SUN1, ZNF423,
CACNB2, EML4, IL33, NDUFS4, PXMP4, SUN2, ZNF425, CACNB3, EML5, IL34, NDUFS5, PXN,
SUN3, ZNF426, CACNB4, EML6, IL36A, NDUFS6, PXT1, SUN5, ZNF428, CACNG1, EMP1, IL36B,
NDUFS7, PYCARD, SUOX, ZNF429, CACNG2, EMP2, IL36G, NDUFS8, PYCR1, SUPT16H, ZNF43,
CACNG3, EMP3, IL36RN, NDUFV1, PYCR2, SUPT20H, ZNF430, CACNG4, EMR1, IL37, NDUFV2,
PYCRL, SUPT3H, ZNF431, CACNG5, EMR2, IL3RA, NDUFV3, PYDC1, SUPT4H1, ZNF432,
CACNG6, EMR3, IL4, NEB, PYDC2, SUPT5H, ZNF433, CACNG7, EMX1, IL4I1, NEBL, PYGB,
SUPT6H, ZNF436, CACNG8, EMX2, IL4R, NECAB1, PYGL, SUPT7L, ZNF438, CACTIN, EN1, IL5,
NECAB2, PYGM, SUPV3L1, ZNF439, CACUL1, EN2, IL5RA, NECAB3, PYGO1, SURF1, ZNF44,
CACYBP, ENAH, IL6, NECAP1, PYGO2, SURF2, ZNF440, CAD, ENAM, IL6R, NECAP2, PYHIN1,
SURF4, ZNF441, CADM1, ENC1, IL6ST, NEDD1, PYROXD1, SURF6, ZNF442, CADM2, ENDOD1,
IL7, NEDD4, PYROXD2, SUSD1, ZNF443, CADM3, ENDOG, IL7R, NEDD4L, PYURF, SUSD2,
ZNF444, CADM4, ENDOU, IL8, NEDD8, PYY, SUSD3, ZNF445, CADPS, ENDOV, IL9, NEDD8-
MDP1, PZP, SUSD4, ZNF446, CADPS2, ENG, IL9R, NEDD9, QARS, SUSD5, ZNF449, CAGE1,
ENGASE, ILDR1, NEFH, QDPR, SUV39H1, ZNF45, CALB1, ENHO, ILDR2, NEFL, QKI, SUV39H2,
ZNF451, CALB2, ENKD1, ILF2, NEFM, QPCT, SUV420H1, ZNF454, CALCA, ENKUR, ILF3,
NEGR1, QPCTL, SUV420H2, ZNF460, CALCB, ENO1, ILK, NEIL1, QPRT, SUZ12, ZNF461,
CALCOCO1, ENO2, ILKAP, NEIL2, QRFP, SV2A, ZNF462, CALCOCO2, ENO3, ILVBL, NEIL3,
QRFPR, SV2B, ZNF467, CALCR, ENO4, IMMP1L, NEK1, QRICH1, SV2C, ZNF468, CALCRL,
ENOPH1, IMMP2L, NEK10, QRICH2, SVEP1, ZNF469, CALD1, ENOSF1, IMMT, NEK11, QRSL1,
SVIL, ZNF470, CALHM1, ENOX1, IMP3, NEK2, QSER1, SVIP, ZNF471, CALHM2, ENOX2, IMP4,
NEK3, QSOX1, SVOP, ZNF473, CALHM3, ENPEP, IMPA1, NEK4, QSOX2, SVOPL, ZNF474,
CALM1, ENPP1, IMPA2, NEK5, QTRT1, SWAP70, ZNF479, CALM2, ENPP2, IMPACT, NEK6,
QTRTD1, SWI5, ZNF48, CALM3, ENPP3, IMPAD1, NEK7, R3HCC1, SWSAP1, ZNF480, CALML3,
ENPP4, IMPDH1, NEK8, R3HCC1L, SWT1, ZNF483, CALML4, ENPP5, IMPDH2, NEK9, R3HDM1,
SYAP1, ZNF484, CALML5, ENPP6, IMPG1, NELFA, R3HDM2, SYBU, ZNF485, CALML6, ENPP7,
IMPG2, NELFB, R3HDM4, SYCE1, ZNF486, CALN1, ENSA, INA, NELFCD, R3HDML, SYCE1L,
ZNF488, CALR, ENTHD1, INADL, NELFE, RAB10, SYCE2, ZNF490, CALR3, ENTHD2, INCA1,
NELL1, RAB11A, SYCE3, ZNF491, CALU, ENTPD1, INCENP, NELL2, RAB11B, SYCN, ZNF492,
CALY, ENTPD2, INF2, NEMF, RAB11FIP1, SYCP1, ZNF493, CAMK1, ENTPD3, ING1, NENF,
RAB11FIP2, SYCP2, ZNF496, CAMK1D, ENTPD4, ING2, NEO1, RAB11FIP3, SYCP2L, ZNF497,
CAMK1G, ENTPD5, ING3, NES, RAB11FIP4, SYCP3, ZNF500, CAMK2A, ENTPD6, ING4, NET1,
RAB11FIP5, SYDE1, ZNF501, CAMK2B, ENTPD7, ING5, NETO1, RAB12, SYDE2, ZNF502,
CAMK2D, ENTPD8, INHA, NETO2, RAB13, SYF2, ZNF503, CAMK2G, ENY2, INHBA, NEU1,
RAB14, SYK, ZNF506, CAMK2N1, EOGT, INHBB, NEU2, RAB15, SYMPK, ZNF507, CAMK2N2,
EOMES, INHBC, NEU3, RAB17, SYN1, ZNF510, CAMK4, EP300, INHBE, NEU4, RAB18, SYN2,
ZNF511, CAMKK1, EP400, INIP, NEURL1, RAB19, SYN3, ZNF512, CAMKK2, EPAS1, INMT,
NEURL1B, RAB1A, SYNC, ZNF512B, CAMKMT, EPB41, INO80, NEURL2, RAB1B, SYNCRIP,
ZNF513, CAMKV, EPB41L1, INO80B, NEURL3, RAB20, SYNDIG1, ZNF514, CAMLG, EPB41L2,
INO80C, NEURL4, RAB21, SYNDIG1L, ZNF516, CAMP, EPB41L3, INO80D, NEUROD1, RAB22A,
SYNE1, ZNF517, CAMSAP1, EPB41L4A, INO80E, NEUROD2, RAB23, SYNE2, ZNF518A,
CAMSAP2, EPB41L4B, INPP1, NEUROD4, RAB24, SYNE3, ZNF518B, CAMSAP3, EPB41L5,
INPP4A, NEUROD6, RAB25, SYNE4, ZNF519, CAMTA1, EPB42, INPP4B, NEUROG1, RAB26,
SYNGAP1, ZNF521, CAMTA2, EPC1, INPP5A, NEUROG2, RAB27A, SYNGR1, ZNF524, CAND1,
EPC2, INPP5B, NEUROG3, RAB27B, SYNGR2, ZNF526, CAND2, EPCAM, INPP5D, NEXN, RAB28,
SYNGR3, ZNF527, CANT1, EPDR1, INPP5E, NF1, RAB2A, SYNGR4, ZNF528, CANX, EPG5,
INPP5F, NF2, RAB2B, SYNJ1, ZNF529, CAP1, EPGN, INPP5J, NFAM1, RAB30, SYNJ2, ZNF530,
CAP2, EPHA1, INPP5K, NFASC, RAB31, SYNJ2BP, ZNF532, CAPG, EPHA10, INPPL1, NFAT5,
RAB32, SYNJ2BP-COX16, ZNF534, CAPN1, EPHA2, INS, NFATC1, RAB33A, SYNM, ZNF536,
CAPN10, EPHA3, INSC, NFATC2, RAB33B, SYNPO, ZNF540, CAPN11, EPHA4, INSIG1,
NFATC2IP, RAB34, SYNPO2, ZNF541, CAPN12, EPHA5, INSIG2, NFATC3, RAB35, SYNPO2L,
ZNF543, CAPN13, EPHA6, INS-IGF2, NFATC4, RAB36, SYNPR, ZNF544, CAPN14, EPHA7, INSL3,
NFE2, RAB37, SYNRG, ZNF546, CAPN15, EPHA8, INSL4, NFE2L1, RAB38, SYP, ZNF547, CAPN2,
EPHB1, INSL5, NFE2L2, RAB39A, SYPL1, ZNF548, CAPN3, EPHB2, INSL6, NFE2L3, RAB39B,
SYPL2, ZNF549, CAPN5, EPHB3, INSM1, NFIA, RAB3A, SYS1, ZNF550, CAPN6, EPHB4, INSM2,
NFIB, RAB3B, SYT1, ZNF551, CAPN7, EPHB6, INSR, NFIC, RAB3C, SYT10, ZNF552, CAPN8,
EPHX1, INSRR, NFIL3, RAB3D, SYT11, ZNF554, CAPN9, EPHX2, INTS1, NFIX, RAB3GAP1,
SYT12, ZNF555, CAPNS1, EPHX3, INTS10, NFKB1, RAB3GAP2, SYT13, ZNF556, CAPNS2,
EPHX4, INTS12, NFKB2, RAB3IL1, SYT14, ZNF557, CAPRIN1, EPM2A, INTS2, NFKBIA, RAB3IP,
SYT15, ZNF558, CAPRIN2, EPM2AIP1, INTS3, NFKBIB, RAB40A, SYT16, ZNF559, CAPS, EPN1,
INTS4, NFKBID, RAB40AL, SYT17, ZNF559-ZNF177, CAPS2, EPN2, INTS5, NFKBIE, RAB40B,
SYT2, ZNF560, CAPSL, EPN3, INTS6, NFKBIL1, RAB40C, SYT3, ZNF561, CAPZA1, EPO, INTS7,
NFKBIZ, RAB41, SYT4, ZNF562, CAPZA2, EPOR, INTS8, NFRKB, RAB42, SYT5, ZNF563,
CAPZA3, EPPIN, INTS9, NFS1, RAB43, SYT6, ZNF564, CAPZB, EPPIN-WFDC6, INTU, NFU1,
RAB44, SYT7, ZNF565, CARD10, EPPK1, INVS, NFX1, RAB4A, SYT8, ZNF566, CARD11, EPRS,
IP6K1, NFXL1, RAB4B, SYT9, ZNF567, CARD14, EPS15, IP6K2, NFYA, RAB5A, SYTL1, ZNF568,
CARD16, EPS15L1, IP6K3, NFYB, RAB5B, SYTL2, ZNF569, CARD17, EPS8, IPCEF1, NFYC,
RAB5C, SYTL3, ZNF57, CARD18, EPS8L1, IPMK, NGB, RAB6A, SYTL4, ZNF570, CARD6,
EPS8L2, IPO11, NGDN, RAB6B, SYTL5, ZNF571, CARD8, EPS8L3, IPO13, NGEF, RAB6C, SYVN1,
ZNF572, CARD9, EPSTI1, IPO4, NGF, RAB7A, SZT2, ZNF573, CARF, EPT1, IPO5, NGFR, RAB7L1,
T, ZNF574, CARHSP1, EPX, IPO7, NGFRAP1, RAB8A, TAAR1, ZNF575, CARKD, EPYC, IPO8,
NGLY1, RAB8B, TAAR2, ZNF576, CARM1, EQTN, IPO9, NGRN, RAB9A, TAAR5, ZNF577,
CARNS1, ERAL1, IPP, NHEJ1, RAB9B, TAAR6, ZNF578, CARS, ERAP1, IPPK, NHLH1, RABAC1,
TAAR8, ZNF579, CARS2, ERAP2, IQCA1, NHLH2, RABEP1, TAAR9, ZNF580, CARTPT, ERAS,
IQCB1, NHLRC1, RABEP2, TAB1, ZNF581, CASC1, ERBB2, IQCC, NHLRC2, RABEPK, TAB2,
ZNF582, CASC10, ERBB2IP, IQCD, NHLRC3, RABGAP1, TAB3, ZNF583, CASC3, ERBB3, IQCE,
NHLRC4, RABGAP1L, TAC1, ZNF584, CASC4, ERBB4, IQCF1, NHP2, RABGEF1, TAC3,
ZNF585A, CASC5, ERC1, IQCF2, NHP2L1, RABGGTA, TAC4, ZNF585B, CASD1, ERC2, IQCF3,
NHS, RABGGTB, TACC1, ZNF586, CASK, ERCC1, IQCF5, NHSL1, RABIF, TACC2, ZNF587,
CASKIN1, ERCC2, IQCF6, NHSL2, RABL2A, TACC3, ZNF587B, CASKIN2, ERCC3, IQCG, NICN1,
RABL2B, TACO1, ZNF589, CASP1, ERCC4, IQCH, NID1, RABL3, TACR1, ZNF592, CASP10,
ERCC5, IQCJ, NID2, RABL5, TACR2, ZNF593, CASP12, ERCC6, IQCJ-SCHIP1, NIF3L1, RABL6,
TACR3, ZNF594, CASP14, ERCC6L, IQCK, NIFK, RAC1, TACSTD2, ZNF595, CASP16, ERCC6L2,
IQGAP1, NIM1, RAC2, TADA1, ZNF596, CASP2, ERCC6-PGBD3, IQGAP2, NIN, RAC3, TADA2A,
ZNF597, CASP3, ERCC8, IQGAP3, NINJ1, RACGAP1, TADA2B, ZNF598, CASP4, EREG, IQSEC1,
NINJ2, RAD1, TADA3, ZNF599, CASP5, ERF, IQSEC2, NINL, RAD17, TAF1, ZNF600, CASP6,
ERG, IQSEC3, NIP7, RAD18, TAF10, ZNF605, CASP7, ERGIC1, IQUB, NIPA1, RAD21, TAF11,
ZNF606, CASP8, ERGIC2, IRAK1, NIPA2, RAD21L1, TAF12, ZNF607, CASP8AP2, ERGIC3,
IRAK1BP1, NIPAL1, RAD23A, TAF13, ZNF608, CASP9, ERH, IRAK2, NIPAL2, RAD23B, TAF15,
ZNF609, CASQ1, ERI1, IRAK3, NIPAL3, RAD50, TAF1A, ZNF610, CASQ2, ERI2, IRAK4, NIPAL4,
RAD51, TAF1B, ZNF611, CASR, ERI3, IREB2, NIPBL, RAD51AP1, TAF1C, ZNF613, CASS4,
ERICH1, IRF1, NIPSNAP1, RAD51AP2, TAF1D, ZNF614, CAST, ERICH2, IRF2, NIPSNAP3A,
RAD51B, TAF1L, ZNF615, CASZ1, ERLEC1, IRF2BP1, NIPSNAP3B, RAD51C, TAF2, ZNF616,
CAT, ERLIN1, IRF2BP2, NISCH, RAD51D, TAF3, ZNF618, CATSPER1, ERLIN2, IRF2BPL, NIT1,
RAD52, TAF4, ZNF619, CATSPER2, ERMAP, IRF3, NIT2, RAD54B, TAF4B, ZNF620, CATSPER3,
ERMARD, IRF4, NKAIN1, RAD54L, TAF5, ZNF621, CATSPER4, ERMN, IRF5, NKAIN2, RAD54L2,
TAF5L, ZNF622, CATSPERB, ERMP1, IRF6, NKAIN3, RAD9A, TAF6, ZNF623, CATSPERD, ERN1,
IRF7, NKAIN4, RAD9B, TAF6L, ZNF624, CATSPERG, ERN2, IRF8, NKAP, RADIL, TAF7, ZNF625,
CAV1, ERO1L, IRF9, NKAPL, RAE1, TAF7L, ZNF626, CAV2, ERO1LB, IRG1, NKD1, RAET1E,
TAF8, ZNF627, CAV3, ERP27, IRGC, NKD2, RAET1G, TAF9, ZNF628, CBFA2T2, ERP29, IRGM,
NKG7, RAET1L, TAF9B, ZNF629, CBFA2T3, ERP44, IRGQ, NKIRAS1, RAF1, TAGAP, ZNF630,
CBFB, ERRFI1, IRS1, NKIRAS2, RAG1, TAGLN, ZNF638, CBL, ERVFRD-1, IRS2, NKPD1, RAG2,
TAGLN2, ZNF639, CBLB, ERVMER34-1, IRS4, NKRF, RAI1, TAGLN3, ZNF641, CBLC, ERVV-1,
IRX1, NKTR, RAI14, TAL1, ZNF644, CBLL1, ERVV-2, IRX2, NKX1-2, RAI2, TAL2, ZNF645,
CBLN1, ESAM, IRX3, NKX2-1, RALA, TALDO1, ZNF646, CBLN2, ESCO1, IRX4, NKX2-2, RALB,
TAMM41, ZNF648, CBLN3, ESCO2, IRX5, NKX2-3, RALBP1, TANC1, ZNF649, CBLN4, ESD,
IRX6, NKX2-4, RALGAPA1, TANC2, ZNF652, CBR1, ESF1, ISCA1, NKX2-5, RALGAPA2,
TANGO2, ZNF653, CBR3, ESM1, ISCA2, NKX2-6, RALGAPB, TANGO6, ZNF654, CBR4, ESPL1,
ISCU, NKX2-8, RALGDS, TANK, ZNF655, CBS, ESPN, ISG15, NKX3- 1, RALGPS1, TAOK1,
ZNF658, CBWD1, ESPNL, ISG20, NKX3-2, RALGPS2, TAOK2, ZNF660, CBWD2, ESR1, ISG20L2,
NKX6-1, RALY, TAOK3, ZNF662, CBWD3, ESR2, ISL1, NKX6-2, RALYL, TAP1, ZNF664, CBWD5,
ESRP1, ISL2, NKX6-3, RAMP1, TAP2, ZNF664-FAM101A, CBWD6, ESRP2, ISLR, NLE1, RAMP2,
TAPBP, ZNF665, CBWD7, ESRRA, ISLR2, NLGN1, RAMP3, TAPBPL, ZNF667, CBX1, ESRRB,
ISM1, NLGN2, RAN, TAPT1, ZNF668, CBX2, ESRRG, ISM2, NLGN3, RANBP1, TARBP1, ZNF669,
CBX3, ESX1, ISOC1, NLGN4X, RANBP10, TARBP2, ZNF670, CBX4, ESYT1, ISOC2, NLGN4Y,
RANBP17, TARDBP, ZNF671, CBX5, ESYT2, ISPD, NLK, RANBP2, TARM1, ZNF672, CBX6,
ESYT3, IST1, NLN, RANBP3, TARP, ZNF674, CBX7, ETAA1, ISX, NLRC3, RANBP3L, TARS,
ZNF675, CBX8, ETF1, ISY1, NLRC4, RANBP6, TARS2, ZNF676, CBY1, ETFA, ISY1-RAB43,
NLRC5, RANBP9, TARSL2, ZNF677, CBY3, ETFB, ISYNA1, NLRP1, RANGAP1, TAS1R1, ZNF678,
CC2D1A, ETFDH, ITCH, NLRP10, RANGRF, TAS1R2, ZNF679, CC2D1B, ETHE1, ITFG1, NLRP11,
RAP1A, TAS1R3, ZNF680, CC2D2A, ETNK1, ITFG2, NLRP12, RAP1B, TAS2R1, ZNF681, CC2D2B,
ETNK2, ITFG3, NLRP13, RAP1GAP, TAS2R10, ZNF682, CCAR1, ETNPPL, ITGA1, NLRP14,
RAPIGAP2, TAS2R13, ZNF683, CCAR2, ETS1, ITGA10, NLRP2, RAP1GDS1, TAS2R14, ZNF684,
CCBE1, ETS2, ITGA11, NLRP3, RAP2A, TAS2R16, ZNF687, CCBL1, ETV1, ITGA2, NLRP4,
RAP2B, TAS2R19, ZNF688, CCBL2, ETV2, ITGA2B, NLRP5, RAP2C, TAS2R20, ZNF689,
CCDC101, ETV3, ITGA3, NLRP6, RAPGEF1, TAS2R3, ZNF69, CCDC102A, ETV3L, ITGA4, NLRP7,
RAPGEF2, TAS2R30, ZNF691, CCDC102B, ETV4, ITGA5, NLRP8, RAPGEF3, TAS2R31, ZNF692,
CCDC103, ETV5, ITGA6, NLRP9, RAPGEF4, TAS2R38, ZNF695, CCDC104, ETV6, ITGA7, NLRX1,
RAPGEF5, TAS2R39, ZNF696, CCDC105, ETV7, ITGA8, NMB, RAPGEF6, TAS2R4, ZNF697,
CCDC106, EVA1A, ITGA9, NMBR, RAPGEFL1, TAS2R40, ZNF699, CCDC107, EVA1B, ITGAD,
NMD3, RAPH1, TAS2R41, ZNF7, CCDC108, EVA1C, ITGAE, NME1, RAPSN, TAS2R42, ZNF70,
CCDC109B, EVC, ITGAL, NME1-NME2, RARA, TAS2R43, ZNF700, CCDC11, EVC2, ITGAM,
NME2, RARB, TAS2R46, ZNF701, CCDC110, EVI2A, ITGAV, NME3, RARG, TAS2R5, ZNF703,
CCDC112, EVI2B, ITGAX, NME4, RARRES1, TAS2R50, ZNF704, CCDC113, EVI5, ITGB1, NME5,
RARRES2, TAS2R60, ZNF705A, CCDC114, EVI5L, ITGB1BP1, NME6, RARRES3, TAS2R7,
ZNF705B, CCDC115, EVL, ITGB1BP2, NME7, RARS, TAS2R8, ZNF705D, CCDC116, EVPL, ITGB2,
NME8, RARS2, TAS2R9, ZNF705E, CCDC117, EVPLL, ITGB3, NME9, RASA1, TASP1, ZNF705G,
CCDC12, EVX1, ITGB3BP, NMI, RASA2, TAT, ZNF706, CCDC120, EVX2, ITGB4, NMNAT1,
RASA3, TATDN1, ZNF707, CCDC121, EWSR1, ITGB5, NMNAT2, RASA4, TATDN2, ZNF708,
CCDC122, EXD1, ITGB6, NMNAT3, RASA4B, TATDN3, ZNF709, CCDC124, EXD2, ITGB7,
NMRAL1, RASAL1, TAX1BP1, ZNF71, CCDC125, EXD3, ITGB8, NMRK1, RASAL2, TAX1BP3,
ZNF710, CCDC126, EXO1, ITGBL1, NMRK2, RASAL3, TAZ, ZNF711, CCDC127, EXO5, ITIH1,
NMS, RASD1, TBATA, ZNF713, CCDC129, EXOC1, ITIH2, NMT1, RASD2, TBC1D1, ZNF714,
CCDC13, EXOC2, ITIH3, NMT2, RASEF, TBC1D10A, ZNF716, CCDC130, EXOC3, ITIH4, NMU,
RASGEF1A, TBC1D10B, ZNF717, CCDC132, EXOC3L1, ITIH5, NMUR1, RASGEF1B, TBC1D10C,
ZNF718, CCDC134, EXOC3L2, ITIH6, NMUR2, RASGEF1C, TBCID12, ZNF720, CCDC135,
EXOC3L4, ITK, NNAT, RASGRF1, TBC1D13, ZNF721, CCDC136, EXOC4, ITLN1, NNMT,
RASGRF2, TBC1D14, ZNF726, CCDC137, EXOC5, ITLN2, NNT, RASGRP1, TBC1D15, ZNF727,
CCDC138, EXOC6, ITM2A, NOA1, RASGRP2, TBC1D16, ZNF728, CCDC14, EXOC6B, ITM2B,
NOB1, RASGRP3, TBC1D17, ZNF729, CCDC140, EXOC7, ITM2C, NOBOX, RASGRP4, TBC1D19,
ZNF730, CCDC141, EXOC8, ITPA, NOC2L, RASIP1, TBC1D2, ZNF732, CCDC142, EXOG, ITPK1,
NOC3L, RASL10A, TBC1D20, ZNF735, CCDC144A, EXOSC1, ITPKA, NOC4L, RASL10B,
TBC1D21, ZNF736, CCDC144NL, EXOSC10, ITPKB, NOD1, RASL11A, TBC1D22A, ZNF737,
CCDC146, EXOSC2, ITPKC, NOD2, RASL11B, TBC1D22B, ZNF74, CCDC147, EXOSC3, ITPR1,
NODAL, RASL12, TBC1D23, ZNF740, CCDC148, EXOSC4, ITPR2, NOG, RASSF1, TBC1D24,
ZNF746, CCDC149, EXOSC5, ITPR3, NOL10, RASSF10, TBC1D25, ZNF747, CCDC15, EXOSC6,
ITPRIP, NOL11, RASSF2, TBC1D26, ZNF749, CCDC150, EXOSC7, ITPRIPL1, NOL12, RASSF3,
TBC1D27, ZNF750, CCDC151, EXOSC8, ITPRIPL2, NOL3, RASSF4, TBC1D28, ZNF75A, CCDC152,
EXOSC9, ITSN1, NOL4, RASSF5, TBC1D29, ZNF75D, CCDC153, EXPH5, ITSN2, NOL6, RASSF6,
TBC1D2B, ZNF76, CCDC154, EXT1, IVD, NOL7, RASSF7, TBC1D3, ZNF761, CCDC155, EXT2,
IVL, NOL8, RASSF8, TBC1D30, ZNF763, CCDC157, EXTL1, IVNS1ABP, NOL9, RASSF9,
TBC1D31, ZNF764, CCDC158, EXTL2, IWS1, NOLC1, RAVER1, TBC1D32, ZNF765, CCDC159,
EXTL3, IYD, NOM1, RAVER2, TBC1D3B, ZNF766, CCDC160, EYA1, IZUMO1, NOMO1, RAX,
TBC1D3C, ZNF768, CCDC166, EYA2, IZUMO2, NOMO2, RAX2, TBC1D3F, ZNF77, CCDC167,
EYA3, IZUMO3, NOMO3, RB1, TBC1D3G, ZNF770, CCDC168, EYA4, IZUMO4, NONO, RB1CC1,
TBC1D3H, ZNF771, CCDC169, EYS, JADE1, NOP10, RBAK, TBC1D4, ZNF772, CCDC169-
SOHLH2, EZH1, JADE2, NOP14, RBAK-RBAKDN, TBC1D5, ZNF773, CCDC17, EZH2, JADE3,
NOP16, RBBP4, TBC1D7, ZNF774, CCDC170, EZR, JAG1, NOP2, RBBP5, TBC1D8, ZNF775,
CCDC171, F10, JAG2, NOP56, RBBP6, TBC1D8B, ZNF776, CCDC172, F11, JAGN1, NOP58, RBBP7,
TBC1D9, ZNF777, CCDC173, F11R, JAK1, NOP9, RBBP8, TBC1D9B, ZNF778, CCDC174, F12,
JAK2, NOS1, RBBP8NL, TBCA, ZNF780A, CCDC175, F13A1, JAK3, NOS1AP, RBBP9, TBCB,
ZNF780B, CCDC176, F13B, JAKMIP1, NOS2, RBCK1, TBCC, ZNF781, CCDC177, F2, JAKMIP2,
NOS3, RBFA, TBCCD1, ZNF782, CCDC178, F2R, JAKMIP3, NOSIP, RBFOX1, TBCD, ZNF783,
CCDC179, F2RL1, JAM2, NOSTRIN, RBFOX2, TBCE, ZNF784, CCDC18, F2RL2, JAM3, NOTCH1,
RBFOX3, TBCEL, ZNF785, CCDC180, F2RL3, JARID2, NOTCH2, RBKS, TBCK, ZNF786,
CCDC181, F3, JAZF1, NOTCH2NL, RBL1, TBK1, ZNF787, CCDC19, F5, JDP2, NOTCH3, RBL2,
TBKBP1, ZNF789, CCDC22, F7, JHDM1D, NOTCH4, RBM10, TBL1X, ZNF79, CCDC23, F8,
JKAMP, NOTO, RBM11, TBL1XR1, ZNF790, CCDC24, F8A1, JMJD1C, NOTUM, RBM12, TBL1Y,
ZNF791, CCDC25, F8A2, JMJD4, NOV, RBM12B, TBL2, ZNF792, CCDC27, F8A3, JMJD6, NOVA1,
RBM14, TBL3, ZNF793, CCDC28A, F9, JMJD7, NOVA2, RBM14-RBM4, TBP, ZNF799, CCDC28B,
FA2H, JMJD7-PLA2G4B, NOX1, RBM15, TBPL1, ZNF8, CCDC3, FAAH, JMJD8, NOX3, RBM15B,
TBPL2, ZNF80, CCDC30, FAAH2, JMY, NOX4, RBM17, TBR1, ZNF800, CCDC33, FABP1, JOSD1,
NOX5, RBM18, TBRG1, ZNF804A, CCDC34, FABP12, JOSD2, NOXA1, RBM19, TBRG4, ZNF804B,
CCDC36, FABP2, JPH1, NOXO1, RBM20, TBX1, ZNF805, CCDC37, FABP3, JPH2, NOXRED1,
RBM22, TBX10, ZNF806, CCDC38, FABP4, JPH3, NPAP1, RBM23, TBX15, ZNF808, CCDC39,
FABP5, JPH4, NPAS1, RBM24, TBX18, ZNF81, CCDC40, FABP6, JRK, NPAS2, RBM25, TBX19,
ZNF812, CCDC41, FABP7, JRKL, NPAS3, RBM26, TBX2, ZNF813, CCDC42, FABP9, JSRP1,
NPAS4, RBM27, TBX20, ZNF814, CCDC42B, FADD, JTB, NPAT, RBM28, TBX21, ZNF816,
CCDC43, FADS1, JUN, NPB, RBM3, TBX22, ZNF816-ZNF321P, CCDC47, FADS2, JUNB, NPBWR1,
RBM33, TBX3, ZNF821, CCDC50, FADS3, JUND, NPBWR2, RBM34, TBX4, ZNF823, CCDC51,
FADS6, JUP, NPC1, RBM38, TBX5, ZNF827, CCDC53, FAF1, KAAG1, NPC1L1, RBM39, TBX6,
ZNF829, CCDC54, FAF2, KAL1, NPC2, RBM4, TBXA2R, ZNF83, CCDC57, FAH, KALRN, NPDC1,
RBM41, TBXAS1, ZNF830, CCDC58, FAHD1, KANK1, NPEPL1, RBM42, TC2N, ZNF831, CCDC59,
FAHD2A, KANK2, NPEPPS, RBM43, TCAIM, ZNF835, CCDC6, FAHD2B, KANK3, NPFF, RBM46,
TCAP, ZNF836, CCDC60, FAIM, KANK4, NPFFR1, RBM47, TCEA1, ZNF837, CCDC61, FAIM2,
KANSL1, NPFFR2, RBM48, TCEA2, ZNF839, CCDC62, FAIM3, KANSL1L, NPHP1, RBM4B,
TCEA3, ZNF84, CCDC63, FAM101A, KANSL2, NPHP3, RBM5, TCEAL1, ZNF841, CCDC64,
FAM101B, KANSL3, NPHP4, RBM6, TCEAL2, ZNF843, CCDC64B, FAM102A, KARS, NPHS1,
RBM7, TCEAL3, ZNF844, CCDC65, FAM102B, KAT2A, NPHS2, RBM8A, TCEAL4, ZNF845,
CCDC66, FAM103A1, KAT2B, NPIPA1, RBMS1, TCEAL5, ZNF846, CCDC67, FAM104A, KAT5,
NPIPA2, RBMS2, TCEAL6, ZNF85, CCDC68, FAM104B, KAT6A, NPIPA3, RBMS3, TCEAL7,
ZNF850, CCDC69, FAM105A, KAT6B, NPIPA5, RBMX, TCEAL8, ZNF852, CCDC7, FAM105B,
KAT7, NPIPA7, RBMX2, TCEANC, ZNF853, CCDC70, FAM107A, KAT8, NPIPA8, RBMXL1,
TCEANC2, ZNF860, CCDC71, FAM107B, KATNA1, NPIPB11, RBMXL2, TCEB1, ZNF862,
CCDC71L, FAM109A, KATNAL1, NPIPB15, RBMXL3, TCEB2, ZNF865, CCDC73, FAM109B,
KATNAL2, NPIPB3, RBMY1A1, TCEB3, ZNF878, CCDC74A, FAM110A, KATNB1, NPIPB4,
RBMY1B, TCEB3B, ZNF879, CCDC74B, FAM110B, KATNBL1, NPIPB5, RBMY1D, TCEB3C,
ZNF880, CCDC77, FAM110C, KAZALD1, NPIPB6, RBMY1E, TCEB3CL, ZNF883, CCDC78,
FAM110D, KAZN, NPIPB8, RBMY1F, TCEB3CL2, ZNF888, CCDC79, FAM111A, KBTBD11,
NPIPB9, RBMY1J, TCERG1, ZNF891, CCDC8, FAM111B, KBTBD12, NPL, RBP1, TCERG1L,
ZNF90, CCDC80, FAM114A1, KBTBD13, NPLOC4, RBP2, TCF12, ZNF91, CCDC81, FAM114A2,
KBTBD2, NPM1, RBP3, TCF15, ZNF92, CCDC82, FAM115A, KBTBD3, NPM2, RBP4, TCF19,
ZNF93, CCDC83, FAM115C, KBTBD4, NPM3, RBP5, TCF20, ZNF98, CCDC84, FAM117A,
KBTBD6, NPNT, RBP7, TCF21, ZNF99, CCDC85A, FAM117B, KBTBD7, NPPA, RBPJ, TCF23,
ZNFX1, CCDC85B, FAM118A, KBTBD8, NPPB, RBPJL, TCF24, ZNHIT1, CCDC85C, FAM118B,
KCMF1, NPPC, RBPMS, TCF25, ZNHIT2, CCDC86, FAM120A, KCNA1, NPR1, RBPMS2, TCF3,
ZNHIT3, CCDC87, FAM120AOS, KCNA10, NPR2, RBX1, TCF4, ZNHIT6, CCDC88A, FAM120B,
KCNA2, NPR3, RC3H1, TCF7, ZNRD1, CCDC88B, FAM120C, KCNA3, NPRL2, RC3H2, TCF7L1,
ZNRF1, CCDC88C, FAM122A, KCNA4, NPRL3, RCAN1, TCF7L2, ZNRF2, CCDC89, FAM122B,
KCNA5, NPS, RCAN2, TCFL5, ZNRF3, CCDC9, FAM122C, KCNA6, NPSR1, RCAN3, TCHH,
ZNRF4, CCDC90B, FAM124A, KCNA7, NPTN, RCBTB1, TCHHL1, ZP1, CCDC91, FAM124B,
KCNAB1, NPTX1, RCBTB2, TCHP, ZP2, CCDC92, FAM126A, KCNAB2, NPTX2, RCC1, TCIRGI,
ZP3, CCDC93, FAM126B, KCNAB3, NPTXR, RCC2, TCL1A, ZP4, CCDC94, FAM127A, KCNB1,
NPVF, RCCD1, TCL1B, ZPBP, CCDC96, FAM127B, KCNB2, NPW, RCE1, TCN1, ZPBP2, CCDC97,
FAM127C, KCNC1, NPY, RCHY1, TCN2, ZPLD1, CCER1, FAM129A, KCNC2, NPY1R, RCL1,
TCOF1, ZRANB1, CCHCR1, FAM129B, KCNC3, NPY2R, RCN1, TCP1, ZRANB2, CCIN, FAM129C,
KCNC4, NPY4R, RCN2, TCP10, ZRANB3, CCK, FAM131A, KCND1, NPY5R, RCN3, TCP10L,
ZRSR2, CCKAR, FAM131B, KCND2, NQO1, RCOR1, TCP10L2, ZSCAN1, CCKBR, FAM131C,
KCND3, NQO2, RCOR2, TCP11, ZSCAN10, CCL1, FAM132A, KCNE1, NR0B1, RCOR3, TCP11L1,
ZSCAN12, CCL11, FAM132B, KCNE1L, NR0B2, RCSD1, TCP11L2, ZSCAN16, CCL13, FAM133A,
KCNE2, NR1D1, RCVRN, TCP11X1, ZSCAN18, CCL14, FAM133B, KCNE3, NR1D2, RD3,
TCP11X2, ZSCAN2, CCL15, FAM134A, KCNE4, NR1H2, RD3L, TCTA, ZSCAN20, CCL16,
FAM134B, KCNF1, NR1H3, RDH10, TCTE1, ZSCAN21, CCL17, FAM134C, KCNG1, NR1H4,
RDH11, TCTE3, ZSCAN22, CCL18, FAM135A, KCNG2, NR1I2, RDH12, TCTEX1D1, ZSCAN23,
CCL19, FAM135B, KCNG3, NR113, RDH13, TCTEX1D2, ZSCAN25, CCL2, FAM136A, KCNG4,
NR2C1, RDH14, TCTEX1D4, ZSCAN26, CCL20, FAM13A, KCNH1, NR2C2, RDH16, TCTN1,
ZSCAN29, CCL21, FAM13B, KCNH2, NR2C2AP, RDH5, TCTN2, ZSCAN30, CCL22, FAM13C,
KCNH3, NR2E1, RDH8, TCTN3, ZSCAN31, CCL23, FAM149A, KCNH4, NR2E3, RDM1, TDG,
ZSCAN32, CCL24, FAM149B1, KCNH5, NR2F1, RDX, TDGF1, ZSCAN4, CCL25, FAM150A,
KCNH6, NR2F2, REC8, TDO2, ZSCAN5A, CCL26, FAM150B, KCNH7, NR2F6, RECK, TDP1,
ZSCAN5B, CCL27, FAM151A, KCNH8, NR3C1, RECQL, TDP2, ZSCAN9, CCL28, FAM151B,
KCNIP1, NR3C2, RECQL4, TDRD1, ZSWIM1, CCL3, FAM153A, KCNIP2, NR4A1, RECQL5,
TDRD10, ZSWIM2, CCL3L1, FAM153B, KCNIP3, NR4A2, REEP1, TDRD12, ZSWIM3, CCL3L3,
FAM154A, KCNIP4, NR4A3, REEP2, TDRD3, ZSWIM4, CCL4, FAM154B, KCNJ1, NR5A1, REEP3,
TDRD5, ZSWIM5, CCL4L1, FAM155A, KCNJ10, NR5A2, REEP4, TDRD6, ZSWIM6, CCL4L2,
FAM155B, KCNJ11, NR6A1, REEP5, TDRD7, ZSWIM7, CCL5, FAM156A, KCNJ12, NRAP, REEP6,
TDRD9, ZSWIM8, CCL7, FAM156B, KCNJ13, NRARP, REG1A, TDRKH, ZUFSP, CCL8, FAM157A,
KCNJ14, NRAS, REG1B, TDRP, ZW10, CCM2, FAM157B, KCNJ15, NRBF2, REG3A, TEAD1,
ZWILCH, CCM2L, FAM159A, KCNJ16, NRBP1, REG3G, TEAD2, ZWINT, CCNA1, FAM159B,
KCNJ18, NRBP2, REG4, TEAD3, ZXDA, CCNA2, FAM160A1, KCNJ2, NRCAM, REL, TEAD4,
ZXDB, CCNB1, FAM160A2, KCNJ3, NRD1, RELA, TEC, ZXDC, CCNB1IP1, FAM160B1, KCNJ4,
NRDE2, RELB, TECPR1, ZYG11A, CCNB2, FAM160B2, KCNJ5, NREP, RELL1, TECPR2, ZYG11B,
CCNB3, FAM161A, KCNJ6, NRF1, RELL2, TECR, ZYX, CCNC, FAM161B, KCNJ8, NRG1, RELN,
TECRL, ZZEF1, CCND1, FAM162A, KCNJ9, NRG2, RELT, TECTA, ZZZ3, CCND2, FAM162B,
KCNK1, NRG, RHODOPSIN, RdCVF, RdCVFL, GIRK, DUX4, and DBET (or DBET lncRNA).

### Heterologous Polynucleotide

In some embodiments, the systems and compositions of the present disclosure further comprises a heterologous polynucleotide (e.g., encoding a gene of interest, such as one or more genes selected from Table 1) that is introduced to the cell without being interested into a genome of the cell via action of the heterologous endonuclease of the present disclosure. In some cases, such heterologous polynucleotide encoding the gene of interest can be interested into the genome of the cell via other means, e.g., via adeno-associated virus vectors (e.g., AAV2 or AAV8). Alternatively, such heterologous polynucleotide encoding the gene of interest may be introduced to the intracellular portion of the cell and remain achromosomal (e.g., as an achromosomal plasmid).

Thus, the systems and compositions can comprise the non-disease causing wild type or variant of the target gene, as abovementioned. Alternatively or in addition to, the systems and compositions can comprise a heterologous polynucleotide sequence encoding (or comprising) at least the non-disease causing wild type or variant of the target gene (e.g., that of the endogenous target gene) as disclosed herein.

### Systems and Composition

In some aspects, the present disclosure provides a system or a composition comprising (i) the engineered gene effector as disclosed herein (e.g., the engineered gene activator, the engineered gene repressor) and, optionally, one or more of the following: (ii) the heterologous endonuclease as disclosed herein, (iii) the guide nucleic acid as disclosed herein, and/or (iv) the heterologous polynucleotide (e.g., encoding one or more genes from Table 1), for use in any of the methods as disclosed herein. The system or the composition can comprise one or more polynucleotides that encode any of the members (i)-(iv) abovementioned. The subject composition can be usable for modifying a cell in vitro, ex vivo, or in vivo. The subject composition can be usable for treating or enhancing a condition of a subject, as disclosed herein.

The composition as disclosed herein can comprise an active ingredient (e.g., the engineered gene effector, the heterologous endonuclease, the guide nucleic acid, etc.) and optionally an additional ingredient (e.g., excipient). If necessary and/or desirable, the composition can be divided, shaped and/or packaged into a desired single- or multi-dose unit or single- or multi-implantation unit.

In some embodiments, the composition can comprise one or more heterologous polynucleotides encoding the active ingredients as disclosed herein. When there are different members within the active ingredients, each member can be encoded by a different heterologous polynucleotide. Alternatively, two or more (e.g., all of) the ingredients can be encoded by a single heterologous polynucleotide. In some cases, a heterologous polynucleotide can encode the engineered gene effector as disclosed herein. In some cases, a single heterologous polynucleotide can encode (i) the engineered gene effector, (ii) the heterologous endonuclease, and (iii) one or more guide nucleic acids (e.g., at least 1, at least 2, at least 3, at least 4, at least 5, or more guide nucleic acids) for targeting specific region(s) or sequence(s) of the target gene.

The one or more heterologous polynucleotides can further comprise one or more promoters (or one or more transcriptional control elements, as used interchangeably herein). Different active ingredients encoded by the one or more heterologous polynucleotides can be under the control of the same promoter or different promoters. A promoter as disclosed herein can be active in a eukaryotic, mammalian, non-human mammalian or human cell. The promoter can be an inducible or constitutively active promoter. Alternatively or additionally, the promoter can be tissue or cell specific. Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression. In some cases, a promoter as disclosed herein can be a promoter specific for any of the tissues provided herein, or a promoter specific for any of the cell types provided herein.

A heterologous polynucleotide of the one or more heterologous polynucleotides (e.g., the single heterologous polynucleotide) can have a size of at least or up to about 2.5 kilobases, at least or up to about 2.6 kilobases, at least or up to about 2.7 kilobases, at least or up to about 2.8 kilobases, at least or up to about 2.9 kilobases, at least or up to about 3.0 kilobases, at least or up to about 3.1 kilobases, at least or up to about 3.2 kilobases, at least or up to about 3.3 kilobases, at least or up to about 3.4 kilobases, at least or up to about 3.5 kilobases, at least or up to about 3.6 kilobases, at least or up to about 3.7 kilobases, at least or up to about 3.8 kilobases, at least or up to about 3.9 kilobases, at least or up to about 4.0 kilobases, at least or up to about 4.1 kilobases, at least or up to about 4.2 kilobases, at least or up to about 4.3 kilobases, at least or up to about 4.4 kilobases, at least or up to about 4.5 kilobases, at least or up to about 4.6 kilobases, at least or up to about 4.7 kilobases, at least or up to about 4.8 kilobases, at least or up to about 4.9 kilobases, at least or up to about 5.0 kilobases, at least or up to about 5.5 kilobases, at least or up to about 6.0 kilobases, at least or up to about 6.5 kilobases, at least or up to about 7.0 kilobases, at least or up to about 7.5 kilobases, at least or up to about 8.0 kilobases, at least or up to about 9.0 kilobases, or at least or up to about 10 kilobases. In some cases, the heterologous polynucleotide of the one or more heterologous polynucleotides (e.g., the single heterologous polynucleotide) can have a size of between about 3 kilobases and about 5 kilobases, between about 3 kilobases and about 4.8 kilobases, between about 3 kilobases and about 4.6 kilobases, between about 3 kilobases and about 4.4 kilobases, between about 3 kilobases and about 4.2 kilobases, between about 3 kilobases and about 4.0 kilobases, between about 3 kilobases and about 3.5 kilobases, between about 3.5 kilobases and about 5 kilobases, between about 3.5 kilobases and about 4.8 kilobases, between about 3.5 kilobases and about 4.6 kilobases, between about 3.5 kilobases and about 4.4 kilobases, between about 3.5 kilobases and about 4.2 kilobases, between about 3.5 kilobases and about 4 kilobases, between about 4 kilobases and about 5 kilobases, between about 4 kilobases and about 4.9 kilobases, between about 4 kilobases and about 4.8 kilobases, between about 4 kilobases and about 4.7 kilobases, between about 4 kilobases and about 4.6 kilobases, between about 4 kilobases and about 4.5 kilobases, between about 4 kilobases and about 4.4 kilobases, between about 4 kilobases and about 4.3 kilobases, between about 4 kilobases and about 4.2 kilobases, or between about 4 kilobases and about 4.1 kilobases.

A method of delivery of the one or more heterologous polynucleotides provided herein to the cell can involve viral delivery methods or non-viral delivery methods. Thus, the one or more heterologous polynucleotides can be one or more viral vectors (e.g., one or more AAV vectors). Alternatively, the one or more heterologous polynucleotides can be non-viral vectors that are complexed with or encapsulated by non-viral delivery moieties, such as cationic lipids and/or lipid particles (e.g., lipid nanoparticles (LNP)).

Methods of non-viral delivery of nucleic acids can include lipofection, nucleofection, microinjection, biolistics, virosomes, liposomes, immunoliposomes, polycation or lipid: nucleic acid conjugates, naked DNA, artificial virions, and agent-enhanced uptake of DNA. Cationic and neutral lipids that are suitable for efficient receptor-recognition lipofection of polynucleotides can be used. Delivery can be to cells (e.g. in vitro or ex vivo administration) or target tissues (e.g. in vivo administration).

In some embodiments, the compositions and systems provided herein are delivered to a subject using a viral vector. In some cases, the viral vector is an adeno-associated viral (AAV) vector. The term “AAV” is an abbreviation for adeno-associated virus, and may be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle. In some cases, the AAV vector is selected based on the tropism of viral vector. In some embodiments, an AAV vector with tropism for the target tissue may be used to deliver polynucleotides encoding the compositions and systems provided herein to the target tissue.

RNA or DNA viral based systems can be used to target specific cells in the body and trafficking the viral payload to the nucleus of the cell. Viral vectors can be administered directly (in vivo), or they can be used to treat cells in vitro, and the modified cells can optionally be administered (ex vivo). Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene. High transduction efficiencies can be observed in many different cell types and target tissues.

The tropism of a retrovirus can be altered by incorporating foreign envelope proteins, expanding the potential target population of target cells. Lentiviral vectors are retroviral vectors that can transduce or infect non-dividing cells and produce high viral titers. Selection of a retroviral gene transfer system can depend on the target tissue. Retroviral vectors can comprise cis-acting long terminal repeats with packaging capacity for up to 6-10 kb of foreign sequence. The minimum cis-acting LTRs can be sufficient for replication and packaging of the vectors, which can be used to integrate the therapeutic gene into the target cell to provide permanent transgene expression. Retroviral vectors can include those based upon murine leukemia virus (MuLV), gibbon ape leukemia virus (GaLV), Simian Immuno deficiency virus (SIV), human immuno deficiency virus (HIV), and combinations thereof.

An adenoviral-based systems can be used. Adenoviral-based systems can lead to transient expression of the transgene. Adenoviral based vectors can have high transduction efficiency in cells and may not require cell division. High titer and levels of expression can be obtained with adenoviral based vectors. Adeno-associated virus (“AAV”) vectors can be used to transduce cells with target nucleic acids, e.g., in the in vitro production of nucleic acids and peptides, and for in vivo and ex vivo gene therapy procedures.

Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV.

A host cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. In some embodiments, a cell transiently transfected with the compositions of the disclosure (such as by transient transfection of one or more vectors, or transfection with RNA), and modified through the activity of the heterologous polypeptide comprising the engineered gene effector and the heterologous endonuclease as disclosed herein, is used to establish a new cell line comprising cells containing the modification but lacking any other exogenous sequence.

Any suitable vector compatible with the host cell can be used with the methods of the disclosure. Non-limiting examples of vectors for eukaryotic host cells include pXT1, pSG5 (Stratagene™), pSVK3, pBPV, pMSG, and pSVLSV40 (Pharmacia™).

In some embodiments, the additional ingredient of the composition as disclosed herein can comprise an excipient. Non-limiting examples of the excipient can include solvents, dispersion media, diluents, or other liquid vehicles, dispersion or suspension aids, surface active agents, isotonic agents, thickening or emulsifying agents, preservatives, lipidoids, liposomes, lipid nanoparticles, polymers, lipoplexes, core-shell nanoparticles, peptides, proteins, hyaluronidase, nanoparticle mimics, inert diluents, buffering agents, lubricating agents, oils, and combinations thereof. In some examples, the composition as disclosed herein can include one or more excipients, each in an amount that together increases the stability of (i) the heterologous polypeptide or the heterologous gene encoding thereof and/or (ii) cells or modified cells.

In some aspects, the present disclosure provides a kit comprising such composition and instructions directing (i) contacting the cell with the composition (e.g., in vitro, ex vivo, or in vivo), or (ii) administration of cells comprising any one of the compositions disclosed herein to a subject. The subject may have or may be suspected of having a condition, such as a hereditary disease.

In some embodiments, any of the compositions as disclosed herein, can be administered to the subject via orally, intraperitoneally, intravenously, intraarterially, transdermally, intramuscularly, liposomally, via local delivery by catheter or stent, subcutaneously, intraadiposally, or intrathecally. In particular aspects, the compositions and systems provided herein (including polynucleotides encoding said compositions and systems, e.g., contained in an AAV vector) can be administered to a subject via intravenous administration.

The compositions (e.g., pharmaceutical compositions) as disclosed herein can be suitable for administration to humans. In addition, such compositions can be suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

### Cells

In some embodiments, a cell as provided herein may be referred to as a target cell. In some embodiments, the systems, compositions, and methods as provided herein can be applied to modify a target cell (e.g., modify expression profile of a target gene of the target cell, such as one or genes in Table 1). A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

The cell (or target cell) can be engineered to comprise (or exhibit) any one of the systems or compositions as disclosed herein or can be treated by any one of the methods disclosed herein in vitro or ex vivo, then administered to the subject, e.g., to treat a condition of the subject. For example, any subject modified cell product can be administered to the subject to treat a condition of a bodily tissue of the subject. In some cases, the cell can be resident inside the subject's body, and any of the systems or compositions thereof can be administered to the subject, to contact the cell by the systems/compositions (e.g., to engineer the cell with the systems/compositions).

### EMBODIMENTS

The following non-limiting embodiments provide illustrative examples of the invention, but do not limit the scope of the invention.

Embodiment 1. An engineered gene effector comprising a polypeptide, wherein:
the polypeptide is heterologous to any of the members selected from the group consisting of VP16, VP64, p65, and Rta;
the engineered gene effector has a size of at most about 500 amino acid residues; and
the engineered gene effector is capable of activating expression level of a target gene in a cell, wherein the expression level of the target gene that is activated via the engineered gene effector is at least about 80% as compared to that activated by a VP64-p65-Rta fusion polypeptide (VPR) in a control cell,
optionally wherein:
(1) the expression level of the target gene that is activated by the engineered gene effector is at least about 90% to that activated by the VPR in the control cell; and/or
(2) the expression level of the target gene that is activated by the engineered gene effector is at least about 95% to that activated by the VPR in the control cell; and/or
(3) the expression level of the target gene that is activated by the engineered gene effector is at least about 100% to that activated by the VPR in the control cell; and/or
(4) the expression level of the target gene that is activated by the engineered gene effector is at least about 5% greater than that activated by the VPR in the control cell; and/or
(5) the expression level of the target gene that is activated by the engineered gene effector is at least about 10% greater than that activated by the VPR in the control cell; and/or
(6) the expression level of the target gene that is activated via the engineered gene effector persists for a duration of at least about 9 days; and/or
(7) the expression level of the target gene that is activated via the engineered gene effector persists for a duration of at least about 12 days; and/or
(8) the expression level of the target gene that is activated via the engineered gene effector persists for a duration of at least about 18 days; and/or
(9) the expression level of the target gene that is activated by the engineered gene effector is at least about 80% to that activated by the VPR in the control cell, and the expression level persist for a duration of at least about 9 days; and/or
(10) the expression level of the target gene that is activated by the engineered gene effector is at least about 90% to that activated by the VPR in the control cell, and the expression level persist for a duration of at least about 12 days; and/or
(11) the polypeptide comprises an amino acid sequence that exhibits at least about 70% sequence identity to the polypeptide sequence of SEQ ID NO: 1; and/or
(12) the amino acid sequence of the polypeptide exhibits at least about 80% sequence identity to the polypeptide sequence of SEQ ID NO: 1; and/or
(13) the amino acid sequence of the polypeptide exhibits at least about 90% sequence identity to the polypeptide sequence of SEQ ID NO: 1; and/or
(14) the amino acid sequence of the polypeptide exhibits at least about 95% sequence identity to the polypeptide sequence of SEQ ID NO: 1; and/or
(15) the engineered gene effector further comprises an amino acid sequence having at least about 70% sequence identity to the polypeptide sequence to a member selected from the group consisting of SEQ ID NOs: 23, 24, 31, 79-84, 86-92, 94-103, 116, 119-120, 122, 123, and 125; and/or
(16) a size of the amino acid sequence of the polypeptide is between about 10 amino acid residues and about 50 amino acid residues; and/or
(17) the size of the amino acid sequence of the polypeptide is between about 20 amino acid residues and about 40 amino acid residues; and/or
(18) a size of the engineered gene effector is less than or equal to about 400 amino acid residues; and/or
(19) the size of the engineered gene effector is less than or equal to about 300 amino acid residues; and/or
(20) the size of the engineered gene effector is less than or equal to about 250 amino acid residues; and/or
(21) the size of the engineered gene effector is less than or equal to about 150 amino acid residues; and/or
(22) the size of the engineered gene effector is less than or equal to about 100 amino acid residues; and/or
(23) the target gene is endogenous to the cell.

Embodiment 2. An engineered gene effector comprising a polypeptide coupled to an additional polypeptide, wherein:
the polypeptide comprises an amino acid sequence having at least about 70% sequence identity to the polypeptide sequence of SEQ ID NO: 1;
the additional polypeptide comprises at least a portion of one or more members selected from the group consisting of VP16, VP64, p65, and Rta; and
the engineered gene effector has a size less than or equal to about 250 amino acid residues, optionally wherein:
(1) the additional polypeptide comprise VP16; and/or
(2) the additional polypeptide comprise VP64; and/or
(3) the additional polypeptide comprise P65; and/or
(4) the additional polypeptide comprise Rta; and/or
(5) the amino acid sequence of the polypeptide comprises one or more members selected from the group consisting of C4, L5, M7, and L19, when aligned to the polypeptide sequence of SEQ ID NO: 1; and/or
(6) the amino acid sequence of the polypeptide exhibits at least about 80% sequence identity to the polypeptide sequence of SEQ ID NO: 1; and/or
(7) the amino acid sequence of the polypeptide exhibits at least about 90% sequence identity to the polypeptide sequence of SEQ ID NO: 1; and/or
(8) the amino acid sequence of the polypeptide exhibits at least about 95% sequence identity to the polypeptide sequence of SEQ ID NO: 1; and/or
(9) the engineered gene effector further comprises an amino acid sequence having at least about 70% sequence identity to the polypeptide sequence to a member selected from the group consisting of SEQ ID NOs: 23, 24, 31, 79-84, 86-92, 94-103, 116, 119-120, 122, 123, and 125; and/or
(10) the engineered gene effector is capable of activating expression level of a target gene in a cell; and/or
(11) the size of the engineered gene effector is less than or equal to about 150 amino acid residues; and/or
(12) the size of the engineered gene effector is less than or equal to about 100 amino acid residues; and/or
(13) the polypeptide is fused to the additional polypeptide; and/or
(14) the polypeptide is not identical to any one of SEQ ID NO: 2 and SEQ ID NO: 3; and/or
(15) the at least the portion comprises an amino acid sequence having at least about 70% sequence identity to the polypeptide sequence of SEQ ID NO: 5; and/or
(16) the at least the portion comprises an amino acid sequence having at least about 70% sequence identity to the polypeptide sequence of SEQ ID NO: 6; and/or
(17) the at least the portion comprises an amino acid sequence having at least about 70% sequence identity to the polypeptide sequence of SEQ ID NO: 7; and/or
(18) the at least the portion comprises an amino acid sequence having at least about 70% sequence identity to the polypeptide sequence of SEQ ID NO: 8.

Embodiment 3. An engineered gene effector comprising a polypeptide, wherein the polypeptide comprises an amino acid sequence having at least about 70% sequence identity to the polypeptide sequence of SEQ ID NO: 1,
wherein the engineered gene effector is not identical to any one of SEQ ID NO: 2, SEQ ID NO: 3, and SEQ ID NO: 4,
optionally wherein:
(1) the amino acid sequence of the polypeptide comprises one or more members selected from the group consisting of C4, L5, M7, and L19, when aligned to the polypeptide sequence of SEQ ID NO: 1; and/or
(2) the engineered gene effector is capable of activating expression level of a target gene in a cell; and/or
(3) the engineered gene effector is capable of activating expression level of a target gene in a cell for at least about 9 days; and/or
(4) the engineered gene effector further comprises an additional polypeptide that is heterologous to the polypeptide, further optionally wherein:
   (i) the polypeptide is coupled to the additional polypeptide; and/or
   (ii) the polypeptide is fused to the additional polypeptide; and/or
   (iii) the additional polypeptide alone encodes at least one gene effector, further optionally wherein the at least one gene effector of the additional polypeptide comprises at least one transcriptional activator; further optionally wherein:
      (a) the at least one transcriptional activator of the additional polypeptide comprises one or more members selected from the group consisting of VP16, VP64, VP48, VP160, p65, EDLL, TAL, SET1A, SET1B, MLL, ASH1, SYMD2, NSD1, JHDM2a/b, UTX, JMJD3, GCN5, PCAF, CBP, p300, TAF1, TIP60/PLIP, MOZMYST3, MORFMYST4, SRC1, ACTR, PI60, CLOCK, TETDME, DML1, DML2, ROS1, and a fragment thereof; and/or
      (b) the at least one transcriptional activator of the additional polypeptide does not comprise two or more members selected from the group consisting of VP64, p65, and Rta; and/or
   (iv) the additional polypeptide alone does not encode any gene effector; and/or
   (v) the additional polypeptide comprises a filler polypeptide sequence; and/or
   (vi) the additional polypeptide comprises a linker sequence; and/or
(5) the amino acid sequence of the polypeptide exhibits at least about 80% sequence identity to the polypeptide sequence of SEQ ID NO: 1; and/or
(6) the amino acid sequence of the polypeptide exhibits at least about 90% sequence identity to the polypeptide sequence of SEQ ID NO: 1; and/or
(7) the amino acid sequence of the polypeptide exhibits at least about 95% sequence identity to the polypeptide sequence of SEQ ID NO: 1; and/or
(8) the engineered gene effector further comprises an amino acid sequence having at least about 70% sequence identity to the polypeptide sequence to a member selected from the group consisting of SEQ ID NOs: 23, 24, 31, 79-84, 86-92, 94-103, 116, 119-120, 122, 123, and 125.

Embodiment 4. An engineered gene effector comprising a plurality of polypeptide domains, wherein each polypeptide domain of the plurality of polypeptide domain comprises a polypeptide comprising an amino acid sequence that exhibits at least about 70% sequence identity to the polypeptide sequence of SEQ ID NO: 1,
optionally wherein:
(1) the amino acid sequence of the polypeptide comprises one or more members selected from the group consisting of C4, L5, M7, and L19, when aligned to the polypeptide sequence of SEQ ID NO: 1; and/or
(2) the amino acid sequence of the polypeptide exhibits at least about 80% sequence identity to the polypeptide sequence of SEQ ID NO: 1; and/or
(3) the amino acid sequence of the polypeptide exhibits at least about 90% sequence identity to the polypeptide sequence of SEQ ID NO: 1; and/or
(4) the amino acid sequence of the polypeptide exhibits at least about 95% sequence identity to the polypeptide sequence of SEQ ID NO: 1; and/or
(5) the engineered gene effector is capable of activating expression level of a target gene in a cell; and/or
(6) the engineered gene effector is capable of activating expression level of a target gene in a cell for at least about 9 days; and/or
(7) the plurality of polypeptide domain comprises three or more polypeptides; and/or
(8) the plurality of polypeptide domains comprises a first polypeptide domain and a second polypeptide domain that are coupled to each other, further optionally wherein the first polypeptide domain is fused to the second polypeptide domain; and/or
(9) the engineered gene effector further comprises an amino acid sequence having at least about 70% sequence identity to the polypeptide sequence to a member selected from the group consisting of SEQ ID NOs: 23, 24, 31, 79-84, 86-92, 94-103, 116, 119-120, 122, 123, and 125; and/or
(10) the engineered gene effector further comprises an additional polypeptide that is heterologous to the each polypeptide domain, further optionally wherein:
(i) the additional polypeptide is positioned adjacent to C-terminus of at least one of the plurality of polypeptide domains; and/or
(ii) the additional polypeptide is positioned adjacent to N-terminus of at least one of the plurality of polypeptide domains; and/or
(iii) the additional polypeptide is flanked by two polypeptide domains of the plurality of polypeptide domains; and/or
(iv) the additional polypeptide comprises at least 1 amino acid residue; and/or
(v) the additional polypeptide comprises at most 8 amino acid residues; and/or
(vi) the additional polypeptide has a length between about 1 and about 8 amino acid residues; and/or
(vii) the additional polypeptide alone does not encode any gene effector; and/or
(viii) the additional polypeptide comprises a filler polypeptide sequence; and/or
(ix) the additional polypeptide comprises a linker sequence.

Embodiment 5. An engineered gene effector comprising an amino acid sequence having at least about 70% sequence identity to the polypeptide sequence to a member selected from the group consisting of SEQ ID NOs: 23, 24, 31, 79-84, 86-92, 94-103, 116, 119-120, 122, 123, and 125;
optionally wherein:
(1) the amino acid sequence having at least about 70% sequence identity to the polypeptide sequence to a member selected from the group consisting of SEQ ID NOs: 79-84, 88, 90, 91 and 95; and/or
(2) the engineered gene effector comprises a polypeptide comprising an amino acid sequence that exhibits at least about 70% sequence identity to the polypeptide sequence of SEQ ID NO: 1; and/or
(3) the amino acid sequence of the polypeptide exhibits at least about 80%, at least about 90%, at least about 95%, or at least about 99% sequence identity to the polypeptide sequence of SEQ ID NO: 1; and/or
(4) the polypeptide exhibits at most about 80%, at most about 60%, or at most about 40% sequence identity to the amino acid sequence of SEQ ID NO: 2; and/or
(5) the polypeptide exhibits at most about 80%, at most about 60%, or at most about 40% sequence identity to the amino acid sequence of SEQ ID NO: 3; and/or
(6) a size of the amino acid sequence of the polypeptide is at most about 50 amino acid residues, at most about 40 amino acid residues, or at most about 35 amino acid residues; and/or
(7) the size of the amino acid sequence of the polypeptide is at least about 20 amino acid residues, at least about 25 amino acid residues, or at least about 30 amino acid residues; and/or
(8) the size of the amino acid sequence of the additional polypeptide is at least about 5 amino acid residues, at least about 10 amino acid residues, at least about 20 amino acid residues, at least about 30 amino acid residues, at least about 40 amino acid residues, or at least about 50 amino acid residues; and/or
(9) a size of the engineered gene effector is less than about 680 AA, less than about 500 AA, less than about 400 AA, less than about 300 AA, or less than about 200 AA; and/or
(10) the size of the engineered gene effector is less than about 150 AA; and/or
(11) the engineered gene effector is capable of activating expression level of a target gene in a cell for at least about 9 days; and/or
(12) the engineered gene effector does not comprise VPR; and/or
(13) the target gene is heterologous to the cell; and/or
(14) the target gene is endogenous to the cell; and/or
(15) the engineered gene effector exhibits at least about 70% sequence identity, at least about 80% sequence identity, at least about 90% sequence identity, or at least about 95% sequence identity to a member selected from the group consisting of SEQ ID NOs: 12-31; and/or
(16) the engineered gene effector exhibits at least about 70% sequence identity, at least about 80% sequence identity, at least about 90% sequence identity, or at least about 95% sequence identity to a member selected from the group consisting of SEQ ID NOs: 19, 20, 23, 24, 30, 31, 79, 80, 81, 82, 83, 84, 88, 90, 91 and 95; and/or
(17) the engineered gene effector exhibits at least about 70% sequence identity, at least about 80% sequence identity, at least about 90% sequence identity, or at least about 95% sequence identity to a member selected from the group consisting of SEQ ID NOs: 23, 24, 31, 79, 80, 81, 82, 83, 84, 88, 90, 91 and 95; and/or
(18) the engineered gene effector is coupled to a heterologous endonuclease; and/or
(19) the engineered gene effector is fused to the heterologous endonuclease, further optionally wherein:
   (i) the heterologous endonuclease comprises a Cas protein; and/or
   (ii) the Cas protein comprises the polypeptide sequence of SEQ ID NO: 10 or a modification thereof, further optionally wherein the Cas protein is a deactivated Cas (dCas) protein, further optionally wherein the dCas protein comprises the polypeptide sequence of SEQ ID NO: 11 or a modification thereof.

Embodiment 6. An engineered gene effector comprising a polypeptide, wherein the polypeptide comprises an amino acid sequence that exhibits at least about 70% sequence identity to the polypeptide sequence of SEQ ID NO: 1, and wherein the engineered gene effector is characterized by one or more members selected from the group consisting of: (i) the engineered gene effector has a size less than or equal to about 72 amino acid residues; (ii) the engineered gene effector further comprises an additional polypeptide having a size of less than or equal to about 40 amino acid residues; (iii) the engineered gene effector further comprises an additional polypeptide comprising at most about 5 glycine-serine (GS) linkers; (iv) the engineered gene effector further comprises an additional polypeptide comprising at most about 10 glycine (G) residues; (v) the engineered gene effector is capable of reducing expression level of a target gene in a cell,
optionally wherein:
(1) the engineered gene effector is characterized by (i); and/or
(2) the engineered gene effector has a size less than or equal to about 70 amino acid residues; and/or
(3) the engineered gene effector has a size less than or equal to about 65 amino acid residues; and/or
(4) the engineered gene effector is characterized by (ii); and/or
(5) the size of the additional polypeptide is less than or equal to about 35 amino acid residues; and/or
(6) the size of the additional polypeptide is at most about 30% greater than the size of the polypeptide; and/or
(7) the additional polypeptide is positioned adjacent to N-terminus of the polypeptide; and/or
(8) the engineered gene effector is characterized by (iii); and/or
(9) the additional polypeptide comprises at most about 4 GS linkers; and/or
(10) the additional polypeptide is heterologous to SEQ ID NO: 3; and/or
(11) the engineered gene effector is characterized by (iv); and/or
(12) the additional polypeptide comprises at most about 9 G residues; and/or
(13) the engineered gene effector is characterized by (v); and/or
(14) the expression level of the target gene that is reduced by the engineered gene effector is comparable to that reduced by KRAB; and/or
(15) the engineered gene effector is characterized by two or more members selected from the group consisting of (i)-(v); and/or
(16) the engineered gene effector is characterized by three or more members selected from the group consisting of (i)-(v); and/or
(17) the engineered gene effector is characterized by four or more members selected from the group consisting of (i)-(v); and/or
(18) the engineered gene effector is characterized by all of (i)-(v); and/or
(19) a central amino acid sequence of the polypeptide is closer to C-terminus of the engineered gene effector, as compared to N-terminus of the engineered gene effector; and/or
(20) the amino acid sequence of the polypeptide exhibits at least about 80%, at least about 90%, at least about 95%, or at least about 99% sequence identity to the polypeptide sequence of SEQ ID NO: 1; and/or
(21) the polypeptide exhibits at most about 80%, at most about 60%, or at most about 40% sequence identity to the amino acid sequence of SEQ ID NO: 2; and/or
(22) the polypeptide exhibits at most about 80%, at most about 60%, or at most about 40% sequence identity to the amino acid sequence of SEQ ID NO: 3.

Embodiment 7. An engineered gene effector comprising a polypeptide, wherein the polypeptide comprises an amino acid sequence having at least about 70% sequence identity to the polypeptide sequence of SEQ ID NO: 52, wherein the engineered gene effector is not identical to any one of SEQ ID NO: 53 and SEQ ID NO: 54;
optionally wherein:
(1) the engineered gene effector is capable of reducing expression level of a target gene in a cell; and/or
(2) the expression level of the target gene that is reduced by the engineered gene effector is comparable to that reduced by KRAB; and/or
(3) the amino acid sequence of the polypeptide has at least about 80%, at least about 90%, at least about 95%, or at least about 99% sequence identity to the polypeptide sequence of SEQ ID NO: 52; and/or
(4) the engineered gene effector further comprises an additional polypeptide, wherein the polypeptide comprises an amino acid sequence having at least about 70% sequence identity to the polypeptide sequence of SEQ ID NO: 52; and/or
(5) the engineered gene effector further comprises a different polypeptide that is heterologous to any one of SEQ ID NO: 53 and SEQ ID NO: 54, further optionally wherein the different polypeptide comprises an amino acid sequence having at least about 70% sequence identity to a member selected from the group consisting of SEQ ID NO: 55, SEQ ID NO: 58, and SEQ ID NO: 61; and/or
(6) the engineered gene effector does not comprise the amino acid sequence of SEQ ID NO: 1.

Embodiment 8. An engineered gene effector comprising a polypeptide, wherein the polypeptide comprises an amino acid sequence having at least about 70% sequence identity to the polypeptide sequence of SEQ ID NO: 55,
wherein the engineered gene effector is not identical to any one of SEQ ID NO: 56 and SEQ ID NO: 57,
optionally wherein:
(1) the engineered gene effector is capable of reducing expression level of a target gene in a cell; and/or
(2) the expression level of the target gene that is reduced by the engineered gene effector is comparable to that reduced by KRAB; and/or
(3) the amino acid sequence of the polypeptide has at least about 80%, at least about 90%, at least about 95%, or at least about 99% sequence identity to the polypeptide sequence of SEQ ID NO: 55; and/or
(4) the engineered gene effector further comprises an additional polypeptide, wherein the polypeptide comprises an amino acid sequence having at least about 70% sequence identity to the polypeptide sequence of SEQ ID NO: 55; and/or
(5) the engineered gene effector further comprises a different polypeptide that is heterologous to any one of SEQ ID NO: 56 and SEQ ID NO: 57; and/or
(6) the different polypeptide comprises an amino acid sequence having at least about 70% sequence identity to a member selected from the group consisting of SEQ ID NO: 52, SEQ ID NO: 58, and SEQ ID NO: 61; and/or
(7) the engineered gene effector does not comprise the amino acid sequence of SEQ ID NO: 1.

Embodiment 9. An engineered gene effector comprising a polypeptide, wherein the polypeptide comprises an amino acid sequence having at least about 70% sequence identity to the polypeptide sequence of SEQ ID NO: 58,
wherein the engineered gene effector is not identical to any one of SEQ ID NO: 59 and SEQ ID NO: 60,
optionally wherein:
(1) the engineered gene effector is capable of reducing expression level of a target gene in a cell; and/or
(2) the expression level of the target gene that is reduced by the engineered gene effector is comparable to that reduced by KRAB; and/or
(3) the amino acid sequence of the polypeptide has at least about 80%, at least about 90%, at least about 95%, or at least about 99% sequence identity to the polypeptide sequence of SEQ ID NO: 58; and/or
(4) the engineered gene effector further comprises an additional polypeptide, wherein the polypeptide comprises an amino acid sequence having at least about 70% sequence identity to the polypeptide sequence of SEQ ID NO: 58; and/or
(5) the engineered gene effector further comprises a different polypeptide that is heterologous to any one of SEQ ID NO: 59 and SEQ ID NO: 60; and/or
(6) the different polypeptide comprises an amino acid sequence having at least about 70% sequence identity to a member selected from the group consisting of SEQ ID NO: 52, SEQ ID NO: 55, and SEQ ID NO: 61; and/or
(7) the engineered gene effector does not comprise the amino acid sequence of SEQ ID NO: 1.

Embodiment 10. An engineered gene effector comprising a polypeptide, wherein the polypeptide comprises an amino acid sequence having at least about 70% sequence identity to the polypeptide sequence of SEQ ID NO: 61,
wherein the engineered gene effector is not identical to any one of SEQ ID NO: 62 and SEQ ID NO: 63,
optionally wherein:
(1) the engineered gene effector is capable of reducing expression level of a target gene in a cell; and/or
(2) the expression level of the target gene that is reduced by the engineered gene effector is comparable to that reduced by KRAB; and/or
(3) the amino acid sequence of the polypeptide has at least about 80%, at least about 90%, at least about 95%, or at least about 99% sequence identity to the polypeptide sequence of SEQ ID NO: 61; and/or
(4) the engineered gene effector further comprises an additional polypeptide, wherein the polypeptide comprises an amino acid sequence having at least about 70% sequence identity to the polypeptide sequence of SEQ ID NO: 61; and/or
(5) the engineered gene effector further comprises a different polypeptide that is heterologous to any one of SEQ ID NO: 62 and SEQ ID NO: 63; and/or
(6) the different polypeptide comprises an amino acid sequence having at least about 70% sequence identity to a member selected from the group consisting of SEQ ID NO: 52, SEQ ID NO: 55, and SEQ ID NO: 58; and/or
(7) the engineered gene effector does not comprise the amino acid sequence of SEQ ID NO: 1; and/or
(8) a size of the amino acid sequence of the polypeptide is at most about 50 amino acid residues, at most about 40 amino acid residues, or at most about 35 amino acid residues; and/or
(9) the size of the amino acid sequence of the polypeptide is at least about 20 amino acid residues, at least about 25 amino acid residues, or at least about 30 amino acid residues; and/or
(10) a size of the engineered gene effector is less than about 680 AA, less than about 500 AA, less than about 400 AA, less than about 300 AA, or less than about 200 AA; and/or
(11) the size of the engineered gene effector is less than about 150 AA; and/or
(12) the engineered gene effector does not comprise KRAB; and/or
(13) the target gene is heterologous to the cell; and/or
(14) the target gene is endogenous to the cell; and/or
(15) the engineered gene effector exhibits at least about 70% sequence identity, at least about 80% sequence identity, at least about 90% sequence identity, or at least about 95% sequence identity to a member selected from the group consisting of SEQ ID NOs: 32-51; and/or
(16) the engineered gene effector exhibits at least about 70% sequence identity, at least about 80% sequence identity, at least about 90% sequence identity, or at least about 95% sequence identity to a member selected from the group consisting of SEQ ID NOs: 32-35, 38, and 39; and/or
(17) the engineered gene effector exhibits at least about 70% sequence identity, at least about 80% sequence identity, at least about 90% sequence identity, or at least about 95% sequence identity to a member selected from the group consisting of SEQ ID NOs: 44-51; and/or
(18) the engineered gene effector is coupled to a heterologous endonuclease; and/or
(19) the engineered gene effector is fused to the heterologous endonuclease; and/or
(20) the heterologous endonuclease comprises a Cas protein, further optionally wherein the Cas protein comprises the polypeptide sequence of SEQ ID NO: 10 or a modification thereof, further optionally wherein:
   (i) the Cas protein is a deactivated Cas (dCas) protein; and/or
   (ii) the dCas protein comprises the polypeptide sequence of SEQ ID NO: 11 or a modification thereof.

Embodiment 11. A system comprising the engineered gene effector of any one of the embodiments,
optionally wherein:
(1) the system comprises the engineered gene effector that is coupled to the heterologous endonuclease; and/or
(2) the system further comprises a guide nucleic acid capable of forming a complex comprising the engineered gene effector and the heterologous endonuclease, wherein the complex exhibits specific binding to the target gene.

Embodiment 12. One or more polynucleotides encoding the system of any one of the preceding embodiments.

Embodiment 13. A composition comprising the system of any one of the preceding embodiments.

Embodiment 14. A cell comprising the system of any one of the preceding embodiments.

Embodiment 15. A method of controlling a target gene in a cell, the method comprising contacting the cell with the system of any one of the preceding embodiments,
optionally wherein:
(1) the controlling comprises activating expression level of the target gene; and/or
(2) the controlling comprises reducing expression level of the target gene; and/or
(3) the target gene is endogenous to the cell.

### EXAMPLES

### Example 1: Engineered Gene Effectors

One or more of the engineered effectors as disclosed herein can be used, either alone or in combination with another agent (e.g., Cas, dCas, sgRNA, etc.) to modulate (e.g., activate, repress) expression and/or activity level of a target gene in a cell (e.g., an endogenous target gene, a heterologous target gene).

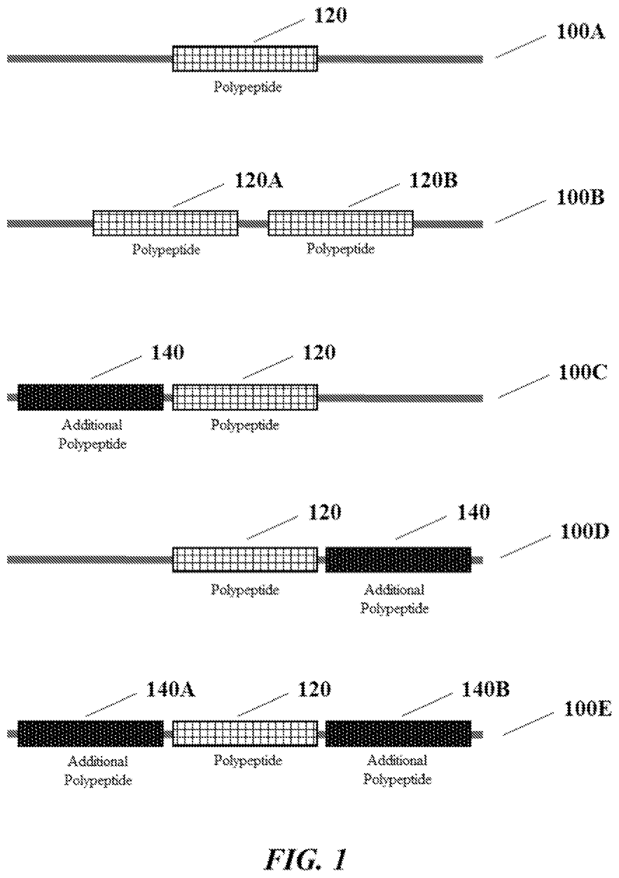

FIG. 1 schematically illustrates examples of the engineered gene effector 100 (e.g., 100A, 100B, 100C, 100D, and 100E) of the present disclosure. The engineered gene effector 100A can comprise the polypeptide 120 of the present disclosure (e.g., comprising at least a portion of the polypeptide sequence of SEQ ID NO: 1). The engineered gene effector 100B can comprise a plurality of the polypeptide 120 (120A and 120B) of the present disclosure (e.g., each comprising at least a portion of the polypeptide sequence of SEQ ID NO: 1). The engineered gene effector 100C can comprise the polypeptide 120 of the present disclosure and the additional polypeptide 140 of the present disclosure (e.g., not comprising the polypeptide sequence of SEQ ID NO: 1), wherein the additional polypeptide 140 is disposed adjacent to the N-terminus of the polypeptide 120. The engineered gene effector 100D can comprise the polypeptide 120 of the present disclosure and the additional polypeptide 140 of the present disclosure (e.g., not comprising the polypeptide sequence of SEQ ID NO: 1), wherein the additional polypeptide 140 is disposed adjacent to the C-terminus of the polypeptide 120. The engineered gene effector 100E can comprise the polypeptide 120 of the present disclosure and a plurality of the additional polypeptide 140 (140A and 140B) of the present disclosure (e.g., each not comprising the polypeptide sequence of SEQ ID NO: 1), wherein the polypeptide 120 is flanked by the additional polypeptide 140A and the additional polypeptide 140B.

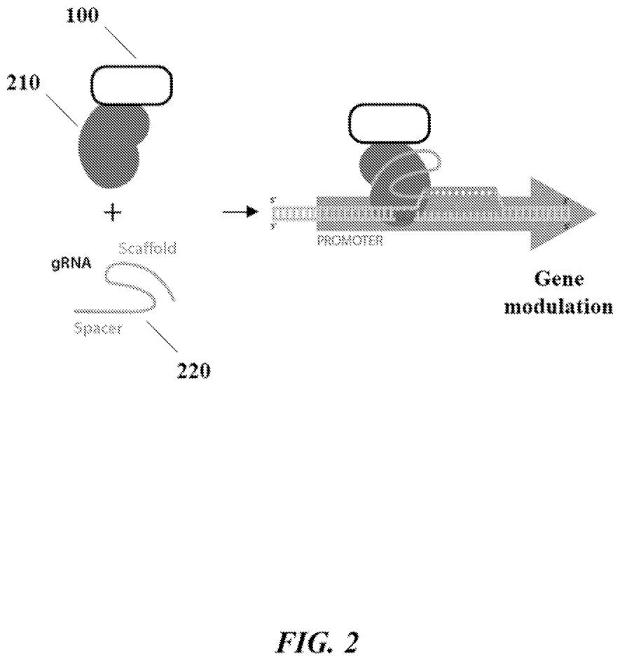

FIG. 2 schematically illustrates a complex comprising (i) the engineered gene effector 100 of the present disclosure that is coupled to a heterologous endonuclease (e.g., dCas, such as dCas9 or dCasMini) 210 and (ii) a guide RNA (gRNA) 220, wherein the complex can be capable of binding a target gene to modulate expression and/or activity level of the target gene.

### Example 2: Identification of Active Gene Effector Domain

The polypeptide of SEQ ID NO: 2 (or EPIC-XV1.8) and the polypeptide of SEQ ID NO: 3 (or EPIC-XV1.5) are polypeptide domains from wild type viral IRF2 (vIRF2). When operatively coupled to a deactivated Cas protein (e.g., dCas9, dCasMini) and a small guide nucleic acid molecule (sgRNA) against a target gene, each of the polypeptide of SEQ ID NO: 2 and the polypeptide of SEQ ID NO: 3 can modulate (e.g., activate) expression and/or activity level of the target gene. Because the polypeptide of SEQ ID NO: 2 and the polypeptide of SEQ ID NO: 3 can share a number of amino acid residues, one or more amino acid sequences that are shared between the polypeptide of SEQ ID NO: 2 and the polypeptide of SEQ ID NO: 3 can be identified to be important for modulating expression and/or activity of a target gene.

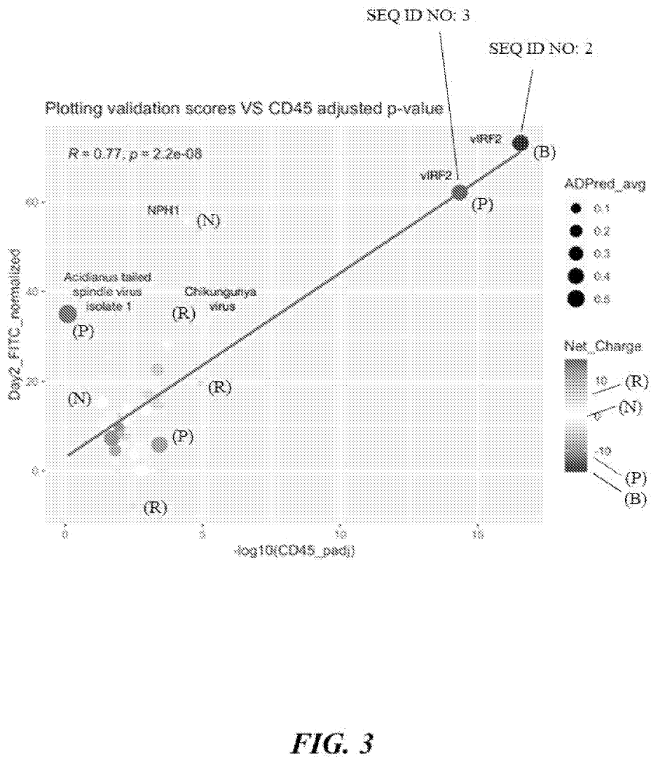

For example, a library of different gene effector candidates, including the polypeptide of SEQ ID NO: 2 and the polypeptide of SEQ ID NO: 3, was tested in conjunction with a dCas/anti-CD45 sgRNA complex in HEK293T cells, to screen for one or more gene effectors candidates capable of effecting modulation (e.g., activation) of the CD45 gene in the HEK293T cells. Each of the polypeptide of SEQ ID NO: 2 and the polypeptide of SEQ ID NO: 3 exhibited the ability to activate expression of the CD45 gene in the HEK293T cells. FIG. 3 shows a visualization of the correlation between validation experiments performed in HEK293T cells and results from the high-throughput CD45 screening assay as described. The plot in FIG. 3 is annotated with net charge and average scores from ADPred. ADPred is a deep learning model to predict acidic transcription activation domains (ADs) within protein sequences. The x-axis represents the statistical significance associated with CD45 activation ability of individual modulators and the y-axis represents the fluorescence intensity of activation at TRE3G-GFP in HEK293T in validation experiments. A trend line is added (R=0.77, p=2.2e-08) indicating the positive correlation between validation experiments (TREG-GFP activation) and results from the initial high-throughput modulator screen (CD45 activation). Each modulator is further annotated with net charge (red (R) indicates basic peptides and blue (B) or purple (P) indicate acidic peptides) and average ADPred score associated with the modulator's amino acid sequence (e.g., larger circles indicate greater likelihood that the peptide contains an activation domain predicted by ADPred). The plot in FIG. 3 shows that the original screen (CD45 activation) results correlated with the validation experiments (TREG-GFP activation), largely driven by the high performance of the two overlapping vIRF2 tiles, e.g., the polypeptide of SEQ ID NO: 2 and the polypeptide of SEQ ID NO: 3. Without wishing to be bound by theory, analysis of charge and activation domain prediction as demonstrated herein suggests that such properties can be helpful predictors of potent activators (e.g., see the polypeptide of SEQ ID NO: 2 and the polypeptide of SEQ ID NO: 3), and that additional potential gene effectors can exist, e.g., without net negative charge or high ADPred score (e.g. NPH1 and Chikungunya tiles). Without wishing to be bound by theory, potent gene effectors, such as potent gene activators, can exhibit a depletion of basic amino acid residues or basic peptides.

FIG. 4 shows a graphical representation of the ADPred output for a vIRF2 tile. In this case, the 85 amino acid residues vIRF2 tile that consistently acts as a potent activator contains a 32 amino acid residue domain (SEQ ID NO: 1, represented as 400) with extremely high ADPred scores, suggesting that this region may be the functional activation domain. Additionally, the two vIRF2 tiles from the original screen overlap in a region containing the predicted 32 amino acid residue activation domain, thus supporting the hypothesis that this may be the region responsible for activation. In FIG. 4, the overlapping sequences can be seen above highlighted in red (400). Also shown in FIG. 4 are individual amino acids plotted based on basic biochemical properties (e.g., hydrophobic, aromatic, neutral, acidic, basic), e.g., to illustrate that the predicted activation domain comprising the 32 amino acid residues may be enriched for hydrophobic and acidic residues.

In some embodiments, the identified active gene modulation domain 400 (SEQ ID NO: 1) can be engineered (e.g., modified) to form one or more of the engineered gene effectors as provided in the present disclosure.

### Example 3: Design of Engineered Gene Effectors

One or more of the engineered effectors as disclosed herein can comprise a polypeptide sequence that is derived from vIRF2. The engineered effector can comprise at least a portion of the polypeptide sequence of SEQ ID NO: 1. The engineered effector can comprise one or more polypeptide sequences derived from the vIRF2. Alternatively, the engineered effector can comprise one or more polypeptide sequences derived from the vIRF2 and one or more additional polypeptide sequences that are heterologous to vIRF2.

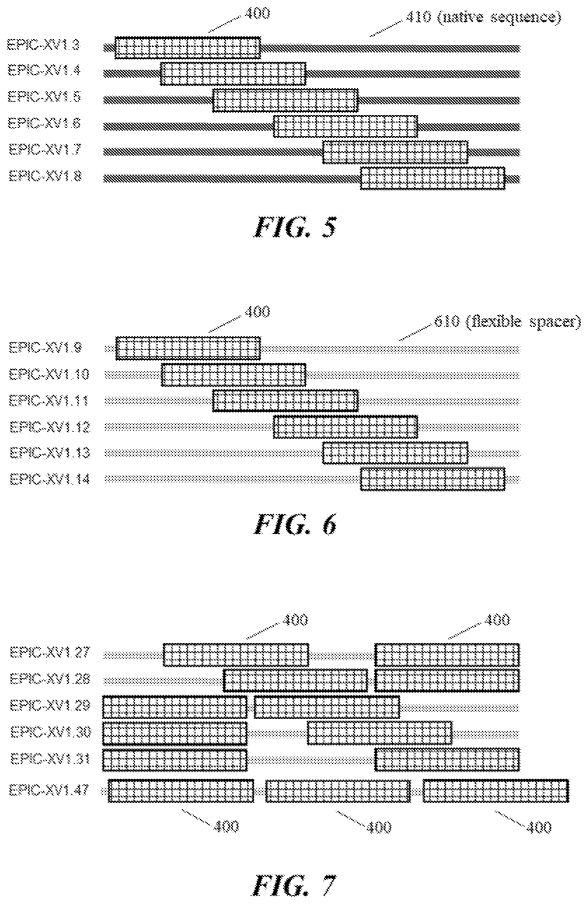

As shown in FIG. 5, example engineered effectors EPIC-XV 1.3 through 1.8 can each comprise a polypeptide sequence (e.g., about 85 amino acid long polypeptide sequence) that is taken directly from the native vIRF2 amino acid sequence from Human herpesvirus 8. The engineered effectors EPIC-XV 1.3-1.8 can be taken from different regions (or from a sliding window) of the native vIRF2 amino acid sequence, such that each of the engineered effectors are different but each comprises (i) the polypeptide sequence of SEQ ID NO: 1 (domain 400) and (ii) an additional native sequence(s) (domain(s) 410).

As shown in FIG. 6, example engineered effectors EPIC-XV 1.9 through 1.14 can each comprise a polypeptide sequence (e.g., about 85 amino acid long polypeptide sequence) that is at least partially derived from the native vIRF2 amino acid sequence from Human herpesvirus 8. Each of the engineered effectors EPIC-XV 1.9-1.14 can comprise (i) at least a portion of the polypeptide sequence of SEQ ID NO: 1 (domain 400) and (ii) one or more additional flexible spacer sequences (domain(s) 610), with a position of the domain 400 varied from being closer to N-terminal (e.g., EPIC-XV 1.9) to being closer to C-terminus (EPIC-XV 1.14).

As shown in FIG. 7, example engineered effectors EPIC-XV 1.27 through 1.31 can each comprise a polypeptide sequence (e.g., about 85 amino acid long polypeptide sequence) that is at least partially derived from the native vIRF2 amino acid sequence from Human herpesvirus 8. Each of the engineered effectors EPIC-XV 1.27-1.31 can comprise a plurality of domains 400 (e.g., at least two domains 400), wherein each domain 400 comprises at least a portion of the polypeptide sequence of SEQ ID NO: 1. The plurality of domains 400 can be separated by a linker (e.g., a flexible linker) along the length of the engineered effector. EPIC-XV 1.47 can comprise a polypeptide sequence (e.g., 108 amino acid sequence long) that comprises three predicted vIRF2 activation domains 400 separated by flexible linker sequence.

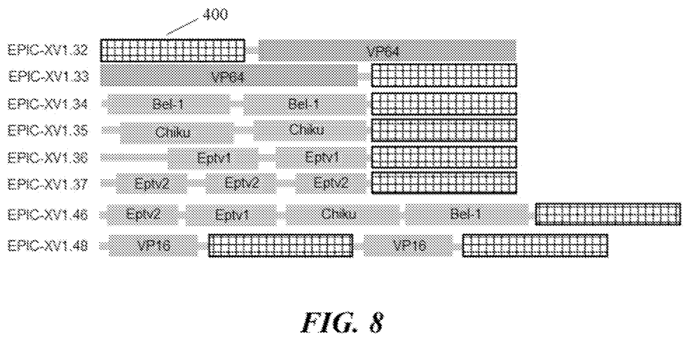

As shown in FIG. 8, example engineered effectors EPIC-XV 1.32 through 1.48 can each comprise a polypeptide sequence that is at least partially derived from the native vIRF2 amino acid sequence from Human herpesvirus 8. Each of the engineered effectors EPIC-XV 1.32-1.48 can comprise (i) at least a portion of the polypeptide sequence of SEQ ID NO: 1 (domain 400) and (ii) one or more additional polypeptide sequences that is not identical to the at least the portion of the polypeptide sequence of SEQ ID NO: 1. The polypeptide sequences (i) and (ii) can be separated by a flexible linker. In some embodiments, the engineered effectors EPIC-XV 1.32-1.48 can be designed to assess the combinatorial effect of (i) domain 400 and (ii) one or more additional effector domains (e.g., activation domains). Non-limiting examples of the one or more additional effector domains can include VP64 (EPIC-XV 1.32, EPIC-XV 1.33), VP16 (EPIC-XV 1.48), at least a portion of Bel-1 (transactivating Bel-1 protein in the Retroviridae Simian foamy virus genome) (EPIC-XV 1.34), at least a portion of Chiku (a non-structural polyprotein in the Chikungunya virus genome) (EPIC-XV 1.35), at least a portion of Eptvl (A-type inclusion protein (A0A220T6J0_9POXV) in the Eptesipox virus genome) (EPIC-XV 1.36), at least a portion of Eptv2 (an uncharacterized protein (A0A220T671_9POXV) in the Eptesipox virus genome) (EPIC-XV 1.37), and/or a combination thereof (EPIC-XV 1.46).

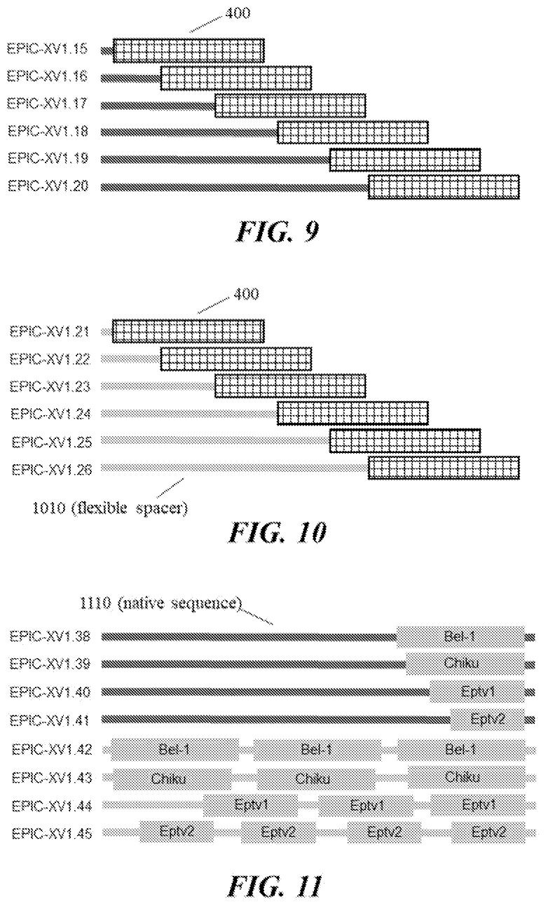

As shown in FIG. 9, example engineered effectors EPIC-XV 1.15 through 1.20 can each comprise a polypeptide sequence that is at least partially derived from the native vIRF2 amino acid sequence from Human herpesvirus 8. Each of the engineered effectors EPIC-XV 1.15-1.20 can comprise at least a portion of the polypeptide sequence of SEQ ID NO: 1 (domain 400) preceded by a varied length of the native amino acid sequence of vIRF2.

As shown in FIG. 10, example engineered effectors EPIC-XV 1.21 through 1.26 can each comprise a polypeptide sequence that is at least partially derived from the native vIRF2 amino acid sequence from Human herpesvirus 8. Each of the engineered effectors EPIC-XV 1.21-1.26 can comprise at least a portion of the polypeptide sequence of SEQ ID NO: 1 (domain 400) preceded by a varied length a flexible linker (1010).

As shown in FIG. 11, example engineered effectors EPIC-XV 1.38 through 1.41 can each comprise a polypeptide sequence comprising one or more domains derived from one or more members selected from the group consisting of Bel-1, Chiku, Eptv1, and Eptv2. In some embodiments, the engineered effector can consist of a native polypeptide sequence derived from Bel-1, Chiku, Eptv1, or Eptv2 (e.g., EPIC-XV 1.38-1.41, respectively). In some embodiments, different domains within each of the engineered effector can be separated by a flexible linker (e.g., EPIC-XV 1.42-1.45).

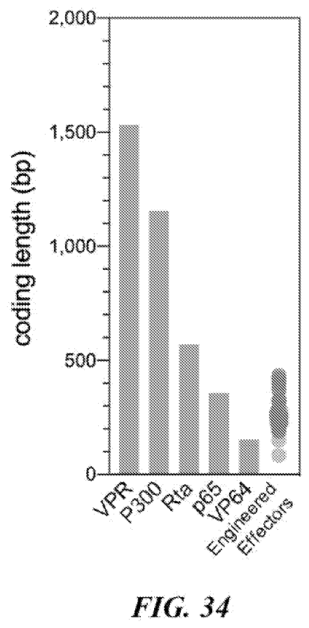

As shown in FIG. 34, the size (in bp) of the engineered gene effects with other benchmark activators such as VPR, P300, Rta, p65, and VP64.

### Example 4: Engineered Gene Effectors as Gene Activators

The engineered gene effectors as disclosed herein can be utilized (e.g., when in complex with a heterologous endonuclease, such as CRISPR/Cas protein, and optionally a guide nucleic acid sequence) to modulate (e.g., edit, activate, suppress) a target gene (e.g., heterologous gene, endogenous gene) in a cell. For example, the engineered gene effectors can effect activation of a target gene.

### A. Activation of a Heterologous Target Gene

HEK293T cells bearing a stably integrated TRE3G promoter-driven GFP expression reporter were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgTET) across all wells. Each well received 100 ng of modulator plasmid and 50 ng sgRNA plasmid, and experiments were performed in technical triplicate. Negative controls were dCas9 and dCasMini expression plasmids without any modulator fusion, co-transfected with targeting and non-targeting sgRNA plasmids. About 2 days (e.g., 48 hours) post-transfection, cells were analyzed by flow cytometry (Cytoflex LX) to monitor GFP expression, with analysis gates (FlowJo) to ensure measurements of live, singlet, and double-transfected cells to verify both modulator and sgRNA plasmid expression via mCherry and BFP fluorescence, respectively. Geometric mean of GFP fluorescence for each modulator was normalized against that of negative controls and reported as fold-change relative to negative controls.

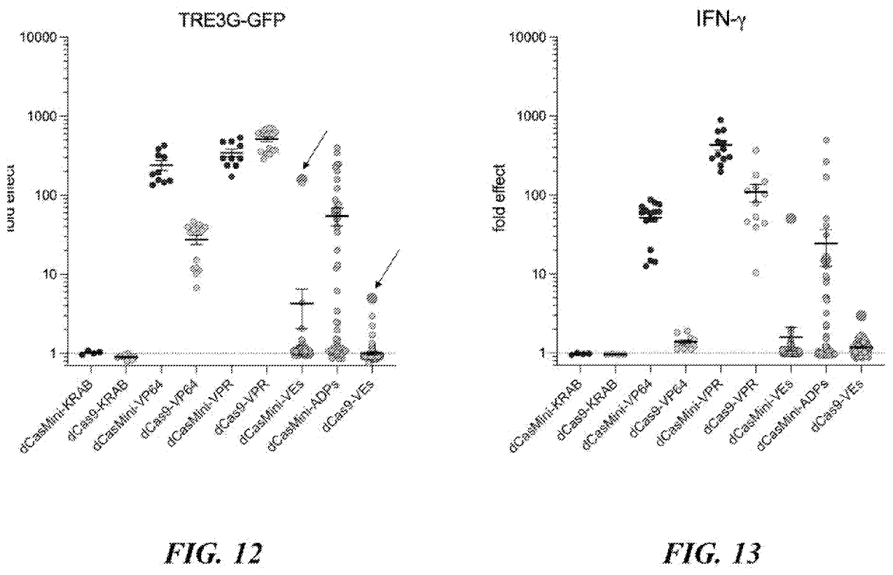

Referring to FIG. 12, plotted data displays relative GFP activation for dCas9- and dCasMini-canonical repressor controls (KRAB fusions) and canonical activator controls (VP64, VPR) for comparison against engineered gene effectors as disclosed herein. Individual VEs (unmodified screen hit modulators, n=95 independent candidates) show correlated activation independent of CRISPR system, i.e. dCasMini or dCas9. ADPs (engineered modulator variants or engineered effectors, as used interchangeably herein, n=48 independent candidates) show enhanced activation relative to original VEs, and performance comparable to or exceeding that of VPR fusions. Large dot (as indicated by the arrows) suggests EPIC-XV 1 unmodified modulator.

### B. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgIFNG) across all wells. Each well received 100 ng of modulator plasmid and 50 ng sgRNA plasmid, and experiments were performed in technical triplicate. Negative controls were dCas9 and dCasMini expression plasmids without any modulator fusion, co-transfected with targeting and non-targeting sgRNA plasmids. About 3 days (e.g., 72 hours) post-transfection, cell supernatants were collected to monitor IFNG protein expression by ELISA, after verifying both modulator and sgRNA plasmid expression via mCherry and BFP fluorescence, respectively, by fluorescence microscopy (EVOS FL). IFNG protein concentration for each modulator was normalized against that of negative controls and reported as fold-change relative to negative controls.

Referring to FIG. 13, plotted data displays relative IFNG activation for dCas9- and dCasMini-canonical repressor controls (KRAB fusions) and canonical activator controls (VP64, VPR) for comparison against engineered gene effectors. Individual VEs (unmodified screen hit modulators, n=95 independent candidates) show correlated activation independent of CRISPR system, i.e. dCasMini or dCas9. ADPs (engineered modulator variants or engineered effectors, as used interchangeably herein, n=48 independent candidates) show enhanced activation relative to original VEs, and performance comparable to or exceeding that of VPR fusions. Large dot indicates EPIC-XV 1 unmodified modulator.

### C. Activation of a Heterologous Target Gene

HEK293T cells bearing a stably integrated TRE3G promoter-driven GFP expression reporter were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgTET) across all wells. Each well received 100 ng of modulator plasmid and 50 ng sgRNA plasmid, and experiments were performed in technical triplicate. Negative controls were dCas9 and dCasMini expression plasmids without any modulator fusion, co-transfected with targeting and non-targeting sgRNA plasmids. 5 days post-transfection, cells were analyzed by flow cytometry (Cytoflex LX) to monitor GFP expression, with analysis gates (FlowJo) to ensure measurements of live, singlet, and double-transfected cells to verify both modulator and sgRNA plasmid expression via mCherry and BFP fluorescence, respectively. Geometric mean of GFP fluorescence for each modulator was normalized against that of negative controls and reported as fold-change relative to negative controls.

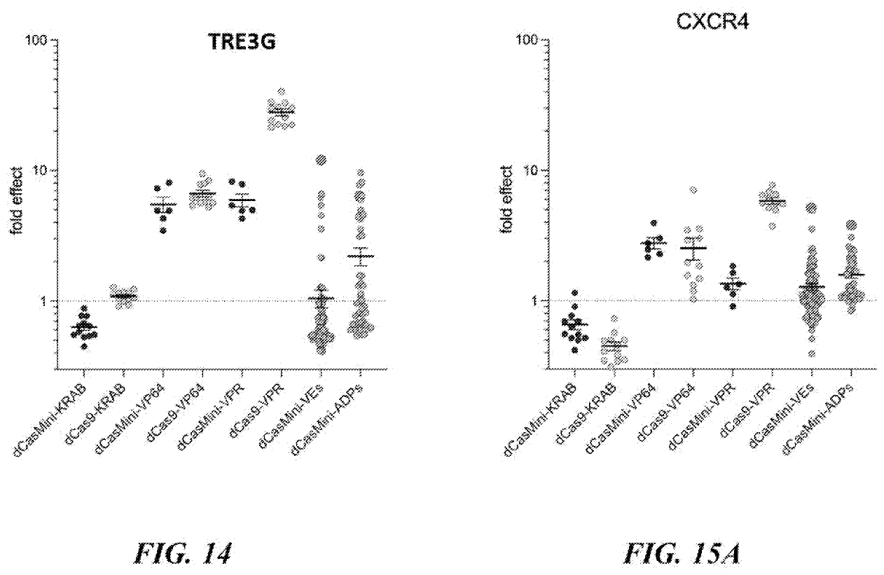

Referring to FIG. 14, plotted data displays relative GFP activation for dCas9- and dCasMini-canonical repressor controls (KRAB fusions) and canonical activator controls (VP64, VPR) for comparison against engineered gene effectors. Individual VEs (unmodified screen hit modulators, n=95 independent candidates) show correlated activation independent of CRISPR system, i.e. dCasMini or dCas9. ADPs (engineered modulator variants or engineered effectors, as used interchangeably herein, n=48 independent candidates) show enhanced activation relative to original VEs, and performance comparable to or exceeding that of VPR fusions. Large dot indicates EPIC-XV 1 unmodified modulator.

### D. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgCXCR4) across all wells. Each well received 100 ng of modulator plasmid and 50 ng sgRNA plasmid, and experiments were performed in technical triplicate. Negative controls were dCas9 and dCasMini expression plasmids without any modulator fusion, co-transfected with targeting and non-targeting sgRNA plasmids. 7 days post-transfection, cells were stained with CXCR4-APC conjugated antibody (BioLegend) and analyzed by flow cytometry (Cytoflex LX) to monitor CXCR4 protein expression, with analysis gates (FlowJo) to ensure measurements of live, singlet, and double-transfected cells to verify both modulator and sgRNA plasmid expression via mCherry and BFP fluorescence, respectively. Geometric mean of APC fluorescence for each modulator was normalized against that of negative controls and reported as fold-change relative to negative controls.

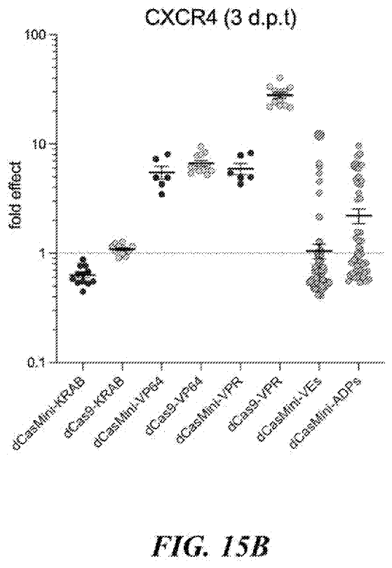

Referring to FIG. 15A, plotted data displays relative CXCR4 activation for dCas9- and dCasMini-canonical repressor controls (KRAB fusions) and canonical activator controls (VP64, VPR) for comparison against engineered gene effectors. Individual VEs (unmodified screen hit modulators, n=95 independent candidates) show correlated activation independent of CRISPR system, i.e. dCasMini or dCas9. ADPs (engineered modulator variants or engineered effectors, as used interchangeably herein, n=48 independent candidates) show enhanced activation relative to original VEs, and performance comparable to or exceeding that of VPR fusions. Large dot indicates EPIC-XV 1 unmodified modulator.

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgCXCR4) across all wells. Each well received 100 ng of modulator plasmid and 50 ng sgRNA plasmid, and experiments were performed in technical triplicate. Negative controls were dCas9 and dCasMini expression plasmids without any modulator fusion, co-transfected with targeting and non-targeting sgRNA plasmids. 72 hours post-transfection, cells were stained with CXCR4-APC conjugated antibody (BioLegend) and analyzed by flow cytometry (Cytoflex LX) to monitor CXCR4 protein expression, with analysis gates (FlowJo) to ensure measurements of live, singlet, and double-transfected cells to verify both modulator and sgRNA plasmid expression via mCherry and BFP fluorescence, respectively. Geometric mean of APC fluorescence for each modulator was normalized against that of negative controls and reported as fold-change relative to negative controls.

Referring to FIG. 15B, plotted data displays relative CXCR4 activation for dCas9- and dCasMini-canonical repressor controls (KRAB fusions) and canonical activator controls (VP64, VPR) for comparison against engineered effectors. Individual VEs (unmodified screen hit modulators, n=95 independent candidates) show correlated activation independent of CRISPR system, i.e. dCasMini or dCas9. ADPs (engineered modulator variants or engineered effectors, as used interchangeably herein, n=48 independent candidates) show enhanced activation relative to original VEs, and performance comparable to or exceeding that of VPR fusions. Large dot indicates EPIC-XV 1 unmodified modulator.

### E. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgCD2) across all wells. Each well received 100 ng of modulator plasmid and 50 ng sgRNA plasmid, and experiments were performed in technical triplicate. Negative controls were dCasMini expression plasmid without any modulator fusion, co-transfected with targeting and non-targeting sgRNA plasmids. 72 hours post-transfection, cells were stained with CD2-APC conjugated antibody (BioLegend) and analyzed by flow cytometry (Cytoflex LX) to monitor CD2 protein expression, with analysis gates (FlowJo) to ensure measurements of live, singlet, and double-transfected cells to verify both modulator and sgRNA plasmid expression via mCherry and BFP fluorescence, respectively. Geometric mean of APC fluorescence for each modulator was normalized against that of negative controls and reported as fold-change relative to negative controls.

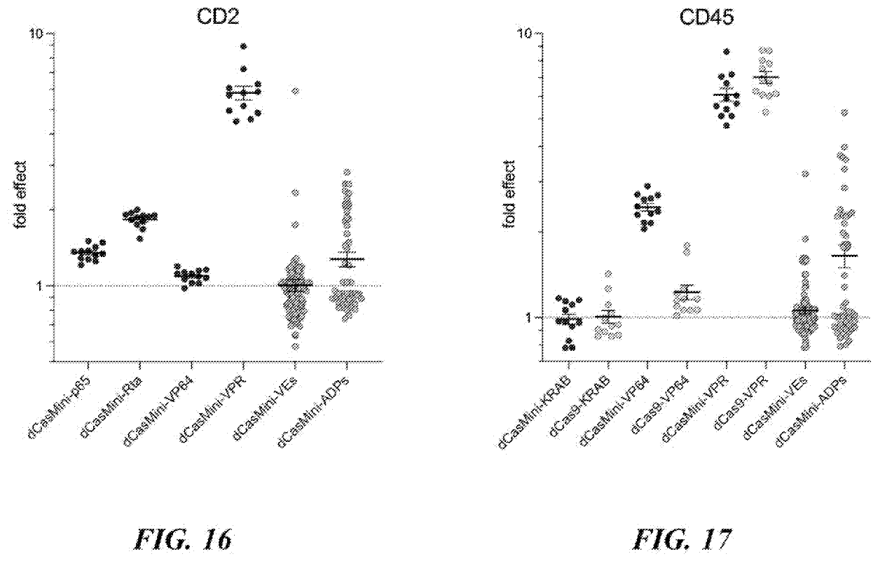

Referring to FIG. 16, plotted data displays relative CD2 activation for dCasMini-canonical activator controls (p65, Rta, VP64, VPR) for comparison against engineered gene effectors. Individual VEs (unmodified screen hit modulators, n=95 independent candidates) show activation comparable to or exceeding controls. ADPs (engineered modulator variants or engineered effectors, as used interchangeably herein, n=48 independent candidates) show enhanced activation relative to original VEs, and performance comparable to or exceeding that of VPR fusions. Large dot indicates EPIC-XV 1 unmodified modulator.

### F. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgCD45) across all wells. Each well received 100 ng of modulator plasmid and 50 ng sgRNA plasmid, and experiments were performed in technical triplicate. Negative controls were dCas9 and dCasMini expression plasmids without any modulator fusion, co-transfected with targeting and non-targeting sgRNA plasmids. 48 hours post-transfection, cells were stained with CD45-APC conjugated antibody (BioLegend) and analyzed by flow cytometry (Cytoflex LX) to monitor CD45 protein expression, with analysis gates (FlowJo) to ensure measurements of live, singlet, and double-transfected cells to verify both modulator and sgRNA plasmid expression via mCherry and BFP fluorescence, respectively. Geometric mean of APC fluorescence for each modulator was normalized against that of negative controls and reported as fold-change relative to negative controls.

Referring to FIG. 17, plotted data displays relative CD45 activation for dCas9- and dCasMini-canonical repressor controls (KRAB fusions) and canonical activator controls (VP64, VPR) for comparison against engineered gene effectors. Individual VEs (unmodified screen hit modulators, n=95 independent candidates) show correlated activation independent of CRISPR system, i.e. dCasMini or dCas9. ADPs (engineered modulator variants or engineered effectors, as used interchangeably herein, n=48 independent candidates) show enhanced activation relative to original VEs, and performance comparable to or exceeding that of VP64 fusions. Large dot indicates EPIC-XV 1 unmodified modulator.

### G. Activation of a Heterologous Target Gene

HEK293T cells bearing a stably integrated TRE3G promoter-driven GFP expression reporter were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgTET) across all wells. Each well received 100 ng of modulator plasmid and 50 ng sgRNA plasmid, and experiments were performed in technical triplicate. Negative controls were dCasMini expression plasmids without any modulator fusion, co-transfected with targeting and non-targeting sgRNA plasmids. 48 hours post-transfection, cells were analyzed by flow cytometry (Cytoflex LX) to monitor GFP expression, with analysis gates (FlowJo) to ensure measurements of live, singlet, and double-transfected cells to verify both modulator and sgRNA plasmid expression via mCherry and BFP fluorescence, respectively. Geometric mean of GFP fluorescence for each modulator was normalized against that of negative controls and reported as fold-change relative to negative controls.

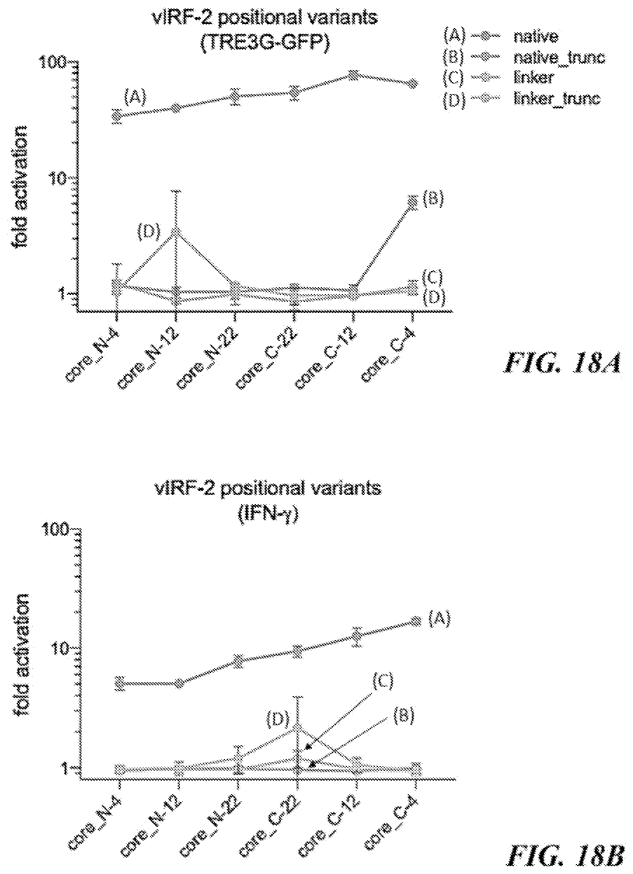

Referring to FIG. 18A, plotted data displays relative GFP activation for dCasMini-ADPs (engineered modulator variants or engineered effectors, as used interchangeably herein) comprising the predicted (ADPred) core domain of vIRF2 flanked either by its native genomic sequence, or by GS linker sequence. Nomenclature corresponds to flanking sequence length up- or downstream of the core domain, ranging from 4-22 amino acids distance from the N- or C-terminal. Truncated variants are also indicated, in which a STOP codon is inserted immediately following the core domain to shorten the overall construct length. Activation performance indicates positional effects with greater strength as the core domain approaches the C-terminal, i.e. distal from the dCasMini.

### H. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgIFNG) across all wells. Each well received 100 ng of modulator plasmid and 50 ng sgRNA plasmid, and experiments were performed in technical triplicate. Negative controls were dCasMini expression plasmids without any modulator fusion, co-transfected with targeting and non-targeting sgRNA plasmids. 72 hours post-transfection, cell supernatants were collected to monitor IFNG protein expression by ELISA, after verifying both modulator and sgRNA plasmid expression via mCherry and BFP fluorescence, respectively, by fluorescence microscopy (EVOS FL). IFNG protein concentration for each modulator was normalized against that of negative controls and reported as fold-change relative to negative controls.

Referring to FIG. 18B, plotted data displays relative IFNG (IFN gamma) activation for dCasMini-ADPs (engineered modulator variants or engineered effectors, as used interchangeably herein) consisting of the predicted (ADPred) core domain of vIRF2 flanked either by its native genomic sequence, or by GS linker sequence. Nomenclature corresponds to flanking sequence length up- or downstream of the core domain, ranging from 4-22 amino acids distance from the N- or C-terminal. Truncated variants are also indicated, in which a STOP codon is inserted immediately following the core domain to shorten the overall construct length. Activation performance indicates positional effects with greater strength as the core domain approaches the C-terminal, i.e. distal from the dCasMini.

### I. Activation of a Heterologous Target Gene

HEK293T cells bearing a stably integrated TRE3G promoter-driven GFP expression reporter were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgTET) across all wells. Each well received 100 ng of modulator plasmid and 50 ng sgRNA plasmid, and experiments were performed in technical triplicate. Negative controls were dCasMini expression plasmids without any modulator fusion, co-transfected with targeting and non-targeting sgRNA plasmids. 48 hours post-transfection, cells were analyzed by flow cytometry (Cytoflex LX) to monitor GFP expression, with analysis gates (FlowJo) to ensure measurements of live, singlet, and double-transfected cells to verify both modulator and sgRNA plasmid expression via mCherry and BFP fluorescence, respectively. Geometric mean of GFP fluorescence for each modulator was normalized against that of negative controls and reported as fold-change relative to negative controls.

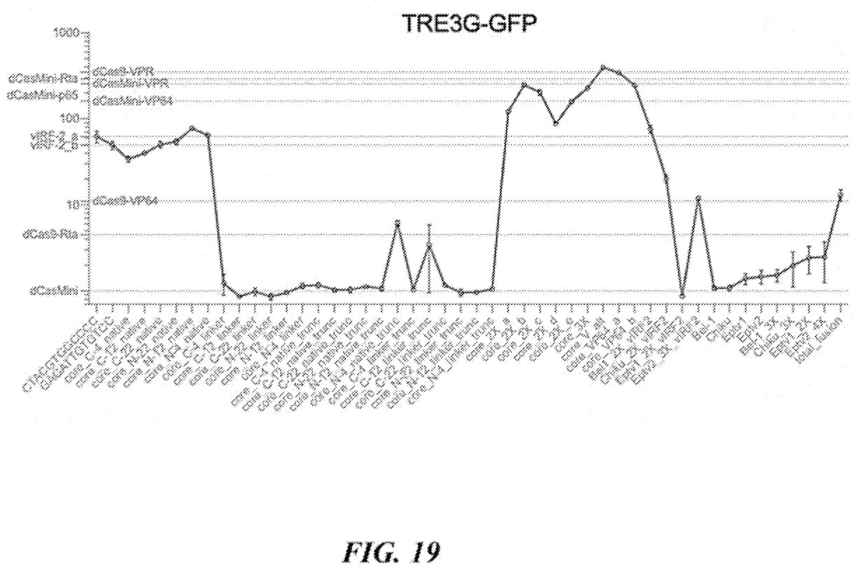

Referring to FIG. 19, potted data displays relative GFP activation for dCasMini-ADPs (engineered modulator variants or engineered effectors, as used interchangeably herein), relative to control activators as indicated by horizontal bars. Strongest activation is achieved by ADP variants consisting of the predicted core domain flanked by native sequence, or arranged in tandem repeat array either with itself or other core domains as indicated.

### J. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgIFNG) across all wells. Each well received 100 ng of modulator plasmid and 50 ng sgRNA plasmid, and experiments were performed in technical triplicate. Negative controls were dCasMini expression plasmids without any modulator fusion, co-transfected with targeting and non-targeting sgRNA plasmids. 72 hours post-transfection, cell supernatants were collected to monitor IFNG protein expression by ELISA, after verifying both modulator and sgRNA plasmid expression via mCherry and BFP fluorescence, respectively, by fluorescence microscopy (EVOS FL). IFNG protein concentration for each modulator was normalized against that of negative controls and reported as fold-change relative to negative controls.

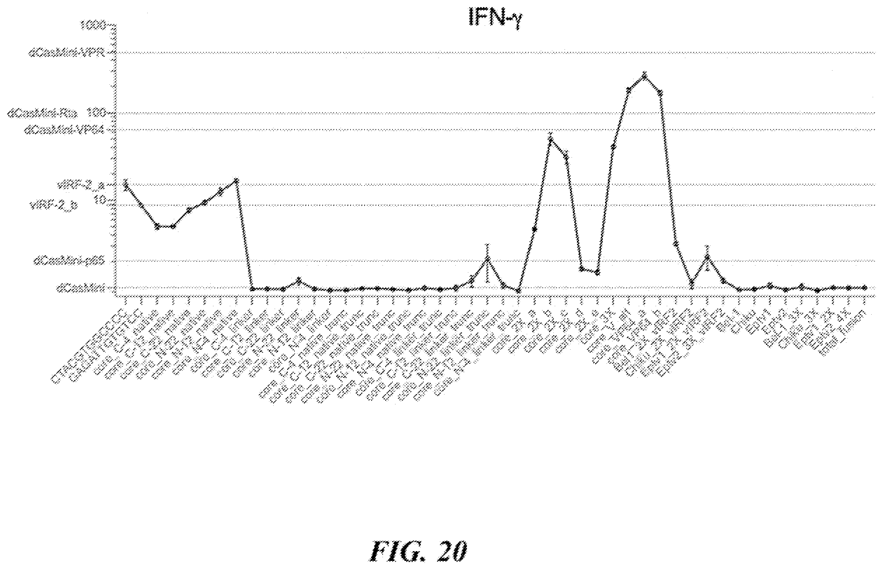

Referring to FIG. 20, plotted data displays relative IFNG activation for dCasMini-ADPs (engineered modulator variants or engineered effectors, as used interchangeably herein), relative to control activators as indicated by horizontal bars. Strongest activation is achieved by ADP variants consisting of the predicted core domain flanked by native sequence, or arranged in tandem repeat array either with itself or other core domains as indicated.

### K. Activation of an Endogenous Target Gene

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA (sgCXCR4) across all wells. Each well received 100 ng of modulator plasmid and 50 ng sgRNA plasmid, and experiments were performed in technical triplicate. Negative controls were dCasMini expression plasmid without any modulator fusion, co-transfected with targeting and non-targeting sgRNA plasmids. 7 days post-transfection, cells were stained with CXCR4-APC conjugated antibody (BioLegend) and analyzed by flow cytometry (Cytoflex LX) to monitor CXCR4 protein expression, with analysis gates (FlowJo) to ensure measurements of live, singlet, and double-transfected cells to verify both modulator and sgRNA plasmid expression via mCherry and BFP fluorescence, respectively. Geometric mean of APC fluorescence for each modulator was normalized against that of negative controls and reported as fold-change relative to negative controls.

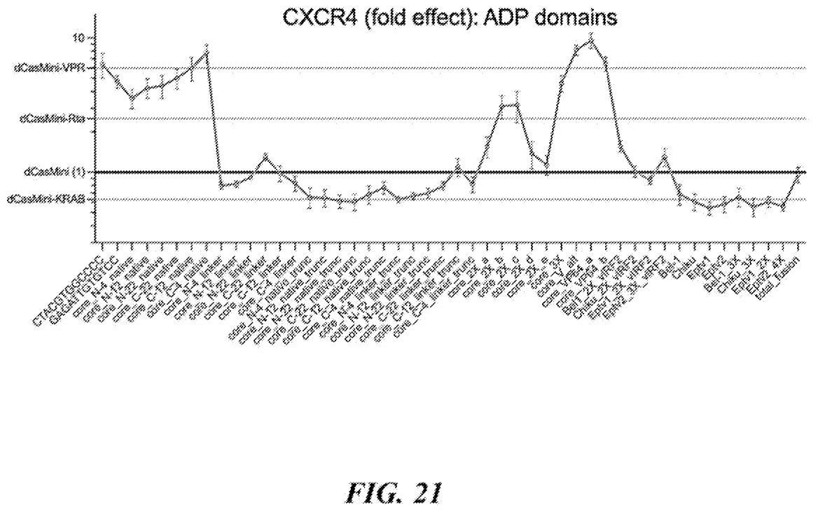

Referring to FIG. 21, plotted data displays relative CXCR4 activation for dCasMini-ADPs (engineered modulator variants or engineered effectors, as used interchangeably herein), relative to control activators as indicated by horizontal bars. Strongest activation is achieved by ADP variants consisting of the predicted core domain flanked by native sequence, or arranged in tandem repeat array either with itself or other core domains as indicated.

### Example 5: Mean Performance of Engineered Gene Effectors for Modulating Heterologous and Endogenous Genes

Efficacy of the engineered gene effectors as disclosed herein (e.g., as gene activators) can be represented by generating a mean performance of each engineered gene effector in modulating (e.g., activating) a plurality of genes (e.g., including one or more heterologous genes, such as the TRE3G-GFP as disclosed herein, and one or more endogenous genes, such as IFN-gamma, CXCR4, CD2, CD45, etc. as disclosed herein), as compared to a control gene activator (e.g., VPR).

Wild-type or GFP reporter HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA across all wells. Each well received 100 ng of modulator plasmid and 50 ng sgRNA plasmid, and experiments were performed in technical triplicate. Negative controls were dCasMini expression plasmid without any modulator fusion, co-transfected with targeting and non-targeting sgRNA plasmids.

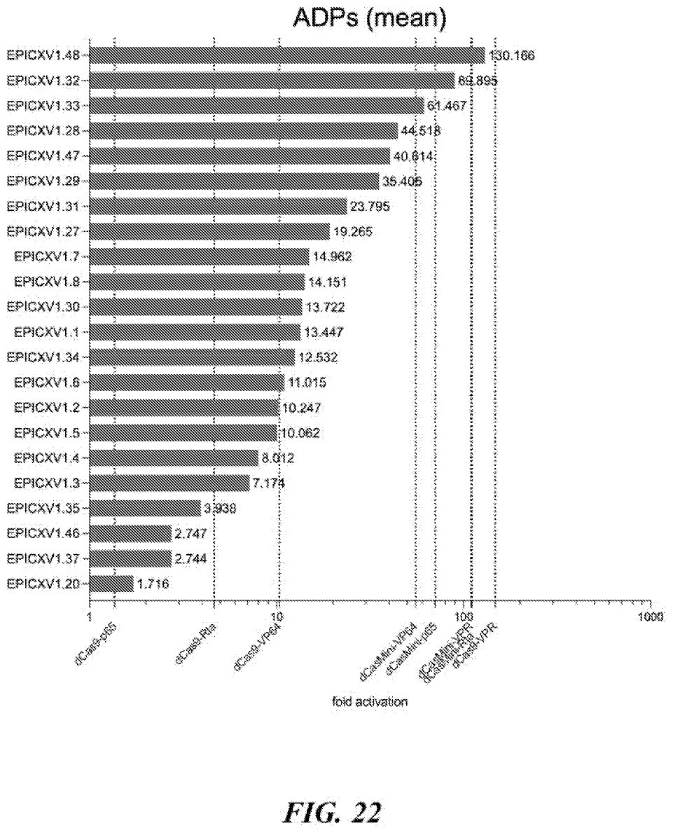

Referring to FIG. 22, plotted data represents mean activation of all targets tested by the indicated ADP modulator variants, and that of benchmark controls is represented as vertical bars for relative performance. Variants displayed are those that exceeding the minimal activator benchmark, dCas9-p65. Interestingly, control activator domains p65 and Rta vary in performance with increased strength when fused to dCasMini rather than dCas9. Plotted values of the control gene effectors (e.g., dCasMini coupled with VPR, Rta, VP64, or p65) are shown in TABLE 5.

TABLE 5

| | |
|---|---|
| dCas9-VPR | 147.564 |
| dCasMini-Rta | 110.729 |
| dCasMini-VPR | 109.346 |
| dCasMini-p65 | 70.404 |
| dCasMini-VP64 | 55.323 |
| dCas9-VP64 | 10.360 |
| dCas9-Rta | 4.637 |
| dCas9-p65 | 1.360 |

### Example 6: Mean Performance of Engineered Gene Effectors for Modulating Endogenous Genes

Efficacy of the engineered gene effectors as disclosed herein (e.g., as gene activators) can be represented by generating a mean performance of each engineered gene effector in modulating (e.g., activating) a plurality of endogenous genes (e.g., IFN-gamma, CXCR4, CD2, CD45, etc. as disclosed herein), as compared to a control gene activator (e.g., VPR).

Wild-type HEK293T cells were seeded in 96-well plates at a density of 20,000 cells per well. 16 hours after seeding, cells were co-transfected with equal mass of modulator- and sgRNA-expressing plasmids in arrayed format such that each well received a single modulator construct to be tested, but the same targeting sgRNA across all wells. Each well received 100 ng of modulator plasmid and 50 ng sgRNA plasmid, and experiments were performed in technical triplicate. Negative controls were dCasMini expression plasmid without any modulator fusion, co-transfected with targeting and non-targeting sgRNA plasmids.

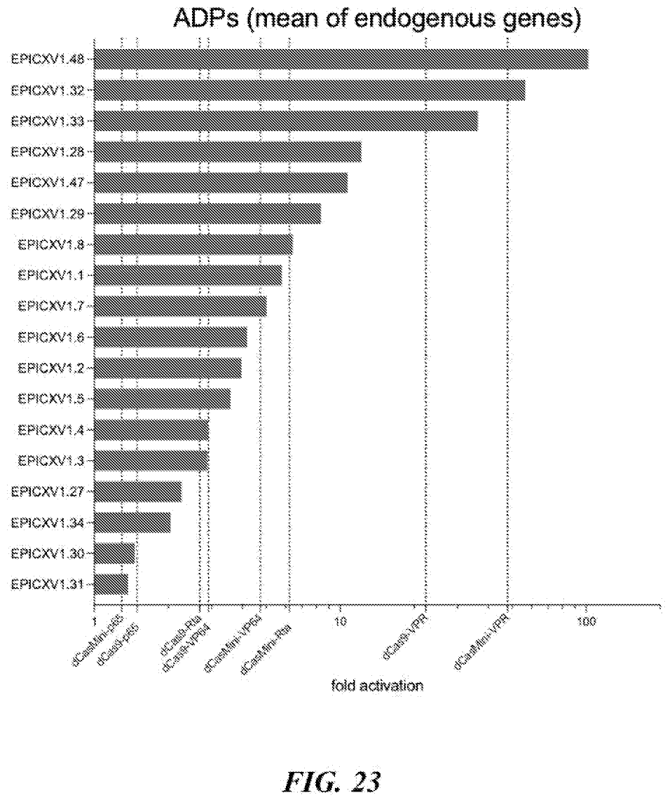

Referring to FIG. 23, plotted data represents mean activation of all targets tested by the indicated ADP modulator variants, and that of benchmark controls is represented as vertical bars for relative performance. Variants displayed are those that exceeding the minimal activator benchmark, dCas9-p65. Interestingly, control activator domains p65 and Rta vary in performance with increased strength when fused to dCasMini rather than dCas9. Top 3 novel modulators outperform even the strongest benchmarks (VPR). Plotted values of the control gene effectors (e.g., dCasMini coupled with VPR, Rta, VP64, or p65) are shown in TABLE 6.

TABLE 6

| | |
|---|---|
| dCasMini-VPR | 47.854 |
| dCas9-VPR | 22.286 |
| dCasMini-Rta | 6.207 |
| dCasMini-VP64 | 4.735 |
| dCas9-VP64 | 2.913 |
| dCas9-Rta | 2.684 |
| dCas9-p65 | 1.494 |
| dCasMini-p65 | 1.293 |

### Example 7: Engineered Gene Effectors Induce Persistent and Sustained Gene Activation and Gene Suppression

Persistent and sustained gene modulations (e.g., durable gene modulations) can be advantageous as compared to transient modulation, e.g., when a prolonged control over the gene expression profile is needed to elicit a desired cellular effect.

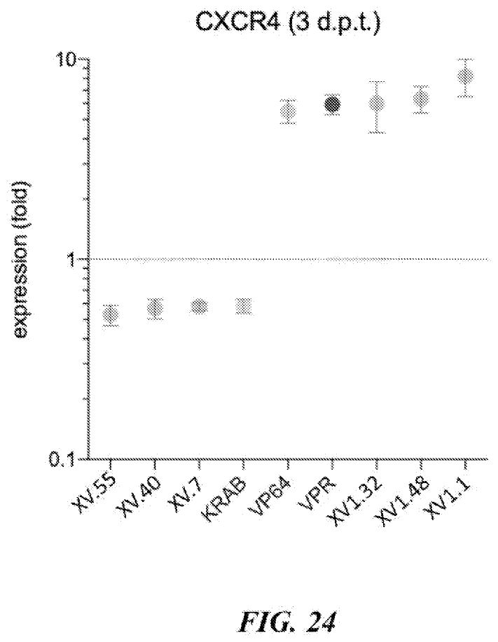

As demonstrated in FIGS. 24-30, the engineered modulators described herein exhibited persistent and sustained modulation (e.g., gene activation or gene suppression) of various target genes.

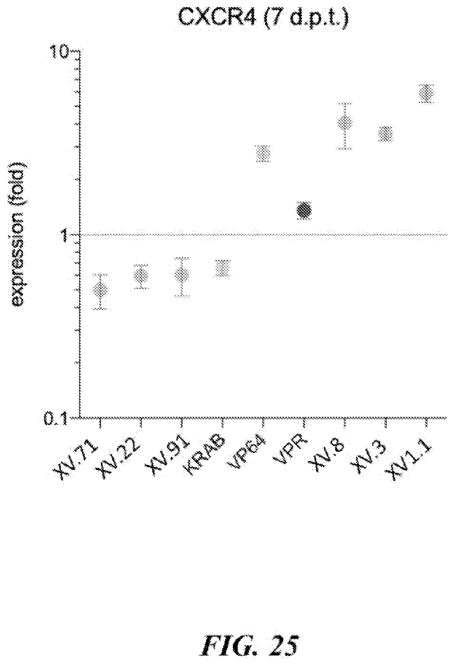

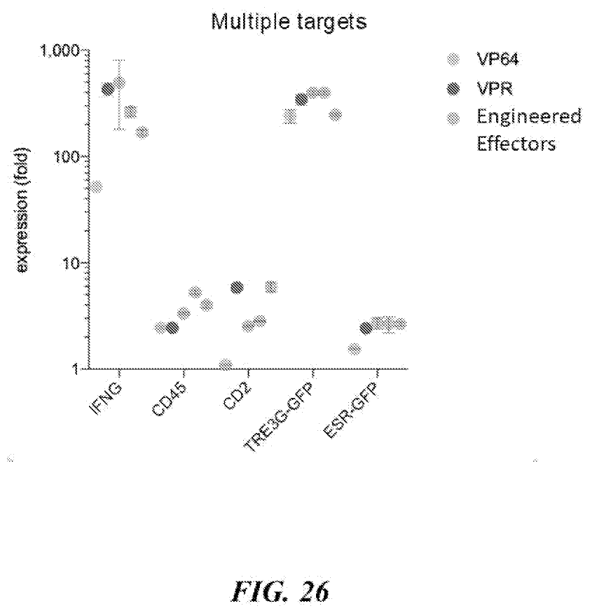

Referring to FIGS. 24-25, plotted data shows the fold changes in CXCR4 expression 3 days or 7 days post transfection by engineered gene activators (XV1.32, XV1.48, XV1.1), screened activators (XV.8, XV.3), screened gene repressor (XV.55, XV.40, XV.71, XV.22, XV.91), canonical repressor control (KRAB), and canonical activator controls (VP64, VPR). FIG. 26 demonstrates the effects of engineered gene effectors as disclosed herein on modulating a plurality of genes (e.g., including one or more heterologous genes, such as the TRE3G-GFP, ESR-GFP as disclosed herein, and one or more endogenous genes, such as IFN-gamma, CD2, CD45, etc. as disclosed herein), as compared to the control gene activators (e.g., VPR, VP64).

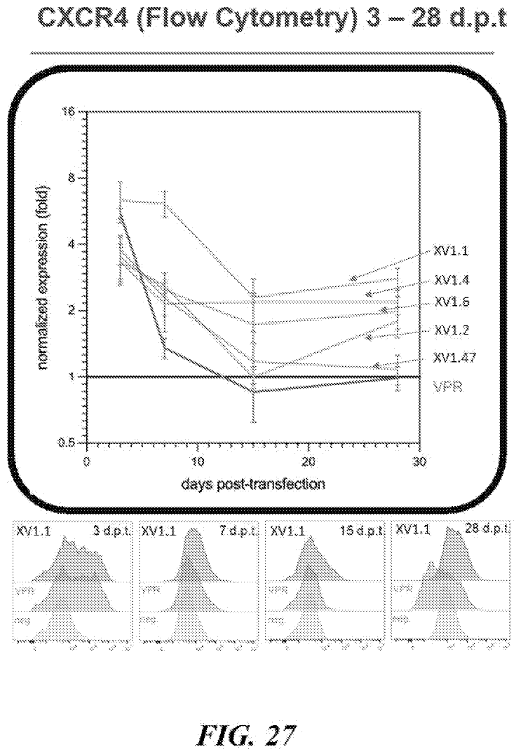

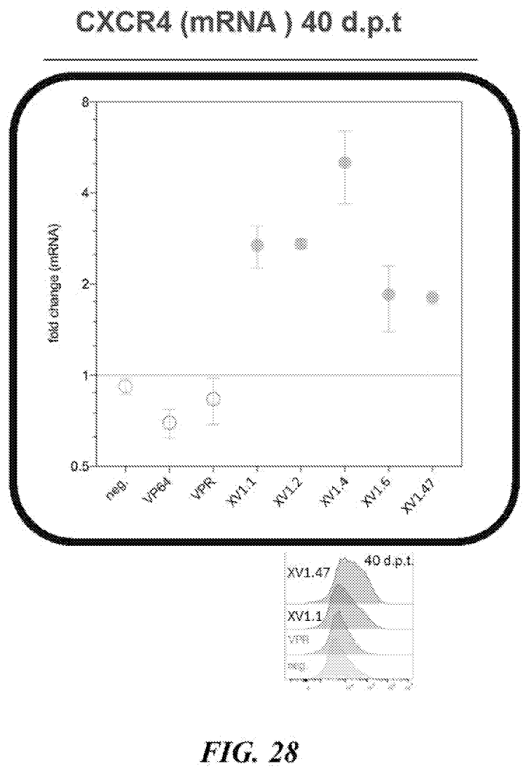

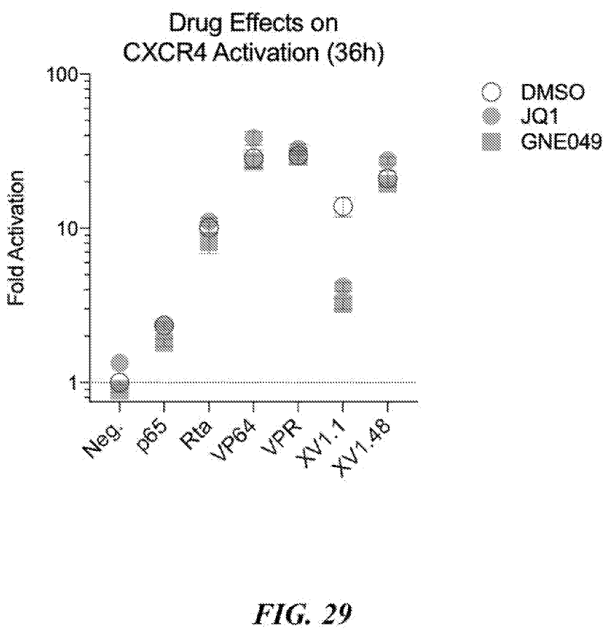

Referring to FIGS. 27-28, plotted data displays a persistent and sustainable gene activation (e.g., CXCR4 as disclosed herein) by the engineered gene effectors (e.g., engineered gene activator, such as XV1.1, XV1.4, XV1.6, XV1.2, XV1.47) 28 days and 40 days post transfection. Referring to FIG. 29, the effects of engineered gene activators (XV1.1, XV1.48) on a target gene expression (CXCR4) was at least partially reversed upon treatment with small molecules inhibitors: JQ1 or GNE049. Upon treatment with JQ1 or GNE049, the observed activation of the engineered gene effectors (e.g., XV1.1) was reversed, but not for some of the engineered gene effectors (e.g., XV1.48) and the control gene activators (p65, Rta, VP64 and VPR).

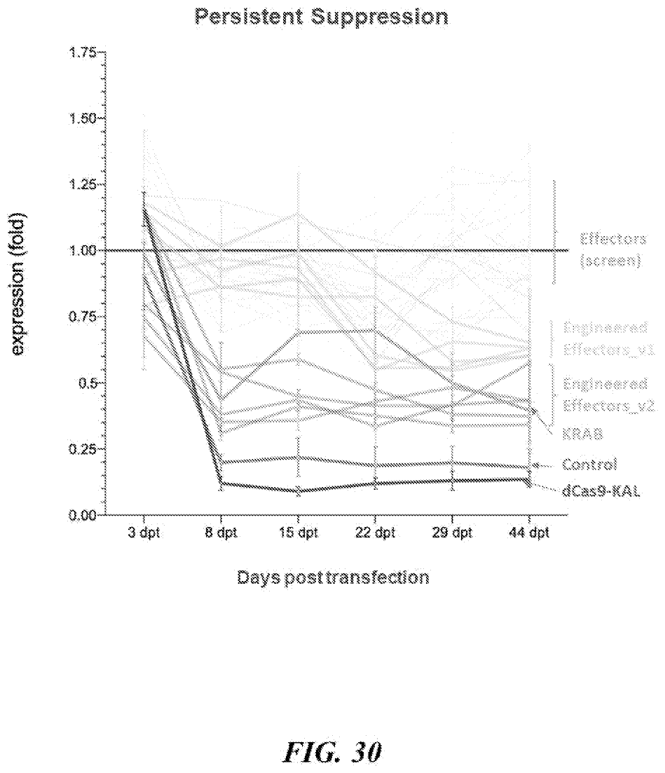

Referring to FIG. 30, plotted data displays a persistent and sustainable gene repression by the engineered gene effectors (e.g., engineered gene repressor, such as Engineered Effectors-v1, Engineered Effectors-v2) and repressor controls (KRAB and KAL) 3 days, 8 days, 15 days, 22 days, 29 days and 44 days post transfection.

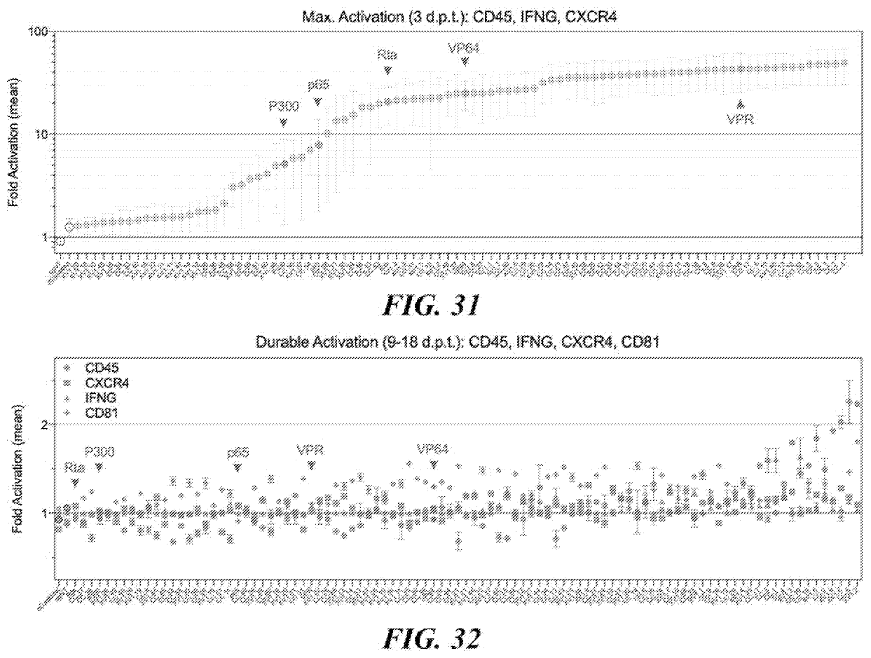

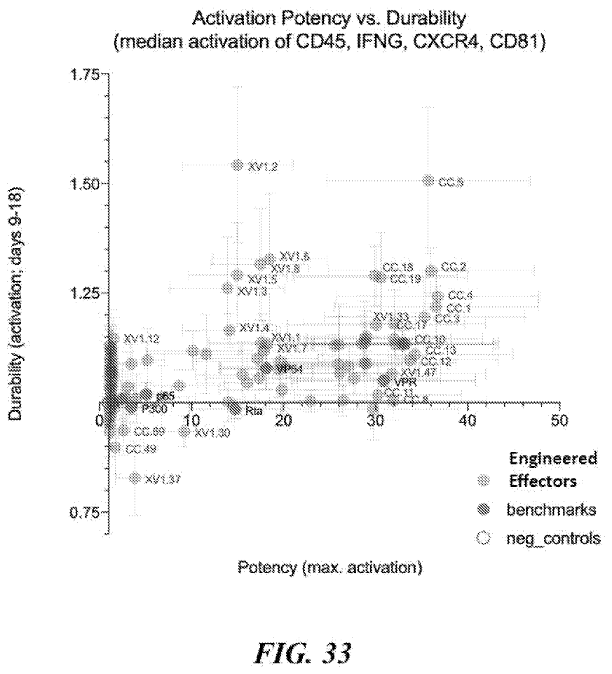

As demonstrated in FIGS. 31-33, the engineered effectors described herein show varying gene activation and durability profiles. Referring to FIG. 31, plotted data shows the mean activation (e.g., at 3 days post transfection) of CD45, IFNG, and CXCR4 by the engineered gene effectors (e.g., gene activator) and activator controls (e.g., P300, p65, Rta, VP64 and VPR). Referring to FIG. 32, plotted data shows sustained gene activations (e.g., durable activation) of CD45, IFNG, CXCR4, and CD81 by the engineered gene effectors (e.g., gene activator) and activator controls at 9-18 days post transfection (dpt). Referring to FIG. 33, plotted data shows the median activation of CD45, IFNG, CXCR4, and CD81 by the engineered gene effector and activator controls at early (day 3) and at late (day 9-18) time points.

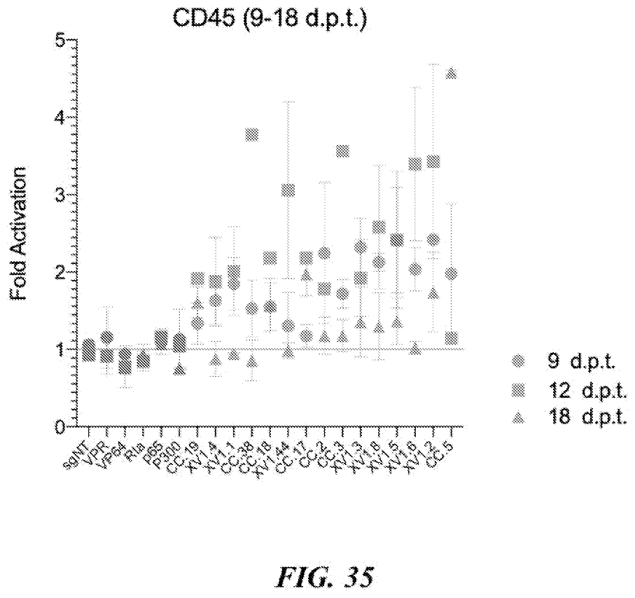

Referring to FIG. 35, plotted data shows CD45 expression level at 9 days, 12, days, or 18 days post transfection by the engineered gene effectors (e.g., CC.5, XV 1.2) and activator controls (e.g., VPR, VP64, Rta, p65, P300), confirming the persistent and sustainable gene activation by the engineered gene effectors.

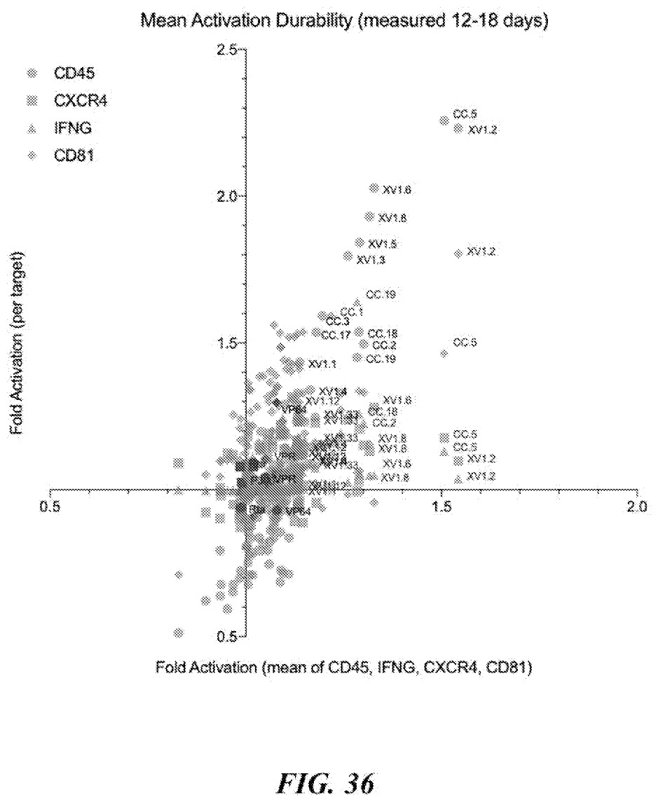

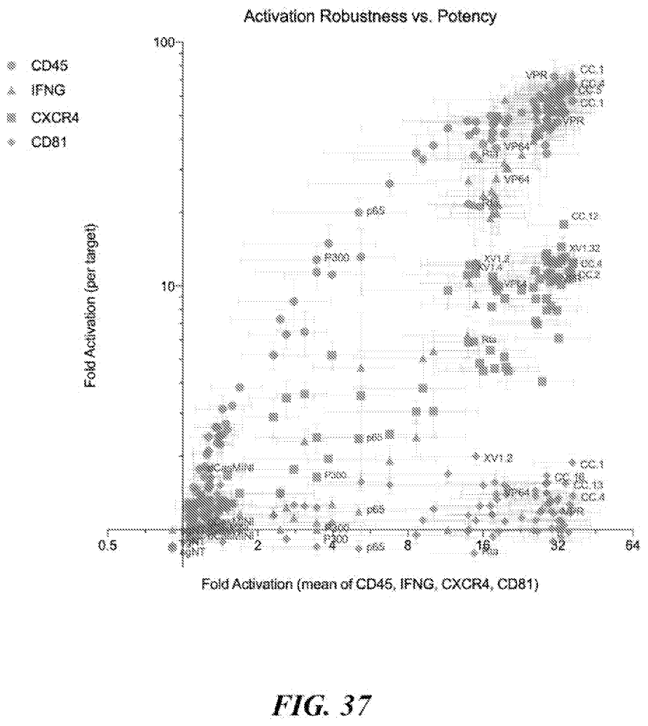

Referring to FIGS. 36-37, some engineered gene effectors showed high potency (e.g., activation) but low durability (e.g., persistency), while some other engineered gene effectors showed high potency and high durability. The data further confirms that the initial potency can, but does not always, correlate with durability. FIG. 36 shows the mean activation durability of the engineered gene effectors by comparing the robustness (mean performance across multiple target genes: CD45, IFN-g, CXCR4 and CD81) and the potency (fold activation per each target) at 12-18 days post transfection. The top-performing modulators include CC.5, XV1.2, and XV.16. FIG. 37 shows activation robustness and potency of the engineered gene effectors by measuring the robustness (mean performance across multiple target genes: CD45, IFN-g, CXCR4 and CD81) and the potency (fold activation per each target) at 3 days post transfection. The top-performing modulators include CC.1, CC.4, CC.5, XV1.32, and XV1.2.

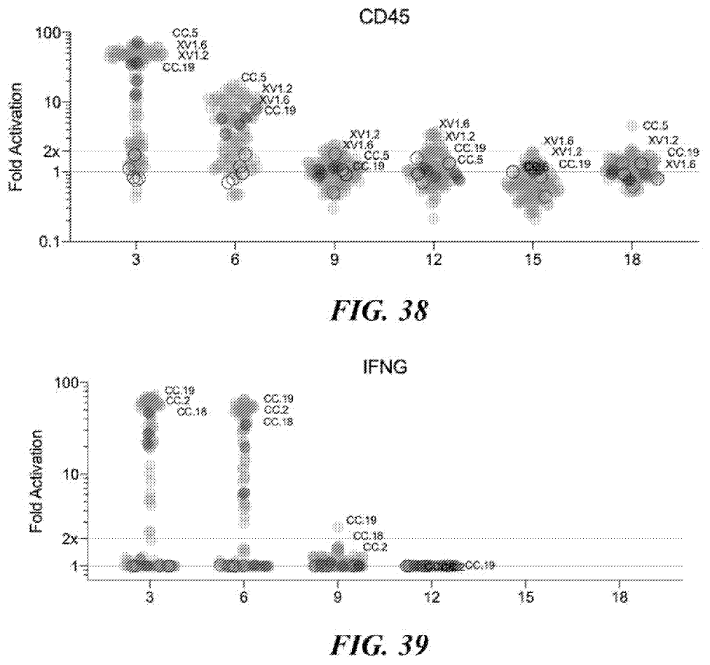

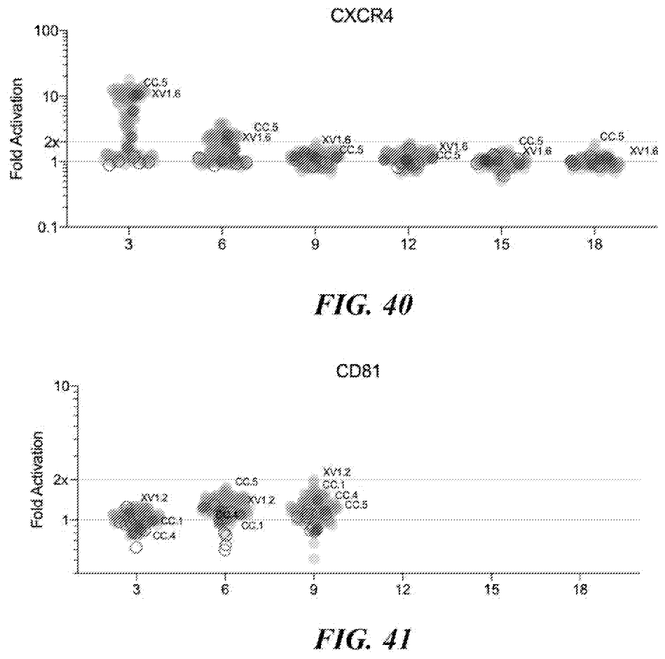

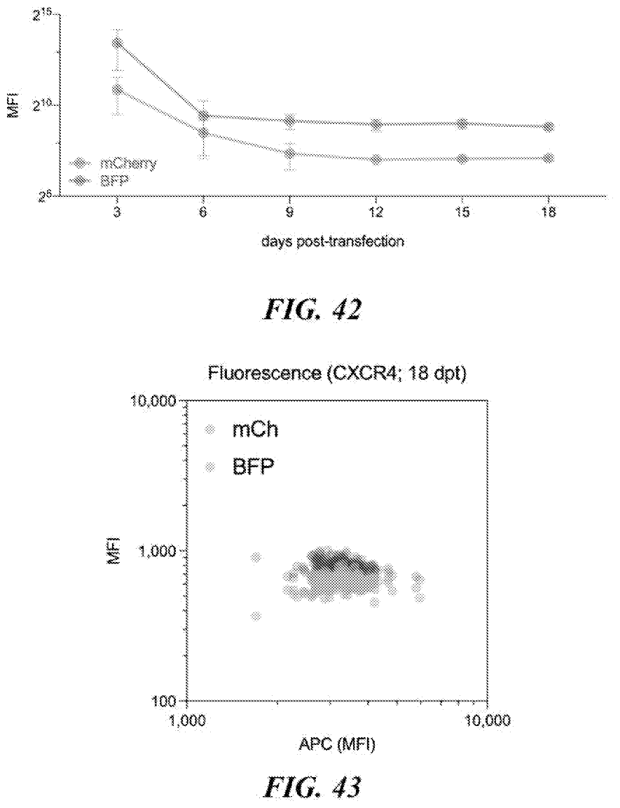

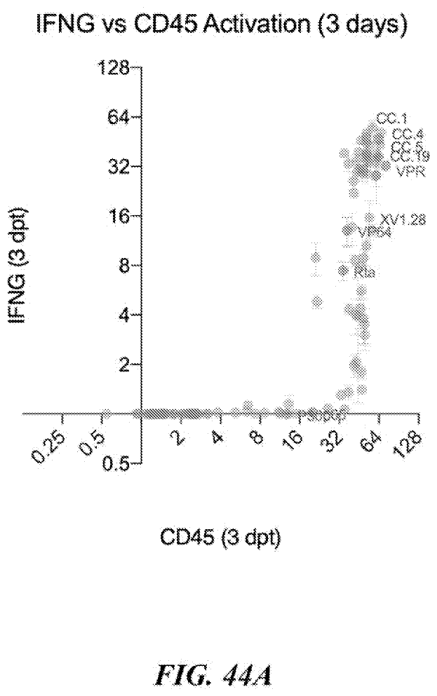

Referring to FIGS. 38-43, plotted data shows that the engineered gene effectors (such as CC.5, XV1.6, XV1.2, CC.19, CC.2, and CC18) resulted in persistent and sustained activation of target genes (e.g., CD45, IFNy, CXCR4, and CD81) for up to 18 days post-transfection, following transient delivery of the system described herein. This was demonstrated by the continued activation of the target genes (FIGS. 38-41), even though the expression of the engineered modulator (mCherry) and sgRNA (BFP) plasmids fluorescence were reduced by at least 2-fold in the system.

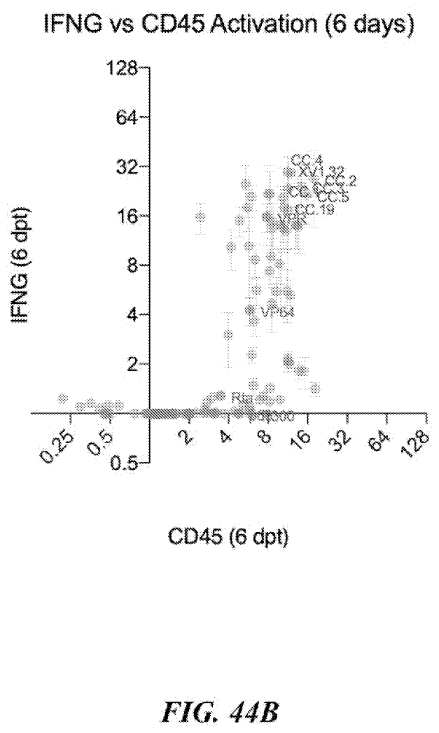

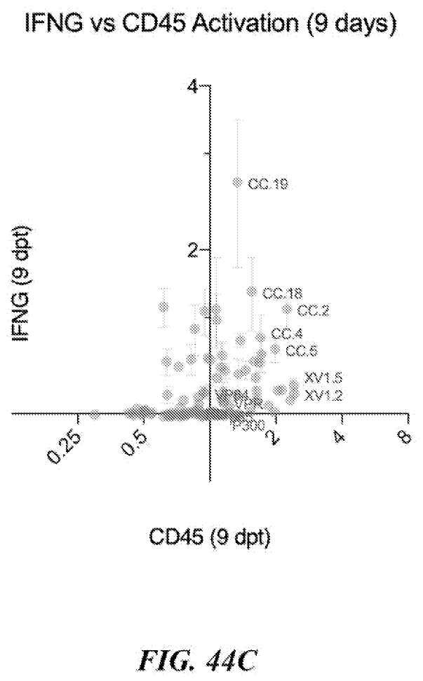

Referring to FIGS. 44A-C, plotted data further demonstrates the engineered gene effectors' persistent and sustained gene activation of CD45 and IFNG. As previously demonstrated herein, some of the engineered gene effectors have high potency and high durability compared to that of activator controls (e.g., VPR, VP64). However, some of the engineered gene effectors demonstrated varying potency and durability when targeting different genes, even when the target genes are similarly expressed at baseline. This illustrates the importance of screening engineered gene effectors at multiple targets.

### Example 8: Structural Analysis of Engineered Gene Effectors

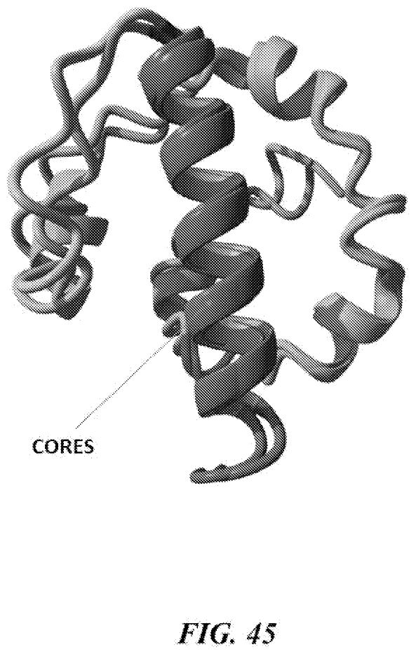

FIGS. 45-48 illustrate the predicted structure of various engineered gene effectors using Evolutionary Scale Modeling (ESMfold) structural analysis.

FIG. 45 illustrates the predicted structure of CC.2, CC.4, and CC.5, which showed high activation of CD45 at 3 days post transfection. The cores (as noted in FIG. 45) of CC.2, CC.4 and CC.5 shared vIRF2 core domain, LDDCLPMVDHIEGCLLDLLSDVGQELPDLGDL (SEQ ID NO:1), and aligned with less than 1 Armstrong root-mean square deviation (RMSD).

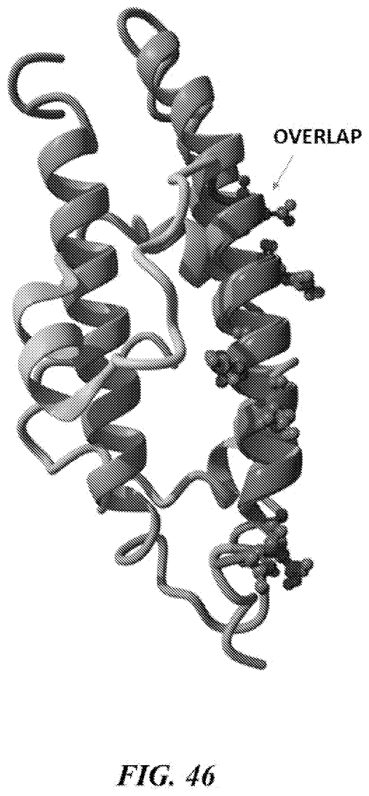

FIG. 46 illustrates the predicted structure of XV1.48 aligned to CC.2. XV1.48, a triple core sequence with a different structure than CC.2-. 5, forms a stable triple helix structure, which showed a high CD45 activation at 3 days post transfection. The overlapping region, as noted in FIG. 46, corresponds to the region that contains the predicted vIRF2 core domain, and aligns well with CC.2-.5. The ball-and-stick residues correspond to the acidic residues.

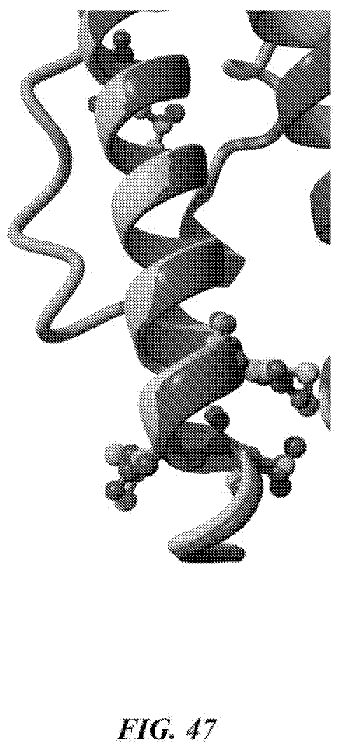

FIG. 47 illustrates the predicted structure of CC.32. CC.32 is a stable, double core, antiparallel mutated structure, but has showed low activation of CD45 at 3 days post transfection. Despite the mutated region, the alignment with sequences containing the core is aligned well, less than 1 Armstrong root-mean square deviation (RMSD). It was shown that it is critical for to retain C4, L5, M7 and L19 residues in the core regions (e.g., vIRF2 core domain).

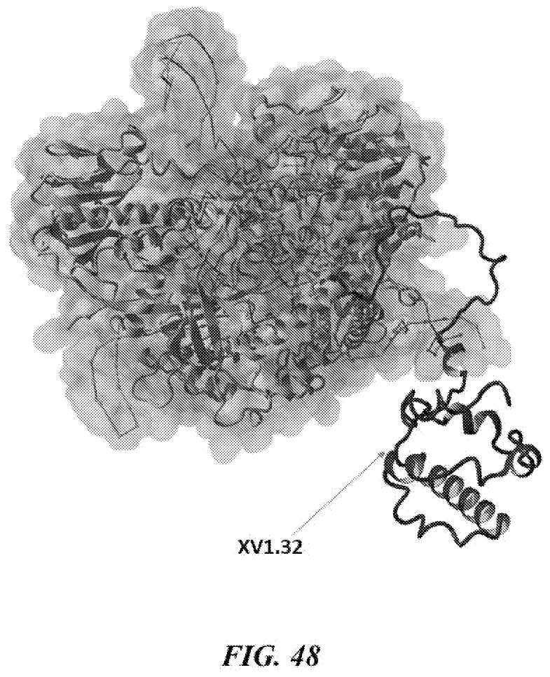

FIG. 48 illustrates ESMfold predicted structure of dCas protein coupled to XV1.32.

### Example 9: Screening of Engineered Gene Effectors

ParSeqSim from the “protr” package were used to generate all-by-all sequence homology maps for the activation hits coming from viral tiles (341) and human tiles (219) respectively. By comparing similarity at a sequence level, activation motifs were identified. From these large homology maps, DECIPHER package (http://www2.decipher.codes/) were used to visualize sequence alignments.

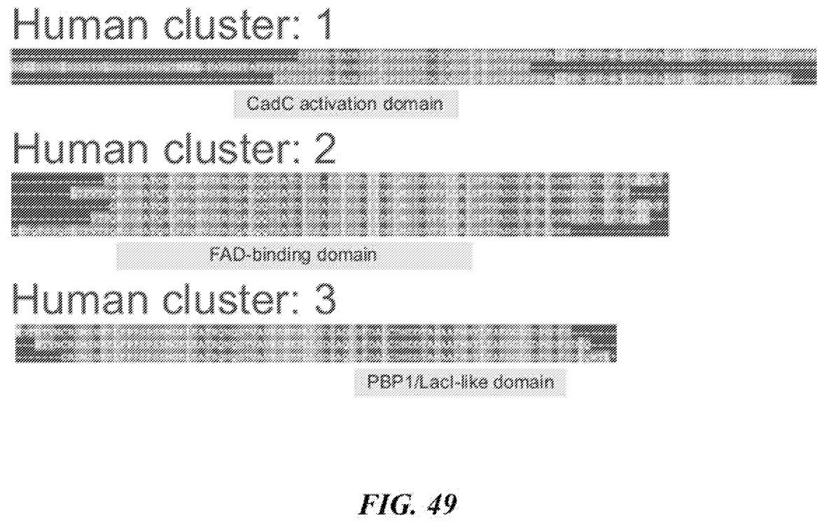

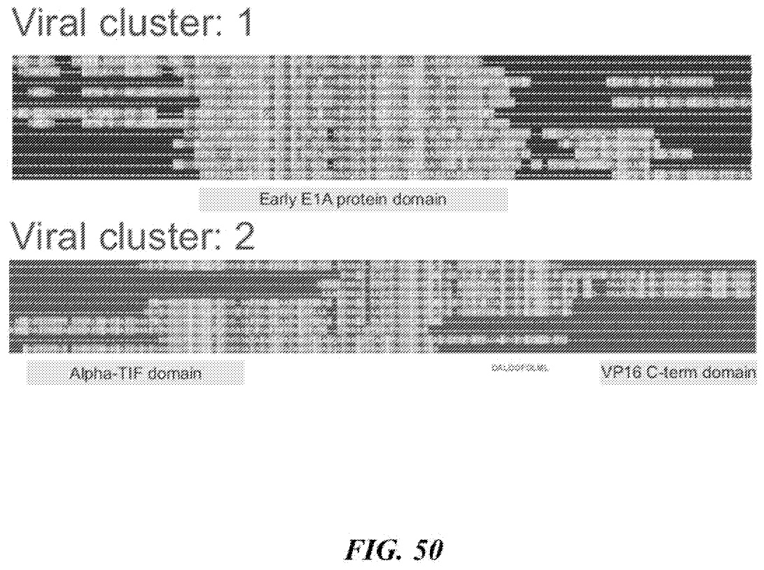

Referring to FIGS. 49 and 50, sequence homology alignment of 85aa activator tiles identified in the human (H) and viral (V) screen reveals that many hits deriving from the same gene (or gene families) had significant overlap in their peptide sequence. Since multiple independent tiles with overlapping sequences all appeared as hits in the activation screen, this suggested that the activation domain is within the overlapping peptide sequences. FIG. 49 shows an example of overlapping human tiles (from NHSL1, E2F5, and ARNTL2 respectively) and FIG. 50 shows an example of overlapping viral tiles derived from the same gene families across different viral serotypes (e.g. E1A and VP16).

A regularized logistic regression model (ElasticNet) were trained to predict which 85aa peptide tiles can be activators based on sequence alone (where peptide sequence was represented using OneHot encoding). Then, extracted feature importance for the 20 amino acids can identify which residue types were predictive of activator tiles. The bioinformatic package DagLogo can further identify peptide-level motif enrichment among activator hits (separated by human vs viral origin) and then colored it by different properties (i.e. charge).

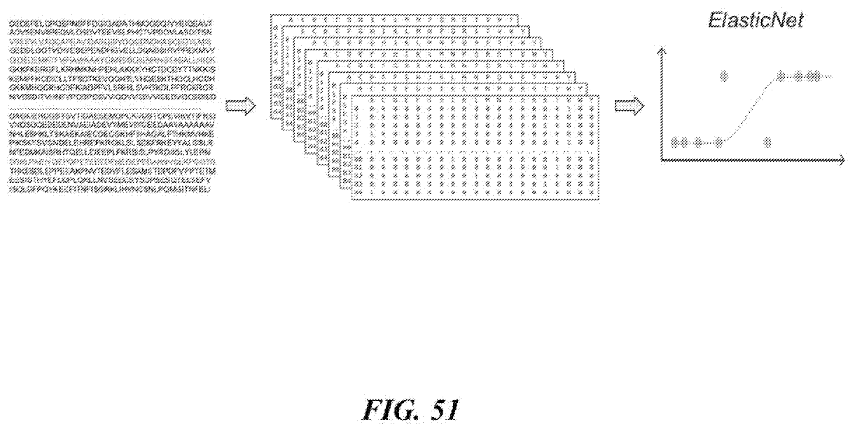

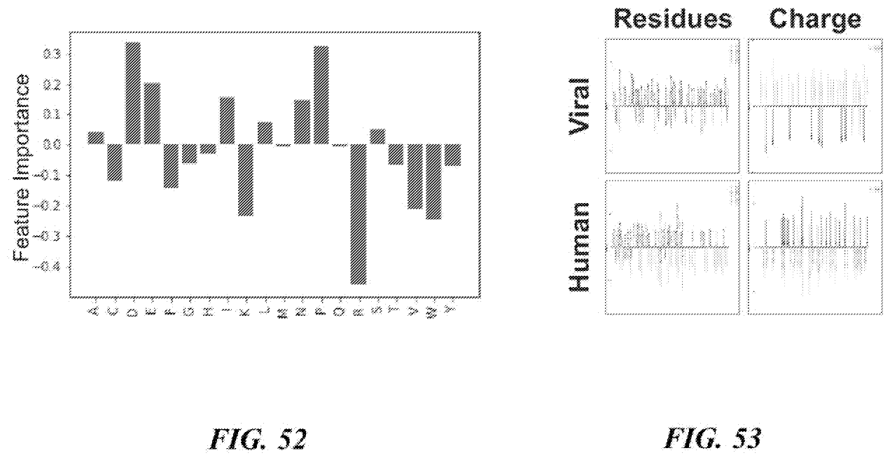

Referring to FIGS. 51-53, a machine learning-based approach found that acidic residues (D,E) were among the most predictive of activation tiles whereas basic residues (R,H,K) were predictive of tiles lacking activation ability. A different bioinformatic enrichment approach found that viral activation tiles were specifically enriched for acidic residues whereas human activation tiles were instead depleted of basic residues.

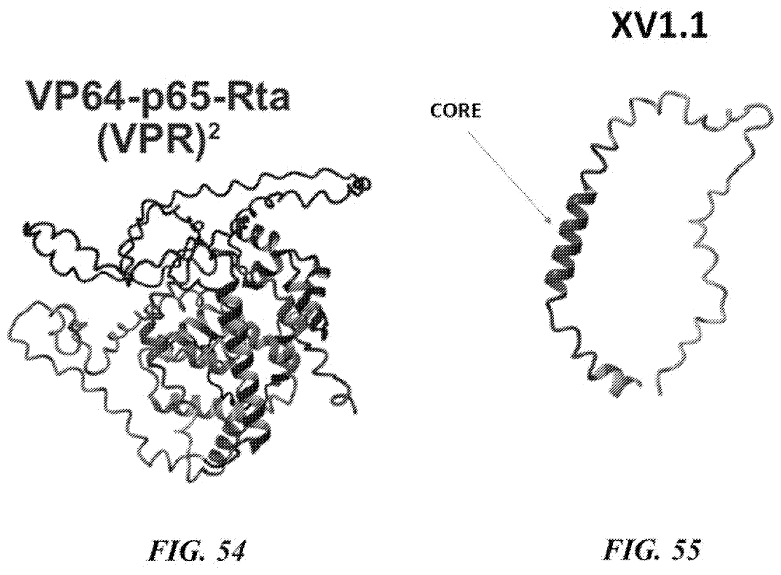

Referring to FIGS. 54 and 55, ESMFold was used to visualize the predicted 3-dimensional folded structure of VPR (FIG. 54) and one of the hypercompact vIRF2 tiles, XV1.1 (FIG. 55, e.g., engineered gene effectors). As noted in FIG. 55, the core region shows 32 aa core domain predicted to be responsible for activation.

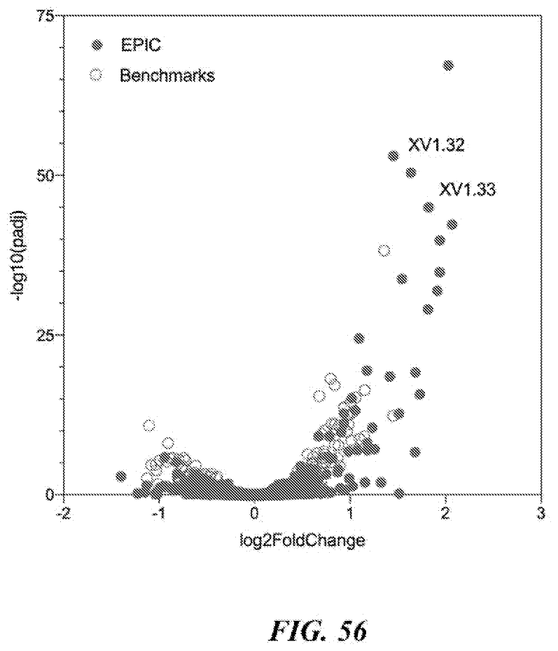

Referring to FIG. 56, a volcano plot illustrates the results of follow-up screening of engineered gene effectors, which confirms robust activation by the engineered gene effectors that contain minimal core domain.

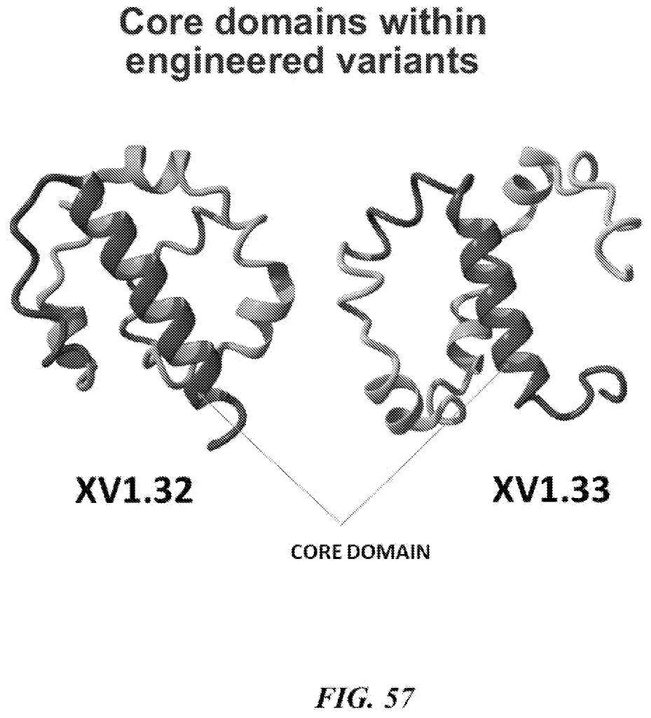

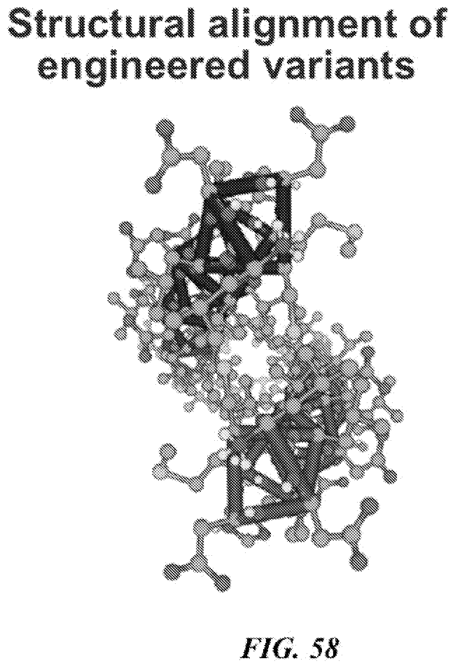

Referring to FIGS. 57-58, ESMFold was used to visualize the predicted 3-dimensional folded structure of some representative examples of engineered variants (e.g., XV1.32 and XV1.33). As noted in FIG. 87, the engineered gene effectors contained vIRF2 core domain sequence. FIG. 58 shows a “barrel” view of a coiled coil variant showing alpha helices arranged around each other with acidic residues positioned on the outside.

### Example 10: Potency and Durability of Engineered Gene Effectors

Wildtype HEK293T cells were transiently co-transfected with sgRNAs targeting the indicated human genes and dCas Protein-modulator (e.g., engineered gene effectors) expression plasmids using Mirus X2 reagent. At 3 days post-transfection, cells were processed for surface protein detection (CD45, CXCR4, CD2, CD71, CD81) (Biolegend), then analyzed by flow cytometry (CytoFlex LX). IFNG secretion in cell supernatants was measured by ELISA assay (BioLegend).

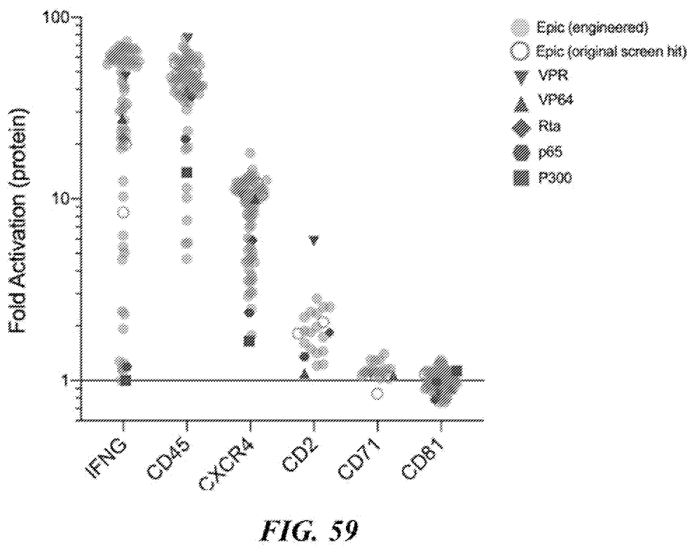

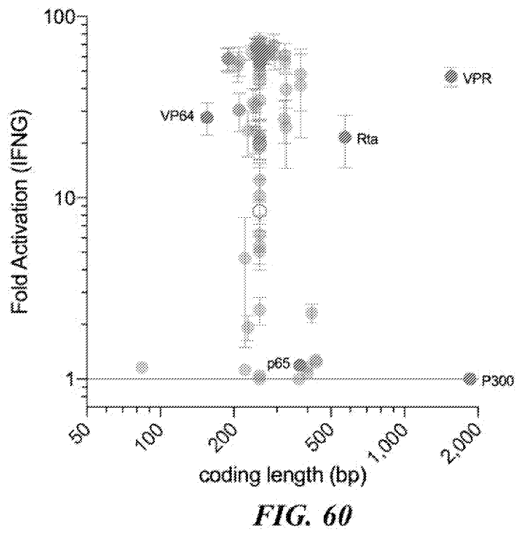

Referring to FIGS. 59-60, hypercompact, engineered vIRF2 variants (e.g., engineered gene effectors) showed similar or greater potency to benchmarks (e.g., VPR, VP64, Rta, p65, or p300) in activating multiple target genes (e.g., IFNG, CD56, CXCR4, CX2, CX71 and CD81), and demonstrated improved potency over the original sequences discovered in high-throughput screens. Subsets of vIRF2 variants (e.g., engineered gene effectors) were able to increase expression of already very highly expressed genes CD71 and CD81, while benchmarks were not.

Activation by an exemplary engineered gene effector or a benchmark (e.g., VPR) were measured at multiple time points. Wildtype HEK293T cells were transiently co-transfected with sgRNAs targeting the indicated human genes and dCas Protein-modulator (e.g., engineered gene effectors or VPR) expression plasmids using Mirus X2 reagent. At 3-18 days post-transfection, cells were processed for surface protein detection (CD45, CXCR4, CD81) (Biolegend), then analyzed by flow cytometry (CytoFlex LX). IFNG secretion in cell supernatants was measured by ELISA assay (BioLegend). The CXCR4 flow cytometry experiment proceeded to 28 days. Line plots demonstrate performance of a single vIRF2 variant (e.g., engineered gene effectors) over time at each indicated gene, while dot plots (below) show performance of the full set of variants relative to benchmarks.

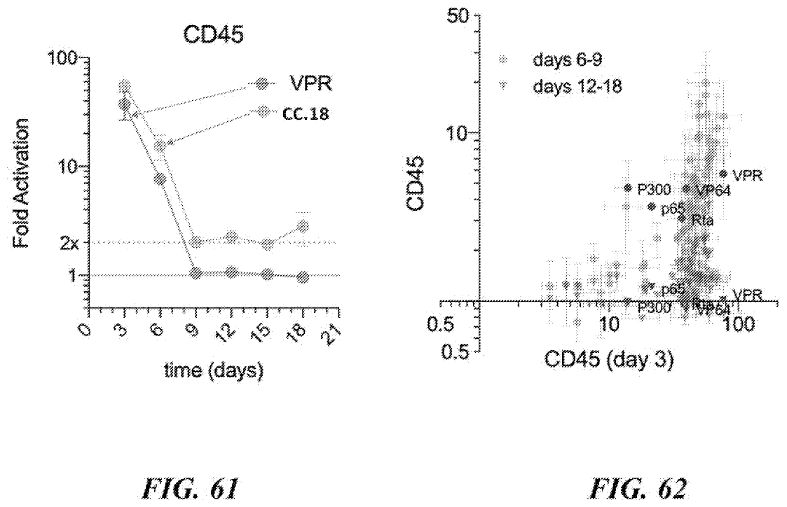

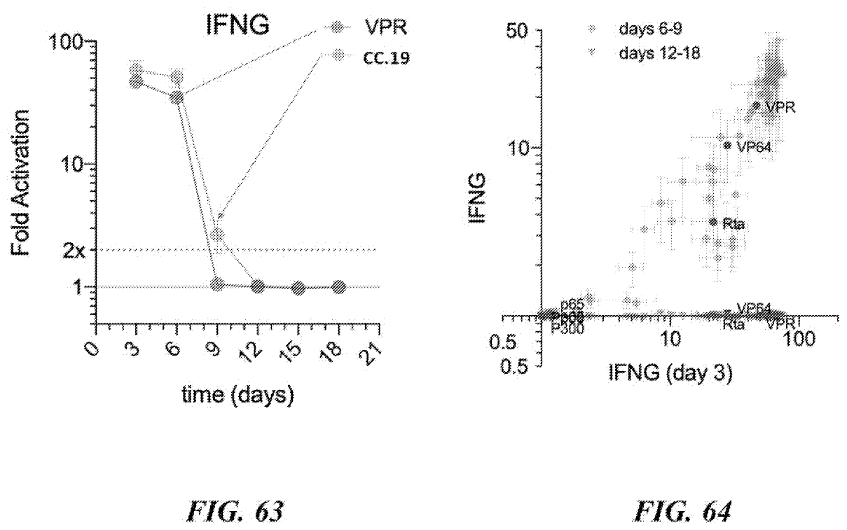

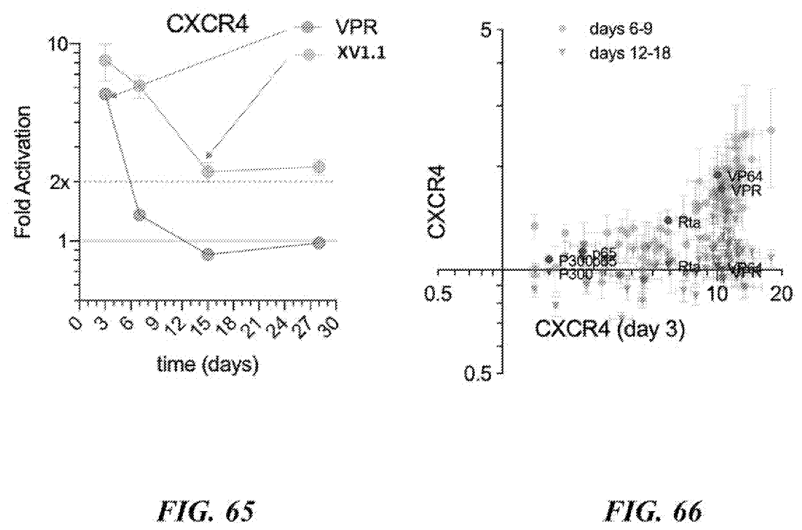

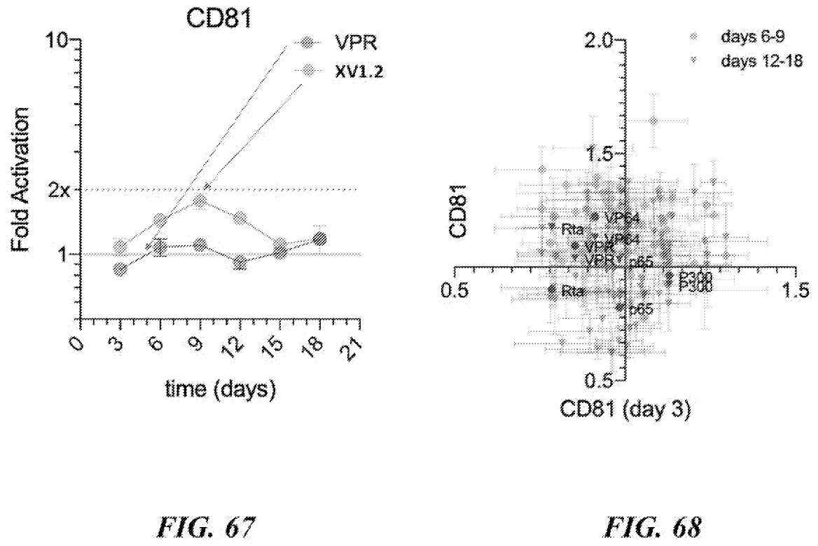

Referring to FIGS. 61-68, Hypercompact, engineered vIRF2 variants (e.g., CC.18, CC.19, XV1.1, or XV1.2) showed similar or greater potency to benchmarks in activating multiple target genes (e.g., CD45, IFNy, CXCR4, and CD81) at early time points, and subsets of vIRF2 variants (e.g., engineered gene effectors) were additionally able to durably maintain activation (e.g., persistent activation) over longer time frames than that of benchmarks (e.g., p300, p65, VPR, VP64, Rta).

mRNA measurement can be an important validation of flow cytometry/ELISA-based protein measurement, particularly at late time points as it is a more highly sensitive assay and is more suggestive of active transcription at late time points

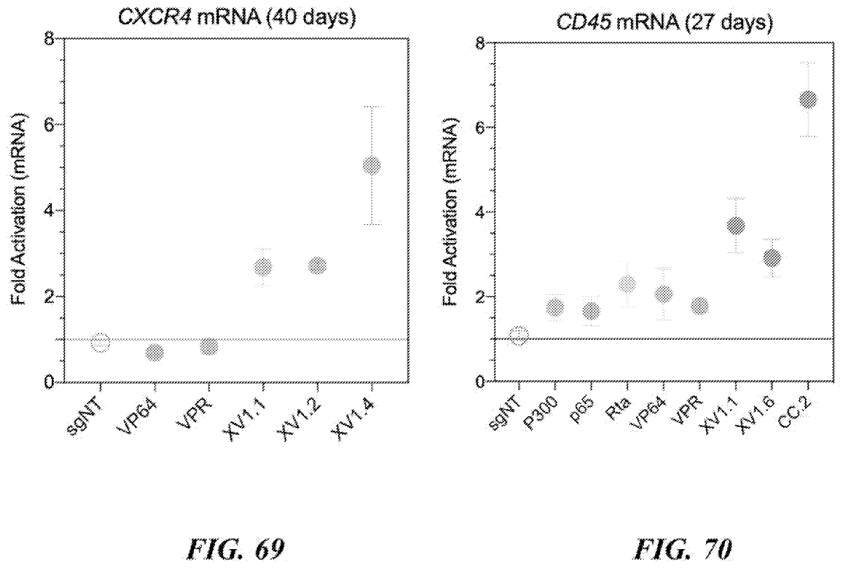

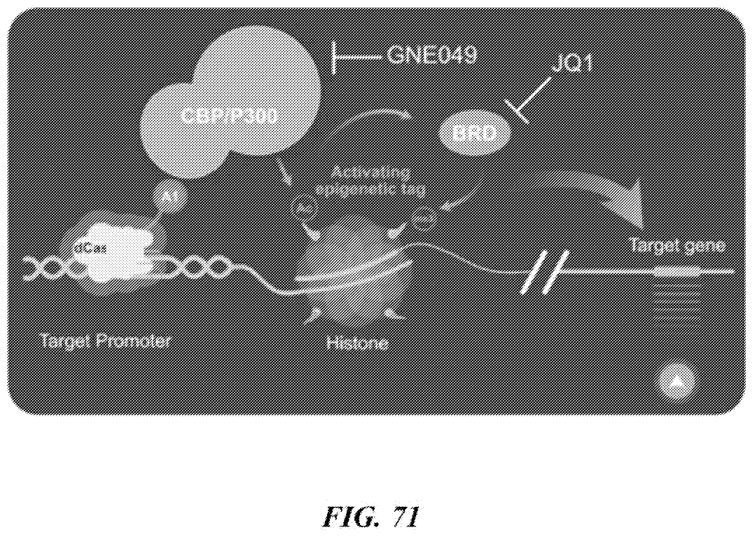

FIGS. 69-70 shows measurement of CXCR4 and CD45 mRNA level. Wildtype HEK293T cells were transiently co-transfected with CXCR4- or CD45-targeting sgRNA dCas Protein-modulator expression plasmids using Mirus X2 reagent. At 40 or 27 days post-transfection, respectively, cells were processed for mRNA quantification by RT-qPCR using One-Step Cells-to-Ct assay (ThermoFisher).

Referring to FIGS. 69-70, dCas Protein-vIRF2 fusions (e.g., dCas protein coupled to the engineered gene effectors) maintained higher levels of target mRNA activation at later time points for CD45 and CXCR4, compared to VPR and other benchmarks (e.g., VPR, p65, Rta).

### Example 11: Epigenetic Mechanisms of Persistent Activation

The epigenetic mechanisms by which modulators affect target gene expression was analyzed by applying well-characterized pharmacological inhibitors of various cellular epigenetic machinery enzymes, then by measuring these drugs' effects on modulator-dependent activation. Three commercially-available drugs that inhibit CBP/P300-associated BET bromodomains, including BRD2/4 were used. These enzymes can be responsible for the reading of activation-associated histone marks such as H3K27Ac, and therefore presumably would be important cellular requisites for the propagation of transcriptional activation (epigenetic “memory”) across successive mitotic cell divisions following transient delivery of a dCas-modulator (A1), as shown in FIG. 71. FIG. 71 illustrates an activation by novel modulators (e.g, engineered gene effectors, A1) can be dependent on CBP/P300-associated BET/BRD bromodomains, implicating H3K27Ac in the propagation of activation memory through mitotic cell divisions.

Wildtype HEK293T cells were transiently co-transfected with CD45-targeting sgRNA and dCas Protein-modulator expression plasmids using Mirus X2 reagent. From 5-9 days post-transfection, cells were treated with JQ1, GNE049, or SGC-CBP30 (Selleck Chem). At 9 days post-transfection, cells were processed for CD45 surface protein detection using CD45-APC antibody (Biolegend), then analyzed by flow cytometry (CytoFlex LX).

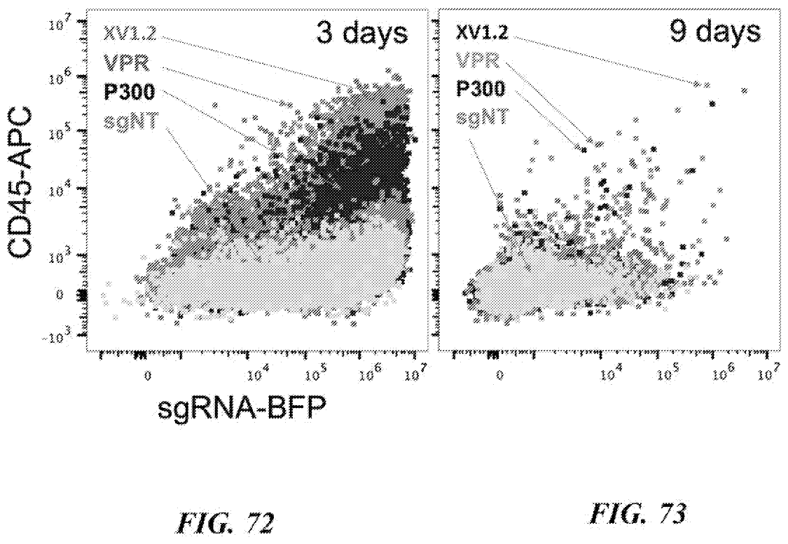

Referring to FIGS. 72 and 73, the activation of CD45 by engineered gene effectors (e.g., XV_1.2) persisted for at least 9 days post transfection. In comparison to other control benchmark modulators (e.g., VPR or p300), a higher proportion of cells transfected with the engineered gene effectors (e.g., XV1.2) showed persistent and sustained target gene (e.g., CD45) activation.

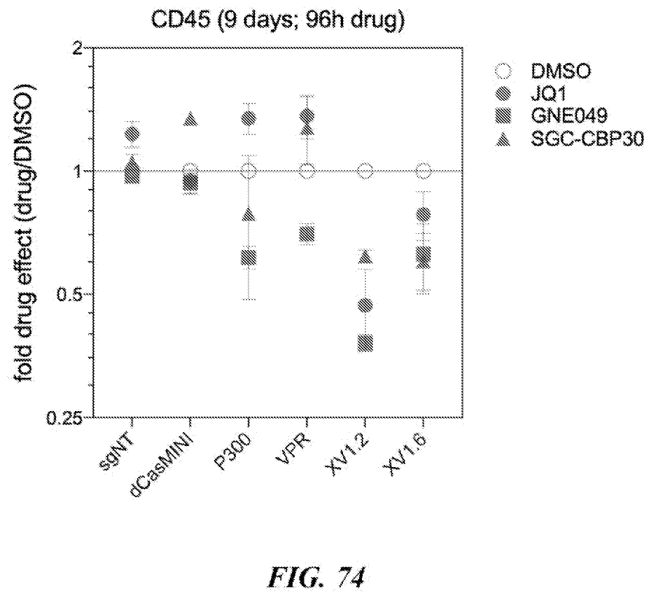

Referring to FIG. 74, the engineered gene effectors comprising vIRF2 core domain (e.g., XV1.2 or XV1.6)-induced CD45 activation was substantially reduced following exposure to CBP/P300-associated BET bromodomain inhibitors (e.g., JQ1, GNE049, SGC-CBP30). The same drugs when applied to VPR-induced conditions had less impact on observed CD45 activation. Results were consistent with the hypothesis that the engineered gene effectors comprising vIRF2 core domain (e.g., XV1.2 or XV1.6)-induced CD45 activation, in contrast to that of VPR, is dependent on CBP/P300-associated BET bromodomain function.

### Example 12: Mitotic Durable Gene Activation by Engineered Gene Effectors

Modulator-dependent differences in cell cycle rates were analyzed by a pulse-chase assay that can be used to label S-phase (e.g., dividing) cells during CD45 activation via a Cas-modulator/sgRNA system.

Wildtype HEK293T cells were transiently co-transfected with CD45-targeting sgRNA and dCas-modulator (e.g., gene effector) expression plasmids using Mirus X2 reagent. At 8 days post-transfection, cells were pulsed with 1 micromolar (μM) 5-ethynyl-2′-deoxyuridine (EdU) for 1 hour to label S-phase cells, followed by PBS wash and replacement of fresh DMEM/FBS media. At 9 days post-transfection, cells were processed for CD45 surface protein detection using CD45-APC antibody and EdU detection using a Click chemistry-based AlexaFluor-488 assay, then analyzed by flow cytometry.

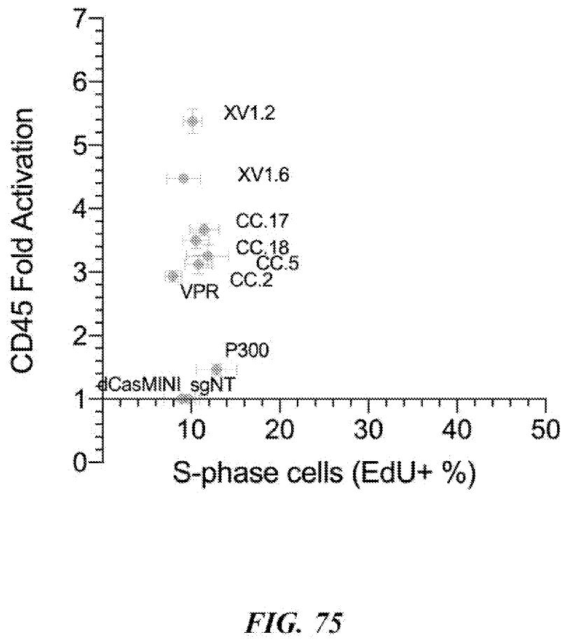

Referring to FIG. 75, major differences in cell growth rate were not observed among modulator conditions. Also, a simple linear regression analysis comparing dividing cell rates versus observed CD45 expression shows no significant effect of cell growth rate on observed CD45 activation. The results suggest that observed CD45 activation is not an artifact of slower-dividing cells, and support the notion that observed durability of CD45 activation at later time points is an effect that is memorized and transmitted through multiple rounds of mitosis (e.g., mitotic durable gene activation).

It shall be understood that different aspects of the invention can be appreciated individually, collectively, or in combination with each other. Various aspects of the invention described herein may be applied to any of the particular applications disclosed herein. The compositions of matter disclosed herein in the composition section of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An engineered gene effector comprising a polypeptide, wherein:
the polypeptide is heterologous to any of the members selected from the group consisting of VP16, VP64, p65, and Rta;
the engineered gene effector has a size of at most 500 amino acid residues;
the engineered gene effector is capable of activating an expression level of a target gene in a cell, wherein the expression level of the target gene that is activated via the engineered gene effector is at least 80% as compared to that activated by a VP64-p65-Rta fusion polypeptide (VPR) in a control cell; and
the polypeptide comprises an amino acid sequence that exhibits at least 90% sequence identity to the polypeptide sequence of SEQ ID NO: 1.

2.-52. (canceled)

53. The engineered gene effector of claim 1, wherein the amino acid sequence of the polypeptide comprises one or more members selected from the group consisting of C4, L5, M7, and L19, when aligned to the polypeptide sequence of SEQ ID NO: 1.

54. The engineered gene effector of claim 1, wherein the expression level of the target gene that is activated via the engineered gene effector persists for a duration of at least 9 days.

55. The engineered gene effector of any claim 1, wherein the target gene is endogenous to the cell.

56. An engineered gene effector comprising a polypeptide coupled to an additional polypeptide, wherein:
the polypeptide comprises an amino acid sequence having at least 90% sequence identity to the polypeptide sequence of SEQ ID NO: 1;
the additional polypeptide comprises at least a portion of one or more members selected from the group consisting of VP16, VP64, p65, and Rta; and
the engineered gene effector has a size less than or equal to about 250 amino acid residues.

57. The engineered gene effector of claim 56, wherein the additional polypeptide comprises VP16 or an amino acid sequence having at least 90% sequence identity to the polypeptide sequence of SEQ ID NO: 5.

58. The engineered gene effector of claim 56, wherein the additional polypeptide comprises VP64 or an amino acid sequence having at least 90% sequence identity to the polypeptide sequence of SEQ ID NO: 6.

59. The engineered gene effector of claim 56, wherein the polypeptide is not identical to any one of SEQ ID NO: 2 and SEQ ID NO: 3.

60. The engineered gene effector of claim 56, wherein the additional polypeptide is positioned C-terminal of the polypeptide.

61. The engineered gene effector of claim 56, wherein the additional polypeptide is positioned N-terminal of the polypeptide.

62. The engineered gene effector of claim 56, wherein the amino acid sequence of the polypeptide comprises one or more members selected from the group consisting of C4, L5, M7, and L19, when aligned to the polypeptide sequence of SEQ ID NO: 1.

63. An engineered gene effector comprising a polypeptide comprising an amino acid sequence having at least 90% sequence identity to the polypeptide sequence of SEQ ID NO: 1, wherein the engineered gene effector is not identical to any one of SEQ ID NO: 2, SEQ ID NO: 3, or SEQ ID NO: 4.

64. An engineered gene effector comprising a plurality of polypeptide domains, wherein each polypeptide domain of the plurality of polypeptide domain comprises a polypeptide comprising an amino acid sequence that exhibits at least about 90% sequence identity to the polypeptide sequence of SEQ ID NO: 1.

65. The engineered gene effector of claim 64, wherein the plurality of polypeptide domain comprises three or more polypeptide domains.

66. The engineered gene effector of claim 64, wherein the size of the engineered gene effector is less than or equal to about 150 amino acid residues.

67. A system comprising:
the engineered gene effector of claim 1; and
a heterologous endonuclease coupled to the engineered gene effector.

68. One or more polynucleotides encoding the system of claim 67.

69. A cell comprising the system of claim 67.

70. A method of controlling a target gene in a cell, the method comprising contacting the cell with the system of claim 67.


SEQUENCE LISTING (138 sequences)

SEQ ID NO: 1 - protein, 32 aa, Human gammaherpesvirus 8
LDDCLPMVDHIEGCLLDLLSDVGQELPDLGDL

SEQ ID NO: 2 - protein, 85 aa, Human gammaherpesvirus 8
TPTDLCLPTGGLPSPVIFPHETQGLLAPPAGQSQTPFSPEGPVPSHVSGLDDCLPMVDHIEGCLLDLLSDVGQELPDLGDLGELL

SEQ ID NO: 3 - protein, 85 aa, Human gammaherpesvirus 8
PPAGQSQTPFSPEGPVPSHVSGLDDCLPMVDHIEGCLLDLLSDVGQELPDLGDLGELLCETASPQGPMQSEGGEEGSTESVSVLP

SEQ ID NO: 4 - protein, 680 aa, Human gammaherpesvirus 8
MPRYTESEWLTDFIIDALDSGRFWGVGWLDEQKRIFTVPGRNRRERMPEGFDDFYEAFLEERRRHGLPEIPETETGLGCFGRLLRTANRARQERPFTIYKGKMKLNRWIMTPRPYKGCEGCLVYLTQEPAMKNMLKALFGIYPHDDKHREKALRRSLRKKAQREAARKQAAAVATPTTSSAAEVSSRSQSEDTESSDSENELWVGAQGFVGRDMHSLFFEEPEPSGFGSSGQSSSLLAPDSPRPSTSQVQGPLHVHTPTDLCLPTGGLPSPVIFPHETQGLLAPPAGQSQTPFSPEGPVPSHVSGLDDCLPMVDHIEGCLLDLLSDVGQELPDLGDLGELLCETASPQGPMQSEGGEEGSTESVSVLPATHPLESSAPGASVMGSGQELPDLGDLSELLCETASPQGPMQSEGGEEGSTESVSVLPATHPLESSAPGASVMGSSFQASDNVDDFIDCIPPLCRDDRDVEDQEKADQTFYWYGSDMRPKVLTATQSVAAYLSKKQAIYKVGDKLVPLVVEVYYFGEKVKTHFDLTGGIVICSQVPEASPEHICQTVPPYKCLLPRTAHCSVDANRTLEQTLDRFSMGVVAIGTNMGIFLKGLLEYPAYFVGNASRRRIGKCRPLSHRHEIQQAFDVERHNREPEGSRYASLFLGRRPSPEYDWDHYPVILHIYLAPFYHRD

SEQ ID NO: 5 - protein, 11 aa, unidentified
  source 1..11: note = Herpes simplex virus
DALDDFDLDML

SEQ ID NO: 6 - protein, 50 aa, synthetic construct
DALDDFDLDMLGSDALDDFDLDMLGSDALDDFDLDMLGSDALDDFDLDML

SEQ ID NO: 7 - protein, 265 aa, Homo sapiens
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 8 - protein, 190 aa, Human gammaherpesvirus 4
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 9 - protein, 518 aa, synthetic construct
DALDDFDLDMLGSDALDDFDLDMLGSDALDDFDLDMLGSDALDDFDLDMLGSGGSGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 10 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 11 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 12 - protein, 85 aa, synthetic construct
GGGSLDDCLPMVDHIEGCLLDLLSDVGQELPDLGDLGSAGSAAGSGEFGGSGSGSGGSGGSGSAGSAAGSGEFGGSGGSGSGGGS

SEQ ID NO: 13 - protein, 85 aa, synthetic construct
GSAGSAAGSGEFLDDCLPMVDHIEGCLLDLLSDVGQELPDLGDLGSAGSAAGSGEFGGSGSGSGGSGGSGSAGSAAGSGEFGSGS

SEQ ID NO: 14 - protein, 85 aa, synthetic construct
GSAGSAAGSGEFGSGSGSGSGSLDDCLPMVDHIEGCLLDLLSDVGQELPDLGDLGSAGSAAGSGEFGGSGSGSAGSAAGSGEFGS

SEQ ID NO: 15 - protein, 85 aa, synthetic construct
GSAGSAAGSGEFGGSGSGSAGSAAGSGEFGSLDDCLPMVDHIEGCLLDLLSDVGQELPDLGDLGSAGSAAGSGEFGSGSGSGSGS

SEQ ID NO: 16 - protein, 85 aa, synthetic construct
GSAGSAAGSGEFGGSGSGSGGSGGSGSAGSAAGSGEFGSGSLDDCLPMVDHIEGCLLDLLSDVGQELPDLGDLGSAGSAAGSGEF

SEQ ID NO: 17 - protein, 85 aa, synthetic construct
GSAGSAAGSGEFGGSGSGSGGSGGSGSAGSAAGSGEFGGSGGSGSGGGSLDDCLPMVDHIEGCLLDLLSDVGQELPDLGDLGGGS

SEQ ID NO: 18 - protein, 85 aa, synthetic construct
GSAGSAAGSGEFLDDCLPMVDHIEGCLLDLLSDVGQELPDLGDLGGSGGSGGSLDDCLPMVDHIEGCLLDLLSDVGQELPDLGDL

SEQ ID NO: 19 - protein, 85 aa, synthetic construct
GSAGSAAGSGEFGGSGGGSLDDCLPMVDHIEGCLLDLLSDVGQELPDLGDLGSLDDCLPMVDHIEGCLLDLLSDVGQELPDLGDL

SEQ ID NO: 20 - protein, 85 aa, synthetic construct
LDDCLPMVDHIEGCLLDLLSDVGQELPDLGDLGSLDDCLPMVDHIEGCLLDLLSDVGQELPDLGDLGGGSGGSGSAGSAAGSGEF

SEQ ID NO: 21 - protein, 85 aa, synthetic construct
LDDCLPMVDHIEGCLLDLLSDVGQELPDLGDLGGSGGSGGSLDDCLPMVDHIEGCLLDLLSDVGQELPDLGDLGSAGSAAGSGEF

SEQ ID NO: 22 - protein, 85 aa, synthetic construct
LDDCLPMVDHIEGCLLDLLSDVGQELPDLGDLGSAGSAAGSGEFGSGSGSGGSLDDCLPMVDHIEGCLLDLLSDVGQELPDLGDL

SEQ ID NO: 23 - protein, 85 aa, synthetic construct
LDDCLPMVDHIEGCLLDLLSDVGQELPDLGDLGGSDALDDFDLDMLGSDALDDFDLDMLGSDALDDFDLDMLGSDALDDFDLDML

SEQ ID NO: 24 - protein, 85 aa, synthetic construct
DALDDFDLDMLGSDALDDFDLDMLGSDALDDFDLDMLGSDALDDFDLDMLGGSLDDCLPMVDHIEGCLLDLLSDVGQELPDLGDL

SEQ ID NO: 25 - protein, 85 aa, synthetic construct
QGLLGESSNLPDLEVHMSGGPFWGGSQGLLGESSNLPDLEVHMSGGPFWGGGSLDDCLPMVDHIEGCLLDLLSDVGQELPDLGDL

SEQ ID NO: 26 - protein, 90 aa, synthetic construct
NVECFKKFACNQEYWEEFAASPIGSAGSAAGSGEFNVECFKKFACNQEYWEEFAASPILDDCLPMVDHIEGCLLDLLSDVGQELPDLGDL

SEQ ID NO: 27 - protein, 85 aa, synthetic construct
NVELYEEYFNFIVSSDTYFNGGSNVELYEEYFNFIVSSDTYFNGGGSGGGSGSLDDCLPMVDHIEGCLLDLLSDVGQELPDLGDL

SEQ ID NO: 28 - protein, 85 aa, synthetic construct
ANEKIIREDLPLSFYGSANEKIIREDLPLSFYGSANEKIIREDLPLSFYGGGSLDDCLPMVDHIEGCLLDLLSDVGQELPDLGDL

SEQ ID NO: 29 - protein, 133 aa, synthetic construct
GGGSANEKIIREDLPLSFYGGGSNVELYEEYFNFIVSSDTYFNGGGSNVECFKKFACNQEYWEEFAASPIGGGSQGLLGESSNLPDLEVHMSGGPFWGGGSLDDCLPMVDHIEGCLLDLLSDVGQELPDLGDL

SEQ ID NO: 30 - protein, 108 aa, synthetic construct
GGGSLDDCLPMVDHIEGCLLDLLSDVGQELPDLGDLGGGSLDDCLPMVDHIEGCLLDLLSDVGQELPDLGDLGGGSLDDCLPMVDHIEGCLLDLLSDVGQELPDLGDL

SEQ ID NO: 31 - protein, 98 aa, synthetic construct
GGSDALDDFDLDMLGGSLDDCLPMVDHIEGCLLDLLSDVGQELPDLGDLGGSDALDDFDLDMLGGSLDDCLPMVDHIEGCLLDLLSDVGQELPDLGDL

SEQ ID NO: 32 - protein, 36 aa, synthetic construct
HVSGLDDCLPMVDHIEGCLLDLLSDVGQELPDLGDL

SEQ ID NO: 33 - protein, 44 aa, synthetic construct
SPEGPVPSHVSGLDDCLPMVDHIEGCLLDLLSDVGQELPDLGDL

SEQ ID NO: 34 - protein, 54 aa, synthetic construct
PPAGQSQTPFSPEGPVPSHVSGLDDCLPMVDHIEGCLLDLLSDVGQELPDLGDL

SEQ ID NO: 35 - protein, 63 aa, synthetic construct
PHETQGLLAPPAGQSQTPFSPEGPVPSHVSGLDDCLPMVDHIEGCLLDLLSDVGQELPDLGDL

SEQ ID NO: 36 - protein, 73 aa, synthetic construct
TGGLPSPVIFPHETQGLLAPPAGQSQTPFSPEGPVPSHVSGLDDCLPMVDHIEGCLLDLLSDVGQELPDLGDL

SEQ ID NO: 37 - protein, 81 aa, synthetic construct
TPTDLCLPTGGLPSPVIFPHETQGLLAPPAGQSQTPFSPEGPVPSHVSGLDDCLPMVDHIEGCLLDLLSDVGQELPDLGDL

SEQ ID NO: 38 - protein, 36 aa, synthetic construct
GGGSLDDCLPMVDHIEGCLLDLLSDVGQELPDLGDL

SEQ ID NO: 39 - protein, 44 aa, synthetic construct
GSAGSAAGSGEFLDDCLPMVDHIEGCLLDLLSDVGQELPDLGDL

SEQ ID NO: 40 - protein, 54 aa, synthetic construct
GSAGSAAGSGEFGGGSGSGGGSLDDCLPMVDHIEGCLLDLLSDVGQELPDLGDL

SEQ ID NO: 41 - protein, 63 aa, synthetic construct
GSAGSAAGSGEFGGSGSGSAGSAAGSGEFGSLDDCLPMVDHIEGCLLDLLSDVGQELPDLGDL

SEQ ID NO: 42 - protein, 72 aa, synthetic construct
GSAGSAAGSGEFGGSGSGSGGSGSGSAGSAAGSGEFGSGSLDDCLPMVDHIEGCLLDLLSDVGQELPDLGDL

SEQ ID NO: 43 - protein, 81 aa, synthetic construct
GSAGSAAGSGEFGGSGSGSGGSGGSGSAGSAAGSGEFGGSGGSGSGGGSLDDCLPMVDHIEGCLLDLLSDVGQELPDLGDL

SEQ ID NO: 44 - protein, 85 aa, synthetic construct
RHDPVLRCDMFEKHHKPRPKRSRKRSIDHESCASSGDTVANESGPLCTNTFWTPGPVLQGLLGESSNLPDLEVHMSGGPFWKEVY

SEQ ID NO: 45 - protein, 85 aa, synthetic construct
PSKLRSYPKQHAYHAPSIRSAVPSPFQNTLQNVLAAATKRNCNVTQMRELPTLDSAVFNVECFKKFACNQEYWEEFAASPIRITT

SEQ ID NO: 46 - protein, 85 aa, synthetic construct
DKDSKEIVSILTKFVNVISNIHSKYKCKYMFVGIPAIILFNKLDSTDVQKLYSLFSTKLNTNVELYEEYFNFIVSSDTYFNKKKF

SEQ ID NO: 47 - protein, 85 aa, synthetic construct
GSAGSAAGSGEFSGGSGSGGSGSAGSAAGSGEFMGAYTSIMNINDFNKIYAYKQDYRCKYNVSYELANEKIIREDLPLSFYIESF

SEQ ID NO: 48 - protein, 85 aa, synthetic construct
GGGSQGLLGESSNLPDLEVHMSGGPFWGSGSQGLLGESSNLPDLEVHMSGGPFWGGGSQGLLGESSNLPDLEVHMSGGPFWGSGS

SEQ ID NO: 49 - protein, 85 aa, synthetic construct
GGGSNVECFKKFACNQEYWEEFAASPIGSGSNVECFKKFACNQEYWEEFAASPIGSGSNVECFKKFACNQEYWEEFAASPIGSGS

SEQ ID NO: 50 - protein, 85 aa, synthetic construct
GGGSGGSGSGGGSGSGSNVELYEEYFNFIVSSDTYFNGGGSNVELYEEYFNFIVSSDTYFNGGGSNVELYEEYFNFIVSSDTYFN

SEQ ID NO: 51 - protein, 85 aa, synthetic construct
GGGSGGSGSANEKIIREDLPLSFYGGGSANEKIIREDLPLSFYGGGSANEKIIREDLPLSFYGGGSANEKIIREDLPLSFYGGGS

SEQ ID NO: 52 - protein, 23 aa, Simian foamy virus
QGLLGESSNLPDLEVHMSGGPFW

SEQ ID NO: 53 - protein, 85 aa, Simian foamy virus; identical to SEQ ID NO: 44

SEQ ID NO: 54 - protein, 300 aa, Simian foamy virus
MDSYQEEEPVASTSGLQDLQTLSELVGPENAGEGDLVIAEEPEENPRRPRRYTKRDVKCVSYHAYKELEDKHPHHIKLQDWIPKPEEMSKSICKRLILCGLYSGEKAREILKKPFTVSWEQSETNPDCFIVSYTCIFCDAVIHDPMPVVWDSEVEIWVKYKPLRGIVGSAVFIMEKHQKNCSLVKPSTSCPEGPKPRRRHDPVLRCDMFEKHHKPRPKRSRKRSIDHESCASSGDTVANESGPLCTNTFWTPGPVLQGLLGESSNLPDLEVHMSGGPFWKEVYGDSILGPPSGSGEHSVL

SEQ ID NO: 55 - protein, 23 aa, Chikungunya virus
NVECFKKFACNQEYWEEFAASPI

SEQ ID NO: 56 - protein, 85 aa, Chikungunya virus; identical to SEQ ID NO: 45

SEQ ID NO: 57 - protein, 2474 aa, Chikungunya virus
MDPVYVDIDADSAFLKALQRAYPMFEVEPRQVTPNDHANARAFSHLAIKLIEQEIDPDSTILDIGSAPARRMMSDRKYHCVCPMRSAEDPERLANYARKLASAAGKVLDRNISGKIGDLQAVMAVPDTETPTFCLHTDVSCRQRADVAIYQDVYAVHAPTSLYHQAIKGVRLAYWVGFDTTPFMYNAMAGAYPSYSTNWADEQVLKAKNIGLCSTDLTEGRRGKLSIMRGKKLEPCDRVLFSVGSTLYPESRKLLKSWHLPSVFHLKGKLSFTCRCDTVVSCEGYVVKRITMSPGLYGKTTGYAVTHHADGFLMCKTTDTVDGERVSFSVCTYVPATICDQMTGILATEVTPEDAQKLLVGLNQRIVVNGRTQRNTNTMKNYMIPVVAQAFSKWAKECRKDMEDEKLLGVRERTLTCCCLWAFKKQKTHTVYKRPDTQSIQKVQAEFDSFVVPSLWSSGLSIPLRTRIKWLLSKVPKTDLTPYSGDAQEARDAEKEAEEEREAELTLEALPPLQAAQEDVQVEIDVEQLEDRAGAGIIETPRGAIKVTAQPTDHVVGEYLVLSPQTVLRSQKLSLIHALAEQVKTCTHSGRAGRYAVEAYDGRVLVPSGYAISPEDFQSLSESATMVYNEREFVNRKLHHIAMHGPALNTDEESYELVRAERTEHEYVYDVDQRRCCKKEEAAGLVLVGDLTNPPYHEFAYEGLKIRPACPYKIAVIGVFGVPGSGKSAIIKNLVTRQDLVTSGKKENCQEITTDVMRQRGLEISARTVDSLLLNGCNRPVDVLYVDEAFACHSGTLLALIALVRPRQKVVLCGDPKQCGFFNMMQMKVNYNHNICTQVYHKSISRRCTLPVTAIVSSLHYEGKMRTTNEYNKPIVVDTTGSTKPDPGDLVLTCFRGWVKQLQIDYRGHEVMTAAASQGLTRKGVYAVRQKVNENPLYASTSEHVNVLLTRTEGKLVWKTLSGDPWIKTLQNPPKGNFKATIKEWEVEHASIMAGICSHQMTFDTFQNKANVCWAKSLVPILETAGIKLNDRQWSQIIQAFKEDKAYSPEVALNEICTRMYGVDLDSGLFSKPLVSVYYADNHWDNRPGGKMFGFNPEAASILERKYPFTKGKWNINKQICVTTRRIEDFNPTTNIIPANRRLPHSLVAEHRPVKGERMEWLVNKINGHHVLLVSGCSLALPTKRVTWVAPLGVRGADYTYNLELGLPATLGRYDLVVINIHTPFRIHHYQQCVDHAMKLQMLGGDSLRLLKPGGSLLIRAYGYADRTSERVICVLGRKFRSSRALKPPCVTSNTEMFFLFSNFDNGRRNFTTHVMNNQLNAAFVGQATRAGCAPSYRVKRMDIAKNDEECVVNAANPRGLPGDGVCKAVYKKWPESFKNSATPVGTAKTVMCGTYPVIHAVGPNFSNYSESEGDRELAAAYREVAKEVTRLGVNSVAIPLLSTGVYSGGKDRLTQSLNHLFTAMDSTDADVVIYCRDKEWEKKISEAIQMRTQVELLDEHISIDCDVVRVHPDSSLAGRKGYSTTEGALYSYLEGTRFHQTAVDMAEIYTMWPKQTEANEQVCLYALGESIESIRQKCPVDDADASSPPKTVPCLCRYAMTPERVTRLRMNHVTSIIVCSSFPLPKYKIEGVQKVKCSKVMLFDHNVPSRVSPREYRPSQESVQEASTTTSLTHSQFDLSVDGKILPVPSDLDADAPALEPALDDGAIHTLPSATGNLAAVSDWVMSTVPVAPPRRRRGRNLTVTCDEREGNITPMASVRFFRAELCPVVQETAETRDTAMSLQAPPSTATELSHPPISFGAPSETFPITFGDFNEGEIESLSSELLTFGDFLPGEVDDLTDSDWSTCSDTDDELRLDRAGGYIFSSDTGPGHLQQKSVRQSVLPVNTLEEVHEEKCYPPKLDEAKEQLLLKKLQESASMANRSRYQSRKVENMKATIIQRLKRGCRLYLMSETPKVPTYRTTYPAPVYSPPINVRLSNPESAVAACNEFLARNYPTVSSYQITDEYDAYLDMVDGSESCLDRATFNPSKLRSYPKQHAYHAPSIRSAVPSPFQNTLQNVLAAATKRNCNVTQMRELPTLDSAVFNVECFKKFACNQEYWEEFAASPIRITTENLTTYVTKLKGPKAAALFAKTHNLLPLQEVPMDRFTVDMKRDVKVTPGTKHTEERPKVQVIQAAEPLATAYLCGIHRELVRRLNAVLLPNVHTLFDMSAEDFDAIIAAHFKPGDTVLETDIASFDKSQDDSLALTALMLLEDLGVDHSLLDLIEAAFGEISSCHLPTGTRFKFGAMMKSGMFLTLFVNTLLNITIASRVLEDRLTKSACAAFIGDDNIIHGVVSDELMAARCATWMNMEVKIIDAVVSQKAPYFCGGFILHDIVTGTACRVADPLKRLFKLGKPLAAGDEQDEDRRRALADEVVRWQRTGLIDELEKAVYSRYEVQGISVVVMSMATFASSRSNFEKLRGPVVTLYGGPK

SEQ ID NO: 58 - protein, 20 aa, Eptesipox virus
NVELYEEYFNFIVSSDTYFN

SEQ ID NO: 59 - protein, 85 aa, Eptesipox virus; identical to SEQ ID NO: 46

SEQ ID NO: 60 - protein, 1002 aa, Eptesipox virus
MEDLFSITCDLHMNAFIEEVEKLWSSSLNNTSSLSRKSKNVIRNLFREVTNSNISSSCYDILLKKQLHGDSINAVYRSLYGSGSDIDKRVDTVGKYILFVVVTYLAILIDDKDSKEIVSILTKFVNVISNIHSKYKCKYMFVGIPAIILFNKLDSTDVQKLYSLFSTKLNTNVELYEEYFNFIVSSDTYFNKKKFVKFSYGPVSFASSISVPDFVMEGLTFRSCDRIEKSEDIDDVYVFITVESDPKTYAFSKLTKPLYEGGLVVETDDLDDATALVIFDAITTFDKFRNKALLLTLESIVNKQVIDPTLNIDRYPTDENISDDTGENVITPIKVGSSIGIVDYRLVINKLTEWLNQCEEKCEGAVSPEVKELRERITDLEKQLDEATSGKTNCEYEKTKIKDLESRLDSERQRVTRLLDDLQKARDGKCDSDSNDKAIIEELRKEIQNEQNRRMELMKELDKVRNGDGTSSCERELDLTRQWLHERDNELREMSKQAKHFERELERERIKNKQCDKYKKELDEAKSKIIRLETDLDKCLSQQNGSSDEVRKLTSQIESLERDLRECRASSGGDEKLLADIDLLKQQIGILAQQLEQCETRGDEKLLAEIEFLKEHINSLAQQLEQCEASGGSGGSDTSKLQERITYLENELDKYIKGEGNVNFTLLNEINRLRDKNAELQNQLDQAQSQDKNNSYYKRALERERAKIIELENELQKCFDNNSGSEYIIKMEQMERKIKSLEAELRLCKDTDHDTEKIYKDKIAELQRELDKCKQSGGSSNSHTEIKVFYDVECRTESARLQQRINELNDELNRLRKEDKTDSYYKREVDRQRKKVIELEHELEKYFNDDKIITYKKEMDAMQVVISEMRQELEKCKRDASCSSSSDCSFEQKRIELLELELRKTKEMVKQLEKFIEFSSAQKEYADKLEREKIARLDAEHALERERARKDCGGNMCEQELELERNKNKKLELYLETEKDKANFYKRELEKERFIKSSSQEE

SEQ ID NO: 61 - protein, 15 aa, Eptesipox virus
ANEKIIREDLPLSFY

SEQ ID NO: 62 - protein, 85 aa, Eptesipox virus
VSYELANEKIIREDLPLSFYIESFMRWYAQQRIQSVYTVNPVYKECLSCVNYARAKIPLSKFNKAINHYNSDNNSVYLVDVRKYN

SEQ ID NO: 63 - protein, 158 aa, Eptesipox virus
MGAYTSIMNINDFNKIYAYKQDYRCKYNVSYELANEKIIREDLPLSFYIESFMRWYAQQRIQSVYTVNPVYKECLSCVNYARAKIPLSKFNKAINHYNSDNNSVYLVDVRKYNNMQKLQLYIGFYSYVTTKLKSEDAIKRCIHTCTYISDEKTKLEFM

SEQ ID NO: 64 - protein, 96 aa, synthetic construct
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 65 - protein, 5 aa, synthetic construct
GGGGS

SEQ ID NO: 66 - protein, 4 aa, synthetic construct
GGGS

SEQ ID NO: 67 - skipped

SEQ ID NO: 68 - protein, 18 aa, synthetic construct
KESGSVSSEQLAQFRSLD

SEQ ID NO: 69 - protein, 14 aa, synthetic construct
EGKSSGSGSESKST

SEQ ID NO: 70 - protein, 12 aa, synthetic construct
GSAGSAAGSGEF

SEQ ID NO: 71 - protein, 5 aa, synthetic construct
EAAAK

SEQ ID NO: 72 - protein, 5 aa, synthetic construct
EAAAR

SEQ ID NO: 73 - protein, 5 aa, synthetic construct
PAPAP

SEQ ID NO: 74 - protein, 12 aa, synthetic construct
AEAAAKEAAAKA

SEQ ID NO: 75 - protein, 85 aa, synthetic construct
HVSGLDDCLPMVDHIEGCLLDLLSDVGQELPDLGDLGELLCETASPQGPMQSEGGEEGSTESVSVLPATHPLESSAPGASVMGSG

SEQ ID NO: 76 - protein, 85 aa, synthetic construct
SPEGPVPSHVSGLDDCLPMVDHIEGCLLDLLSDVGQELPDLGDLGELLCETASPQGPMQSEGGEEGSTESVSVLPATHPLESSAP

SEQ ID NO: 77 - protein, 85 aa, synthetic construct
PHETQGLLAPPAGQSQTPFSPEGPVPSHVSGLDDCLPMVDHIEGCLLDLLSDVGQELPDLGDLGELLCETASPQGPMQSEGGEEG

SEQ ID NO: 78 - protein, 85 aa, synthetic construct
TGGLPSPVIFPHETQGLLAPPAGQSQTPFSPEGPVPSHVSGLDDCLPMVDHIEGCLLDLLSDVGQELPDLGDLGELLCETASPQG

SEQ ID NO: 79 - protein, 85 aa, synthetic construct; identical to SEQ ID NO: 23

SEQ ID NO: 80 - protein, 84 aa, synthetic construct
LDDCLPMVDHIEGCLLDLLSDVGQELPDLGDLGSDALDDFDLDMLGSDALDDFDLDMLGSDALDDFDLDMLGSDALDDFDLDML

SEQ ID NO: 81 - protein, 83 aa, synthetic construct
LDDCLPMVDHIEGCLLDLLSDVGQELPDLGDLGDALDDFDLDMLGSDALDDFDLDMLGSDALDDFDLDMLGSDALDDFDLDML

SEQ ID NO: 82 - protein, 82 aa, synthetic construct
LDDCLPMVDHIEGCLLDLLSDVGQELPDLGDLDALDDFDLDMLGSDALDDFDLDMLGSDALDDFDLDMLGSDALDDFDLDML

SEQ ID NO: 83 - protein, 86 aa, synthetic construct
LDDCLPMVDHIEGCLLDLLSDVGQELPDLGDLGSGSDALDDFDLDMLGSDALDDFDLDMLGSDALDDFDLDMLGSDALDDFDLDML

SEQ ID NO: 84 - protein, 87 aa, synthetic construct
LDDCLPMVDHIEGCLLDLLSDVGQELPDLGDLGGSGSDALDDFDLDMLGSDALDDFDLDMLGSDALDDFDLDMLGSDALDDFDLDML

SEQ ID NO: 85 - protein, 88 aa, synthetic construct
LDDCLPMVDHIEGCLLDLLSDVGQELPDLGDLGGSGGSDALDDFDLDMLGSDALDDFDLDMLGSDALDDFDLDMLGSDALDDFDLDML

SEQ ID NO: 86 - protein, 82 aa, synthetic construct
LDDCLPMVDHIEGCLLDLLSDVGQELPDLGDLGGSDALDDFDLDMLGDALDDFDLDMLGDALDDFDLDMLGDALDDFDLDML

SEQ ID NO: 87 - protein, 79 aa, synthetic construct
LDDCLPMVDHIEGCLLDLLSDVGQELPDLGDLGGSDALDDFDLDMLDALDDFDLDMLDALDDFDLDMLDALDDFDLDML

SEQ ID NO: 88 - protein, 88 aa, synthetic construct
LDDCLPMVDHIEGCLLDLLSDVGQELPDLGDLGGSDALDDFDLDMLGGSDALDDFDLDMLGGSDALDDFDLDMLGGSDALDDFDLDML

SEQ ID NO: 89 - protein, 91 aa, synthetic construct
LDDCLPMVDHIEGCLLDLLSDVGQELPDLGDLGGSDALDDFDLDMLGSGSDALDDFDLDMLGSGSDALDDFDLDMLGSGSDALDDFDLDML

SEQ ID NO: 90 - protein, 94 aa, synthetic construct
LDDCLPMVDHIEGCLLDLLSDVGQELPDLGDLGGSDALDDFDLDMLGGSGSDALDDFDLDMLGGSGSDALDDFDLDMLGGSGSDALDDFDLDML

SEQ ID NO: 91 - protein, 97 aa, synthetic construct
LDDCLPMVDHIEGCLLDLLSDVGQELPDLGDLGGSDALDDFDLDMLGGSGGSDALDDFDLDMLGGSGGSDALDDFDLDMLGGSGGSDALDDFDLDML

SEQ ID NO: 92 - protein, 69 aa, synthetic construct
LDDCLPMVDHIEGCLLDLLSDVGQELPDLGDLGGSDDFDLDLGSDDFDLDLGSDDFDLDLGSDDFDLDL

SEQ ID NO: 93 - protein, 63 aa, synthetic construct
LDDCLPMVDHIEGCLLDLLSDVGQELPDLGDLGGSDDFDLDLDDFDLDLDDFDLDLDDFDLDL

SEQ ID NO: 94 - protein, 70 aa, synthetic construct
LDDCLPMVDHIEGCLLDLLSDVGQELPDLGDLDLDLDDFDLDLGSDDFDLDLGSDDFDLDLGSDDFDLDL

SEQ ID NO: 95 - protein, 64 aa, synthetic construct
LDDCLPMVDHIEGCLLDLLSDVGQELPDLGDLDLDLDDFDLDLDDFDLDLDDFDLDLDDFDLDL

SEQ ID NO: 96 - protein, 85 aa, synthetic construct
LDDLLPMVDHIEGCLLDLLSDVGQELPDLGDLGGSDALDDFDLDMLGSDALDDFDLDMLGSDALDDFDLDMLGSDALDDFDLDML

SEQ ID NO: 97 - protein, 85 aa, synthetic construct
LDDCDPMVDHIEGCLLDLLSDVGQELPDLGDLGGSDALDDFDLDMLGSDALDDFDLDMLGSDALDDFDLDMLGSDALDDFDLDML

SEQ ID NO: 98 - protein, 85 aa, synthetic construct
LDDCLPDVDHIEGCLLDLLSDVGQELPDLGDLGGSDALDDFDLDMLGSDALDDFDLDMLGSDALDDFDLDMLGSDALDDFDLDML

SEQ ID NO: 99 - protein, 85 aa, synthetic construct
LDDCLPMVDHIEGCLLDLDSDVGQELPDLGDLGGSDALDDFDLDMLGSDALDDFDLDMLGSDALDDFDLDMLGSDALDDFDLDML

SEQ ID NO: 100 - protein, 85 aa, synthetic construct
LDDCLPMVDHIEGCLLDLLSDVGQLLPDLGDLGGSDALDDFDLDMLGSDALDDFDLDMLGSDALDDFDLDMLGSDALDDFDLDML

SEQ ID NO: 101 - protein, 85 aa, synthetic construct
LDDCLPMVDHIEGCLLDLLSDVGQEDPDLGDLGGSDALDDFDLDMLGSDALDDFDLDMLGSDALDDFDLDMLGSDALDDFDLDML

SEQ ID NO: 102 - protein, 85 aa, synthetic construct
LDDLDPDVDHIEGCLLDLDSDVGQLDPDLGDLGGSDALDDFDLDMLGSDALDDFDLDMLGSDALDDFDLDMLGSDALDDFDLDML

SEQ ID NO: 103 - protein, 63 aa, synthetic construct
LDDLDPDVDHIEGCLLDLDSDVGQLDPDLGDLDLDLDDFDLDLDDFDLDLDDFDLDLDDFDLD

SEQ ID NO: 104 - protein, 70 aa, synthetic construct
LDDCLPMVDHIEGCLLDLLSDVGQELPDLGDLGGSGGSLDDCLPMVDHIEGCLLDLLSDVGQELPDLGDL

SEQ ID NO: 105 - protein, 82 aa, synthetic construct
GSAGSAAGSGEFLDDCLPMVDHIEGCLLDLLSDVGQELPDLGDLGGSGGSLDDCLPMVDHIEGCLLDLLSDVGQELPDLGDL

SEQ ID NO: 106 - protein, 76 aa, synthetic construct
GGSGGSLDDCLPMVDHIEGCLLDLLSDVGQELPDLGDLGGSGGSLDDCLPMVDHIEGCLLDLLSDVGQELPDLGDL

SEQ ID NO: 107 - protein, 70 aa, synthetic construct
LDDCLPMVDHIEGCLLDLLSDVGQELPDLGDLGGSGGSLDGLDPLEQGVDSLLDLLCGEIHDVMPLCDDL

SEQ ID NO: 108 - protein, 82 aa, synthetic construct
GSAGSAAGSGEFLDDCLPMVDHIEGCLLDLLSDVGQELPDLGDLGGSGGSLDGLDPLEQGVDSLLDLLCGEIHDVMPLCDDL

SEQ ID NO: 109 - protein, 76 aa, synthetic construct
GGSGGSLDDCLPMVDHIEGCLLDLLSDVGQELPDLGDLGGSGGSLDGLDPLEQGVDSLLDLLCGEIHDVMPLCDDL

SEQ ID NO: 110 - protein, 70 aa, synthetic construct
LDDLDPDVDHIEGCLLDLDSDVGQLDPDLGDLGGSGGSLDDLDPDVDHIEGCLLDLDSDVGQLDPDLGDL

SEQ ID NO: 111 - protein, 82 aa, synthetic construct
GSAGSAAGSGEFLDDLDPDVDHIEGCLLDLDSDVGQLDPDLGDLGGSGGSLDDLDPDVDHIEGCLLDLDSDVGQLDPDLGDL

SEQ ID NO: 112 - protein, 76 aa, synthetic construct
GGSGGSLDDLDPDVDHIEGCLLDLDSDVGQLDPDLGDLGGSGGSLDDLDPDVDHIEGCLLDLDSDVGQLDPDLGDL

SEQ ID NO: 113 - protein, 70 aa, synthetic construct
LDDLDPDVDHIEGCLLDLDSDVGQLDPDLGDLGGSGGSLDGLDPDLQGVDSDLDLLCGEIHDVDPDLDDL

SEQ ID NO: 114 - protein, 82 aa, synthetic construct
GSAGSAAGSGEFLDDLDPDVDHIEGCLLDLDSDVGQLDPDLGDLGGSGGSLDGLDPDLQGVDSDLDLLCGEIHDVDPDLDDL

SEQ ID NO: 115 - protein, 76 aa, synthetic construct
GGSGGSLDDLDPDVDHIEGCLLDLDSDVGQLDPDLGDLGGSGGSLDGLDPDLQGVDSDLDLLCGEIHDVDPDLDDL

SEQ ID NO: 116 - protein, 108 aa, synthetic construct
LDDCLPMVDHIEGCLLDLLSDVGQELPDLGDLGGSGGSLDGLDPLEQGVDSLLDLLCGEIHDVMPLCDDLGGSGGSLDDCLPMVDHIEGCLLDLLSDVGQELPDLGDL

SEQ ID NO: 117 - protein, 108 aa, synthetic construct
LDDLDPDVDHIEGCLLDLDSDVGQLDPDLGDLGGSGGSLDDLDPDVDHIEGCLLDLDSDVGQLDPDLGDLGGSGGSLDDLDPDVDHIEGCLLDLDSDVGQLDPDLGDL

SEQ ID NO: 118 - protein, 108 aa, synthetic construct
LDDLDPDVDHIEGCLLDLDSDVGQLDPDLGDLGGSGGSLDGLDPDLQGVDSDLDLLCGEIHDVDPDLDDLGGSGGSLDDLDPDVDHIEGCLLDLDSDVGQLDPDLGDL

SEQ ID NO: 119 - protein, 89 aa, synthetic construct
LDDCLPMVDHIEGCLLDLLSDVGQELPDLGDLGGSDALDDFDLDMLGSLMDLDFDDLADGGSGGSDALDDFDLDMLGSLMDLDFDDLAD

SEQ ID NO: 120 - protein, 89 aa, synthetic construct
LDDCLPMVDHIEGCLLDLLSDVGQELPDLGDLGGSDALDDFDLDMLGSDALDDFDLDMLGGSGGSLMDLDFDDLADGSLMDLDFDDLAD

SEQ ID NO: 121 - protein, 123 aa, synthetic construct
LDDCLPMVDHIEGCLLDLLSDVGQELPDLGDLGGSDALDDFDLDMLGSDALDDFDLDMLGGSGGSLMDLDFDDLADGSLMDLDFDDLADGSLDGLDPLEQGVDSLLDLLCGEIHDVMPLCDDL

SEQ ID NO: 122 - protein, 125 aa, synthetic construct
GGSDALDDFDLDMLGSLDDCLPMVDHIEGCLLDLLSDVGQELPDLGDLGSDALDDFDLDMLGGSGGSDALDDFDLDMLGSLDDCLPMVDHIEGCLLDLLSDVGQELPDLGDLGSDALDDFDLDML

SEQ ID NO: 123 - protein, 125 aa, synthetic construct
GGSDALDDFDLDMLGSLDDCLPMVDHIEGCLLDLLSDVGQELPDLGDLGSDALDDFDLDMLGGSGGSLMDLDFDDLADGSLDGLDPLEQGVDSLLDLLCGEIHDVMPLCDDLGSLMDLDFDDLAD

SEQ ID NO: 124 - protein, 107 aa, synthetic construct
LDDCLPMVDHIEGCLLDLLSDVGQELPDLGDLGGSLDDFDLDGSLDDFDLDGGSGGSDLDFDDLGSDLDFDDLGSLDGLDPLEQGVDSLLDLLCGEIHDVMPLCDDL

SEQ ID NO: 125 - protein, 109 aa, synthetic construct
GGSLDDFDLDGSLDDCLPMVDHIEGCLLDLLSDVGQELPDLGDLGSLDDFDLDGGSGGSLDDFDLDGSLDDCLPMVDHIEGCLLDLLSDVGQELPDLGDLGSLDDFDLD

SEQ ID NO: 126 - protein, 109 aa, synthetic construct
GGSLDDFDLDGSLDDCLPMVDHIEGCLLDLLSDVGQELPDLGDLGSLDDFDLDGGSGGSDLDFDDLGSLDGLDPLEQGVDSLLDLLCGEIHDVMPLCDDLGSDLDFDDL

SEQ ID NO: 127 - protein, 68 aa, synthetic construct
LDDFDLDLDDFDLDLDDFDLDLDDFDLDGSAGSAAGSGEFLDDFDLDLDDFDLDLDDFDLDLDDFDLD

SEQ ID NO: 128 - protein, 80 aa, synthetic construct
GSAGSAAGSGEFLDDFDLDLDDFDLDLDDFDLDLDDFDLDGSAGSAAGSGEFLDDFDLDLDDFDLDLDDFDLDLDDFDLD

SEQ ID NO: 129 - protein, 74 aa, synthetic construct
GGSGGSLDDFDLDLDDFDLDLDDFDLDLDDFDLDGSAGSAAGSGEFLDDFDLDLDDFDLDLDDFDLDLDDFDLD

SEQ ID NO: 130 - protein, 68 aa, synthetic construct
LDDFDLDLDDFDLDLDDFDLDLDDFDLDGSAGSAAGSGEFDLDFDDLDLDFDDLDLDFDDLDLDFDDL

SEQ ID NO: 131 - protein, 80 aa, synthetic construct
GSAGSAAGSGEFLDDFDLDLDDFDLDLDDFDLDLDDFDLDGSAGSAAGSGEFDLDFDDLDLDFDDLDLDFDDLDLDFDDL

SEQ ID NO: 132 - protein, 74 aa, synthetic construct
GGSGGSLDDFDLDLDDFDLDLDDFDLDLDDFDLDGSAGSAAGSGEFDLDFDDLDLDFDDLDLDFDDLDLDFDDL

SEQ ID NO: 133 - protein, 132 aa, synthetic construct
LDDLDPDVDHIEGCLLDLDSDVGQLDPDLGDLDLDLDDFDLDLDDFDLDLDDFDLDLDDFDLDGGSGGSLDDLDPDVDHIEGCLLDLDSDVGQLDPDLGDLDLDLDDFDLDLDDFDLDLDDFDLDLDDFDLD

SEQ ID NO: 134 - protein, 144 aa, synthetic construct
GSAGSAAGSGEFLDDLDPDVDHIEGCLLDLDSDVGQLDPDLGDLDLDLDDFDLDLDDFDLDLDDFDLDLDDFDLDGGSGGSLDDLDPDVDHIEGCLLDLDSDVGQLDPDLGDLDLDLDDFDLDLDDFDLDLDDFDLDLDDFDLD

SEQ ID NO: 135 - protein, 138 aa, synthetic construct
GGSGGSLDDLDPDVDHIEGCLLDLDSDVGQLDPDLGDLDLDLDDFDLDLDDFDLDLDDFDLDLDDFDLDGGSGGSLDDLDPDVDHIEGCLLDLDSDVGQLDPDLGDLDLDLDDFDLDLDDFDLDLDDFDLDLDDFDLD

SEQ ID NO: 136 - protein, 133 aa, synthetic construct
LDDLDPDVDHIEGCLLDLDSDVGQLDPDLGDLDLDLDDFDLDLDDFDLDLDDFDLDLDDFDLDGGSGGSDLDFDDLDLDFDDLDLDFDDLDLDFDDLLDLDLDGLDPDLQGVDSDLDLLCGEIHDVDPDLDDL

SEQ ID NO: 137 - protein, 145 aa, synthetic construct
GSAGSAAGSGEFLDDLDPDVDHIEGCLLDLDSDVGQLDPDLGDLDLDLDDFDLDLDDFDLDLDDFDLDLDDFDLDGGSGGSDLDFDDLDLDFDDLDLDFDDLDLDFDDLLDLDLDGLDPDLQGVDSDLDLLCGEIHDVDPDLDDL

SEQ ID NO: 138 - protein, 139 aa, synthetic construct
GGSGGSLDDLDPDVDHIEGCLLDLDSDVGQLDPDLGDLDLDLDDFDLDLDDFDLDLDDFDLDLDDFDLDGGSGGSDLDFDDLDLDFDDLDLDFDDLDLDFDDLLDLDLDGLDPDLQGVDSDLDLLCGEIHDVDPDLDDL